(19) United States
(12) Patent Application Publication
(10) Pub. No.: US 2022/0403048 A1
(43) Pub. Date: Dec. 22, 2022

(54) BINDING MOLECULES TO ARGINASE II [ARG2]

(71) Applicants: Cancer Research Technology Limited, Greater London (GB); Medimmune Limited, Cambridge (GB)

(72) Inventors: Alexandra Addyman, London (GB); Mark Austin, Cambridge (GB); Michelle Barnard, Greater London (GB); Vincenzo Cerundolo, Oxford (GB); Denice Chan, Greater London (GB); Agata Diamandakis, Greater London (GB); Sebastian Fielder, Cambridge (GB); Maria Groves, Cambridge (GB); Stuart Haynes, Greater London (GB); Sarah Holt, Greater London (GB); Lesley Jenkinson, Greater London (GB); Stephanie Keswick, Greater London (GB); Fiona McLaughlin, Amersham (GB); Pooja Sharma, Greater London (GB); Yoko Shibata, Greater London (GB); Louise Slater, Greater London (GB); Jessica Whitehouse, Greater London (GB); Mark D Carr, Leicester (GB); Daniel Burschowsky, Leicester (GB); Chitra Seewooruthun, Leicester (GB)

(73) Assignees: Cancer Research Technology Limited, Greater London (GB); Medimmune Limited, Cambridge (GB)

(21) Appl. No.: 17/637,052

(22) PCT Filed: Aug. 21, 2020
(86) PCT No.: PCT/EP2020/073579

§ 371 Date: Feb. 21, 2022

(30) Foreign Application Priority Data
Aug. 21, 2019 (GB) 19120302

(51) Int. Cl. C07K 16/40; C12N 9/78; A61P 43/00

(52) U.S. Cl. CPC C07K 16/40; C12N 9/78; A61P 43/00; C12Y 305/03001; A61K 2039/505

(57) ABSTRACT

An isolated antigen-binding protein characterised in that it is capable of binding specifically to human Arginase II (ARG2) and inhibiting the enzyme activity of human ARG2.

### SEQUENCE LISTING

The instant application contains a Sequence Listing which has been submitted electronically in ASCII format via EFS-Web and hereby incorporated by reference in its entirety. Said ASCII copy, created on 15 Aug. 2022, is named CMAL001WO1_ST25_CORR.TXT and is 172,032 bytes in size.

### TECHNICAL FIELD

The invention relates to antigen-binding proteins, e.g., antibodies, which bind specifically to and inhibit human arginase II (ARG2). The invention also relates to such anti-ARG2 antibodies for use in treating conditions associated with arginase activation and upregulation, such as cancer, immune cell dysfunction, infection, vascular disease, cardiovascular disease, endothelial dysfunction, ageing and cellular senescence, CNS disease and injury; diabetes-associated disease or cystic fibrosis.

### BACKGROUND ART

Arginase (ARG) activation and upregulation has been implicated in many disease states, including cancer, immune cell dysfunction, infection, vascular disease, cardiovascular disease, endothelial dysfunction, ageing and cellular senescence, CNS disease and injury; diabetes associated disease and cystic fibrosis or infection associated with cystic fibrosis (Munder, 2009; Caldwell et al., 2015; Caldwell et al., 2018). Arginase is a binuclear manganese metalloenzyme that catalyses the hydrolysis of L-arginine to L-ornithine and urea. In mammals, two ARG isoenzymes (Arginase I and II) have been identified. They catalyse the same biochemical reaction, but differ in cellular expression, regulation and subcellular localization (Jenkinson et al., 1996).

Arginase II (ARG2) is expressed as a mitochondrial protein in a variety of peripheral mammalian tissues, most prominently in kidney, prostate, small intestine and the lactating mammary gland. ARG2 is inducible in many other cell types including endothelial cells and macrophages (Ming et al., 2012; Yepuri et al., 2012). The primary role for ARG2 appears to be in L-arginine homeostasis (Morris, 2007; Durante et al., 2007) where it plays a crucial role in regulating the availability of L-arginine (or L-ornithine) for subsequent biosynthetic transformation(s). L-ornithine can be further metabolized to a variety of metabolically-important products including polyamines, such as putrescine, spermidine, and spermine, that participate in a variety of cellular functions e.g., proliferation, cell membrane transport. L-arginine also serves as a substrate for nitric oxide synthase (NOS) leading to nitric oxide (NO) and other reactive nitrogen intermediates (e.g., peroxynitrite). As such, ARG2 activity, together with the recycling of L-arginine from L-citrulline via argininosuccinate synthase (ASS) and argininosuccinate lyase (ASL) play a crucial role in both L-arginine and NO availability with important therapeutic implications including the following examples: extracellular L-arginine depletion by arginase can result in the impairment of lymphocyte responses to antigen during immune response (Bronte and Zanavello, 2005); depletion of NO can lead to increased risk of infection and endothelial dysfunction (Lewis et al., 2011; Sankaralingham et al., 2010; Xu et al.; 2004; Bivalacqua et al., 2001)

The isoform arginase I (ARG1) is expressed in the liver as one of the enzymes of the urea cycle, which forms the major route for the detoxification of ammonia in mammals. The cycle is distributed over two cellular compartments (mitochondrion/cytosol) with arginase acting as a cytosolic protein (Jenkinson et al., 1996). Non-malignant immature myeloid cells, called myeloid-derived suppressor cells (MDSCs) have been described in cancer, infectious disease and inflammation, and have been shown to be capable of suppressing T cell proliferation through the upregulated expression and production of immune suppressive factors such as arginase I (Monu et al., 2012) and inducible nitric oxide synthase (iNOS; also known as NOS2) (Jayaraman et al., 2012).

From the available literature, the emerging picture is that ARG1 is mainly expressed in tumour-infiltrating myeloid cells, whereas ARG2 is detected primarily in cancerous cells. Although many studies lack immunohistochemical evidence, the frequent finding of ARG in cancer suggests an important role for this enzyme in tumour biology and development. ARG-dependent tumour-promoting actions can range from proangiogenic activity, lymphocyte suppression, assistance in tumour cell proliferation, to stroma remodelling, all properties that have been assigned to tumour-associated macrophages with an alternative activation profile (Mantovani et al., 2002; Balkwill et al., 2005). Several examples, both in preclinical tumour models and in clinical studies, emphasize this multifaceted ARG activity (Bronte and Zanovello, 2005).

In acute myeloid leukaemia (AML), it has been shown that patient-derived AML blasts suppress T-cell proliferation, an effect mediated by the secretion of ARG2 (Mussai et al., 2015). AML blasts also demonstrate an arginase-dependent ability to polarize surrounding monocytes into a suppressive M2-like phenotype and to suppress the proliferation and differentiation of hematopoietic progenitor cells. The study also showed that the described immunosuppressive activity of AML blasts can be modulated by small-molecule inhibitors of ARG2 and iNOS. Together, these results support the hypothesis that the secretion of ARG2 by AML blasts plays a key role in the creation of an immunosuppressive microenvironment both in the bone marrow and in blood and in causing the pancytopenia often observed in patients with AML. The identification of the ability of AML blasts to induce an immune suppressive phenotype mechanistically positions AML blasts such that their role is reminiscent of MDSCs implicated in a number of human diseases including cancer.

The identification by Mussai et al. (ibid.) that AML blasts express ARG2 (an arginase isoform of arginase that has been less well characterized in the context of immunosuppression) represents a novel mechanism through which malignancies can deplete arginine from the microenvironment. They showed that ARG2 derived from AML blasts is enzymatically active, converting arginine into urea, and can suppress T cell proliferation. Notably, ARG2 (but not ARG1) was released from AML blasts and this resulted in presence of significant concentrations of ARG2 in the plasma of AML patients. As a result, both plasma from AML patients and supernatant of cultured AML blasts showed enhanced ARG activity resulting in:
i) suppression of T-cell proliferation,
ii) polarisation of surrounding monocytes into a suppressive M2-like phenotype in vitro and in engrafted NOD-SCID mice, and
iii) suppression of proliferation (leading to quiescence) of haematopoietic precursor cells (human CD34+ progenitors and murine GMP progenitors) in vitro (pancytopenia).

This study provided strong preclinical evidence for how AML blasts avoid immune detection and prompted further work to understand how the immunosuppressive environment in cancer patients can be overcome to improve overall survival.

The clinical development of ARG inhibitors is relevant across many disease indications. There is accumulated data on the role of ARG in tumour-associated MDSC and its pathogenetic role in inflammation-induced immunosuppression as described above. ARG2 specifically has been implicated in several cancers including osteosarcoma (Setty et al., 2016), HCMV-driven GBM (Costa et al., 2016), pancreatic cancer (Ino et al., 2013), head and neck squamous cell carcinoma (Bron et al., 2013), thyroid (Sousa et al., 2010), prostate (Mumenthaler et al., 2008), neuroblastoma (Mussai et al., 2015) and breast cancer (Polat et al., 2002).

Several specific ARG small molecule inhibitors have already been developed and tested in vitro. The intermediate of NO-synthesis, NG-hydroxy-L-arginine (NOHA) is a well-known arginase inhibitor (Hecker et al., 1995). It was successfully employed in human prostate carcinoma organ cultures, inhibited ARG activity and restored reactivity of tumour infiltrating lymphocytes in cooperation with a NOS inhibitor (Bronte et al., 2005). The L-arginine derivative Nw-hydroxynor-L-arginine (nor-NOHA) was, for example, able to completely reverse PMN-mediated T cell suppression in purulent inflammation (Munder et al., 2006) and to restore airway responsiveness in an arginase-mediated asthma animal model (Maarsingh et al., 2006). The boronic acids 2(S)-amino-6-boronohexanoic acid (ABH) and S-(2-boronoethyl)-L-cysteine (BEC) are potent inhibitors of both ARG isoforms at physiologic pH (with binding constant (K_{D}) of approximately 0.3 μM), binding with much higher affinity than the natural substrate (L-arginine Kₘ is 5 mM) (Ash et al., 2004; Christianson et al., 2005). Human ARG1 was crystallized in association with both inhibitors and new insights into the catalytic mechanism have been gained (Di Costanzo et al., 2005).

However, limitations remain in arginase-based therapies, due to concerns that existing small molecule inhibitors of ARG (NOHA and L-NMMA) may inhibit both ARG1 and ARG2 and consequently may inhibit the urea cycle in healthy cells, leading to hyperammonemia.

WO2018236828 discloses that ARG2 expression and activity is a strong effector of Treg immunosuppressive activity; impaired ARG2 is associated with aberrant autoimmune processes, while strong ARG2 activity is associated with detrimental suppression of immune response to tumours. It is proposed that modulation of Treg suppressive activity may be used to treat disease states mediated by Treg dysfunction and by Treg suppressive activity.

WO2018236828 discusses inhibiting suppressive Treg activity by inhibition or ablation of ARG2. The use of an ARG2 inhibitor for the treatment of cancer is disclosed; potential ARG2 inhibitors are listed as CB 1158, 2(S)-amino-6-boronohexanoic Acid (ABH), (2S)-5,29,59-trihydroxy-7,8-dimethoxy flavanone, R)-2-amino-6-borono-2-(2-(piperidin-1-yl)ethyl)hexanoic acid], and piceatannol-3′-O-β-d-glucopyranoside, an antibody, a composition of matter which facilitates the selective ubiquitination and proteolytic degradation of ARG2 or reduces ARG2 gene expression, and a CRISPR/Cas 9 construct for the selective inhibition of the ARG2 gene. WO2018236828 discloses a preference that the ARG2 inhibitor is selective for ARG2 and does not substantially inhibit arginase 1 activity, it is said that the ARG2 inhibitor which selectively binds and inactivates ARG2 can be a composition of matter which selectively binds and inactivates ARG2, an antibody, an intrabody, a molecule which facilitates the selective ubiquitination and proteolytic degradation of ARG2, a composition of matter which reduces ARG2 gene expression, an inhibitor of ARG2 gene expression, e.g., comprising a short interfering RNA, a hairpin RNA, a zinc finger nuclease, a transcription activator-like effector nuclease, or a CRISPR/Cas 9 construct. The only exemplification of ARG2 inhibition is depletion of ARG2 in primary human Tregs using Crispr-Cas9 ribonucleoprotein (RNP) technology. WO2018236828 does not exemplify an antibody that inhibits the activity of ARG2.

Antibodies to ARG2 are known in the art, while these bind to ARG2, hitherto no antibodies that inhibit the activity of ARG2 have been identified.

Accordingly, there is a need for an approach to target and to inhibit ARG2 specifically.

### SUMMARY OF INVENTION

The use of an antigen-binding protein, such as an antibody, e.g., a monoclonal antibody, to target ARG2 has several advantages over a small molecule approach, because it may enable both inhibition and depletion of ARG2 (via Fc mediated clearance of the antibody-antigen (Ab-Ag) complex). An antibody that specifically targets extracellular ARG2 may alleviate concerns about toxicity and reduce the risk of unwanted adverse effects in patients resulting from inhibition of intracellular arginase. An antibody may also distinguish between the two arginase isomers enabling selectivity for ARG2 over ARG1 and thus specific inhibition of ARG2 in a way that a small molecule would not be able to achieve due to the high degree of similarity between the active sites of ARG1 and ARG2. Finally, an antibody therapeutic may have better pharmacological properties than small molecule inhibitors including increased bioavailability and sustained action in the bloodstream of patients, e.g., AML patients.

The invention provides:

1. An isolated antigen-binding protein characterised in that it is capable of binding specifically to human Arginase II (ARG2) and inhibiting the enzyme activity of human ARG2.

2. An antigen-binding protein according to clause 1, wherein the antigen-binding protein is capable of binding specifically to and inhibiting monomeric and/or trimeric human ARG2.

3. An antigen-binding protein according to any one of the preceding clauses, wherein the antigen-binding protein binds trimeric human ARG2 with a dissociation constant (K_{D}) of less than 10 nM, less than 1 nM, less than 500 pM, less than 300 pM, or less than 150 pM, when assessed by Bio-Layer Interferometry (BLI).

5. An antigen-binding protein according to any of the preceding clauses, wherein the antigen-binding protein is selective for binding and inhibiting human ARG2 over human ARG1.

6. An antigen-binding protein according to clause 5, wherein selectivity for binding human ARG2 over human ARG1 is assessed by Bio-Layer Interferometry (BLI).

7. An antigen-binding protein according to any one of the preceding clauses, wherein the antigen-binding protein does not measurably bind human ARG1 when assessed by Bio-Layer Interferometry (BLI).

8. An antigen-binding protein according to any one of the preceding clauses, wherein the antigen-binding protein binds human ARG2 with a 1:1 or 3:1 stoichiometry (antigen binding protein:human ARG2).

9. An antigen-binding protein according to any of the preceding clauses, wherein the antigen-binding protein is capable of binding specifically to and inhibiting the enzyme activity of cynomolgus ARG2.

10. An antigen-binding protein according to any of the preceding clauses, wherein the antigen-binding protein inhibits ARG2 by a mode of action that is not competitive.

11. An antigen-binding protein according to any of the preceding clauses, wherein the antigen-binding protein restores T-cell proliferation in vitro in the presence of ARG2.

12. An antigen-binding protein according to any of the preceding clauses, comprising:

(a) a VH domain comprising a set of HCDRs: HCDR1, HCDR2 and HCDR3, interspersed with framework (FW) regions (HFW1-HCDR1-HFW2-HCDR2-HFW3-HCDR3-HFW4), wherein the amino acid sequence of HCDR3 (amino acids 95-102) is LRADLGLYMDL (SEQ ID NO: 315) and optionally further comprising

(b) a VL domain comprising a set of LCDRs: LCDR1, LCDR2 and LCDR3, interspersed with framework (FW) regions (LFW1-LCDR1-LFW2-LCDR2-LFW3-LCDR3-LFW4), wherein the amino acid sequence of LCDR1 (amino acids 24-34) is SGSSSNIGNHYVS (SEQ ID NO: 318), wherein the sequences are defined by Kabat nomenclature.

13. An antigen-binding protein according to any of the preceding clauses, comprising:

(a) a VH domain comprising a set of HCDRs: HCDR1, HCDR2 and HCDR3, interspersed with framework regions (HFW1-HCDR1-HFW2-HCDR2-HFW3-HCDR3-HFW4), wherein the set of HCDRs is selected from those of antibody C0021158fgl2 (SEQ ID NO: 313, 314 and 315), C0021181 (SEQ ID NO: 343, 344, and 345), C0021180 (SEQ ID NO: 333, 334 and 335), C0021177 (SEQ ID NO: 323, 324 and 325), C0021158 (SEQ ID NO: 273, 274 and 275), C0021158 IgG (SEQ ID NO: 283, 284 and 285), C0021158fgl (SEQ ID NO: 303, 304 and 305), C0021158dr (SEQ ID NO: 293, 294 and 295), C0021061 (SEQ ID NO: 63, 64 and 65), C0020187 (SEQ ID NO: 13, 14 and 15), C0021155 (SEQ ID NO: 263, 264 and 265), C0021144 (SEQ ID NO: 253, 254 and 255), C0021142 (SEQ ID NO: 233, 234 and 235), C0021142 IgG (SEQ ID NO: 243, 244 and 245), C0021141 (SEQ ID NO: 223, 224 and 225), C0021139 (SEQ ID NO: 213, 214 and 215), C0021135 (SEQ ID NO: 203, 204 and 205), C0021133 (SEQ ID NO: 193, 194 and 195), C0021131 (SEQ ID NO: 183, 184 and 185), C0021129 (SEQ ID NO: 173, 174 and 175), C0021128 (SEQ ID NO: 163, 164 and 165), C0021124 (SEQ ID NO: 153, 154 and 155), C0021118 (SEQ ID NO: 143, 144 and 145), C0021101 (SEQ ID NO: 133, 134 and 135), C0021098 (SEQ ID NO: 123, 124 and 125), C0021097 (SEQ ID NO: 113, 114 and 115), C0021096 (SEQ ID NO: 103, 104 and 105), C0021092 (SEQ ID NO: 93, 94 and 95), C0021089 (SEQ ID NO: 83, 84 and 85), C0021065 (SEQ ID NO: 73, 74 and 75), C0021032 (SEQ ID NO: 53, 54 and 55), C0021022 (SEQ ID NO: 43, 44 and 45), C0021021 (SEQ ID NO: 33, 34 and 35), C0021017 (SEQ ID NO: 23, 24 and 25) and C0020065 (SEQ ID NO: 3, 4 and 5); and/or

(b) a VL domain comprising a set of LCDRs: LCDR1, LCDR2 and LCDR3, interspersed with framework regions (LFW1-LCDR1-LFW2-LCDR2-LFW3-LCDR3-LFW4), wherein the set of LCDRs is selected from those of antibody C0021158fgl2 (SEQ ID NO: 318, 319 and 320), C0021181 (SEQ ID NO: 348, 349, and 350), C0021180 (SEQ ID NO: 338, 339 and 340), C0021177 (SEQ ID NO: 328, 329 and 330), C0021158 (SEQ ID NO: 278, 279 and 280), C0021158 IgG (SEQ ID NO: 288, 289 and 290), C0021158fgl (SEQ ID NO: 308, 309 and 310), C0021158dr (SEQ ID NO: 298, 299 and 300), C0021061 (SEQ ID NO: 68, 69 and 70), C0020187 (SEQ ID NO: 18, 19 and 20), C0021155 (SEQ ID NO: 268, 269 and 270), C0021144 (SEQ ID NO: 258, 259 and 260), C0021142 (SEQ ID NO: 238, 239 and 240), C0021142 IgG (SEQ ID NO: 248, 249 and 250), C0021141 (SEQ ID NO: 228, 229 and 230), C0021139 (SEQ ID NO: 218, 219 and 220), C0021135 (SEQ ID NO: 208, 209 and 210), C0021133 (SEQ ID NO: 198, 199 and 200), C0021131 (SEQ ID NO: 188, 189 and 190), C0021129 (SEQ ID NO: 178, 179 and 180), C0021128 (SEQ ID NO: 168, 169 and 170), C0021124 (SEQ ID NO: 158, 159 and 160), C0021118 (SEQ ID NO: 148, 149 and 150), C0021101 (SEQ ID NO: 138, 139 and 140), C0021098 (SEQ ID NO: 128, 129 and 130), C0021097 (SEQ ID NO: 118, 119 and 120), C0021096 (SEQ ID NO: 108, 109 and 110), C0021092 (SEQ ID NO: 98, 99 and 100), C0021089 (SEQ ID NO: 88, 89 and 90), C0021065 (SEQ ID NO: 78, 79 and 80), C0021032 (SEQ ID NO: 58, 59 and 60), C0021022 (SEQ ID NO: 48, 49 and 50), C0021021 (SEQ ID NO: 38, 39 and 40), C0021017 SEQ ID NO: 28, 29 and 30) and C0020065 (SEQ ID NO: 8, 9 and 10); wherein the sequences are defined by Kabat nomenclature.

14. An antigen-binding protein according to any of the preceding clauses, comprising:

a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021158fgl2;

a VH domain comprising HCDR1 (SEQ ID NO: 343), HCDR2 (SEQ ID NO: 344) and HCDR3 (SEQ ID NO: 345) and a VL domain comprising LCDR1 (SEQ ID NO: 348), LCDR2, (SEQ ID NO: 349) and LCDR3 (SEQ ID NO: 350) of C0021181;

a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021180;

a VH domain comprising HCDR1 (SEQ ID NO: 323), HCDR2 (SEQ ID NO: 324) and HCDR3 (SEQ ID NO: 325) and a VL domain comprising LCDR1 (SEQ ID NO: 328), LCDR2, (SEQ ID NO: 329) and LCDR3 (SEQ ID NO: 330) of C0021177;

a VH domain comprising HCDR1 (SEQ ID NO: 273), HCDR2 (SEQ ID NO: 274) and HCDR3 (SEQ ID NO: 275) and a VL domain comprising LCDR1 (SEQ ID NO: 278), LCDR2, (SEQ ID NO: 279) and LCDR3 (SEQ ID NO: 280) of C0021158;

a VH domain comprising HCDR1 (SEQ ID NO: 283), HCDR2 (SEQ ID NO: 284) and HCDR3 (SEQ ID NO: 285) and a VL domain comprising LCDR1 (SEQ ID NO: 288), LCDR2, (SEQ ID NO: 289) and LCDR3 (SEQ ID NO: 290) of C0021158 IgG;

a VH domain comprising HCDR1 (SEQ ID NO: 303), HCDR2 (SEQ ID NO: 304) and HCDR3 (SEQ ID NO: 305) and a VL domain comprising LCDR1 (SEQ ID NO: 308), LCDR2, (SEQ ID NO: 309) and LCDR3 (SEQ ID NO: 310) of C0021158fgl;

a VH domain comprising HCDR1 (SEQ ID NO: 293), HCDR2 (SEQ ID NO: 294) and HCDR3 (SEQ ID NO: 295) and a VL domain comprising LCDR1 (SEQ ID NO: 298), LCDR2, (SEQ ID NO: 299) and LCDR3 (SEQ ID NO: 300) of C0021158dr;

a VH domain comprising HCDR1 (SEQ ID NO: 63), HCDR2 (SEQ ID NO: 64) and HCDR3 (SEQ ID NO: 65) and a VL domain comprising LCDR1 (SEQ ID NO: 68), LCDR2, (SEQ ID NO: 69) and LCDR3 (SEQ ID NO: 70) of C0021061;

a VH domain comprising HCDR1 (SEQ ID NO: 13), HCDR2 (SEQ ID NO: 14) and HCDR3 (SEQ ID NO: 15) and a VL domain comprising LCDR1 (SEQ ID NO: 18), LCDR2, (SEQ ID NO: 19) and LCDR3 (SEQ ID NO: 20) of C0020187;

a VH domain comprising HCDR1 (SEQ ID NO: 263), HCDR2 (SEQ ID NO: 264) and HCDR3 (SEQ ID NO: 265) and a VL domain comprising LCDR1 (SEQ ID NO: 268), LCDR2, (SEQ ID NO: 269) and LCDR3 (SEQ ID NO: 270) of C0021155;

a VH domain comprising HCDR1 (SEQ ID NO: 253), HCDR2 (SEQ ID NO: 254) and HCDR3 (SEQ ID NO: 255) and a VL domain comprising LCDR1 (SEQ ID NO: 258), LCDR2, (SEQ ID NO: 259) and LCDR3 (SEQ ID NO: 260) of C0021144;

a VH domain comprising HCDR1 (SEQ ID NO: 233), HCDR2 (SEQ ID NO: 234) and HCDR3 (SEQ ID NO: 235) and a VL domain comprising LCDR1 (SEQ ID NO: 238), LCDR2, (SEQ ID NO: 239) and LCDR3 (SEQ ID NO: 240) of C0021142;

a VH domain comprising HCDR1 (SEQ ID NO: 243), HCDR2 (SEQ ID NO: 244) and HCDR3 (SEQ ID NO: 245) and a VL domain comprising LCDR1 (SEQ ID NO: 248), LCDR2, (SEQ ID NO: 249) and LCDR3 (SEQ ID NO: 250) of C0021142 IgG;

a VH domain comprising HCDR1 (SEQ ID NO: 223), HCDR2 (SEQ ID NO: 224) and HCDR3 (SEQ ID NO: 225) and a VL domain comprising LCDR1 (SEQ ID NO: 228), LCDR2, (SEQ ID NO: 229) and LCDR3 (SEQ ID NO: 230) of C0021141;

a VH domain comprising HCDR1 (SEQ ID NO: 213), HCDR2 (SEQ ID NO: 214) and HCDR3 (SEQ ID NO: 215) and a VL domain comprising LCDR1 (SEQ ID NO: 218), LCDR2, (SEQ ID NO: 219) and LCDR3 (SEQ ID NO: 220) of C0021139;

a VH domain comprising HCDR1 (SEQ ID NO: 203), HCDR2 (SEQ ID NO: 204) and HCDR3 (SEQ ID NO: 205) and a VL domain comprising LCDR1 (SEQ ID NO: 208), LCDR2, (SEQ ID NO: 209) and LCDR3 (SEQ ID NO: 210) of C0021135;

a VH domain comprising HCDR1 (SEQ ID NO: 193), HCDR2 (SEQ ID NO: 194) and HCDR3 (SEQ ID NO: 195) and a VL domain comprising LCDR1 (SEQ ID NO: 198), LCDR2, (SEQ ID NO: 199) and LCDR3 (SEQ ID NO: 200) of C0021133;

a VH domain comprising HCDR1 (SEQ ID NO: 183), HCDR2 (SEQ ID NO: 184) and HCDR3 (SEQ ID NO: 185) and a VL domain comprising LCDR1 (SEQ ID NO: 188), LCDR2, (SEQ ID NO: 189) and LCDR3 (SEQ ID NO: 190) of C0021131;

a VH domain comprising HCDR1 (SEQ ID NO: 173), HCDR2 (SEQ ID NO: 174) and HCDR3 (SEQ ID NO: 175) and a VL domain comprising LCDR1 (SEQ ID NO: 178), LCDR2, (SEQ ID NO: 179) and LCDR3 (SEQ ID NO: 180) of C0021129;

a VH domain comprising HCDR1 (SEQ ID NO: 163), HCDR2 (SEQ ID NO: 164) and HCDR3 (SEQ ID NO: 165) and a VL domain comprising LCDR1 (SEQ ID NO: 168), LCDR2, (SEQ ID NO: 169) and LCDR3 (SEQ ID NO: 170) C0021128;

a VH domain comprising HCDR1 (SEQ ID NO: 153), HCDR2 (SEQ ID NO: 154) and HCDR3 (SEQ ID NO: 155) and a VL domain comprising LCDR1 (SEQ ID NO: 158), LCDR2, (SEQ ID NO: 159) and LCDR3 (SEQ ID NO: 160) of C0021124;

a VH domain comprising HCDR1 (SEQ ID NO: 143), HCDR2 (SEQ ID NO: 144) and HCDR3 (SEQ ID NO: 145) and a VL domain comprising LCDR1 (SEQ ID NO: 148), LCDR2, (SEQ ID NO: 149) and LCDR3 (SEQ ID NO: 150) of C0021118;

a VH domain comprising HCDR1 (SEQ ID NO: 133), HCDR2 (SEQ ID NO: 134) and HCDR3 (SEQ ID NO: 135) and a VL domain comprising LCDR1 (SEQ ID NO: 138), LCDR2, (SEQ ID NO: 139) and LCDR3 (SEQ ID NO: 140) of C0021101;

a VH domain comprising HCDR1 (SEQ ID NO: 123), HCDR2 (SEQ ID NO: 124) and HCDR3 (SEQ ID NO: 125) and a VL domain comprising LCDR1 (SEQ ID NO: 128), LCDR2, (SEQ ID NO: 129) and LCDR3 (SEQ ID NO: 130) of C0021098;

a VH domain comprising HCDR1 (SEQ ID NO: 113), HCDR2 (SEQ ID NO: 114) and HCDR3 (SEQ ID NO: 115) and a VL domain comprising LCDR1 (SEQ ID NO: 118), LCDR2, (SEQ ID NO: 119) and LCDR3 (SEQ ID NO: 120) of C0021097;

a VH domain comprising HCDR1 (SEQ ID NO: 103), HCDR2 (SEQ ID NO: 104) and HCDR3 (SEQ ID NO: 105) and a VL domain comprising LCDR1 (SEQ ID NO: 108), LCDR2, (SEQ ID NO: 109) and LCDR3 (SEQ ID NO: 110) of C0021096;

a VH domain comprising HCDR1 (SEQ ID NO: 93), HCDR2 (SEQ ID NO: 94) and HCDR3 (SEQ ID NO: 95) and a VL domain comprising LCDR1 (SEQ ID NO: 98), LCDR2, (SEQ ID NO: 99) and LCDR3 (SEQ ID NO: 100) of C0021092;

a VH domain comprising HCDR1 (SEQ ID NO: 83), HCDR2 (SEQ ID NO: 84) and HCDR3 (SEQ ID NO: 85) and a VL domain comprising LCDR1 (SEQ ID NO: 88), LCDR2, (SEQ ID NO: 89) and LCDR3 (SEQ ID NO: 90) of C0021089;

a VH domain comprising HCDR1 (SEQ ID NO: 73), HCDR2 (SEQ ID NO: 74) and HCDR3 (SEQ ID NO: 75) and a VL domain comprising LCDR1 (SEQ ID NO: 78), LCDR2, (SEQ ID NO: 79) and LCDR3 (SEQ ID NO: 80) of C0021065;

a VH domain comprising HCDR1 (SEQ ID NO: 53), HCDR2 (SEQ ID NO: 54) and HCDR3 (SEQ ID NO: 55) and a VL domain comprising LCDR1 (SEQ ID NO: 58), LCDR2, (SEQ ID NO: 59) and LCDR3 (SEQ ID NO: 60) of C0021032;

a VH domain comprising HCDR1 (SEQ ID NO: 43), HCDR2 (SEQ ID NO: 44) and HCDR3 (SEQ ID NO:45) and a VL domain comprising LCDR1 (SEQ ID NO: 48), LCDR2, (SEQ ID NO: 49) and LCDR3 (SEQ ID NO: 50) of C0021022;

a VH domain comprising HCDR1 (SEQ ID NO: 33), HCDR2 (SEQ ID NO: 34) and HCDR3 (SEQ ID NO: 35) and a VL domain comprising LCDR1 (SEQ ID NO: 38), LCDR2, (SEQ ID NO: 39) and LCDR3 (SEQ ID NO: 40) of C0021021;

a VH domain comprising HCDR1 (SEQ ID NO: 23), HCDR2 (SEQ ID NO: 24) and HCDR3 (SEQ ID NO: 25) and a VL domain comprising LCDR1 (SEQ ID NO: 28), LCDR2, (SEQ ID NO: 29) and LCDR3 (SEQ ID NO: 30) of C0021017; or a VH domain comprising HCDR1 (SEQ ID NO: 3), HCDR2 (SEQ ID NO: 4) and HCDR3 (SEQ ID NO: 5) and a VL domain comprising LCDR1 (SEQ ID NO: 8), LCDR2, (SEQ ID NO: 9) and LCDR3 (SEQ ID NO: 10) of C0020065; wherein the sequences are defined by Kabat nomenclature.

15. An antigen-binding protein according to any of the preceding clauses, comprising

(a) (i) a VH domain comprising the C0021158fgl2 set of HCDRs (HCDR1 SEQ ID NO: 313, HCDR2 SEQ ID NO: 314 and HCDR3 SEQ ID NO: 315), and/or, (ii) a VL domain comprising the C0021158fgl2 set of LCDRs (LCDR1 SEQ ID NO: 318, LCDR2 SEQ ID NO: 319 and LCDR3 SEQ ID NO: 320);

(b) (i) a VH domain comprising the C0021133 set of HCDRs (HCDR1 SEQ ID NO: 193, HCDR2 SEQ ID NO: 194 and HCDR3 SEQ ID NO: 195), and/or, (ii) a VL domain comprising the C0021133 set of LCDRs (LCDR1 SEQ ID NO: 198, LCDR2 SEQ ID NO: 199 and LCDR3 SEQ ID NO: 200); or

(c) (i) a VH domain comprising the C0020187 set of HCDRs (HCDR1 SEQ ID NO: 13, HCDR2 SEQ ID NO: 14 and HCDR3 SEQ ID NO: 15, and/or, (ii) a VL domain comprising the C0020187 set of LCDRs (LCDR1 SEQ ID NO: 18, LCDR2 SEQ ID NO: 19, and LCDR3 SEQ ID NO: 20);

(d) (i) a VH domain comprising the C0020065 set of HCDRs (HCDR1 SEQ ID NO: 3, HCDR2 SEQ ID NO: 4 and HCDR3 SEQ ID NO: 5, and/or, (ii) a VL domain comprising the C0020187 set of LCDRs (LCDR1 SEQ ID NO: 8, LCDR2 SEQ ID NO: 9, and LCDR3 SEQ ID NO: 10); wherein the sequences are defined by Kabat nomenclature.

16. An isolated antigen-binding protein according to any of the preceding clauses, comprising:

(a) a VH domain selected from a VH domain of antibody C0021158 fgl2 (SEQ ID NO: 312), C0021181 (SEQ ID NO: 342), C0021180 (SEQ ID NO: 332), C0021177 (SEQ ID NO: 322), C0021158 (SEQ ID NO: 272), C0021158 IgG (SEQ ID NO: 282), C0021158fgl (SEQ ID NO: 302), C0021158dr (SEQ ID NO: 292), C0021061 (SEQ ID NO: 62), C0020187 (SEQ ID NO: 12), C0021155 (SEQ ID NO: 262), C0021144 (SEQ ID NO: 252), C0021142 (SEQ ID NO: 232), C0021142 IgG (SEQ ID NO: 242), C0021141 (SEQ ID NO: 227), C0021139 (SEQ ID NO: 217), C0021135 (SEQ ID NO: 207), C0021133 (SEQ ID NO: 197), C0021131 (SEQ ID NO: 187), C0021129 (SEQ ID NO: 177), C0021128 (SEQ ID NO: 167), C0021124 (SEQ ID NO: 157), C0021118 (SEQ ID NO: 147), C0021101 (SEQ ID NO: 137), C0021098 (SEQ ID NO: 127), C0021097 (SEQ ID NO: 117), C0021096 (SEQ ID NO: 107), C0021092 (SEQ ID NO: 97), C0021089 (SEQ ID NO: 87), C0021065 (SEQ ID NO: 77), C0021032 (SEQ ID NO: 57), C0021022 (SEQ ID NO: 47), C0021021 (SEQ ID NO: 37), C0021017 (SEQ ID NO: 27) and C0020065 (SEQ ID NO: 7), or a germlined version thereof, or a VH domain with at least 80%, 81%, 82%, 83%, 84%, 85%, 86%, 87%, 88%, 89%, 90%, 91%, 92%, 93%, 94%, 95%, 96%, 97%, 98%, or 99% homology thereto; and/or

(b) a VL domain selected from a VL domain of antibody C0021158 fgl2 (SEQ ID NO: 317), C0021181 (SEQ ID NO: 347), C0021180 (SEQ ID NO: 337), C0021177 (SEQ ID NO: 327), C0021158 (SEQ ID NO: 277), C0021158 IgG (SEQ ID NO: 287), C0021158fgl (SEQ ID NO: 307), C0021158dr (SEQ ID NO: 297), C0021061 (SEQ ID NO: 67), C0020187 (SEQ ID NO: 17), C0021155 (SEQ ID NO: 267), C0021144 (SEQ ID NO: 257), C0021142 (SEQ ID NO: 237), C0021142 IgG (SEQ ID NO: 247), C0021141 (SEQ ID NO: 227), C0021139 (SEQ ID NO: 217), C0021135 (SEQ ID NO: 207), C0021133 (SEQ ID NO: 197), C0021131 (SEQ ID NO: 187), C0021129 (SEQ ID NO: 177), C0021128 (SEQ ID NO: 167), C0021124 (SEQ ID NO: 157), C0021118 (SEQ ID NO: 147), C0021101 (SEQ ID NO: 137), C0021098 (SEQ ID NO: 127), C0021097 (SEQ ID NO: 117), C0021096 (SEQ ID NO: 107), C0021092 (SEQ ID NO: 97), C0021089 (SEQ ID NO: 87), C0021065 (SEQ ID NO: 77), C0021032 (SEQ ID NO: 57), C0021022 (SEQ ID NO: 47), C0021021 (SEQ ID NO: 37), C0021017 (SEQ ID NO: 27) and C0020065 (SEQ ID NO: 7), or a germlined version thereof, or a VL domain with at least 80%, 81%, 82%, 83%, 84%, 85%, 86%, 87%, 88%, 89%, 90%, 91%, 92%, 93%, 94%, 95%, 96%, 97%, 98%, or 99% homology thereto; wherein the sequences are defined by Kabat nomenclature.

17. An antigen-binding protein according to any of the preceding clauses, comprising a VH domain and a VL domain at least 80%, 81%, 82%, 83%, 84%, 85%, 86%, 87%, 88%, 89%, 90%, 91%, 92%, 93%, 94%, 95%, 96%, 97%, 98%, or 99% identical with:

a VH domain (SEQ ID NO: 312) and a VL domain (SEQ ID NO: 317) of C0021158 fgl2,

a VH domain (SEQ ID NO: 342) and a VL domain (SEQ ID NO: 347) of C0021181,

a VH domain (SEQ ID NO: 332) and a VL domain (SEQ ID NO: 337) of C0021180,

a VH domain (SEQ ID NO: 322) and a VL domain (SEQ ID NO: 327) of C0021177,

a VH domain (SEQ ID NO: 272) and a VL domain (SEQ ID NO: 277) of C0021158,

a VH domain (SEQ ID NO: 282) and a VL domain (SEQ ID NO: 287) of C0021158 IgG,

a VH domain (SEQ ID NO: 302) and a VL domain (SEQ ID NO: 307) of C0021158fgl,

a VH domain (SEQ ID NO: 292) and a VL domain (SEQ ID NO: 297) of C0021158dr,

a VH domain (SEQ ID NO: 62) and a VL domain (SEQ ID NO: 67) of C0021061,

a VH domain (SEQ ID NO: 12) and a VL domain (SEQ ID NO: 17) of C0020187,

a VH domain (SEQ ID NO: 262) and a VL domain (SEQ ID NO: 267) of C0021155,

a VH domain (SEQ ID NO: 252) and a VL domain (SEQ ID NO: 257) of C0021144,

a VH domain (SEQ ID NO: 232) and a VL domain (SEQ ID NO: 237) of C0021142,

a VH domain (SEQ ID NO: 242) and a VL domain (SEQ ID NO: 247) of C0021142 IgG,

a VH domain (SEQ ID NO: 222) and a VL domain (SEQ ID NO: 227) of C0021141,

a VH domain (SEQ ID NO: 212) and a VL domain (SEQ ID NO: 217) of C0021139,

a VH domain (SEQ ID NO: 202) and a VL domain (SEQ ID NO: 207) of C0021135,

a VH domain (SEQ ID NO: 192) and a VL domain (SEQ ID NO: 197) of C0021133,

a VH domain (SEQ ID NO: 182) and a VL domain (SEQ ID NO: 187) of C0021131,

a VH domain (SEQ ID NO: 172) and a VL domain (SEQ ID NO: 177) of C0021129,

a VH domain (SEQ ID NO: 162) and a VL domain (SEQ ID NO: 167) of C0021128,

a VH domain (SEQ ID NO: 152) and a VL domain (SEQ ID NO: 157) of C0021124,

a VH domain (SEQ ID NO: 142) and a VL domain (SEQ ID NO: 147) of C0021118,

a VH domain (SEQ ID NO: 132) and a VL domain (SEQ ID NO: 137) of C0021101,

a VH domain (SEQ ID NO: 122) and a VL domain (SEQ ID NO: 127) of C0021098,

a VH domain (SEQ ID NO: 112) and a VL domain (SEQ ID NO: 117) of C0021097,

a VH domain (SEQ ID NO: 102) and a VL domain (SEQ ID NO: 107) of C0021096,

a VH domain (SEQ ID NO: 92) and a VL domain (SEQ ID NO: 97) of C0021092,

a VH domain (SEQ ID NO: 82) and a VL domain (SEQ ID NO: 87) of C0021089,

a VH domain (SEQ ID NO: 72) and a VL domain (SEQ ID NO: 77) of C0021065,

a VH domain (SEQ ID NO: 52) and a VL domain (SEQ ID NO: 57) of C0021032,

a VH domain (SEQ ID NO: 42) and a VL domain (SEQ ID NO: 47) of C0021022,

a VH domain (SEQ ID NO: 32) and a VL domain (SEQ ID NO: 37) of C0021021,

a VH domain (SEQ ID NO: 22) and a VL domain (SEQ ID NO: 27) of C0021017, or

a VH domain (SEQ ID NO: 2) and a VL domain (SEQ ID NO: 7) of C0020065; wherein the sequences are defined by Kabat nomenclature.

18. An antigen-binding protein according to any of the preceding clauses, comprising:

a VH domain (SEQ ID NO: 312) and a VL domain (SEQ ID NO: 317) of C0021158fgl2,

a VH domain (SEQ ID NO: 342) and a VL domain (SEQ ID NO: 347) of C0021181,

a VH domain (SEQ ID NO: 332) and a VL domain (SEQ ID NO: 337) of C0021180,

a VH domain (SEQ ID NO: 322) and a VL domain (SEQ ID NO: 327) of C0021177,

a VH domain (SEQ ID NO: 272) and a VL domain (SEQ ID NO: 277) of C0021158,

a VH domain (SEQ ID NO: 282) and a VL domain (SEQ ID NO: 287) of C0021158 IgG,

a VH domain (SEQ ID NO: 302) and a VL domain (SEQ ID NO: 307) of C0021158fgl,

a VH domain (SEQ ID NO: 292) and a VL domain (SEQ ID NO: 297) of C0021158dr,

a VH domain (SEQ ID NO: 62) and a VL domain (SEQ ID NO: 67) of C0021061,

a VH domain (SEQ ID NO: 12) and a VL domain (SEQ ID NO: 17) of C0020187,

a VH domain (SEQ ID NO: 262) and a VL domain (SEQ ID NO: 267) of C0021155,

a VH domain (SEQ ID NO: 252) and a VL domain (SEQ ID NO: 257) of C0021144,

a VH domain (SEQ ID NO: 232) and a VL domain (SEQ ID NO: 237) of C0021142,

a VH domain (SEQ ID NO: 242) and a VL domain (SEQ ID NO: 247) of C0021142 IgG,

a VH domain (SEQ ID NO: 222) and a VL domain (SEQ ID NO: 227) of C0021141,

a VH domain (SEQ ID NO: 212) and a VL domain (SEQ ID NO: 217) of C0021139,

a VH domain (SEQ ID NO: 202) and a VL domain (SEQ ID NO: 207) of C0021135,

a VH domain (SEQ ID NO: 192) and a VL domain (SEQ ID NO: 197) of C0021133,

a VH domain (SEQ ID NO: 182) and a VL domain (SEQ ID NO: 187) of C0021131,

a VH domain (SEQ ID NO: 172) and a VL domain (SEQ ID NO: 177) of C0021129,

a VH domain (SEQ ID NO: 162) and a VL domain (SEQ ID NO: 167) of C0021128,

a VH domain (SEQ ID NO: 152) and a VL domain (SEQ ID NO: 157) of C0021124,

a VH domain (SEQ ID NO: 142) and a VL domain (SEQ ID NO: 147) of C0021118,

a VH domain (SEQ ID NO: 132) and a VL domain (SEQ ID NO: 137) of C0021101,

a VH domain (SEQ ID NO: 122) and a VL domain (SEQ ID NO: 127) of C0021098,

a VH domain (SEQ ID NO: 112) and a VL domain (SEQ ID NO: 117) of C0021097,

a VH domain (SEQ ID NO: 102) and a VL domain (SEQ ID NO: 107) of C0021096,

a VH domain (SEQ ID NO: 92) and a VL domain (SEQ ID NO: 97) of C0021092,

a VH domain (SEQ ID NO: 82) and a VL domain (SEQ ID NO: 87) of C0021089,

a VH domain (SEQ ID NO: 72) and a VL domain (SEQ ID NO: 77) of C0021065,

a VH domain (SEQ ID NO: 52) and a VL domain (SEQ ID NO: 57) of C0021032,

a VH domain (SEQ ID NO: 42) and a VL domain (SEQ ID NO: 47) of C0021022,

a VH domain (SEQ ID NO: 32) and a VL domain (SEQ ID NO: 37) of C0021021,

a VH domain (SEQ ID NO: 22) and a VL domain (SEQ ID NO: 27) of C0021017, or

a VH domain (SEQ ID NO: 2) and a VL domain (SEQ ID NO: 7) of C0020065; or a germlined version thereof, wherein the sequences are defined by Kabat nomenclature.

19. An antigen-binding protein according to any of the preceding clauses, comprising

(a) the C0021158fgl2 VH domain amino acid sequence SEQ ID NO: 312 and the C0021158fgl2 VL domain amino acid sequence SEQ ID NO: 317,

(b) the C0021133 VH domain amino acid sequence SEQ ID NO: 192 and the C0021133 VL domain amino acid sequence SEQ ID NO: 197,

(c) the C0020187 VH domain amino acid sequence SEQ ID NO: 12 and the C0020187 VL domain amino acid sequence SEQ ID NO: 17,

(d) the C0020065 VH domain amino acid sequence SEQ ID NO: 2 and the C0020065 VL domain amino acid sequence SEQ ID NO: 7.

20. An antigen-binding protein that competes for binding to human ARG2 with an antigen binding protein comprising:

(a) the VH domain of SEQ ID NO: 312 and a VL domain of SEQ ID NO: 317,

(b) the VH domain of SEQ ID NO: 192 and the VL domain of SEQ ID NO: 197,

(c) the VH domain of SEQ ID NO: 12 and VL domain of SEQ ID NO: 17, or

(d) the VH domain of SEQ ID NO: 2 and VL domain of SEQ ID NO: 7, when assessed in an epitope competition assay.

21. An antigen-binding protein according to any preceding clause, characterised in that it is capable of binding specifically to an epitope of human ARG2 and thereby inhibiting the enzyme activity of human ARG2 by an allosteric mechanism.

22. An isolated antigen-binding protein according to any preceding clause, characterised in that it is capable of binding specifically to an epitope of human ARG2 and thereby inducing structural remodelling of residues 33-40 within human ARG2, wherein the sequence numbering is that of the human ARG2 sequence of Uniprot ID #P78540.

23. An isolated antigen-binding protein according to any preceding clause, characterised in that it is capable of binding specifically to an epitope of human ARG2 and thereby inhibiting the enzyme activity of human ARG2 by an allosteric mechanism, wherein the antigen-binding protein binds to an epitope on human ARG2 that induces structural or biophysical changes upon His160 of human ARG2 that confer a reduction in ARG2 enzyme activity or ability to process substrate, wherein the sequence numbering is that of the human ARG2 sequence of Uniprot ID #P78540.

24. An isolated antigen-binding protein according to any preceding clause, characterised in that the antigen-binding protein binds to an epitope on human ARG2 that induces structural changes whereby Arg39 moves into closer proximity with His160 of human ARG2 thereby conferring a reduction in ARG2 enzyme activity or ability to process substrate, wherein the sequence numbering is that of the human ARG2 sequence of Uniprot ID #P78540.

25. An isolated antigen-binding protein according to any preceding clause, characterised in that the antigen-binding protein binds to an epitope on human ARG2 that induces structural changes whereby the ability of His160 to act as a proton donor/acceptor and/or stabilize a catalytically competent bound orientation of the substrate is compromised thereby conferring a reduction in ARG2 enzyme activity.

26. An antigen-binding protein according to any preceding clause that binds to an epitope on human ARG2 comprising a conformational epitope comprising residues from Gln35 to Arg39, residues from Lys78 to Ile86, and/or residues from Leu152 to Pro179, wherein the sequence numbering is that of the human ARG2 sequence of Uniprot ID #P78540.

27. An antigen-binding protein according to any preceding clause that binds to an epitope on human ARG2 comprising one or more residues selected from Gln35, Gly36, Gln37, Lys38, Arg39, Lys78, Asp79, Asp80, Leu81, Tyr82, Asn84, Leu85, Ile86, Leu152, Thr153 Thr154, Ser155, Ser156, Gly157, Leu178 And Pro179.

28. An antigen-binding protein according to clause 27, wherein the epitope is defined as any residue within human ARG2 with a predicted change in solvent accessibility upon Fab complexation resulting from direct protection by the bound antibody as derived from X-ray structural data of ARG2 inhibitory Fab C0020187, and wherein the sequence numbering is that of the human ARG2 sequence of Uniprot ID #P78540

29. An antigen-binding protein according to any one of clauses 1 to 25 that binds to an epitope on human ARG2 comprising a conformational epitope comprising residues from Pro32 to Glu51, residues from Asp70 to Ile86, and/or residues from Pro299 to Ala308, wherein the sequence numbering is that of the human ARG2 sequence of Uniprot ID #P78540.

30. An antigen-binding protein according to clause 29 that binds to an epitope on human ARG2 comprising one or more residues selected from Pro32, Gln37, Lys38, Lys40, Gly41, Glu43, His44, Ala47, Ala48, Glu51, Asp70, Ser72, Phe73, Thr74, Pro75, Lys78, Asp79, Asp80, Leu81, Tyr82, Asn84, Leu85, Ile86, Pro299, Gln300, Glu305 and Ala308.

31. An antigen-binding protein according to clause 29 or clause 30, wherein the epitope is defined as any residue within human ARG2 with a predicted change in solvent accessibility upon Fab complexation resulting from direct protection by the bound antibody as derived from X-ray structural data of ARG2 inhibitory Fab C0021158, and wherein the sequence numbering is that of the human ARG2 sequence of Uniprot ID #P78540.

32. An antigen-binding protein according to clause 31 that binds to an epitope on human ARG2 comprising a conformational epitope comprising residues from Gln37 to Glu51, residues from Asp79 to Ile86, and/or residues from Pro299 to Ala308, wherein the sequence numbering is that of the human ARG2 sequence of Uniprot ID #P78540.

33. An antigen-binding protein according to any one of clauses 1 to 25 that binds to an epitope on recombinant human ARG2 comprising one or more residues selected from Gln37, Lys38, Lys40, Gly41, His44, Ala47, Ala48, Glu51, Asp79, Asp80, Leu81, Tyr82, Asn84, Leu85, Ile86, Pro299, Gln300, Ala302, Thr303, Ser304, Glu305 and Ala308.

34. An antigen-binding protein according to clause 33, wherein the epitope is defined as any residue within human ARG2 with a predicted change in solvent accessibility upon Fab complexation resulting from direct protection by the bound antibody as derived from X-ray structural data of ARG2 inhibitory Fab C0021181, and wherein the sequence numbering is that of the human ARG2 sequence of Uniprot ID #P78540

35. An antigen-binding protein according to clause 33 or clause 34, that binds to an epitope on human ARG2 comprising one or more residues selected from Pro32, Gln35, Gly36, Gln37, Lys38, Arg39, Lys40, Gly41, Glu43, His44, Ala47, Ala48, Glu51, Asp70, Ser72, Phe73, Thr74, Pro75, Lys78, Asp79, Asp80, Leu81, Tyr82, Asn84, Leu85, Ile86, Leu152, Thr153 Thr154, Ser155, Ser156, Gly157, Leu178, Pro179, Pro299, Gln300, Ala302, Thr303, Ser304, Glu305 and Ala308.

36. An antigen-binding protein according to clause 35, wherein the epitope is defined as any residue within human ARG2 with a predicted change in solvent accessibility upon Fab complexation as derived from X-ray structural data of an ARG2 inhibitory Fab selected from C0020187, C0021158 and C0021181 and wherein the sequence numbering is that of the human ARG2 sequence of Uniprot ID #P78540.

37. An antigen-binding protein according to any of the preceding clauses, wherein the antigen-binding protein is an antibody or a fragment thereof, a domain antibody, a protein scaffold, or an aptamer.

38. An antigen-binding protein according to any of the preceding clauses, wherein the antigen-binding protein is a human IgG or a modified human IgG.

39. An antigen-binding protein according to clause 38, wherein the antigen-binding protein is a human IgG1, IgG2, IgG4, or a modified version thereof.

40. An antigen-binding protein according to clause 38 or clause 39, wherein the antigen-binding protein is a human IgG1 or IgG1-YTE.

41. An antigen-binding protein according to any of the preceding clauses, wherein the antigen-binding protein has a modified Fc to confer enhanced effector function and/or half-life extension.

42. A composition comprising an antigen-binding protein according to any of the preceding clauses, and a pharmaceutically acceptable excipient.

43. An antigen-binding protein or composition according to any of the preceding clauses for use in a method of treatment of the human or animal body.

44. An antigen-binding protein or composition according to any one of the preceding clauses for use in the treatment of an individual to restore immunocompetancy, alleviate inflammation-triggered immune dysfunction, inflammation-associated immune suppression, promote T cell immune responses, or to prevent tumour immune escape, fibrosis, and immunopathology of infectious diseases

45. An antigen-binding protein or composition according to any one of the preceding clauses for use in the treatment of an individual to restore T cell proliferation in the presence of ARG2.

46. An antigen-binding protein or composition according to any one of the preceding clauses for use in the treatment of cancer, immune cell dysfunction, autoimmunity or unwanted immune deviation.

47. An antigen-binding protein or composition according to any one of the preceding clauses for use in the treatment of acute myeloid leukemia (AML), osteosarcoma, HCMV-driven GBM, pancreatic cancer, head and neck squamous cell carcinoma, thyroid, prostate, breast, neuroblastoma or ovarian cancer.

48. An antigen-binding protein or composition according to any one of the preceding clauses for use in the treatment of infection (e.g., neonate infection), endothelial dysfunction (e.g. erectile dysfunction), vascular disease, cardiovascular disease, ageing and cellular senescence, CNS disease and injury; diabetes-associated disease or cystic fibrosis or infection associated with cystic fibrosis.

49. A method of treating an individual, comprising administering an antigen-binding protein or composition according to any of clauses 1 to 48 to the individual.

50. An isolated nucleic acid encoding an antigen-binding protein according to any of clauses 1 to 41.

51. A host cell in vitro transformed with nucleic acid according to clause 50.

52. A method of producing an antigen-binding protein according to any of clauses 1 to 41, comprising culturing host cells according to clause 51 under conditions for production of the antigen-binding protein.

53. A method according to clause 52, further comprising isolating and/or purifying the antigen-binding protein.

54. A method according to clause 53, further comprising formulating the antigen-binding protein into a composition comprising at least one additional component.

55. A method for producing an antigen-binding protein that binds specifically to and inhibits human ARG2, the method comprising providing a variant VH domain which is an amino acid sequence variant of a parent VH domain, by way of addition, deletion, substitution or insertion of one or more amino acids in the amino acid sequence of a parent VH domain comprising HCDR1, HCDR2 and HCDR3, wherein the parent VH domain HCDR1, HCDR2 and HCDR3 are a set of HCDRs selected from the set of HCDRs of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, and optionally combining the variant VH domain thus provided with one or more VL domains to provide one or more VH/VL combinations; and testing said variant VH domain which is an amino acid sequence variant of the parent VH domain or the variant VH/VL combination or combinations to identify an antigen-binding protein antigen binding domain for human ARG2.

56. A method according to clause 50, wherein the parent VH domain is selected from C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, or a germlined version thereof.

57. A method according to clause 55 or clause 56, wherein the one or more VL domains is a variant VL domain provided by way of addition, deletion, substitution or insertion of one or more amino acids in the amino acid sequence of a parent VL domain comprising LCDR1, LCDR2 and LCDR3, wherein the parent VL domain LCDR1, LCDR2 and LCDR3 are a set of LCDRs selected from the set of LCDRs of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, producing one or more variant VL domains each of which is an amino acid sequence variant of the parent VL domain.

58. A method according to clause 57, wherein the parent VL domain is the VL domain of any of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, or a germlined version thereof.

59. A method according to any of clauses 55 to 58, further comprising producing the antigen-binding protein antigen-binding domain as a component of an IgG, scFv or Fab antigen-binding protein.

60. A method for producing an antigen-binding protein that binds to and inhibits human ARG2, wherein the method comprises: providing starting nucleic acid encoding a VH domain or a starting repertoire of nucleic acids each encoding a VH domain, wherein the VH domain or VH domains either comprise a HCDR1, HCDR2 and/or HCDR3 to be replaced or lack a HCDR1, HCDR2 and/or HCDR3 encoding region; combining said starting nucleic acid or starting repertoire with donor nucleic acid or donor nucleic acids encoding the amino acid sequence of an HCDR1, HCDR2, and/or HCDR3 selected from the HCDR1, HCDR2 and/or HCDR3 of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, such that said donor nucleic acid is or donor nucleic acids are inserted into the CDR1, CDR2 and/or CDR3 region in the starting nucleic acid or starting repertoire, so as to provide a product repertoire of nucleic acids encoding VH domains; expressing the nucleic acids of said product repertoire to produce product VH domains; optionally combining said product VH domains with one or more VL domains;

selecting an antigen-binding protein for ARG2, wherein the antigen-binding protein comprises a product VH domain and optionally a VL domain; and

recovering the antigen-binding protein or nucleic acid encoding it.

61. A method according to clause 60, wherein the donor nucleic acids are produced by mutation of said HCDR1 and/or HCDR2.

62. A method according to clause 60 or clause 61, wherein the donor nucleic acid is produced by mutation of HCDR3.

63. A method according to any one of clauses 60 to 62, comprising providing the donor nucleic acid by random mutation of nucleic acid.

64. A method according to any of clauses 60 to 63, further comprising attaching a product VH domain that is comprised within the recovered antigen-binding protein to an antigen-binding-protein constant region.

65. A method according to any of clauses 60 to 64, comprising providing an IgG, scFv or Fab antigen-binding protein comprising the product VH domain and a VL domain.

### DETAILED DESCRIPTION

The invention relates to antigen-binding proteins, in particular antibodies and antigen-binding fragments thereof that comprise an antigen-binding site for ARG2. An antibody or antigen-binding fragment thereof of the invention may be produced by recombinant means.

A “recombinant antibody” is an antibody, which has been produced by a recombinantly engineered host cell. An antibody or antigen-binding fragment thereof in accordance with the invention is optionally isolated or purified.

The term “ARG2” may refer to human ARG2, and/or cynomolgus monkey ARG2, unless the context requires otherwise. Preferably the term “ARG2” refers to human ARG2 (Uniprot ID: P78540), unless the context requires otherwise.

The term “antibody” describes an immunoglobulin whether natural or partly or wholly synthetically produced. The antibody may be human or humanised. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The antibody is preferably a monoclonal antibody, more preferably a human monoclonal antibody. Examples of antibodies are the immunoglobulin isotypes, such as immunoglobulin G, and their isotypic subclasses, such as IgG1, IgG2, IgG3 and IgG4, as well as fragments thereof. The four human subclasses (IgG1, IgG2, IgG3 and IgG4) each contain a different heavy chain; but they are highly homologous and differ mainly in the hinge region and the extent to which they activate the host immune system. IgG1 and IgG4 contain two inter-chain disulphide bonds in the hinge region, IgG2 has 4 and IgG3 has 11 inter-chain disulphide bonds.

The terms “antibody” and “antibody molecule”, as used herein, includes antibody fragments, such as Fab and scFv fragments, provided that said fragments comprise a CDR-based antigen-binding site for ARG2. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; and single-chain antibody molecules (e.g., scFv). Unless the context requires otherwise, the terms “antigen-binding proteins”, “antibody” or “antibody molecule”, as used herein, are thus equivalent to “antibody or antigen-binding fragment thereof”.

Antibodies are immunoglobulins, which have the same basic structure consisting of two heavy and two light chains forming two Fab arms containing identical domains that are attached by a flexible hinge region to the stem of the antibody, the Fc domain, giving the classical ‘Y’ shape. The Fab domains consist of two variable and two constant domains, with a variable heavy (VH) and constant heavy 1 (CH1) domain on the heavy chain and a variable light (VL) and constant light (CL) domain on the light chain. The two variable domains (VH and VL) form the variable fragment (Fv), which provides the CDR-based antigen specificity of the antibody, with the constant domains (CH1 and VL) acting as a structural framework. Each variable domain contains three hypervariable loops, known as complementarity determining regions (CDRs). On each of the VH and VL the three CDRs (CDR1, CDR2, and CDR3) are flanked by four less-variable framework (FR) regions (FR1, FW2, FW3 and FW4) to give a structure FW1-CDR1-FW2-CDR2-FW3-CDR3-FW4. The CDRs provide a specific antigen recognition site on the surface of the antibody.

Generally, unless otherwise indicated (explicitly or by context) amino acid residues are numbered herein according to the Kabat numbering scheme (Kabat et al., 1991).

It is possible to take monoclonal and other antibodies and use techniques of recombinant DNA technology to produce other antibodies or chimeric molecules, which generally retain the specificity of the original antibody. Such techniques may involve introducing the CDRs into a different immunoglobulin framework, or grafting variable regions onto a different immunoglobulin constant region. Introduction of the CDRs of one immunoglobulin into another immunoglobulin is described for example in EP-A-184187, GB2188638A or EP-A-239400. Alternatively, a hybridoma or other cell producing an antibody molecule may be subject to genetic mutation or other changes, which may or may not alter the binding specificity of antibodies produced.

As antibodies can be modified in a number of ways, the term “antibody” should be construed as covering antibody fragments, derivatives, functional equivalents and homologues of antibodies, including any polypeptide comprising an immunoglobulin binding domain, whether natural or wholly or partially synthetic.

An antigen-binding protein, such as an antibody or antigen-binding fragment of the invention binds to and inhibits ARG2, in particular human ARG2. Binding in this context may refer to specific binding. The term “specific” may refer to the situation in which the antigen-binding protein will not show any significant binding to molecules other than its specific binding partner(s), here ARG2. The term “specific” is also applicable where the antibody molecule is specific for particular epitopes, such as epitopes on ARG2, that are carried by a number of antigens in which case the antibody molecule will be able to bind to the various antigens carrying the epitope.

An antibody that binds to the same epitope as, or an epitope overlapping with, a reference antibody refers to an antibody that blocks binding of the reference antibody to its binding partner (e.g., an antigen) in a competition assay by 50% or more, and conversely, the reference antibody blocks binding of the antibody to its binding partner in a competition assay by 50% or more. Such antibodies are said to compete for binding to an epitope of interest.

Amino acids may be referred to by their one letter or three letter codes, or by their full name. The one and three letter codes, as well as the full names, of each of the twenty standard amino acids are set out below.

TABLE 1
Amino acids, one and three-letter codes

| | Three | One |
|---|---|---|
| | letter | letter |
| Amino acid | code | code |
| alanine | Ala | A |
| arginine | Arg | R |
| asparagine | Asn | N |
| aspartic acid | Asp | D |
| asparagine or aspartic acid | Asx | B |
| cysteine | Cys | C |
| glutamic acid | Glu | E |
| glutamine | Gln | Q |
| glutamine or glutamic acid | Glx | Z |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

In preferred embodiments, an ARG2 antibody of the invention comprises HCDR1, HCDR2 and HCDR3 of a VH and/or a LCDR1, LCDR2 and LCDR3 of a VL of an antibody selected from: C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065.

More preferably, an ARG2 antibody of the invention comprises a VH and/or VL with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence homology to an antibody selected from: C0021158fgl2, C0021181, C0021180, C0021177, C0021158, C0021158 IgG, C0021158fgl, C0021158dr, C0021061, C0020187 (parent lead isolation clone), C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065 (alternate lead isolation clone).

In further preferred embodiments, an ARG2 antibody of the invention comprises a VH comprising HCDR1, HCDR2 and HCDR3 and/or VL comprising LCDR1, LCDR2 and LCDR3, wherein the VH and/or VL have at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence homology to the VH and/or VL of an antibody selected from: C0021158fgl2, C0021181, C0021180, C0021177, C0021158, C0021158 IgG, C0021158fgl, C0021158dr, C0021061, C0020187, C0021155, C0021144, C0021142, C0021142 (IgG), C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065.

In particularly preferred embodiments, an ARG2 antibody of the invention comprises a VH and a VL of an antibody selected from: C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065.

Unless otherwise indicated, sequence homology is assessed using the Clustal W method alignment (Thompson, Higgins et al. 1994).

TABLE 2
Sequence identity across the entire VH sequence (Kabat residues 1→113)
of lead isolation antibodies C0020065 and C0020187 described herein.
Percent diversity and percent divergence values derived from
Clustal W method alignment (Thompson, Higgins et al. 1994).

| | | Percent | Percent |
|---|---|---|---|
| Homology of the | | Divergence | Diversity |
| VH Domain | | 1 | 2 |
| C0020065 | 1 | | 82.2 |
| C0020187 | 2 | 20.4 | |

TABLE 3
Sequence identity across the entire VL sequence (Kabat residues 1→107)
of lead isolation antibodies C0020065 and C0020187 described herein.
Percent diversity and percent divergence values derived from
Clustal W method alignment (Thompson, Higgins et al. 1994).

| | | Percent | Percent |
|---|---|---|---|
| Homology of the | | Divergence | Diversity |
| VL Domain | | 1 | 2 |
| C0020065 | 1 | | 81.8 |
| C0020187 | 2 | 20.9 | |

In FIG. 24, Table 4 shows the sequence identity across the entire VH sequence (Kabat residues 1→113) of the parental C0020187 antibody and the thirty-three affinity-matured antibodies described herein. All affinity-matured sequences share at least 90% identity with the parental C0020187 antibody. Percent diversity and percent divergence values derived from Clustal W method alignment (Thompson, Higgins et al. 1994).

In FIG. 25, Table 5 shows the sequence identity across the entire VL sequence (Kabat residues 1→107) of the parental C0020187 antibody and the thirty-three affinity-matured antibodies described herein. All affinity-matured sequences share at least 89.1% identity with the parental C0020187 antibody. Percent diversity and percent divergence values derived from Clustal W method alignment (Thompson, Higgins et al. 1994).

Antibodies of the invention may comprise a VH and/or a VL wherein the amino acid sequence is that of C0021158fgl2, C0021133 or C0020187 at each position below, or optionally other example residues, found in clones described herein selected from: C0021181, C0021180, C0021177, C0021158, C0021158 IgG, C0021158fgl, C0021158dr, C0021061, C0021155, C0021144, C0021142, C0021142 IgG, C0021141, C0021139, C0021135, C0021131, C0021129, C0021128, C0021124, C0021118, C0021101, C0021098, C0021097, C0021096, C0021092, C0021089, C0021065, C0021032, C0021022, C0021021 and C0021017 may be present as specified at a given position as follows:

TABLE 6
A summary of residues for the C0021158fgl2, C0021133
or parental C0020187 antibody observed in the VH sequence
of the thirty-three affinity-matured antibodies described herein.
Amino acid numbering as defined by Kabat (Kabat and Wu 1991).

| | Kabat | | | | Other |
|---|---|---|---|---|---|
| | position | | | | example |
| Domain | number | C0021158fgl2 | C0021133 | C0020187 | residues |
| VHFW1 | 1 | E | E | E | |
| | 2 | V | V | V | |
| | 3 | Q | Q | Q | |
| | 4 | L | L | L | |
| | 5 | L | L | L | |
| | 6 | E | E | E | |
| | 7 | S | S | S | |
| | 8 | G | G | G | |
| | 9 | G | G | G | |
| | 10 | G | G | G | |
| | 11 | L | L | L | |
| | 12 | V | V | V | |
| | 13 | Q | Q | Q | R |
| | 14 | P | P | P | |
| | 15 | G | G | G | |
| | 16 | G | G | G | |
| | 17 | S | S | S | |
| | 18 | L | L | L | |
| | 19 | R | R | R | |
| | 20 | L | L | L | |
| | 21 | S | S | S | |
| | 22 | C | C | C | |
| | 23 | A | A | A | E |
| | 24 | A | A | A | |
| | 25 | S | S | S | |
| | 26 | G | G | G | E |
| | 27 | F | F | F | |
| | 28 | T | T | T | A |
| | 29 | F | F | F | |
| | 30 | R | R | S | P Q R Y |
| VHCDR1 | 31 | Y | Y | S | P R Y |
| | 32 | E | D | Y | D E |
| | 33 | V | H | A | H P V Y |
| | 34 | A | H | M | A E H I W Q |
| | 35 | A | V | S | A G L Q V |
| VHFW2 | 36 | W | W | W | |
| | 37 | V | V | V | |
| | 38 | R | R | R | |
| | 39 | Q | Q | Q | |
| | 40 | A | A | A | |
| | 41 | P | P | P | |
| | 42 | G | G | G | |
| | 43 | K | K | K | |
| | 44 | G | G | G | |
| | 45 | L | L | L | P |
| | 46 | E | E | E | |
| | 47 | W | W | W | |
| | 48 | V | V | V | |
| | 49 | S | S | S | |
| VHCDR2 | 50 | A | A | A | S |
| | 51 | I | I | I | H K S Y |
| | 52 | S | S | S | G L P T- |
| | 52a | G | G | G | F H T Y- |
| | 53 | P | S | S | D G H P N |
| | 54 | I | G | G | A I P |
| | 55 | P | G | G | F P Y |
| | 56 | K | S | S | K |
| | 57 | G | T | T | G P |
| | 58 | Y | Y | Y | F |
| | 59 | Y | Y | Y | |
| | 60 | A | A | A | |
| | 61 | D | D | D | |
| | 62 | S | S | S | P |
| | 63 | V | V | V | |
| | 64 | K | K | K | |
| | 65 | G | G | G | S |
| VHFW3 | 66 | R | R | R | |
| | 67 | F | F | F | |
| | 68 | T | T | T | |
| | 69 | I | I | I | |
| | 70 | S | S | S | |
| | 71 | R | R | R | |
| | 72 | D | D | D | |
| | 73 | N | N | N | |
| | 74 | S | S | S | |
| | 75 | K | K | K | R |
| | 76 | N | N | N | |
| | 77 | T | T | T | |
| | 78 | L | L | L | |
| | 79 | Y | Y | Y | |
| | 80 | L | L | L | |
| | 81 | Q | Q | Q | |
| | 82 | M | M | M | I |
| | 82a | N | N | N | |
| | 82b | S | S | S | |
| | 82c | L | L | L | |
| | 83 | R | R | R | |
| | 84 | A | A | A | |
| | 85 | E | E | E | Q |
| | 86 | D | D | D | |
| | 87 | T | T | T | |
| | 88 | A | A | A | |
| | 89 | V | V | V | |
| | 90 | Y | Y | Y | |
| | 91 | Y | Y | Y | |
| | 92 | C | C | C | |
| | 93 | A | A | A | |
| | 94 | R | R | R | |
| VHCDR3 | 95 | L | L | L | |
| | 96 | R | R | R | |
| | 97 | A | A | A | |
| | 98 | D | D | D | |
| | 99 | L | L | L | |
| | 100 | G | G | G | |
| | 100a | L | L | L | |
| | 100b | Y | Y | Y | |
| | 100c | M | M | M | |
| | 101 | D | D | D | |
| | 102 | L | L | L | |
| VHFW4 | 103 | W | W | W | |
| | 104 | G | G | G | |
| | 105 | R | R | R | |
| | 106 | G | G | G | |
| | 107 | T | T | T | |
| | 108 | L | L | L | |
| | 109 | V | V | V | |
| | 110 | T | T | T | |
| | 111 | V | V | V | |
| | 112 | S | S | S | P |
| | 113 | S | S | S | |

TABLE 7
A summary of residues of the 00021158fgl2, 00021133 or
parental 00020187 antibody observed in the VL sequence of
the thirty-three affinity-matured antibodies described herein.
Amino acid numbering as defined by Kabat (Kabat and Wu 1991).

| | | | | | Other |
|---|---|---|---|---|---|
| | Kabat | | | | example |
| Domain | Number | C0021158fgl2 | C0021133 | C0020187 | residues |
| VLFW1 | 1 | Q | Q | Q | R |
| | 2 | S | S | S | |
| | 3 | V | V | V | E |
| | 4 | L | L | L | |
| | 5 | T | T | T | |
| | 6 | Q | Q | Q | |
| | 7 | P | P | P | L |
| | 8 | P | P | P | |
| | 9 | S | S | S | T |
| | 10 | — | — | — | |
| | 11 | V | V | V | A |
| | 12 | S | S | S | |
| | 13 | A | A | A | |
| | 14 | A | A | A | |
| | 15 | P | P | P | |
| | 16 | G | G | G | |
| | 17 | Q | Q | Q | R |
| | 18 | K | K | K | E |
| | 19 | V | V | V | |
| | 20 | T | T | T | A |
| | 21 | I | V | I | V |
| | 22 | S | S | S | |
| | 23 | C | C | C | |
| VLCDR1 | 24 | S | S | S | |
| | 25 | G | G | G | |
| | 26 | S | S | S | |
| | 27 | S | S | S | |
| | 27a | S | S | S | |
| | 27b | N | N | N | |
| | 28 | I | I | I | |
| | 29 | G | G | G | D |
| | 30 | N | N | N | D S |
| | 31 | H | H | H | |
| | 32 | Y | Y | Y | H |
| | 33 | V | V | V | |
| | 34 | S | S | S | |
| VLFW2 | 35 | W | W | W | |
| | 36 | Y | Y | Y | |
| | 37 | Q | Q | Q | |
| | 38 | Q | Q | Q | |
| | 39 | L | L | L | |
| | 40 | P | P | P | |
| | 41 | G | G | G | |
| | 42 | T | T | T | A |
| | 43 | A | A | A | |
| | 44 | P | P | P | H |
| | 45 | K | K | K | |
| | 46 | L | L | L | |
| | 47 | L | L | L | |
| | 48 | I | I | I | |
| | 49 | Y | Y | Y | |
| VLCDR2 | 50 | D | D | D | N |
| | 51 | N | T | N | S T |
| | 52 | A | T | S | A D E F |
| | | | | | R T V |
| | 53 | E | V | E | A H I R |
| | | | | | S V Y |
| | 54 | R | L | R | E L M P |
| | 55 | T | S | P | A K M S T |
| | 56 | A | S | S | A |
| VLFW3 | 57 | G | G | G | |
| | 58 | I | I | I | V |
| | 59 | P | P | P | |
| | 60 | D | D | D | |
| | 61 | R | R | R | |
| | 62 | F | F | F | |
| | 63 | S | S | S | |
| | 64 | G | G | G | |
| | 65 | S | S | S | |
| | 66 | K | K | K | R |
| | 67 | S | S | S | |
| | 68 | G | G | G | |
| | 69 | T | T | T | |
| | 70 | S | S | S | |
| | 71 | A | A | A | |
| | 72 | T | T | T | |
| | 73 | L | L | L | |
| | 74 | G | G | G | D |
| | 75 | I | I | I | |
| | 76 | T | T | T | |
| | 77 | G | G | G | |
| | 78 | L | L | L | |
| | 79 | Q | Q | Q | |
| | 80 | T | T | T | |
| | 81 | G | G | G | |
| | 82 | D | D | D | |
| | 83 | E | E | E | G |
| | 84 | A | A | A | |
| | 85 | D | D | D | |
| | 86 | Y | Y | Y | |
| | 87 | Y | Y | Y | |
| | 88 | C | C | C | |
| VLCDR3 | 89 | G | G | G | A |
| | 90 | T | T | T | |
| | 91 | W | W | W | Y |
| | 92 | D | D | D | |
| | 93 | E | E | S | E P T |
| | 94 | L | L | S | D L N T |
| | 95 | T | T | L | A R T V |
| | 95a | S | S | S | A V |
| | 95b | N | N | A | E N R |
| | 96 | L | L | L | |
| | 97 | V | V | V | E |
| VLFW4 | 98 | F | F | F | |
| | 99 | G | G | G | |
| | 100 | G | G | G | |
| | 101 | G | G | G | |
| | 102 | T | T | T | |
| | 103 | K | K | K | R |
| | 104 | L | L | L | |
| | 105 | T | T | T | |
| | 106 | V | V | V | |
| | 107 | L | L | L | |

Antibodies of the invention may comprise one or more, e.g., 1, 2, 3, 4, 5, 6, 7, 8, 9 or 10 further amino acid modifications in the VH and/or VL sequences, provided that functional properties of the antibody are retained.

A modification may be an amino acid substitution, deletion or insertion. Preferably, the modification is a substitution.

In preferred embodiments in which one or more amino acids are substituted with another amino acid the substitutions may be conservative substitutions. In some embodiments, variants having one or more amino acid substitutions are provided. Sites of interest for substitutional mutagenesis include the CDRs and FWs. Amino acid substitutions may be introduced into the antibody or the binding polypeptide and the products screened for a desired activity, e.g., retained/improved antigen binding, greater inhibition, or decreased immunogenicity.

Amino acids may be grouped according to common side-chain properties:

(1) hydrophobic: Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Conservative substitutions are made by exchanging members within one of these groups, whereas non-conservative substitutions are made by exchanging a member of one of these groups for a member of another group.

In some embodiments, substitution(s) may be functionally conservative. That is, in some embodiments the substitution may not affect (or may not substantially affect) one or more functional properties (e.g., binding affinity, inhibition of ARG2) of the antibody comprising the substitution as compared to the equivalent unsubstituted antibody.

In a preferred embodiment, a ARG2 antibody of the invention may comprise a VH and/or VL domain sequence of the invention as described herein with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the VH and/or VL sequences of the invention set forth herein.

In preferred embodiments, an antibody of the invention comprises the HCDR3 sequence of C0021158 fgl2 of SEQ ID NO: 315.

All clones of the invention related to parental clone C0020187 described herein share a common HCDR3 sequence as shown in SEQ ID NO: 315 (LRADLGLYMDL). This HCDR3 sequence is believed to be important for determining specificity to, and inhibition of, the enzyme activity of human ARG2.

Preferably an antibody of the invention comprises:

a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021158fgl2;

a VH domain comprising HCDR1 (SEQ ID NO: 343), HCDR2 (SEQ ID NO: 344) and HCDR3 (SEQ ID NO: 345) and a VL domain comprising LCDR1 (SEQ ID NO: 348), LCDR2, (SEQ ID NO: 349) and LCDR3 (SEQ ID NO: 350) of C0021181;

a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021180;

a VH domain comprising HCDR1 (SEQ ID NO: 323), HCDR2 (SEQ ID NO: 324) and HCDR3 (SEQ ID NO: 325) and a VL domain comprising LCDR1 (SEQ ID NO: 328), LCDR2, (SEQ ID NO: 329) and LCDR3 (SEQ ID NO: 330) of C0021177;

a VH domain comprising HCDR1 (SEQ ID NO: 273), HCDR2 (SEQ ID NO: 274) and HCDR3 (SEQ ID NO: 275) and a VL domain comprising LCDR1 (SEQ ID NO: 278), LCDR2, (SEQ ID NO: 279) and LCDR3 (SEQ ID NO: 280) of C0021158;

a VH domain comprising HCDR1 (SEQ ID NO: 283), HCDR2 (SEQ ID NO: 284) and HCDR3 (SEQ ID NO: 285) and a VL domain comprising LCDR1 (SEQ ID NO: 288), LCDR2, (SEQ ID NO: 289) and LCDR3 (SEQ ID NO: 290) of C0021158 IgG;

a VH domain comprising HCDR1 (SEQ ID NO: 303), HCDR2 (SEQ ID NO: 304) and HCDR3 (SEQ ID NO: 305) and a VL domain comprising LCDR1 (SEQ ID NO: 308), LCDR2, (SEQ ID NO: 309) and LCDR3 (SEQ ID NO: 310) of C0021158fgl;

a VH domain comprising HCDR1 (SEQ ID NO: 293), HCDR2 (SEQ ID NO: 294) and HCDR3 (SEQ ID NO: 295) and a VL domain comprising LCDR1 (SEQ ID NO: 298), LCDR2, (SEQ ID NO: 299) and LCDR3 (SEQ ID NO: 300) of C0021158dr;

a VH domain comprising HCDR1 (SEQ ID NO: 63), HCDR2 (SEQ ID NO: 64) and HCDR3 (SEQ ID NO: 65) and a VL domain comprising LCDR1 (SEQ ID NO: 68), LCDR2, (SEQ ID NO: 69) and LCDR3 (SEQ ID NO: 70) of C0021061;

a VH domain comprising HCDR1 (SEQ ID NO: 13), HCDR2 (SEQ ID NO: 14) and HCDR3 (SEQ ID NO: 15) and a VL domain comprising LCDR1 (SEQ ID NO: 18), LCDR2, (SEQ ID NO: 19) and LCDR3 (SEQ ID NO: 20) of C0020187;

a VH domain comprising HCDR1 (SEQ ID NO: 263), HCDR2 (SEQ ID NO: 264) and HCDR3 (SEQ ID NO: 265) and a VL domain comprising LCDR1 (SEQ ID NO: 268), LCDR2, (SEQ ID NO: 269) and LCDR3 (SEQ ID NO: 270) of C0021155;

a VH domain comprising HCDR1 (SEQ ID NO: 253), HCDR2 (SEQ ID NO: 254) and HCDR3 (SEQ ID NO: 255) and a VL domain comprising LCDR1 (SEQ ID NO: 258), LCDR2, (SEQ ID NO: 259) and LCDR3 (SEQ ID NO: 260) of C0021144;

a VH domain comprising HCDR1 (SEQ ID NO: 233), HCDR2 (SEQ ID NO: 234) and HCDR3 (SEQ ID NO: 235) and a VL domain comprising LCDR1 (SEQ ID NO: 238), LCDR2, (SEQ ID NO: 239) and LCDR3 (SEQ ID NO: 240) of C0021142;

a VH domain comprising HCDR1 (SEQ ID NO: 243), HCDR2 (SEQ ID NO: 244) and HCDR3 (SEQ ID NO: 245) and a VL domain comprising LCDR1 (SEQ ID NO: 248), LCDR2, (SEQ ID NO: 249) and LCDR3 (SEQ ID NO: 250) of C0021142 IgG;

a VH domain comprising HCDR1 (SEQ ID NO: 223), HCDR2 (SEQ ID NO: 224) and HCDR3 (SEQ ID NO: 225) and a VL domain comprising LCDR1 (SEQ ID NO: 228), LCDR2, (SEQ ID NO: 229) and LCDR3 (SEQ ID NO: 230) of C0021141;

a VH domain comprising HCDR1 (SEQ ID NO: 213), HCDR2 (SEQ ID NO: 214) and HCDR3 (SEQ ID NO: 215) and a VL domain comprising LCDR1 (SEQ ID NO: 218), LCDR2, (SEQ ID NO: 219) and LCDR3 (SEQ ID NO: 220) of C0021139;

a VH domain comprising HCDR1 (SEQ ID NO: 203), HCDR2 (SEQ ID NO: 204) and HCDR3 (SEQ ID NO: 205) and a VL domain comprising LCDR1 (SEQ ID NO: 208), LCDR2, (SEQ ID NO: 209) and LCDR3 (SEQ ID NO: 210) of C0021135;

a VH domain comprising HCDR1 (SEQ ID NO: 193), HCDR2 (SEQ ID NO: 194) and HCDR3 (SEQ ID NO: 195) and a VL domain comprising LCDR1 (SEQ ID NO: 198), LCDR2, (SEQ ID NO: 199) and LCDR3 (SEQ ID NO: 200) of C0021133;

a VH domain comprising HCDR1 (SEQ ID NO: 183), HCDR2 (SEQ ID NO: 184) and HCDR3 (SEQ ID NO: 185) and a VL domain comprising LCDR1 (SEQ ID NO: 188), LCDR2, (SEQ ID NO: 189) and LCDR3 (SEQ ID NO: 190) of C0021131;

a VH domain comprising HCDR1 (SEQ ID NO: 173), HCDR2 (SEQ ID NO: 174) and HCDR3 (SEQ ID NO: 175) and a VL domain comprising LCDR1 (SEQ ID NO: 178), LCDR2, (SEQ ID NO: 179) and LCDR3 (SEQ ID NO: 180) of C0021129;

a VH domain comprising HCDR1 (SEQ ID NO: 163), HCDR2 (SEQ ID NO: 164) and HCDR3 (SEQ ID NO: 165) and a VL domain comprising LCDR1 (SEQ ID NO: 168), LCDR2, (SEQ ID NO: 169) and LCDR3 (SEQ ID NO: 170) C0021128;

a VH domain comprising HCDR1 (SEQ ID NO: 153), HCDR2 (SEQ ID NO: 154) and HCDR3 (SEQ ID NO: 155) and a VL domain comprising LCDR1 (SEQ ID NO: 158), LCDR2, (SEQ ID NO: 159) and LCDR3 (SEQ ID NO: 160) of C0021124;

a VH domain comprising HCDR1 (SEQ ID NO: 143), HCDR2 (SEQ ID NO: 144) and HCDR3 (SEQ ID NO: 145) and a VL domain comprising LCDR1 (SEQ ID NO: 148), LCDR2, (SEQ ID NO: 149) and LCDR3 (SEQ ID NO: 150) of C0021118;

a VH domain comprising HCDR1 (SEQ ID NO: 133), HCDR2 (SEQ ID NO: 134) and HCDR3 (SEQ ID NO: 135) and a VL domain comprising LCDR1 (SEQ ID NO: 138), LCDR2, (SEQ ID NO: 139) and LCDR3 (SEQ ID NO: 140) of C0021101;

a VH domain comprising HCDR1 (SEQ ID NO: 123), HCDR2 (SEQ ID NO: 124) and HCDR3 (SEQ ID NO: 125) and a VL domain comprising LCDR1 (SEQ ID NO: 128), LCDR2, (SEQ ID NO: 129) and LCDR3 (SEQ ID NO: 130) of C0021098;

a VH domain comprising HCDR1 (SEQ ID NO: 113), HCDR2 (SEQ ID NO: 114) and HCDR3 (SEQ ID NO: 115) and a VL domain comprising LCDR1 (SEQ ID NO: 118), LCDR2, (SEQ ID NO: 119) and LCDR3 (SEQ ID NO: 120) of C0021097;

a VH domain comprising HCDR1 (SEQ ID NO: 103), HCDR2 (SEQ ID NO: 104) and HCDR3 (SEQ ID NO: 105) and a VL domain comprising LCDR1 (SEQ ID NO: 108), LCDR2, (SEQ ID NO: 109) and LCDR3 (SEQ ID NO: 110) of C0021096;

a VH domain comprising HCDR1 (SEQ ID NO: 93), HCDR2 (SEQ ID NO: 94) and HCDR3 (SEQ ID NO: 95) and a VL domain comprising LCDR1 (SEQ ID NO: 98), LCDR2, (SEQ ID NO: 99) and LCDR3 (SEQ ID NO: 100) of C0021092;

a VH domain comprising HCDR1 (SEQ ID NO: 83), HCDR2 (SEQ ID NO: 84) and HCDR3 (SEQ ID NO: 85) and a VL domain comprising LCDR1 (SEQ ID NO: 88), LCDR2, (SEQ ID NO: 89) and LCDR3 (SEQ ID NO: 90) of C0021089;

a VH domain comprising HCDR1 (SEQ ID NO: 73), HCDR2 (SEQ ID NO: 74) and HCDR3 (SEQ ID NO: 75) and a VL domain comprising LCDR1 (SEQ ID NO: 78), LCDR2, (SEQ ID NO: 79) and LCDR3 (SEQ ID NO: 80) of C0021065;

a VH domain comprising HCDR1 (SEQ ID NO: 53), HCDR2 (SEQ ID NO: 54) and HCDR3 (SEQ ID NO: 55) and a VL domain comprising LCDR1 (SEQ ID NO: 58), LCDR2, (SEQ ID NO: 59) and LCDR3 (SEQ ID NO: 60) of C0021032;

a VH domain comprising HCDR1 (SEQ ID NO: 43), HCDR2 (SEQ ID NO: 44) and HCDR3 (SEQ ID NO: 45) and a VL domain comprising LCDR1 (SEQ ID NO: 48), LCDR2, (SEQ ID NO: 49) and LCDR3 (SEQ ID NO: 50) of C0021022;

a VH domain comprising HCDR1 (SEQ ID NO: 33), HCDR2 (SEQ ID NO: 34) and HCDR3 (SEQ ID NO: 35) and a VL domain comprising LCDR1 (SEQ ID NO: 38), LCDR2, (SEQ ID NO: 39) and LCDR3 (SEQ ID NO: 40) of C0021021;

a VH domain comprising HCDR1 (SEQ ID NO: 23), HCDR2 (SEQ ID NO: 24) and HCDR3 (SEQ ID NO: 25) and a VL domain comprising LCDR1 (SEQ ID NO: 28), LCDR2, (SEQ ID NO: 29) and LCDR3 (SEQ ID NO: 30) of C0021017; or a VH domain comprising HCDR1 (SEQ ID NO: 3), HCDR2 (SEQ ID NO: 4) and HCDR3 (SEQ ID NO: 5) and a VL domain comprising LCDR1 (SEQ ID NO: 8), LCDR2, (SEQ ID NO: 9) and LCDR3 (SEQ ID NO: 10) of C0020065.

In preferred embodiments, an antibody of the invention comprises a VH domain comprising a HCDR1 domain of C0021158 fgl2 of SEQ ID NO: 313, a HCDR3 sequence of C0021158 fgl2 of SEQ ID NO: 315 and a HCDR2 sequence of C0021158fgl2 of SEQ ID NO: 314.

In preferred embodiments, an antibody of the invention comprises the VH domain of C0021158 fgl2 of SEQ ID NO: 312 or a VH domain with an amino acid sequence which has at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 312.

In preferred embodiments, an antibody of the invention comprises a VH domain comprising the HCDR3 sequence of SEQ ID NO: 315 and the VH domain has an amino acid sequence of SEQ ID NO: 312 or an amino acid sequence with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 312.

In preferred embodiments, an antibody of the invention comprises a VH domain comprising the HCDR3 of C0021158 fgl2 of SEQ ID NO: 315 and a HCDR2 sequence of C0021158 fgl2 of SEQ ID NO: 314 and the VH domain has an amino acid sequence of SEQ ID NO: 312 or an amino acid sequence with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 312.

In preferred embodiments, an antibody of the invention comprises a VH domain comprising a HCDR3 of C0021158 fgl2 of SEQ ID NO: 315, a HCDR2 domain of C0021158 fgl2 of SEQ ID NO: 314, a HCDR1 domain of C0021158 fgl2 of SEQ ID NO: 313 and the VH domain has an amino acid sequence of SEQ ID NO: 312 or an amino acid sequence with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 312.

Preferably an antibody of the invention comprises a VH domain of C0021158fgl2 (SEQ ID NO: 312), C0021181 (SEQ ID NO: 342), C0021180 (SEQ ID NO: 332), C0021177 (SEQ ID NO: 322), C0021158 (SEQ ID NO: 272), C0021158 IgG (SEQ ID NO: 282), C0021158fgl (SEQ ID NO: 302), C0021158dr (SEQ ID NO: 292), C0021061 (SEQ ID NO: 62), C0020187 (SEQ ID NO: 12), C0021155 (SEQ ID NO: 262), C0021144 (SEQ ID NO: 252), C0021142 (SEQ ID NO: 232), C0021142 IgG (SEQ ID NO: 242), C0021141 (SEQ ID NO: 222), C0021139 (SEQ ID NO: 212), C0021135 (SEQ ID NO: 202), C0021133 (SEQ ID NO: 192), C0021131 (SEQ ID NO: 182), C0021129 (SEQ ID NO: 32), C0021128 (SEQ ID NO: 162), C0021124 (SEQ ID NO: 152), C0021118 (SEQ ID NO: 142), C0021101 (SEQ ID NO: 132), C0021098 (SEQ ID NO: 122), C0021097 (SEQ ID NO: 112), C0021096 (SEQ ID NO: 102), C0021092 (SEQ ID NO: 92), C0021089 (SEQ ID NO: 82), C0021065 (SEQ ID NO: 72), C0021032 (SEQ ID NO: 52), C0021022 (SEQ ID NO: 42), C0021021 (SEQ ID NO: 32), C0021017 (SEQ ID NO: 22), or C0020065 (SEQ ID NO: 2).

In preferred embodiments, an antibody of the invention comprises a VL domain comprising a LCDR1 sequence of SEQ ID NO: 318, LCDR2 sequence of C0021158 fgl2 of SEQ ID NO: 319 and LCDR3 sequence of C0021158 fgl2 of SEQ ID NO: 320.

In a preferred embodiments, an antibody of the invention comprises a VL domain comprising a VL domain of C0021158 fgl2 of SEQ ID NO: 317 or an amino acid sequence with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 317.

In preferred embodiments, an antibody of the invention comprises a VL domain comprising a LCDR1 sequence of SEQ ID NO: 318, LCDR2 sequence of C0021158 fgl2 of SEQ ID NO: 319 and LCDR3 sequence of C0021158 fgl2 of SEQ ID NO: 320 and an amino acid sequence with at least 80%, 81%, 82%, 83%, 84%, 85%, 86%, 87%, 88%, 89%, 90%, 91%, 92%, 93%, 94%, 95%, at least 96%, at least 97%, at least 98%, or at least 99% sequence identity to the sequence of SEQ ID NO: 317.

In preferred embodiments, an antibody of the invention comprises a VL domain comprising a VL sequence of C0021158 fgl2 of SEQ ID NO: 317.

Preferably an antibody of the invention comprises a VL domain of C0021158fgl2 (SEQ ID NO: 317), C0021181 (SEQ ID NO: 347), C0021180 (SEQ ID NO:337), C0021177 (SEQ ID NO: 327), C0021158 (SEQ ID NO: 277), C0021158 IgG (SEQ ID NO: 287), C0021158fgl (SEQ ID NO: 307), C0021158dr (SEQ ID NO:297), C0021061 (SEQ ID NO: 67), C0020187 (SEQ ID NO: 17), C0021155 (SEQ ID NO: 267), C0021144 (SEQ ID NO: 257), C0021142 (SEQ ID NO: 237), C0021142 IgG (SEQ ID NO: 247), C0021141 (SEQ ID NO: 227), C0021139 (SEQ ID NO: 217), C0021135 (SEQ ID NO: 207), C0021133 (SEQ ID NO: 197), C0021131 (SEQ ID NO: 187), C0021129 (SEQ ID NO: 177), C0021128 (SEQ ID NO: 167), C0021124 (SEQ ID NO: 157), C0021118 (SEQ ID NO: 147), C0021101 (SEQ ID NO: 137), C0021098 (SEQ ID NO: 127), C0021097 (SEQ ID NO: 117), C0021096 (SEQ ID NO: 107), C0021092 (SEQ ID NO: 97), C0021089 (SEQ ID NO: 87), C0021065 (SEQ ID NO: 77), C0021032 (SEQ ID NO: 57), C0021022 (SEQ ID NO: 47), C0021021 (SEQ ID NO: 37), C0021017 (SEQ ID NO: 27), or C0020065 (SEQ ID NO: 7).

Preferably an antibody of the invention comprises:

a VH domain (SEQ ID NO: 312) and a VL domain (SEQ ID NO: 317) of C0021158 fgl2,

a VH domain (SEQ ID NO: 342) and a VL domain (SEQ ID NO: 347) of C0021181,

a VH domain (SEQ ID NO: 332) and a VL domain (SEQ ID NO: 337) of C0021180,

a VH domain (SEQ ID NO: 322) and a VL domain (SEQ ID NO: 327) of C0021177,

a VH domain (SEQ ID NO: 272) and a VL domain (SEQ ID NO: 277) of C0021158,

a VH domain (SEQ ID NO: 282) and a VL domain (SEQ ID NO: 287) of C0021158 IgG,

a VH domain (SEQ ID NO: 302) and a VL domain (SEQ ID NO: 307) of C0021158 fgl,

a VH domain (SEQ ID NO: 292) and a VL domain (SEQ ID NO: 297) of C0021158 dr,

a VH domain (SEQ ID NO: 62) and a VL domain (SEQ ID NO: 67) of C0021061,

a VH domain (SEQ ID NO: 12) and a VL domain (SEQ ID NO: 17) of C0020187,

a VH domain (SEQ ID NO: 262) and a VL domain (SEQ ID NO: 267) of C0021155,

a VH domain (SEQ ID NO: 252) and a VL domain (SEQ ID NO: 257) of C0021144,

a VH domain (SEQ ID NO: 232) and a VL domain (SEQ ID NO: 237) of C0021142,

a VH domain (SEQ ID NO: 242) and a VL domain (SEQ ID NO: 247) of C0021142 IgG,

a VH domain (SEQ ID NO: 222) and a VL domain (SEQ ID NO: 227) of C0021141,

a VH domain (SEQ ID NO: 212) and a VL domain (SEQ ID NO: 217) of C0021139,

a VH domain (SEQ ID NO: 202) and a VL domain (SEQ ID NO: 207) of C0021135,

a VH domain (SEQ ID NO: 192) and a VL domain (SEQ ID NO: 197) of C0021133,

a VH domain (SEQ ID NO: 182) and a VL domain (SEQ ID NO: 187) of C0021131,

a VH domain (SEQ ID NO: 172) and a VL domain (SEQ ID NO: 177) of C0021129,

a VH domain (SEQ ID NO: 162) and a VL domain (SEQ ID NO: 167) of C0021128,

a VH domain (SEQ ID NO: 152) and a VL domain (SEQ ID NO: 157) of C0021124,

a VH domain (SEQ ID NO: 142) and a VL domain (SEQ ID NO: 147) of C0021118,

a VH domain (SEQ ID NO: 132) and a VL domain (SEQ ID NO: 137) of C0021101,

a VH domain (SEQ ID NO: 122) and a VL domain (SEQ ID NO: 127) of C0021098,

a VH domain (SEQ ID NO: 112) and a VL domain (SEQ ID NO: 117) of C0021097,

a VH domain (SEQ ID NO: 102) and a VL domain (SEQ ID NO: 107) of C0021096,

a VH domain (SEQ ID NO: 92) and a VL domain (SEQ ID NO: 97) of C0021092,

a VH domain (SEQ ID NO: 82) and a VL domain (SEQ ID NO: 87) of C0021089,

a VH domain (SEQ ID NO: 72) and a VL domain (SEQ ID NO: 77) of C0021065,

a VH domain (SEQ ID NO: 52) and a VL domain (SEQ ID NO: 57) of C0021032,

a VH domain (SEQ ID NO: 42) and a VL domain (SEQ ID NO: 47) of C0021022,

a VH domain (SEQ ID NO: 32) and a VL domain (SEQ ID NO: 37) of C0021021,

a VH domain (SEQ ID NO: 22) and a VL domain (SEQ ID NO: 27) of C0021017, or

a VH domain (SEQ ID NO: 2) and a VL domain (SEQ ID NO: 7) of C0020065.

Sequence identity may be defined using the Bioedit, ClustalW algorithm (Thompson, J. D., et al. (1994). “CLUSTAL W: improving the sensitivity of progressive multiple sequence alignment through sequence weighting, position-specific gap penalties and weight matrix choice.” Nucleic Acids Res 22(22): 4673-4680).

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1 or IgG2. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 or IgG2 hinge region, or part thereof.

The sequence of the CH3 domain, is not particularly limited. Preferably, the CH3 domain is a human immunoglobulin gamma domain, such as a human IgG1, IgG2, IgG3, or IgG4 CH3 domain, most preferably a human IgG1 or IgG2 CH3 domain.

An antibody of the invention may comprise a human IgG1, IgG2, IgG3, or IgG4 constant region. The sequences of human IgG1, IgG2, IgG3, or IgG4 CH3 domains are known in the art. The constant region may be modified, e.g., to extend half-life. The Fc domain of an antibody of the invention may be a human IgG or a modified human IgG. The Fc domain may be modified to achieve the desired antibody function. Fc may be modified to increase or decrease effector function and/or to extend half-life of an antibody.

In preferred embodiments, the Fc domain is a modified Fc domain with enhanced effector function. The potency of antibodies may be increased by enhancement of their ability to mediate cellular cytotoxicity functions such as antibody-dependent cell mediated cytotoxicity (ADCC), antibody-dependent cell mediated phagocytosis (ADCP) and complement-dependent cytotoxicity (CDC). A number of Fc domain modifications have been identified that either directly or indirectly enhance binding of Fc receptors and through this significantly enhance cellular cytotoxicity, for example S239D/A330L/I332E (termed “3M”), F243L or G236A. Modifications of IgG1 that enhance ADCC include F243L/R292P/Y300L/V305I/P396L, S239D/I332E, S239D/A330L/I332E, S298A/E333A/K334A, or L234Y/L235Q/G236W/S239M/H268D/D270E/S298A in one heavy chain and D270E/K326D/A330M/K334E in the opposing heavy chain; modifications of IgG1 that enhance ACDP include G236A/S239D/I332E, modifications of IgG1 that enhance CDC include K326W/E333S or S267E/H268F/S324T. CDC (Increased C1q binding) may also be enhanced by using IgG1/IgG3 cross subclass Fc, or by hexamerisation using a modified IgG1 with E345R/E430G/S440Y.

An alternative approach to enhance effector function involves modified glycosylation of the Fc domain. FcγRs interact with the carbohydrates on the CH2 domain, the composition of the glycans has a substantial effect on effector function activity. Afucosylated (non-fucosylated) antibodies exhibit greatly enhanced ADCC activity through increased binding to FcγRIIIa. ADCC activity of glycosylated IgG1 antibodies is sensitive to the fucosylation status of the Fc glycan, with both in vitro and in vivo ADCC activity increased upon fucose removal (“afucosylation”). The effect of afucosylation on activity of IgG4 antibodies is less well characterized, but it has been shown to increase the in vitro ADCC activity of IgG4 antibody. Accordingly, antibodies of the invention may be afucosylated to enhance effector function.

Modification of the Fc of an antibody of the invention may be made to increase in vivo stability. The IgG4 sub-class undergoes Fab-arm exchange, where heavy chains can be swapped between IgG4 in vivo. The S228P mutation has been shown to prevent this recombination process allowing the design of less unpredictable therapeutic IgG4 antibodies.

IgG naturally persists for a prolonged period in serum due to FcRn-mediated recycling, giving it a typical half life of approximately 21 days. To prolong half life the pH-dependent interaction of the Fc domain with FcRn can be engineered to increase affinity at pH 6.0 while retaining minimal binding at pH 7.4. The mutations T250Q/M428L (“TM”), conferred an approximately 2-fold increase in IgG half-life in rhesus monkeys. The M252Y/S254T/T256E variant (“YTE”), conferred an approximately 4-fold increase in IgG half-life in cynomolgus monkeys. A longer half-life is desirable in some circumstances to decrease the frequency of administration whilst maintaining or improving efficacy of the administered antibody. Antibodies of the invention may be provided as half-life extended variants, engineered to extend half-life in vivo following administration, thus antibodies of the invention may be provided as M252Y/S254T/T256E or T250Q/M428L variants.

The invention also provides a nucleic acid or set of nucleic acids encoding an antibody or antigen-binding fragment of the invention, as well as a vector or vectors comprising such a nucleic acid or set of nucleic acids.

Where the nucleic acid encodes the VH and VL domain, or heavy and light chain, of an antibody molecule of the invention, the two domains or chains may be encoded on two separate nucleic acid molecules or on the same nucleic acid molecule.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g., phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NSO, or HEK cell, for example a HEK293 cell.

A recombinant host cell comprising a nucleic acid or the vector of the invention is also provided. Such a recombinant host cell may be used to produce an antibody of the invention. Thus, also provided is a method of producing an antibody of the invention, the method comprising culturing the recombinant host cell under conditions suitable for production of the antibody. The method may further comprise a step of isolating and/or purifying the antibody molecule.

Thus the invention provides a method of producing an antibody of the invention comprising expressing a nucleic acid encoding the antibody in a host cell and optionally isolating and/or purifying the antibody thus produced. Methods for culturing host cells are well-known in the art. Techniques for the purification of recombinant antibody are well-known in the art and include, for example HPLC, FPLC or affinity chromatography, e.g., using Protein A or Protein L. In some embodiments, purification may be performed using an affinity tag on antibody. The method may also comprise formulating the antibody into a composition with an excipient, such as a pharmaceutical composition with a pharmaceutically acceptable excipient.

The antibodies and compositions of the invention are expected to be useful in in therapeutic and diagnostic applications, in particular in humans for conditions associated with arginase activation and upregulation, such as cancer, immune cell dysfunction, infection, vascular disease, cardiovascular disease, endothelial dysfunction, ageing and cellular senescence, CNS disease and injury; diabetes-associated disease or cystic fibrosis, e.g., infection associated with cystic fibrosis.

The invention further provides an antibody of the invention, for use in a method of treatment. Also provided is a method of treating a patient, wherein the method comprises administering to the patient a therapeutically-effective amount of an antibody according to the invention. Further provided is the use of an antibody according to the invention for use in the manufacture of a medicament. A patient, as referred to herein, is preferably a human patient.

The invention also provides an antibody of the invention, for use in a method of treating cancer in a patient. Also provided is a method of treating cancer in a patient, wherein the method comprises administering to the patient a therapeutically-effective amount of an antibody according to the invention. Further provided is the use of an antibody molecule according to the invention for use in the manufacture of a medicament for the treatment of cancer in a patient.

In other aspects, the invention relates to an antibody that binds to and inhibits human ARG2 for use in:

a) treating cancer,

b) delaying progression of cancer,

c) prolonging the survival of a patient suffering from cancer,

d) stimulating a cell-mediated immune response, or

e) restoring or promoting T cell proliferation.

The invention also provides an antibody or composition of the invention, for use in a method of treating a disease in a patient. Also provided is a method of treating a disease in a patient, wherein the method comprises administering to the patient a therapeutically-effective amount of an antibody or composition according to the invention. Further provided is the use of an antibody according to the invention for use in the manufacture of a medicament for the treatment of a disease in a patient. In each instance, the disease may be a condition associated with ARG activation and upregulation, such as cancer, immune cell dysfunction, infection, vascular disease, cardiovascular disease, endothelial dysfunction, ageing and cellular senescence, CNS disease and injury; diabetes-associated disease or cystic fibrosis, e.g., infection associated with cystic fibrosis.

An antibody as described herein may be for use in a method of treatment of the human or animal body. Related aspects of the invention provide:

(i) an antibody molecule or composition thereof described herein for use as a medicament,

(ii) an antibody molecule or composition thereof described herein for use in a method of treatment of a disease or disorder,

(iii) the use of an antibody molecule or composition thereof described herein in the manufacture of a medicament for use in the treatment of a disease or disorder; and,

(iv) a method of treating a disease or disorder in an individual, wherein the method comprises administering to the individual a therapeutically effective amount of an antibody molecule or composition thereof as described herein.

The individual may be a patient, preferably a human patient. The term “patient” as used herein, may also refer to an animal, such as a mammal. Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e., prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

Whilst an antibody molecule may be administered alone, antibody molecules will usually be administered in the form of a pharmaceutical composition, which may comprise at least one component in addition to the antibody molecule. Another aspect of the invention therefore provides a pharmaceutical composition comprising an antibody molecule as described herein. A method comprising formulating an antibody molecule into a pharmaceutical composition is also provided.

Pharmaceutical compositions may comprise, in addition to the antibody molecule, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by parenteral, for example subcutaneous or intravenous administration, infusion, or any other suitable route.

Administration may be in a “therapeutically effective amount”, this being sufficient to show benefit to an individual. The actual amount administered, and rate and time-course of administration, will depend on the nature and severity of the disease being treated, the particular individual being treated, the clinical condition of the individual, the cause of the disorder, the site of delivery of the composition, the type of antibody molecule, the method of administration, the scheduling of administration and other factors known to medical practitioners. A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule.

In a preferred embodiment, an antibody molecule as described herein may be for use in treating cancer.

Cancer may be characterised by the abnormal proliferation of malignant cancer cells. Where a particular type of cancer, such as breast cancer, is referred to, this refers to an abnormal proliferation of malignant cells of the relevant tissue, such as breast tissue. A secondary cancer which is located in the breast but is the result of abnormal proliferation of malignant cells of another tissue, such as ovarian tissue, is not a breast cancer as referred to herein but an ovarian cancer.

The cancer may be a primary or a secondary cancer. Thus, an antibody molecule as described herein may be for use in a method of treating cancer in an individual, wherein the cancer is a primary tumour and/or a secondary cancer or tumour metastasis.

A tumour of a cancer to be treated using an antibody molecule as described herein may comprise cells that express ARG2. In one embodiment, the tumour may comprise cells that express ARG2. The tumour may comprise cells that express and secrete ARG2. Methods for identifying the expression and/or secretion of an antigen such as ARG2 are well known in the art.

The cancer to be treated using an antibody as described herein may be selected from the group consisting acute myeloid leukaemia (AML), osteosarcoma, HCMV-driven GBM, pancreatic cancer, head and neck squamous cell carcinoma, thyroid (Sousa et al., 2010), prostate (Mumenthaler et al., 2008), neuroblastoma (Mussai et al., 2015) and breast cancer (Polat et al., 2002).

In the context of cancer, treatment may involve activating or enhancing immune responses in an individual to improve the capacity of the individual to resist the cancer.

The antibody molecules of the invention may be useful in the detection of ARG2, thus, the present invention relates to the use of an antibody of the invention for detecting the presence of ARG2 in a sample. Also provided is an in vitro method of detecting ARG2, wherein the method comprises incubating an antibody of the invention with a sample of interest and detecting binding of the antibody to ARG2 within the sample. Binding of the antibody molecule may be detected using an ELISA, for example.

In a preferred embodiment, the present invention relates to an in vitro method of detecting ARG2 in a sample, wherein the method comprises incubating an antibody of the invention with a sample of interest, and determining binding of the antibody to ARG2 present in the sample, wherein binding of the antibody indicates the presence of ARG2 in the sample. Methods for detecting binding of an antibody molecule to its target antigen are known in the art and include ELISA and flow cytometry.

The sample of interest may be a sample obtained from an individual. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

The antibody molecules of the invention may thus be useful in the detection or diagnosis of disease or disorder, e.g., a condition associated with ARG activation and upregulation, such as cancer, immune cell dysfunction, infection, vascular disease, cardiovascular disease, endothelial dysfunction, ageing and cellular senescence, CNS disease and injury; diabetes-associated disease or cystic fibrosis, e.g., infection associated with cystic fibrosis. The antibody molecules of the invention may thus be useful in particular in the detection or diagnosis of cancer. The cancer may be a cancer which may be treated with an antibody molecule of the invention as described herein, e.g., acute myeloid leukaemia (AML), osteosarcoma, HCMV-driven GBM, pancreatic cancer, head and neck squamous cell carcinoma, thyroid, prostate, neuroblastoma or breast cancer.

Related aspects of the invention thus provide;

(i) an antibody molecule described herein for use in vitro as a diagnostic or in an in vitro diagnostic method,

(ii) an antibody molecule described herein for use in an in vitro method of detecting or diagnosing a disease or disorder, such as cancer,

(iii) the use of an antibody molecule described herein in the manufacture of a diagnostic product for use in the in vitro detection or diagnosis of a disease or disorder;

(iv) an in vitro method of detecting or diagnosing a disease or disorder in an individual; and

(v) a kit for use in an in vitro method of detecting or diagnosing a disease or disorder in an individual, the kit comprising an antibody molecule as described herein and optionally further comprising instructions for use and/or one or more reagents.

Further aspects and embodiments of the invention will be apparent to those skilled in the art given the present disclosure, including the following experimental exemplification.

All documents mentioned in this specification are incorporated herein by reference in their entirety.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments, which are described.

Certain aspects and embodiments of the invention will now be illustrated by way of example and with reference to the figures provided herein.

### BRIEF DESCRIPTION OF THE DRAWINGS

FIG. 1 shows the results of the direct binding HTRF™ assay between the purified scFv clones C0020065 and C0020187 and a series of arginase enzymes. FIG. 1A shows the specific binding to biotinylated recombinant human trimeric ARG2 enzyme. FIG. 1B shows that there is no binding to biotinylated recombinant human trimeric ARG1 enzyme. FIG. 1C shows the specific binding to biotinylated recombinant cynomolgus trimeric ARG2 enzyme. C0020065 (Δ) and C0020187 (x) both bound to recombinant human trimeric ARG2 enzyme (FIG. 1A) and to cynomolgus trimeric ARG2 enzyme (FIG. 1C) but showed no binding to recombinant human trimeric ARG1 enzyme (FIG. 1B). The negative control scFv antibody CEA6 (♦) showed no binding to any of the arginase enzymes, as expected.

FIG. 2 Inhibition of trimeric ARG2 by lead isolation scFv antibody fragments. FIG. 2 shows the results of the human trimeric ARG2 enzyme inhibition assay for the purified scFv clones C0020065 and C0020187 (n=4 C0020187, n=2 all other clones). Both clones C0020065 (Δ) and C0020187 (x) inhibited the activity of human trimeric ARG2. The negative control antibody CEA6 (♦) was unable to inhibit the activity of human trimeric ARG2, as expected. The small molecule arginase inhibitor NG-Hydroxy-L-arginine (NHLA) (◯) was included as a positive control and was able to inhibit the activity of human trimeric ARG2.

FIG. 3 Inhibition of trimeric Human ARG2 by lead isolation IgGs. FIG. 3 shows the results of the human trimeric ARG2 enzyme inhibition assay for the purified recombinant human IgG1 clones C0020065 and C0020187 (n=4 C0020187, n=2 all other clones). Both clones C0020065 (Δ) and C0020187 (x) inhibited the activity of human trimeric ARG2. The negative control antibody NIP228 (♦) was unable to inhibit the activity of human trimeric ARG2, as expected. The small molecule arginase inhibitor NG-Hydroxy-L-arginine (NHLA) (◯) was included as a positive control and was able to inhibit the activity of human trimeric ARG2.

FIG. 4 Restoration of T cell proliferation by ARG2 IgGs in the presence of ARG2. FIG. 4 shows the results of the T cell proliferation assay for the purified recombinant human IgG1 clones C0020065 and C0020187. Both clones inhibited the activity of human trimeric ARG2 (exogenous), leading to an increase in T cell proliferation. The negative control antibody NIP228 had no effect upon T cell proliferation, as expected. The small molecule arginase inhibitor NG-Hydroxy-L-arginine (NHLA) was included as a positive control and was able to inhibit the activity of human trimeric ARG2, giving an increase in T cell proliferation.

FIG. 5 Inhibition of ARG2 monomer by lead isolation IgGs. FIG. 5 shows the results of the human monomeric ARG2 enzyme inhibition assay for the purified recombinant human IgG1 clones C0020065 and C0020187 (n=3 C0020187, n=2 all other clones). Both clones C0020065 (Δ) and C0020187 (x) inhibited the activity of human monomeric ARG2. The negative control antibody NIP228 (♦) was unable to inhibit the activity of human monomeric ARG2 as expected. The small molecule arginase inhibitor NG-Hydroxy-L-arginine (NHLA) (∘) was included as a positive control and was able to inhibit the activity of human monomeric ARG2.

FIG. 6 Specific binding to THP1 derived ARG2 by lead isolation IgGs. FIG. 6 shows the results of the ELISA assay for binding of the biotinylated recombinant human IgG1 (huIgG1-biot) clones to THP-1 lysate derived human trimeric ARG2. Both clones C0020065 huIgG1-biot ( [symbol] ) and C0020187 huIgG1-biot ( [symbol] ) showed strong binding to THP-1 lysate derived human trimeric ARG2. No binding to lysates prepared from wild type THP-1 cells was observed. The negative control antibody NIP228 ( [symbol] ) did not bind to recombinant human trimeric ARG2 enzyme as expected.

FIG. 7 C0020187 has a not competitive mode of action on the homotrimer ARG2. FIG. 7 shows the results of a mode of action study for C0020187 recombinant human IgG1 to assess if the antibody is a competitive, non competitive or uncompetitive inhibitor. This study utilised the human trimeric ARG2 enzyme inhibition assay. 10 μM (∘), 3 μM ( [symbol] ), 1 μM (Δ) and 0.3 μM (▾) concentrations of C0020187 recombinant human IgG1 were incubated in the presence of increasing concentrations of the substrate arginine and the impact of each inhibitor concentration on ARG2 Kₘ and Vₘₐₓ assessed. A no inhibitor control (⋄) was also included. Increasing concentrations of C0020187 recombinant human IgG1 had a clear impact on arginine Vₘₐₓ values.

FIG. 8 Epitope competition assay for affinity-matured clones as scFv over parent C0020187 IgG FIG. 8 shows the results from an epitope competition HTRF™ assay in which inhibition of the formation of a complex between biotinylated recombinant human trimeric ARG2 enzyme and C0020187 recombinant human IgG1 by increasing concentrations of competing purified scFv antibodies was measured. All 22 scFv antibodies tested were derived from the process of affinity maturing C0020187. Parent C0020187 scFv was also included in this analysis alongside the negative control scFv antibody CEA6. See below for a key for all scFv antibodies tested. All affinity-matured scFv antibodies tested in this experiment showed improvements in IC₅₀ compared to parent C0020187 scFv.

Key:

| | | | | | | | | |
|---|---|---|---|---|---|---|---|---|
| [symbol] | C0021017 scFv | [symbol] | C0021089 scFv | [symbol] | C0021118 scFv | [symbol] | C0021135 | scFv |
| [symbol] | C0021021 scFv | [symbol] | C0021092 scFv | [symbol] | C0021124 scFv | [symbol] | C0021139 | scFv |
| [symbol] | C0021022 scFv | [symbol] | C0021096 scFv | [symbol] | C0021128 scFv | [symbol] | C0021141 | scFv |
| [symbol] | C0021032 scFv | [symbol] | C0021097 scFv | [symbol] | C0021129 scFv | [symbol] | C0021142 | scFv |
| [symbol] | C0021061 scFv | [symbol] | C0021098 scFv | [symbol] | C0021131 scFv | [symbol] | CEA6 | scFv |
| [symbol] | C0021065 scFv | [symbol] | C0021101 scFv | [symbol] | C0021133 scFv | [symbol] | C0020187 | scFv |

FIG. 9 Enzyme Inhibition assay data for affinity-matured clones as scFv vs parent C0020187 IgG. FIG. 9 shows the results of the human trimeric ARG2 enzyme inhibition assay for a panel of purified scFv antibodies derived from the process of affinity maturing C0020187. Parent C0020187 scFv was also included in this analysis alongside the negative control scFv antibody CEA6. See below for a key for all scFv antibodies tested. All affinity-matured scFv antibodies tested in this experiment showed improvements in IC₅₀ compared to parent C0020187 scFv.

Key:

| | | | | | | | | |
|---|---|---|---|---|---|---|---|---|
| [symbol] | C0021017 scFv | [symbol] | C0021089 scFv | [symbol] | C0021118 scFv | [symbol] | C0021135 | scFv |
| [symbol] | C0021021 scFv | [symbol] | C0021092 scFv | [symbol] | C0021124 scFv | [symbol] | C0021139 | scFv |
| [symbol] | C0021022 scFv | [symbol] | C0021096 scFv | [symbol] | C0021128 scFv | [symbol] | C0021141 | scFv |
| [symbol] | C0021032 scFv | [symbol] | C0021097 scFv | [symbol] | C0021129 scFv | [symbol] | C0021142 | scFv |
| [symbol] | C0021061 scFv | [symbol] | C0021098 scFv | [symbol] | C0021131 scFv | [symbol] | CEA6 | scFv |
| [symbol] | C0021065 scFv | [symbol] | C0021101 scFv | [symbol] | C0021133 scFv | [symbol] | C0020187 | scFv |

FIG. 10 Binding of IgGs to THP-1 derived human ARG2 in the presence of human plasma. FIG. 10 shows the results of the ELISA assay for binding of a panel of biotinylated recombinant human IgG1 (huIgG1-biot) clones to THP-1 lysate derived human trimeric ARG2. Binding to THP-1 lysate derived human trimeric ARG2 was tested in the presence and absence of 12.5% human plasma. See below for a key for all biotinylated recombinant human IgG1 tested. All clones showed strong binding to THP-1 lysate derived human trimeric ARG2 in the presence and absence of 12.5% human plasma. Binding signal was slightly reduced in the presence of 12.5% plasma for all clones tested. Binding to lysates prepared from wild type THP-1 cells was tested as a negative control and no binding was observed for any of the clones. Binding to recombinant human trimeric ARG2 (produced in house as described in example 1, section 1.1) was tested as a positive control and all clones showed strong binding. The negative control antibody R347 did not bind to THP-1 lysate derived human trimeric ARG2 or recombinant human trimeric ARG2 enzyme as expected.

Key:

[symbol] C0020187 hu IgG1—bio

[symbol] C0021022 hu IgG1—bio

[symbol] C0021061 hu IgG1—bio

[symbol] C0021065 hu IgG1—bio

[symbol] C0021092 hu IgG1—bio

[symbol] C0021133 hu IgG1—bio

[symbol] C0021139 hu IgG1—bio

[symbol] R347 hu IgG1—bio

[symbol] no IgG

FIG. 11 Inhibition of THP-1 derived human ARG2 activity by affinity-matured IgGs in the presence of human plasma. FIG. 11 shows the results of the THP-1 lysate derived human trimeric ARG2 enzyme inhibition assay for a panel of recombinant human IgG1 derived from the process of affinity maturing C0020187. See below for a key for all recombinant human IgG1 antibodies tested. All clones inhibited the activity of THP-1 lysate derived human trimeric ARG2 with improved IC₅₀ values compared to parent recombinant human IgG1 C0020187. The negative control antibody R347 was unable to inhibit the activity of THP-1 lysate derived human trimeric ARG2 as expected. The small molecule arginase inhibitor NG-Hydroxy-L-arginine (NHLA) was included as a positive control and was able to inhibit the activity of THP-1 lysate derived human trimeric ARG2.

Key:

| | | | |
|---|---|---|---|
| [symbol] | C0021022 | [symbol] | C0021133 |
| [symbol] | C0021061 | [symbol] | C0020187 |
| [symbol] | C0021065 | [symbol] | R347 |
| [symbol] | C0021092 | [symbol] | NHLA |
| [symbol] | C0021139 | | |

FIG. 12 Restoration of T cell Proliferation by affinity-matured IgGs in the presence of recombinant ARG2. FIG. 12 shows the results of the T cell proliferation assay for the purified recombinant human IgG1 clones C0020187 (Parent), C0021139, C0021133, C0021092, C0021065, C0021061 and C0021022. All clones inhibited the activity of human trimeric ARG2 (exogenous), leading to an increase in T cell proliferation. The negative control antibody R347 had no effect upon T cell proliferation as expected. 1^{st} generation affinity-matured IgG1 clones showed an improvement in potency compared to the parent IgG1 clone in the range of 7-10 fold.

FIG. 13 Inhibition of THP-1 derived human ARG2 activity by second generation affinity-matured IgGs. FIG. 13 shows the results of the THP-1 lysate derived human trimeric ARG2 enzyme inhibition assay for a panel of recombinant human IgG1 derived from the second generation affinity maturation process. See below for a key for all recombinant human IgG1 antibodies tested. All clones inhibited the activity of THP-1 lysate derived human trimeric ARG2 with improved IC₅₀ values compared to the first generation lead recombinant human IgG1 C0021061. The negative control antibody R347 was unable to inhibit the activity of THP-1 lysate derived human trimeric ARG2 as expected. The small molecule arginase inhibitor NG-Hydroxy-L-arginine (NHLA) was included as a positive control and was able to inhibit the activity of THP-1 lysate derived human trimeric ARG2.

Key:

| | | | |
|---|---|---|---|
| [symbol] | C0021144 IgG | [symbol] | C0021181 IgG |
| [symbol] | C0021158 IgG | [symbol] | C0021133 IgG |
| [symbol] | C0021180 IgG | [symbol] | C0021061 IgG (First generation lead) |
| [symbol] | C0021155 IgG | [symbol] | NHLA |
| [symbol] | C0021177 IgG | [symbol] | R347 IgG |

FIG. 14 Restoration of T cell Proliferation by second generation affinity-matured ARG2 specific IgGs in the presence of ARG2. FIG. 14 shows the results of the T cell proliferation assay for the purified recombinant human IgG1 clones C0020187 (Parent), C0021061, C0021158, C0021177, C0021180. All clones inhibited the activity of human trimeric ARG2 (exogenous), leading to an increase in T cell proliferation. The negative control antibody R347 had no effect upon T cell proliferation as expected. 2^{nd} generation affinity-matured IgG1 clones showed an improvement in potency compared to the parent IgG1, C0020187.

FIG. 15 Second generation affinity-matured lead C0021158 has a not competitive MOA. (A) C0021158 IgG—effect on ARG2 Vₘₐₓ and Kₘ. (B) Isothermal titration calorimetry of the ARG2 small-molecule inhibitor S-(2-Boronoethyl)-L-cysteine hydrochloride (BEC) with ARG2 only and ARG2-1158 fab complex, upper and lower panel, respectively. FIG. 15 shows that the 2nd generation affinity-matured lead C0021158 has a not competitive MOA. (A) C0021158 IgG—effect on ARG2 Vₘₐₓ and Kₘ shows the results of a mode of action study for C0021158 recombinant human IgG1 to assess whether the antibody is a competitive, non competitive or uncompetitive inhibitor. This study utilised the human trimeric ARG2 enzyme inhibition assay. 1 μM (∘), 100 nM (□), 30 nM (Δ) and 10 nM (∇) concentrations of C0020187 recombinant human IgG1 were incubated in the presence of increasing concentrations of the substrate arginine and the impact of each inhibitor concentration on ARG2 Kₘ and Vₘₐₓ assessed. A no inhibitor control (⋄) was also included. Increasing concentrations of C0021158 recombinant human IgG1 had a clear impact on arginine Vₘₐₓ values. The impact on arginine Kₘ was less clear. (B) Isothermal titration calorimetry of the ARG2 small-molecule inhibitor S-(2-Boronoethyl)-L-cysteine hydrochloride (BEC) with ARG2 only and ARG2-1158 fab complex, upper and lower panel, respectively. Upper panel: BEC at a concentration of 0.75 mM was titrated to a solution containing 20 μM of human ARG2 (monomer concentration). Data was fitted to a one-to-one binding model using a 68.3% confidence interval as error. Lower panel: BEC at a concentration of 0.50 mM was titrated to a solution containing 20 μM of human ARG2 (monomer concentration) and 20 μM of C0021158 fab. The presence of C0021158 fab prevents BEC from binding.

FIG. 16 Inhibition of THP-1 derived human ARG2 activity by second generation affinity-matured lead C0021158, and its germ-lined and de-risked form C0021158-fgl2, shows the results of the THP-1 lysate derived human trimeric ARG2 enzyme inhibition assay for recombinant human IgG1 C0021158 (□) and its germ-lined and de-risked form C0021158-fgl2 (Δ). The process of de-risking and germ-lining C0021158 had no impact on IC₅₀ value in this assay. Parent recombinant human IgG1 C0020187 and the small molecule arginase inhibitor NG-Hydroxy-L-arginine (NHLA) were included in this assay as positive controls and both were able to inhibit the activity of THP-1 lysate derived human trimeric ARG2 with expected IC₅₀ values.

FIG. 17 Immunoprecipitation (IP) of ARG2 from cell culture medium (CCM) of prostate, sarcoma and ovarian cancer cell line panels. ARG2 is detected in pull downs from LNCaP, A673 and A2780-Cis cell lines, demonstrating that these cell lines release ARG2 into CCM. PNT2 was derived from normal prostate epithelium. Cell viability values (%) determined prior to IP are also shown. This shows the results of immunoprecipitation of ARG2 from CCM of prostate, sarcoma and ovarian cell line panels. The blots show that ARG2 was detected in the pull downs from CCM of LNCaP, A673 and A2780-Cis cell lines. Each blot was validated by detection of recombinant ARG2 (positive control) and not recombinant ARG1 (negative control) in the pull downs. This result demonstrates that ARG2 is released into the CCM from LNCaP, A673 and A2780-Cis cell lines. A high cell viability count underneath the blots indicate that ARG2 is released from live cells.

FIG. 18 Binding of 1st generation IgG C0021061, 2nd generation IgG C0021158, PAD antibody (positive control) and irrelevant IgG control R347 (negative control) to ARG2 from CCM of LNCaP, A673 and A2780-Cis measured using ELISA. Each bar represents mean of absorbance (A460 nm) from duplicate samples+SD. This shows binding of 1^{st} generation IgG C0021061, 2^{nd} generation IgG C0021158 to ARG2 from CCM of LNCaP, A673 and A2780-Cis measured using ELISA. The ARG2 PAD antibody was included as a positive control and R347 as an irrelevant IgG negative control. The significantly high absorbance values of the three CCM samples compared to R347 and media only control clearly demonstrate binding of C0021061 and C0021158 to ARG2 from LNCaP, A673 and A2780-Cis cell lines.

FIG. 19 panels (A) and (B) show top and side views, respectively of the X-ray crystal structure of the ARG2 C0020187 Fab complex, Fabs are coloured light grey and ARG2 is coloured dark grey. (C) a view of the epitope:paratope interface within the ARG2 complex with C0020187 where ARG2 is shown in dark grey, bound Fabs in medium grey (bottom right corner of the panel) and the complex is overlaid with unbound ARG2 in light grey (derived from PDB entry: 4HZE) for the purposes of comparison.

FIG. 20 panels (A) and (B) show top and side views, respectively of the X-ray crystal structure of the ARG2 C0021158 Fab complex, in which Fabs are coloured light grey and ARG2 is coloured dark grey. (C) a view of the epitope:paratope interface within the ARG2 complex with C0021158 where ARG2 is shown in dark grey, bound Fabs in medium grey (bottom right corner of the panel) and the complex is overlaid with unbound ARG2 in light grey (derived from PDB entry: 4HZE) for the purposes of comparison.

FIG. 21 panels (A) and (B) show top and side views, respectively of the X-ray crystal structure of the ARG2 C0021181 Fab complex; in which Fabs are coloured light grey and ARG2 is coloured dark grey. (C) a view of the epitope:paratope interface within the ARG2 complex with C0021181 where ARG2 is shown in dark grey, bound Fabs in medium grey (bottom right corner of the panel) and the complex is overlaid with unbound ARG2 in light grey (derived from PDB entry: 4HZE) for the purposes of comparison.

FIG. 22 shows a view of the ARG2 active site from the X-ray crystal structure of the ARG2 C0021158 Fab complex. ARG2 amino acid side chains as well as key interactions are shown. Residues are numbered according to human ARG2 Uniprot ID: P78540.

FIG. 23 shows that Fab C0021139 occupies all three protomers in ARG2, whereas the Fab of the initial lead C0020187 binds only one ARG2 subunit with high affinity, as analysed by size-exclusion chromatography (A) and dynamic light scattering (B). (A) The size-exclusion profile of 1:1 molar mixtures of ARG2 and C0020187 Fab (empty circles) displays a peak at 150 kDa indicating binding of one fab (˜50 kDa) to one ARG2 trimer (˜120 kDa). In line with this, the ARG2-C0020187 Fab mixture contains considerable amounts of unbound fab (peak at ˜19.2 ml). The size-exclusion profile of 1:1 molar mixtures of ARG2 and LO C0021139 Fab (empty triangles) shows a peak at 275 kDa suggesting binding of three Fabs (˜150 kDa) to one ARG2 trimer (˜120 kDa). Accordingly, the ARG2-C0021139 Fab mixture contains minute amounts of unbound Fab. (B) Dynamic light scattering shows an increase in the average hydrodynamic radius (Rₕ) of ARG2-C0021139 Fab complexes (empty triangles) by 1.5 nm in comparison with Rₕ of ARG2-C0020187 Fab complexes (empty circles).

[symbol] ARG2

[symbol] C0020187 Fab

[symbol] C0021139 Fab

[symbol] ARG2:C0020187 Fab
1:1 mixture

[symbol] ARG2:C0021139 Fab
1:1 mixture

FIG. 24 Table 4 shows the Sequence identity across the entire VH sequence (Kabat residues 1 to 113) of the parental C0020187 antibody and the thirty-three affinity-matured antibodies described herein. All affinity-matured sequences share at least 90% identity with the parental C0020187 antibody. Percent diversity and percent divergence values derived from Clustal W method alignment (Thompson, Higgins et al. 1994).

FIG. 25 Table 5 shows the sequence identity across the entire VL sequence (Kabat residues 1 to 107) of the parental C0020187 antibody and the thirty-three affinity-matured antibodies described herein. All affinity-matured sequences share at least 89.1% identity with the parental C0020187 antibody. Percent diversity and percent divergence values derived from Clustal W method alignment (Thompson, Higgins et al. 1994).

FIG. 26 shows the heavy and light chain amino acid sequence alignments for clones C0020065 and C0020187.

FIG. 27 shows the heavy and light chain amino acid sequence alignments for clones C0020187, C0021017, C0021021, C0021022, C0021032, C0021061, C0021065, C0021089, C0021092, C0021096, C0021097, C0021098, C0021101, C0021118, C0021124, C0021128, C0021129, C0021131, C0021133, C0021135, C0021139, C0021141, C0021142, C0021142 IgG, C0021144, C0021155, C0021158, C0021158dr, C0021158fgl, C0021158 fgl2, C0021158 IgG, C0021177, C0021180 and C0021181.

### DESCRIPTION OF EMBODIMENTS

### Examples

### Example 1. Anti-ARG2 Specific Antibody Isolation and Lead Selection

1.1 Production of Trimeric Recombinant ARG2 and ARG1

Expression vectors compromising a synthetic DNA sequence encoding for human ARG2 (aa23-354), cynomolgus ARG2 (aa23-354) or human ARG1 (aa1-322) that had been codon optimised for expression in E. coli, plus flanking regions encoding for either N-terminal Avi tag (-AGLNDIFEAQKIEWHE- (SEQ ID NO: 351)) and/or C-terminal His10 tag. Amplified inserts (SEQ ID NO: 353: Avi-huARG2-His10, SEQ ID NO: 355: huARG2-His10 and SEQ ID NO: 352 Avi-huARG1-His10) containing the gene sequence of interest and tag(s) were cloned into pET16b vector (Novagen—EMD Millipore) using NcoI and XhoI restriction sites. The resulting vectors pAviHuArg2His10, pHuArg2His10 and pAviHuArg1His10 were transformed into BL21 (DE3) E. coli and a single transformed colony was used to inoculate a culture of 2×TYA, which was grown overnight at 37° C. at 280 rpm.

The overnight culture was used to inoculate 2×TYA cultures in 2 L shake flasks. Each culture was incubated at 37° C. at 280 rpm until an OD600 of 0.6. The cultures were then moved to an incubator at 18° C. before the addition of IPTG (0.5 M), the resulting cultures were then incubated overnight at 18° C. at 280 rpm.

Cultures were pelleted by centrifugation at 12,000 rpm for 20 minutes at room temperature. The supernatant was decanted off and discarded before the pellet was frozen overnight at −80° C. The resulting frozen pellet was thawed and processed for lysis using BugBuster™ Protein extraction reagent with the addition of lysonase bioprocessing reagent (Merck, 71370). Pellets were fully resuspended in BugBuster reagent at 4° C. before the addition of lysonase, after addition of lysonase the pellets were incubated at room temperature on a shaker platform at approximately 120 rpm. Lysis mixture was then pelleted by centrifugation at 20,000 rpm for 45 minutes at 4° C.

Soluble fractions were then decanted and combined into a clean bottle. Ni-NTA resin (pre-washed and equilibrated into tris 50 mM pH 8, 300 mM NaCl) was then added to supernatant and left to bind at 4° C. for 1 hour with gentle mixing. After binding, the Ni resin was filtered from the supernatant by gravity filtration through a Pierce Centrifuge column (ThermoFisher, 89898). The captured resin was washed (Ni wash buffer, tris 50 mM pH 8, 300 mM NaCl, 40 mM imidazole), and then eluted in Ni elution buffer (tris 50 mM pH 8, 300 mM NaCl, 400 mM imidazole).

The elution fraction was further purified on the Hiload 16 60 superdex 200 prep grade SEC column on the AKTAxpress by manual injection into a 5 ml loop. Column was equilibrated in and elution was performed with Tris glycerol storage buffer (25 mM tris pH8, 150 mM NaCl, 10% glycerol) and 1.5 ml fractions were collected across the entire volume of the elution. Fractions containing observable peaks were pooled and analysed by SDS-PAGE and peaks were compared to molecular weight markers to establish trimeric and monomeric fraction(s).

1.2 Production of Monomeric Recombinant ARG2

ARG2 sequence within pAviHuARG2His10, pHuArg2His10 vectors were modified by site directed mutagenesis such that amino acid 275 was mutated from a glutamic acid to a glutamine (arginase residue number is based on human ARG2 Uniprot ID: P78540). These modified vectors were then used to express monomeric ARG2 from E. coli following the protocol used to express trimeric ARG2 as described in section 1.1.

1.3 Phage Display Selections to Isolate ARG2 Specific scFvs

Soluble phage display selections were performed using five naïve libraries (nFL, DP47, CS, BMV and EG3) cloned into a phagemid vector based on the filamentous phage M13 (Vaughan et al., 1996). Anti-ARG2 scFv antibodies were isolated from the phage display libraries using a series of selection cycles on recombinant human biotinylated ARG2 (avi-Arg2-His10, made in house) essentially as previously described (Hawkins et al., 1992; Vaughan et al., 1996). In brief, for the first round of solution-phase selections, biotinylated human ARG2 in Dulbecco's phosphate buffered saline (DPBS, pH 7) was added (final concentration of 100 nM biotinylated human ARG2) to purified phage particles that had been pre-incubated for 1 hour in Marvel-PBS (3% w/v) containing Streptavidin-coupled paramagnetic beads (Dynabeads® M280, Invitrogen Life Sciences, UK). Streptavidin beads were removed prior to addition of antigen. Phage particles that bound to the biotinylated human ARG2 were captured using new Streptavidin-coupled paramagnetic beads, and weakly-bound phage were removed by a series of wash cycles using PBS-Tween (0.1% v/v). Bound phage particles were eluted from the beads using Trypsin (10 ng/ml final concentration diluted in 0.1 M sodium phosphate buffer; pH 7), infected into E. coli TG1 bacteria and rescued for the next round of selection (Vaughan et al., 1996). Two subsequent rounds of selection were carried out as previously described but with a reduced concentration of biotinylated ARG2 antigen, specifically 50 nM and 25 nM of biotinylated human ARG2 at round 2 and round 3, respectively.

1.4 Identification of ARG2 Specific scFv Fragments Using a Direct-Binding Assay (Unpurified scFv)

Unpurified scFv from periplasmic preparations were screened in a homogeneous time-resolved fluorescence (HTRF™, CisBio Bioassays, France) binding assay using a Pherastar plate reader (BMG Labtech, Germany). In this assay, binding of unpurified scFv to recombinant human trimeric ARG2 enzyme was assessed by measuring the fluorescence resonance energy transfer (FRET) between the c-myc tagged scFv and the biotinylated enzyme using streptavidin cryptate and anti-c-myc-XL665 detection reagents (CisBio International, France; cat: 610SAKLB and 61 MYCXLB respectively). Selection outputs were screened as unpurified bacterial periplasmic extracts containing scFv, prepared in 200 mM tris buffer pH 7.4, 0.5 mM EDTA and 0.5 M sucrose. 5 μl of unpurified scFv samples were added to a Greiner® 384 well assay plate (Greiner Bio-one, UK; cat: 784076). This was followed by the addition of 5 μl of 12 nM recombinant human trimeric ARG2 enzyme, 5 μl of 6.67 nM streptavidin cryptate and 5 μl 40 nM anti-c-myc-XL665. Non-specific binding wells (negative controls) were defined for each plate by using a negative control unpurified scFv in place of the test scFv sample. Cross-reactive scFv clones were identified using a concurrent assay with recombinant human trimeric ARG1 enzyme. All dilutions were performed in phosphate buffered saline (ThermoFisher Scientific, UK; cat: 14190-094) containing 0.4 M KF (VWR, UK; cat: 26820.236) and 0.1% bovine serum albumin (Sigma, UK; cat: A9576) (assay buffer). Assay plates were incubated overnight at 4° C. prior to reading time resolved fluorescence on a Pherastar plate reader (PerkinElmer, USA) using an excitation wavelength of 320 nm and measuring the emission at 620 nm and 665 nm (100 flashes).

Data were analysed by calculating % Delta F values for each sample. % Delta F was determined according to equation 1.

$$\% Delta F = \frac{\begin{matrix}( sample 665 nm / 620 nm ratio ) - \\ ( negative control 665 nm / 620 nm ratio )\end{matrix}}{( negative control 665 nm / 620 nm ratio )} \times 100 \qquad Equation 1$$

1.5 Identification of ARG2 Inhibitory scFv Using an Enzyme Inhibition Assay (Unpurified scFv)

Unpurified anti-ARG2 scFv fragments from periplasmic preparations were screened in an ARG2 enzyme inhibition assay using a Pherastar plate reader (BMG Labtech, Germany). In this assay, the ability of unpurified scFv to inhibit the enzymatic activity of trimeric ARG2 was determined by measuring the production of urea, the product of L-arginine turnover by ARG2.

Selection outputs were screened as unpurified bacterial periplasmic extracts containing scFv, prepared in 200 mM TES buffer pH 7.4, 0.5 mM EDTA and 0.5 M sucrose. 10 μl of unpurified scFv were added to a Greiner Bio-One® 384 well assay plate (Greiner Bio-one, UK; cat: 781901). This was followed by the addition of 5 μl of 1.6 μg/ml trimeric human ARG2 that had been diluted in 40 mM MnCl₂, 100 mM tris-HCl, pH7.4 (4× enzyme buffer).

Following an overnight pre-incubation of ARG2 and unpurified scFv at 4° C., the assay plate was brought to room temperature and the enzymatic reaction was started with the addition of 5 μl 100 mM L-arginine substrate and incubated for 60 min at room temperature.

Urea was detected by the simultaneous addition of equal volumes of O-Phthaldialdehyde (1.5 mM O-Phthaldialdehyde, 7% H₂SO₄, 0.03% Brij L23) and N-(1-Napthyl)ethylenediamine (1.66 mM N-(1-Napthyl)ethylenediamine, 81 mM Boric acid, 21% H₂SO₄, 0.03% Brij L23) followed by an 18 minute incubation at room temperature before the colorimetric product was quantified on the Pherastar by measuring absorbance at 505 nm.

Background control wells were defined for each plate by the omission of substrate. Maximum signal control wells were defined for each plate by using CEA6, an irrelevant unpurified scFv in place of the test scFv sample.

Data were analysed by calculating % maximum (max) control for each sample using the 505 nm absorbance data. % max control was determined according to equation 2.

% max control=100*((Test sample−mean maximum control)/(mean background control−mean maximum control)) Equation 2:

1.6 Identification of ARG2 Specific scFv Using a Direct Binding Assay (Purified scFv)

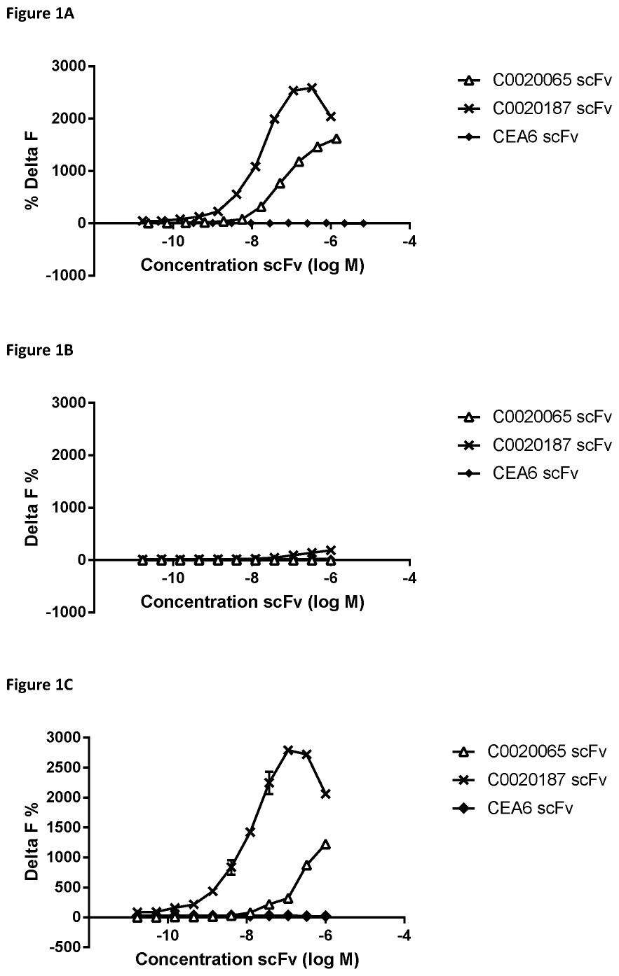

Unpurified scFv periplasm extracts that showed specific binding to biotinylated recombinant human trimeric ARG2 enzyme by HTRF™ assay were subjected to DNA sequencing (Osbourn et al., 1996; Vaughan et al., 1996). The scFv with unique protein sequences were expressed in E. coli and purified by affinity chromatography (essentially as described (Bannister et al., 2006)). The ARG2 binding profile of each purified scFv was determined by testing a dilution series of the purified scFv in the HTRF™ assay described in section 1.4, substituting the unpurified scFv periplasmic preparation with the purified scFv. The purified scFv were tested concurrently for binding to biotinylated recombinant human trimeric ARG2 enzyme, biotinylated recombinant human trimeric ARG1 enzyme and recombinant cynomolgus monkey trimeric ARG2 enzyme. Data were analysed by calculating the % Delta F values as described in section 1.4. Example results for purified C0020187 scFv and purified C0020065 scFv are shown in FIG. 1. These results demonstrate that C0020187 scFv and C0020065 scFv specifically bind to biotinylated recombinant human trimeric ARG2 enzyme and biotinylated recombinant cynomolgus monkey trimeric ARG2 enzyme and not to human recombinant human trimeric ARG1 enzyme.

1.7 Screening for Purified scFv Fragments which Inhibit ARG2 in an Enzyme Inhibition Assay

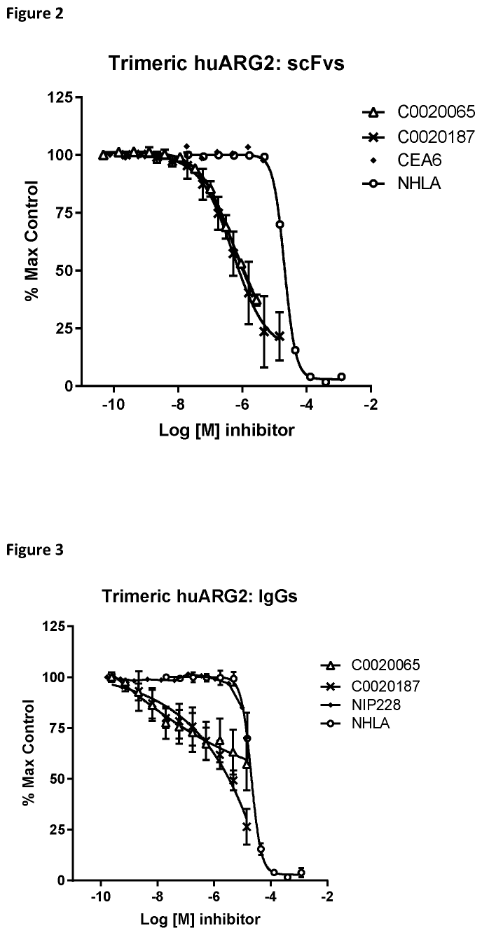

Purified anti-ARG2 scFv fragments were screened for their ability to inhibit enzymatic activity of trimeric ARG2 (FIG. 2) using the assay described in section 1.5.

IC₅₀ values were determined by testing a dilution series of the purified scFv, prepared in TBS, in the ARG2 enzyme inhibition assay described in section 1.5, substituting the unpurified scFv periplasmic preparation with the purified scFv.

Background control wells were defined for each plate by the omission of substrate. Maximum signal control wells were defined for each plate in wells, which contained substrate in the absence of inhibitor. Data were analysed by calculating % maximum (max) control for each sample. % max control was determined according to equation 1 detailed in section 1.5.

Purified scFv were tested alongside N-Hydroxyl-L-Arginine (a small molecule competitive inhibitor of ARG2) as a positive control and CEA6 (irrelevant isotype control antibody) as a negative control. Data points were plotted and IC₅₀s determined using a four-parameter fit in GraphPad Prism.

1.8 Reformatting of ARG2 Specific, Inhibitory Antibodies from scFv to IgG,

Specific clones were converted from scFv to IgG₁ by sub-cloning the variable heavy chain (VH) and variable light chain (VL) domains into vectors expressing whole human antibody heavy and light chains respectively. The variable heavy chains were cloned into a mammalian expression vector (pEU 1.3) containing the human heavy chain constant domains and regulatory elements to express whole IgG₁ heavy chain in mammalian cells. Similarly, the variable light chain domain was cloned into a mammalian expression vector for the expression of the human lambda light chain constant domains (pEU4.4) or human lambda kappa light chain constant domains (pEU3.4) and regulatory elements to express whole IgG light chain in mammalian cells. Vectors for the expression of heavy chains and light chains were originally described in Persic et al., 1997. To obtain clones as IgG₁, the heavy and light chain IgG expression vectors were transiently transfected into ExpiCHO (ThermoScientific UK; cat. number: A29133) cells where the antibody was expressed and secreted into the medium. Harvested media was filtered prior to purification. The IgGs were purified using Protein A chromatography (MabSelect SuRe, GE Healthcare, UK). Culture supernatants were loaded onto an appropriate Protein A column pre-equilibrated in 25 mM tris pH 7.4, 50 mM NaCl. Bound IgG was eluted from the column using 0.1 M Sodium Citrate pH 3.0, 100 mM NaCl. The IgGs were buffer exchanged into TBS 25 mM tris pH 7.4, 150 mM NaCl). The purified IgGs were passed through a 0.2 μm filter and the concentration of IgG was determined by absorbance at 280 nm using an extinction coefficient based on the amino acid sequence of the IgG. The purified IgGs were analysed for aggregation or degradation using SEC-HPLC and SDS-PAGE techniques.

1.9 Confirming Binding Profile of ARG2 Specific Recombinant IgG, to ARG2 in a Direct Binding Assay

The purified scFv fragments that bound specifically to biotinylated recombinant human trimeric ARG2 enzyme were converted to recombinant IgG. Binding of recombinant IgG to human trimeric ARG2 enzyme was confirmed by testing a dilution series of the recombinant IgG in the HTRF™ assay described in section 1.4, substituting the unpurified scFv with the recombinant IgG. A further modification to the assay was the substitution of anti-c-myc-XL665 detection reagent with anti-human Fc XL665 detection reagent (CisBio International, France; cat: 61 HFCXLB) as the recombinant IgG had no c-myc tag. Data were analysed by calculating the % Delta F values as described in section 1.4.

1.10 Confirming the Trimeric ARG2 Inhibition Profile of ARG2 Specific Recombinant IgG, in an Enzyme Inhibition Assay

The purified scFv fragments that inhibited the enzymatic activity of human trimeric ARG2 enzyme were converted to recombinant IgG. ARG2 specific recombinant IgG₁ were screened for their ability to inhibit enzymatic activity of trimeric ARG2 (FIG. 3) using the assay described in section 1.5.

IC₅₀ values were determined by testing a dilution series of the ARG2 specific recombinant IgG₁, prepared in TBS, in the ARG2 enzyme inhibition assay described in section 1.5, substituting the unpurified scFv periplasmic preparation with the ARG2 specific recombinant IgG₁.

ARG2 specific recombinant IgG₁ were tested alongside N-Hydroxyl-L-Arginine (a small molecule competitive inhibitor of ARG2) as a positive control and NIP228 (irrelevant isotype control antibody) as a negative control. Data points were plotted and IC₅₀ values determined using a four parameter fit in GraphPad Prism.

1.11 Restoration of T Cell Proliferation by ARG2 IgGs

Anti-ARG2 antibodies were screened for their ability to restore T cell proliferation by inhibiting enzymatic activity of ARG2 and hence, prevent the depletion of the L-arginine, which is required for T cell proliferation. This assay served to provide in vitro evidence of functional efficacy for each antibody.

Briefly, Leukocyte cones (NHS Blood and Transplant, Watford, UK) underwent Ficoll Plaque (GE, Sweden; Cat: 17-1440-02) gradient separation, PBMC's were extracted and pan T cell population isolated by negative selection using the Human T Cell Enrichment Kit (Stemcell Technologies, France; Cat: 19051) according to the manufacturer's instructions.

40000 cells/well were seeded in complete growth media (RMPI 1640, 5% Human Serum Albumin) into a 96 well clear TC treated microplate (Greiner-Bio One, Germany; Cat: 655180). T Cells were activated by the addition of ImmunoCult CD3/CD28 (Stemcell Technologies, France; Cat: 10971) at a dilution of 1:40 directly into wells and the plate incubated 37° C., 5% CO₂ for 45 minutes.

To determine EC₅₀'s of test antibodies, a dilution series was created in sterile TBS at 8× the final assay concentration was added to the assay plate containing cells. Plate was incubated for 30 minutes at 37° C., 5% CO₂.

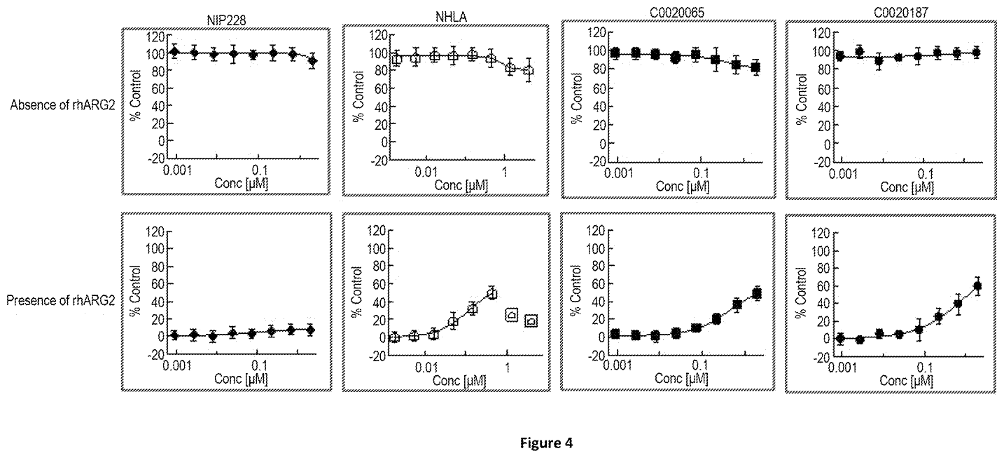

ARG2 was added to relevant wells of the plate at a final concentration of 15 μg/ml. The equivalent volume of ARG2 vehicle and test antibody vehicle was added to relevant control wells. The total volume in each well was 200 μL. Cells were treated with test antibodies for a total of 72 hours at 37° C., 5% CO₂. Antibodies were tested alongside N-Hydroxyl-L-Arginine (a small molecule competitive inhibitor of ARG2) as a positive control and NIP228 (irrelevant isotype control antibody) as a negative control (FIG. 4).

Assessment of T cell proliferation was performed using the BrdU Cell Proliferation Kit (Merck Millipore, UK; Cat: QIA58). BrdU labelling reagent was added after 54 hours of antibody treatment and the plate further incubated for 18 hours at 37° C., 5% CO₂. The remainder of the assay was performed according to the manufacturer's instructions.

Calculated data was expressed as % of control (Equation 3) and graphical plots were constructed using a four parameter fit and EC₅₀'s determined using model 205 in XLFit (IDBS, UK).

All test sample data was corrected for background (mean of no cell control)

% Control=100*((Test Sample−Mean Positive Control)/(Mean Negative Control−Mean Positive Control)) Equation 3:

Mean Positive Control=T Cells in Absence of ARG2

Mean Negative Control=T Cells in Presence of ARG2

TABLE 8
Assessment of T cell proliferation (Figure 4)

| | | | In Presence of rhARG2 | | | In Absence of rhARG2 | | |
|---|---|---|---|---|---|---|---|---|
| | | Top | Maximum % | Maximum % | | Maximum % | Maximum % | |
| Clone ID | Format | Conc | Proliferation | Proliferation | EC₅₀ | Proliferation | Proliferation | EC₅₀ |
| NHLA | SMI | 4 mM | 49* | 8 | ND | 80 | 13 | <[0.0018 mM] |
| NIP228 | hlgG1 | 2 μM | 7 | 7 | >[2 μM] | 90 | 9 | <[0.0003 μM] |
| C0020065 | hlgG1 | 2 μM | 49 | 8 | >[2 μM] | 82 | 9 | <[0.0008 μM] |
| C0020187 | hlgG1 | 2 μM | 59 | 11 | 0.971 | 98 | 6 | <[0.0008 μM] |

*Graphical plot of NHLA had the top two concentration data points excluded, the maximum % proliferation value corresponds to a concentration of 0.444 mM. ND = Not Determined. SMI = Small Molecule Inhibitor. C0020187 n = 2, C0020065 n = 4, NHLA n = 3, NIP228 n = 4.

This report is independent research. NHS Blood & Transport have provided material in support of the research. The views expressed in this publication are those of the author(s) and not necessarily those of NHS Blood & Transport.

1.12 Confirming the Monomeric ARG2 Inhibition Profile of ARG2 Specific Recombinant IgG₁ in an Enzyme Inhibition Assay

ARG2 specific recombinant IgG₁ were screened for their ability to inhibit enzymatic activity of monomeric ARG2. In this assay, the ability of ARG2 specific recombinant IgG₁ to inhibit the enzymatic activity of monomeric ARG2 was determined by measuring the production of urea, the product of L-arginine turnover by monomeric ARG2.

IC₅₀ values were determined by testing a dilution series of the ARG2 specific recombinant IgG₁, prepared in TBS.

10 μl of ARG2 specific recombinant IgG₁ (dilution series) were added to a Greiner Bio-One® 384 well assay plate (Greiner Bio-one, UK; cat: 781901). This was followed by the addition of 5 μl of 1.6 μg/ml monomeric ARG2 that had been diluted in 40 mM MnCl₂, 100 mM tris-HCl, pH7.4 (4× enzyme buffer).

Following an overnight pre-incubation of monomeric ARG2 and ARG2 specific IgG₁ at 4° C., the assay plate was brought to room temperature and the enzymatic reaction was started with the addition of 5 μl 100 mM L-arginine substrate and incubated for 60 min at room temperature.

Urea was detected by the simultaneous addition of equal volumes of O-Phthaldialdehyde (1.5 mM O-Phthaldialdehyde, 7% H₂SO₄, 0.03% Brij L23) and N-(1-Napthyl)ethylenediamine (1.66 mM N-(1-Napthyl)ethylenediamine, 81 mM Boric acid, 21% H₂SO₄, 0.03% Brij L23) followed by an 18 minute incubation at room temperature before the colorimetric product was quantified on the Pherastar by measuring absorbance at 505 nm.

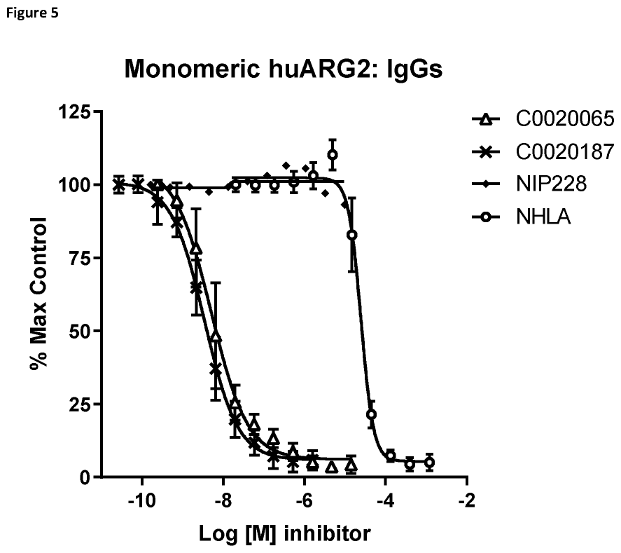

ARG2 specific recombinant IgG₁ were tested alongside N-Hydroxyl-L-Arginine (a small molecule competitive inhibitor of ARG2) as a positive control and NIP228 (irrelevant isotype control antibody) as a negative control. Data points were plotted and IC₅₀ values determined using a four parameter fit in GraphPad Prism (FIG. 5).

1.13 Binding Human ARG2 Produced by THP1 Cells with IgG1

C0020187 and C0020065 IgGs were tested for their ability to bind to mammalian expressed human ARG2 produced in transfected THP-1 cells. Lysates of human ARG2 transfected THP-1 cells were prepared and binding to these lysates measured using an ELISA.

Cell lysates were prepared from THP-1 cells overexpressing human ARG2 and from wild type THP-1 cells. Between 600,000-1,000,000 cells were suspended in 1 ml tris-buffered saline (25 mM tris+150 mM sodium chloride in dH₂O, pH 7.4) and cell lysates prepared using QIAshredders (QIAgen, UK; cat: 79654).

For the ELISA, maxisorp white plates (Nunc; cat: 437796) were coated overnight at 4° C. with rabbit anti-human ARG2 antibody (Abcam, ab137069) at 1 μg/ml in PBS. The next day, the plate was washed with PBS and blocked with casein blocker (Thermo Scientific, 37528).

Prior to addition to the ELISA plate, cell lysates were pre-incubated for 45 minutes (with gentle shaking at room temperature) with biotinylated versions of C0020187 and C0020065 lead antibodies. Antibodies were at a final concentration of 50 μg/ml in the pre-incubation mixtures.

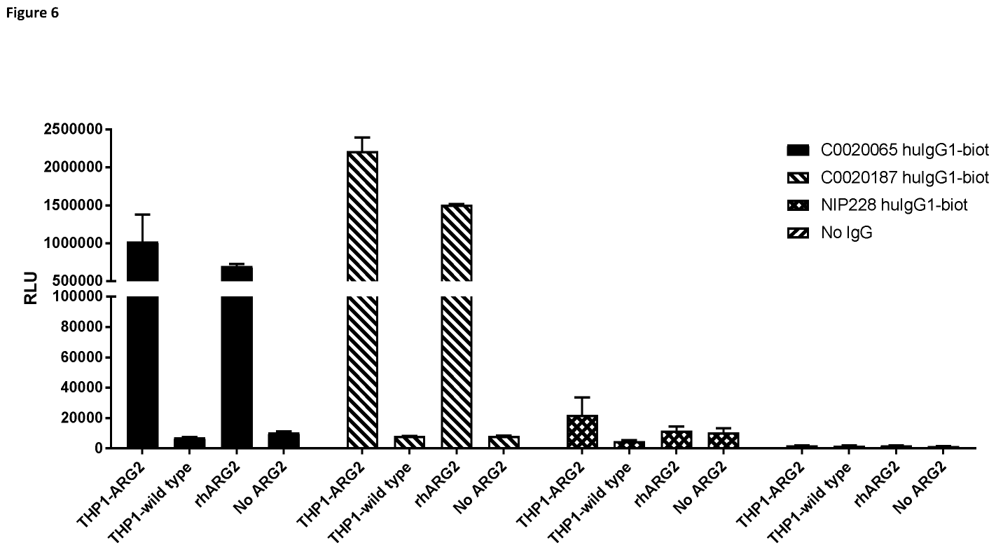

The blocked ELISA plate was washed with PBS and 50 μl of the pre-incubation mixtures were transferred to the ELISA plate and incubated for 90 minutes at room temperature. The ELISA plate was washed five times with PBS-Tween (0.1%) and 50 μl 1/30000 streptavidin-HRP was added to each well. The plate was incubated at room temperature for 1 hour before being washed 6 times with PBS-Tween (0.1%). Next, 100 μl PICO ELISA substrate (Thermo Scientific, 37070) was added to each well, the plate was incubated for 1 minute at room temperature in the dark before being read for luminescence using the Envision plate reader. Data were analysed using GraphPad Prism. Results are shown in FIG. 6.

1.14 Generation of Fab Fragments to Enable Kinetic Profiling of Improved Variants.

The heavy chain mammalian expression vector pEU1.3 was modified using the Q5 SDM kit (New England Biolabs E0554) to remove the human heavy chain constant domains CH2 and CH3 and generate a vector only containing the CH1 domain and part of the Hinge region. The resulting vector is called pEU1.3 fab. Vectors for the expression of heavy chains and light chains were originally described in Persic et al., 1997. Specific clones were converted from scFv to Fab by sub-cloning the variable heavy chain (VH) and variable light chain (VL) domains into vectors expressing human fab heavy and light chains respectively. To obtain clones as IgG1, the heavy and light chain fab expression vectors were transiently transfected into ExpiCHO (ThermoScientific UK; cat. number: A29133) cells where the Fab fragment was expressed and secreted into the medium. Harvested media was filtered prior to purification. The Fabs were purified using IgG1-CH1 chromatography (CaptureSelect™ IgG-CH1 Pre-packed Column, ThermoFisher). Culture supernatants were loaded onto an appropriate CaptureSelect IgG-CH1 column pre equilibrated in 1×DPBS. Bound Fab fragments was eluted from the column using 0.1 M glycine pH 3.0. The Fab fragments were buffer exchanged into TBS (25 mM tris pH 7.4, 150 mM NaCl). The purified Fab fragments were passed through a 0.2 μm filter and the concentration of the Fabs was determined by absorbance at 280 nm using an extinction coefficient based on the amino acid sequence of the Fabs. The purified Fab fragments were analysed for aggregation or degradation using SEC-HPLC and SDS-PAGE techniques.

1.15 Determination of Binding Affinities of Lead Antibody C0020187 to Human ARG2, Cynomolgus ARG2 and Human ARG1 Using Bio-Layer Interferometry

The OctetRED (Pall ForteBio) instrument was used to assess the kinetic parameters of the interactions between C0020187 and recombinantly produced human ARG2, cynomolgus ARG2 and human ARG1. The Octet biosensor uses an optical analytical technique that analyses the interference pattern of white light reflected from two surfaces: a layer of immobilised protein on the sensor tip, and an internal reference layer. Any changes in binding at the biosensor tip result in a shift in interference pattern, which can be measured in real time. Molecules associating with or dissociating from ligands at the biosensor tip shift the interference pattern and generate a response on the Octet system which is recorded by the acquisition software.

Typically, a defined concentration of the analyte species is brought into contact with the coupled ligand and any binding is detected as an increased in signal (association phase). This is followed by a period of buffer rinse, during which dissociation of the analyte species from the surface immobilised ligand can be observed as a decrease in signal (dissociation phase). Repetition of this with a range of analyte concentrations provides data for the analysis of binding kinetics.

An Octet Kinetics Buffer (PBS containing 0.01% BSA and 0.002% Tween20) is typically used as the diluent buffer for the analyte samples and as the flow buffer during the dissociation phase. The experimental data is recorded over time as shift in interference pattern (nm) over time, which is directly proportional to the optical thickness at the biosensor tip, which in turn is an approximate measure of the mass of analyte bound. The proprietary Octet Data Analysis software package can then be used to process data and fit binding models to the data sets. Returned association (kₐ, M⁻¹ s⁻¹) and dissociation (k_{d}, s⁻¹) rate constants allow calculation of dissociation (K_{D}, M) affinity constants.

The affinity of binding between the Fab of C0020187 and human ARG2, cynomolgus ARG2 and human ARG1 was estimated using assays in which the biotinylated ARG antigen was captured on a streptavidin sensor tip. A fresh sensor tip was used for each measurement and no regeneration was used. A series of dilutions of the C0020187 Fab (3.25-240 nM) were individually placed in contact with the ligand surface for a sufficient amount of time to observe sensorgrams that could be fitted to an appropriate binding model with confidence (typically 5 minutes), followed by an appropriate length of dissociation time (typically 10 minutes). Blank reference (0 nM Fab) data were subtracted from each dataset to reduce the impact of any buffer artefacts or non-specific binding effects. An appropriate binding model was then fitted simultaneously to the data from each analyte titration using the Octet Evaluation software.

Example results for C0020187 are shown in Table 9, showing the association rate constants (kₐ), dissociation rate constants (k_{d}) and dissociation constants (K_{D}). These parameters were derived from a 1:1 binding fit to the data.

TABLE 9
Affinity Data for C0020187 and ARG2 interaction

| | | K_{D} | K_{D} | kₐ | kₐ | k_{d} | k_{d} |
|---|---|---|---|---|---|---|---|
| Ligand | Analyte | (M) | Error | (1/Ms) | Error | (1/s) | Error |
| Human | C0020187 | 8.35E−09 | 9.57E−11 | 6.92E+04 | 6.20E+02 | 5.77E−04 | 4.14E−06 |
| ARG2 | Fab | | | | | | |
| Cyno | C0020187 | 6.26E−09 | 7.07E−11 | 1.01E+05 | 8.62E+02 | 6.31E−04 | 4.66E−06 |
| ARG2 | Fab | | | | | | |

| | | |
|---|---|---|
| Human | C0020187 | No binding |
| ARG1 | Fab | |

1.16 Understanding the Mechanism of Action of ARG2 Specific IgGs on the Enzyme ARG2

The mechanism of action of the lead identification clone C0020187 IgG was investigated using the human ARG2 (trimer) enzyme inhibition assay.

The enzyme inhibition assay is described in sections 1.5 and is a biochemical assay that measures production of urea, the product of L-arginine turnover by ARG2. To investigate the mechanism of action of C0020187 IgG, four point titrations of C0020187 IgG (10, 3, 1 and 0.3 μM) were set up in the presence of 11 concentrations of arginine (500, 400, 300, 200, 125, 75, 50, 25, 17, 10 and 5 mM) and the reaction allowed to proceed for 80, 60, 45, 30, 15 or 5 minutes before the addition of the urea detection reagents O-Phthaldialdehyde and N-(1-Napthyl)ethylenediamine.

Raw data from each separate concentration of C0020187 was plotted as a function of time for each concentration of arginine and fitted using a linear fit (GraphPad Prism) to derive initial velocities (slope values).

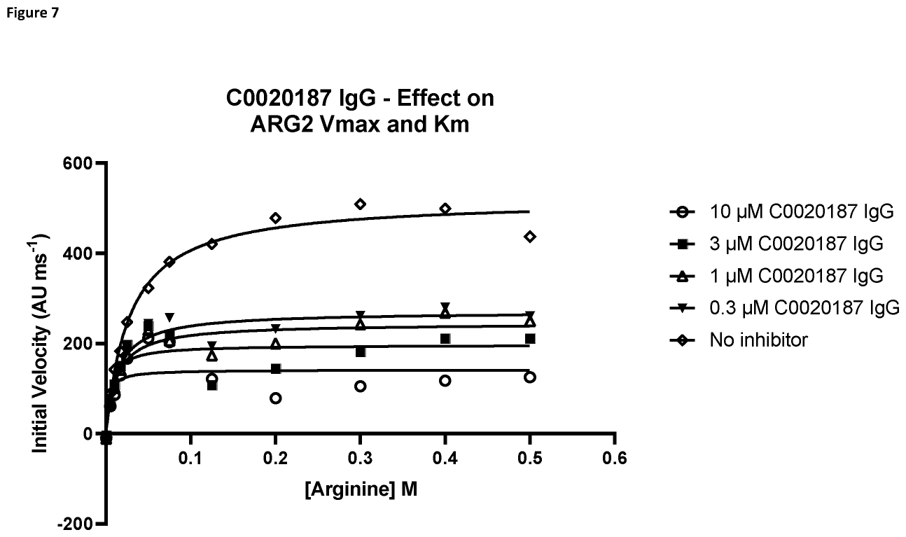

Initial velocity data was plotted against arginine concentration for each concentration of C0020187 IgG see FIG. 7. Each trace was fitted to the Michaelis-Menten model (Y=Vₘₐₓ*X/(Kₘ+X)) in GraphPad Prism to derive maximum velocity (Vₘₐₓ) and enzyme/substrate affinity (Kₙ) values at each inhibitor concentration (Table 10).

TABLE 10

| | Vₘₐₓ (AU ms⁻¹) | Kₘ (mM) | R square |
|---|---|---|---|
| 10 μM C0020187 IgG | 141.4 | 3.16 | 0.5218 |
| 3 μM C0020187 IgG | 196.4 | 5.59 | 0.7072 |
| 1 μM C0020187 IgG | 244.2 | 12.31 | 0.9222 |
| 0.3 μM C0020187 IgG | 270.2 | 13.36 | 0.9360 |
| No inhibitor | 519.8 | 27.97 | 0.9841 |

The mechanism of action of enzyme inhibitors can be broadly divided into competitive, uncompetitive and non-competitive and can be identified as such based on their effects on Kₘ and Vₘₐₓ.

A competitive inhibitor binds only to free enzyme, often at the active site, causing the affinity of the enzyme for the substrate to be reduced (increased Kₘ) with no change to the maximum enzyme rate (Vₘₐₓ).

An uncompetitive inhibitor binds exclusively to the enzyme-substrate complex yielding an inactive enzyme-substrate-inhibitor complex. The enzyme's affinity for the substrate is increased (Kₘ is decreased) and the maximum enzyme rate (Vₘₐₓ) is decreased.

A non-competitive inhibitor binds equally well to free enzyme and enzyme-substrate complex. The enzyme's affinity for the substrate (Kₘ) is unchanged and the maximum enzyme rate (Vₘₐₓ) is decreased.

In the case of C0020187 IgG inhibitor it Vₘₐₓ decreased in the presence of C0020187 IgG inhibitor, ruling out a competitive mode of action and pointing towards an uncompetitive or a non-competitive mode of action. To distinguish non-competitive from uncompetitive modes of action the effect on Kₘ must be assessed. Unfortunately, the initial velocity/arginine concentration plots had an unusual profile where, in the presence of inhibitor, the initial anticipated increase in initial velocity is followed by a drop as arginine concentration increases. This uncharacteristic profile lead to poor fitting of the data making it difficult to derive an accurate Kₘ. The lack of accurate Kₘ data means C0020187 IgG cannot be assigned as uncompetitive or non-competitive. It is only possible to state that the inhibitor is ‘not competitive’.

### Example 2—Affinity Maturation of C0020187 by Targeted and StEP SHUFFLE Mutagenesis Using Phage and Ribosome Display

2.1 Targeted Mutagenesis of Antibody C0020187 by Phage Display

The lead antibody C0020187 was optimised for improved affinity to human ARG2 using a targeted mutagenesis approach with affinity-based phage display selections. Twelve large scFv-phage libraries derived from C0020187 were created by oligonucleotide-directed mutagenesis of the variable heavy (V_{H}) and variable light (V_{L}) chain complementarity determining regions 1, 2 and 3 (CDR1, CDR2, CDR3) using standard molecular biology techniques as described by Clackson and Lowman ((2004) A Practical Approach, Oxford University Press).

Libraries were subjected to affinity-based phage display selections to enrich for variants with higher affinity for human ARG2. The selections were performed essentially as described previously (Hawkins et al., 1992; Schier et al., 1996; Thompson et al., 1996). In brief, the scFv phage particles were incubated with biotinylated human ARG2 (avi-Arg2-His10, made in house) in solution. ScFv-phage that bound to the antigen were then captured on streptavidin-coated paramagnetic beads (Dynabeads® M280, Invitrogen Life Sciences, UK) following the manufacturer's recommendations. The selected scFv-phage particles were then rescued as described previously (Osbourn et al., 1996). The selection process was repeated for five successive rounds, in the presence of decreasing concentrations of biotinylated human ARG2 target antigen (30 nM antigen at round 1 falling to 10 pM antigen by round 5).

2.2 Recombination of Successful Selection Outputs to Produce “Binary” Libraries, and their Subsequent Affinity Optimisation

Collectively, screening of C0020187 CDR-targeted mutagenesis library outputs in the C0020187 epitope competition (Section 2.4) and human ARG2 enzyme inhibition assay (Section 1.5) enabled identification of individual CDR-targeted phage display selection output libraries displaying overall improvements in huARG2 direct binding and inhibition, relative to parent C0020187 scFv. Specifically, four targeted mutagenesis libraries, (covering the V_{H}CDR1, V_{H}CDR2, V_{L}CDR2 and V_{L}CDR3), were recombined in a pair-wise fashion to produce four “binary” recombination libraries in which two of the six CDRs were mutated. For example, the affinity-matured library covering the V_{H}CDR1 was randomly recombined with the affinity-matured V_{L}CDR2 library to generate a V_{H}1:V_{L}2 library. The remaining libraries were produced as: V_{H}1:V_{L}3, V_{H}2:V_{L}2 and V_{H}2:V_{L}3. A subset of each recombination library was sequenced to verify the integrity of each library.

Soluble phage display selections utilising these recombination libraries were completed as previously described (Section 1.3) in the presence of decreasing concentrations of biotinylated human ARG2 (avi-Arg2-His10, made in house; 1 nM decreasing to 5 pM over the course of three successive rounds of selection). As before, all recombination library selection outputs were screened in the C0020187 epitope competition (Section 2.4) and human ARG2 enzyme inhibition assay (Section 1.5)

2.3 Recombination of Phage-Optimised CDR Blocks & Selections Using Ribosome Display

Ribosome display is described in Hanes J and Plückthun A. (1997).

Modest (approx. 8-fold) improvements in C0020187's affinity were achieved using the single CDR targeting and subsequent binary CDR recombination approaches described in Section 2.1. To drive for further improvements in C0020187 affinity, two additional strategies were followed, in parallel. The first strategy corresponded to a targeted CDR recombinatorial approach focusing on two CDRs: V_{H}CDR2 and V_{L}CDR2. The second strategy was a non-biased approach recombining outputs from all six CDRs. This second non-biased strategy enabled the entire scFv sequence space to be sampled, enabling both intra and inter-chain recombination events (Zhao et al., 1998) that would not be possible using standard targeted (biased) recombinatorial approaches.

Recombinatorial PCR was used to combine the phage-optimised V_{H}CDR2 and V_{L}CDR2 repertoires into a single population of full-length scFv clones, to generate a V_{H}CDR2/V_{L}CDR2 recombination library (H2L2). For the non-biased recombination library, the phage-optimised outputs from V_{H}CDR1, V_{H}CDR2, V_{H}CDR3, V_{L}CDR1, V_{L}CDR2 and V_{L}CDR3 were used as template for V_{H}/V_{L} chain shuffling by recombinatorial PCR (Shuffle library). The resulting library was also subjected to random recombination using the Staggered-extension process (StEP) in vitro DNA recombination (Zhao et al., 1998) to promote additional intra-chain recombinations (StEP library).

The libraries of recombined scFv constructs were then modified into the ribosome display format using standard molecular biology methods. The ribosome display construct include the structural features necessary for ribosome display, including a 5′ and 3′ stem loop to prevent degradation of the mRNA transcript by exonucleases, a Shine-Dalgarno sequence to promote ribosome binding to the mRNA transcript, and a geneIII spacer that allows the translated scFv molecule to fold while still remaining attached to the ribosome (Groves et al., 2005).

The libraries were then used in affinity-based soluble ribosome display selections to enrich for variants with higher affinity for human ARG2. The selections were performed essentially as described in Hanes et al., 2000. In brief, each recombination library was individually transcribed into mRNA. Using a process of stalled translation, mRNA-ribosome-scFv tertiary complexes were formed (Hanes et al., 1997). These complexes were then subjected to three rounds of selection incubated in the presence of decreasing concentrations of synthetic biotinylated human ARG2 to select for variants with higher affinity. Those complexes that bound to the antigen were then captured on streptavidin-coated paramagnetic beads (Dynabeads™, Invitrogen, UK; cat: 112-05D) and non-specific ribosome complexes were washed away. The mRNA was subsequently isolated from the bound ribosomal complexes, reverse transcribed to cDNA and then amplified by PCR. This DNA was used for the next round of selection.

After affinity maturation, the selection outputs were cloned out for screening purposes. The scFv isolated by ribosome display were cloned into the phagemid vector pCANTAB6 by NotI/NcoI restriction endonuclease digestion of the ribosome display construct (New England BioLabs, USA; cat: R0189L, R0193L) followed by ligation into NotI/NcoI digested pCANTAB6 using T4 DNA ligase (New England BioLabs, USA; cat: M0202L) essentially as described by McCafferty et al., 1994.

2.4 Identification of Improved Clones from Phage and Ribosome Display Using an Epitope Competition Assay

Two thousand and twenty four scFv chosen at random from selection rounds 3 and 4 of the targeted mutagenesis approach described in section 2.2, 2.3 and 2.4 were expressed in E. coli to produce unpurified periplasmic scFv. Those scFv capable of competing with C0020187 IgG for binding to recombinant human trimeric ARG2 enzyme (i.e., those scFv that bind at or near the epitope bound by C0020187 IgG) were elucidated in a competition format assay, using the HTRF™ platform. Specifically, fluorescence resonance energy transfer (FRET) was measured between streptavidin cryptate (associated with biotinylated recombinant human trimeric ARG2 enzyme) and anti-human Fc XL665 (associated with C0020187 IgG) in the presence of a single concentration of each unpurified periplasmic test scFv. Successful occupation of the C0020187 IgG epitope on the enzyme by scFv resulted in a reduction in FRET, as measured on a fluorescence plate reader.

A ‘Maximum’ binding signal was determined by analysing the binding of C0020187 IgG to biotinylated recombinant human trimeric ARG2 enzyme in the absence of competitor scFv. The ‘Sample’ signals were derived from analysing the binding of C0020187 IgG to biotinylated recombinant human trimeric ARG2 enzyme in the presence of a test scFv sample. Finally, a ‘Background’ signal was determined by analysing the fluorescence generated in the absence of C0020187 IgG.

Unpurified periplasmic scFv were supplied in sample buffer consisting of 200 mM tris base, pH 7.4, 0.5 mM EDTA, and 0.5 M sucrose. 5 μl of each scFv were transferred to the ‘Sample’ wells of a black, shallow, solid bottom, non-binding 384-well assay plate using a liquid handling robot. The remaining reagents (prepared in assay buffer) were added to the assay plate by multichannel pipette in the following order: 5 μl detection cocktail, consisting of 6.6 nM streptavidin cryptate and 40 nM anti-human Fc XL665 (to all wells), 5 μl 12 nM biotinylated recombinant human trimeric ARG2 enzyme (to all wells), 5 μl 16 nM C0020187 IgG (to ‘Sample’ and ‘Maximum’ wells), and 5 μl sample buffer (to background wells). Assay plates were sealed and then incubated overnight at room temperature in the dark, prior to measuring time-resolved fluorescence at 620 and 665 nm emission wavelengths on a fluorescence plate reader.

Data were analysed by calculating % Delta F values for each sample. % Delta F was determined according to equation 1.

$$\% Delta F = \frac{\begin{matrix}( sample 665 nm / 620 nm ratio ) - \\ ( negative control 665 nm / 620 nm ratio )\end{matrix}}{( negative control 665 nm / 620 nm ratio )} \times 100 \qquad Equation 1$$

Delta F values were subsequently used to calculate normalised binding values as described in equation 4.

$$Normalised data ( \% Control ) = \frac{\% Delta F of sample}{\% Delta F of Maximum binding control} \times 100 \qquad Equation 4$$

Unpurified periplasmic scFv demonstrating significant inhibition of C0020187 IgG binding to biotinylated recombinant human trimeric ARG2 enzyme were subjected to DNA sequencing (Osbourn et al., 1996; Vaughan et al., 1996). The scFv found to have unique protein sequences were expressed in E. coli and purified by affinity chromatography followed by buffer exchange.

The potency of each purified scFv was determined by testing a dilution series of the scFv (typically 4 pM-1200 nM) in the epitope competition assay described above. Data were again analysed by calculating the % Delta F and % Control binding values for each sample.

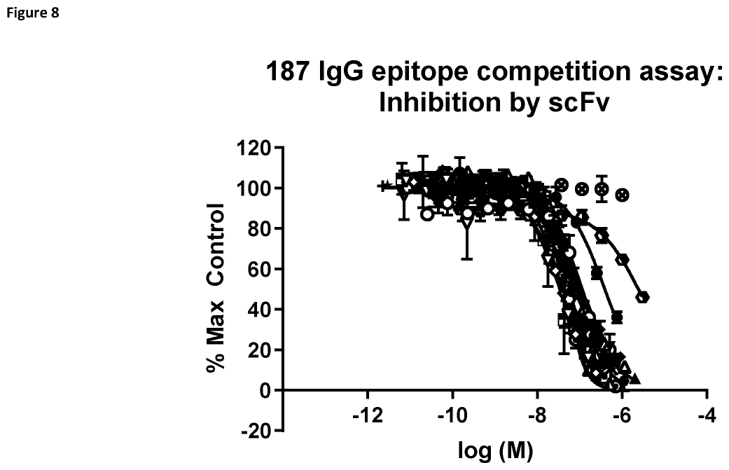

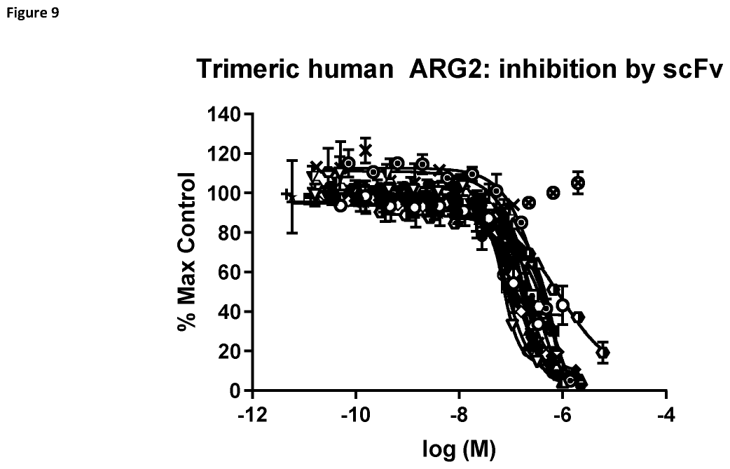

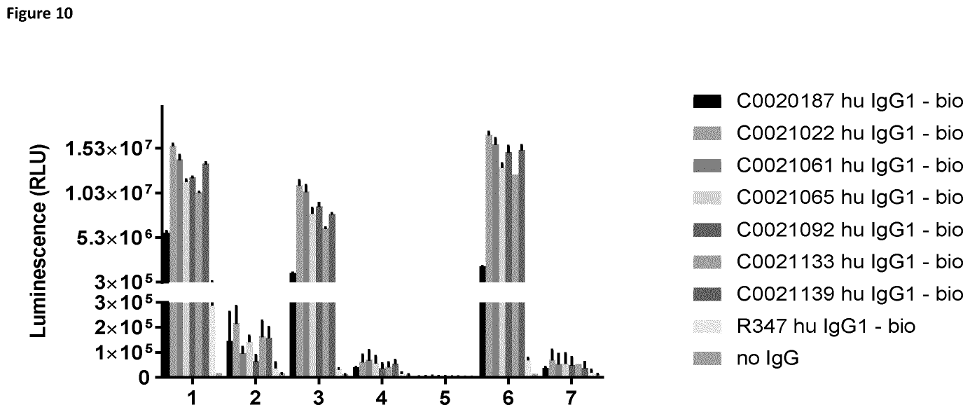

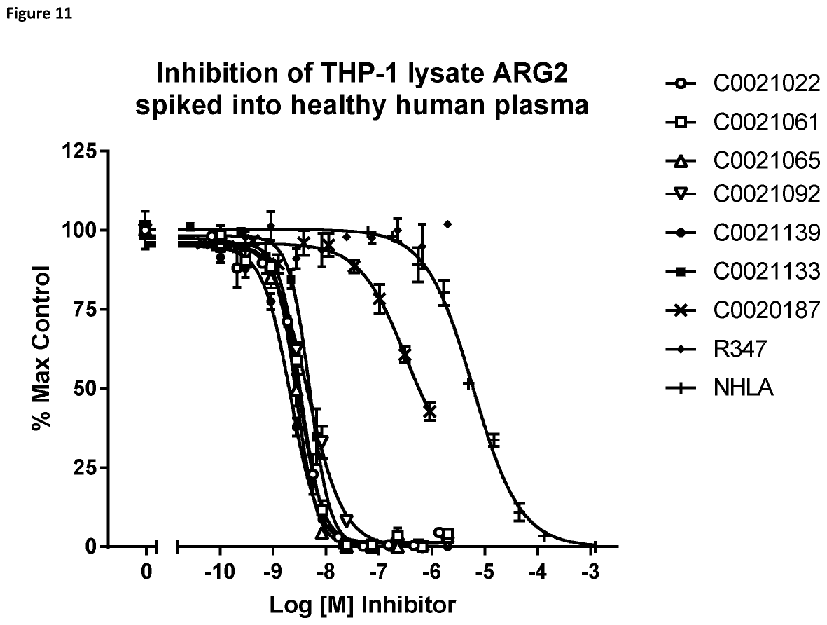

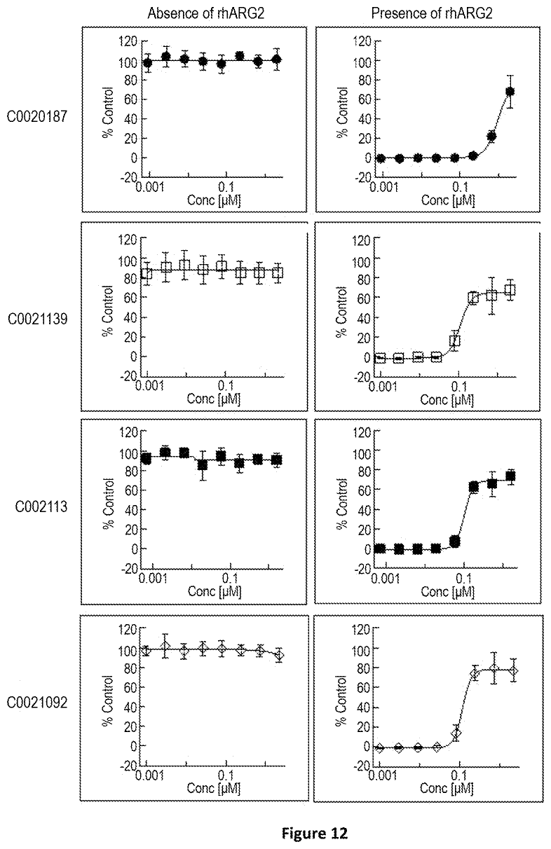

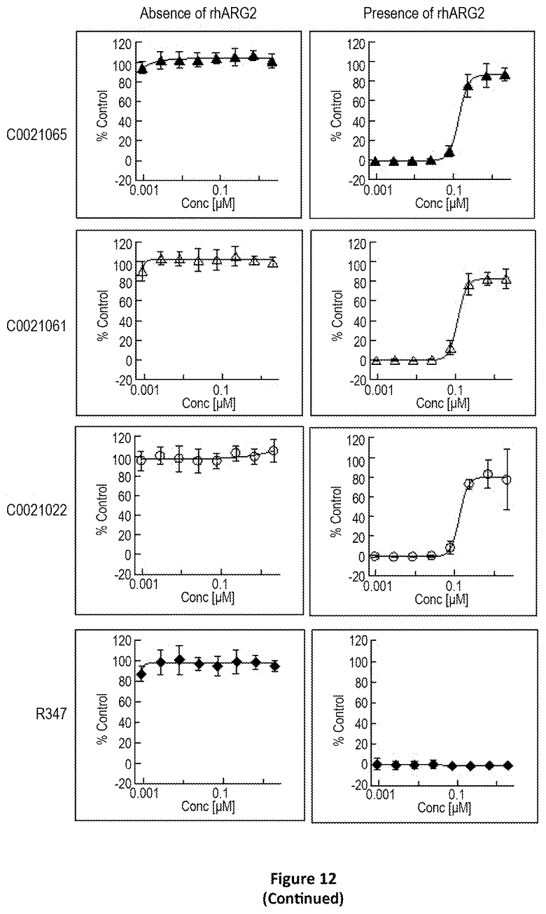

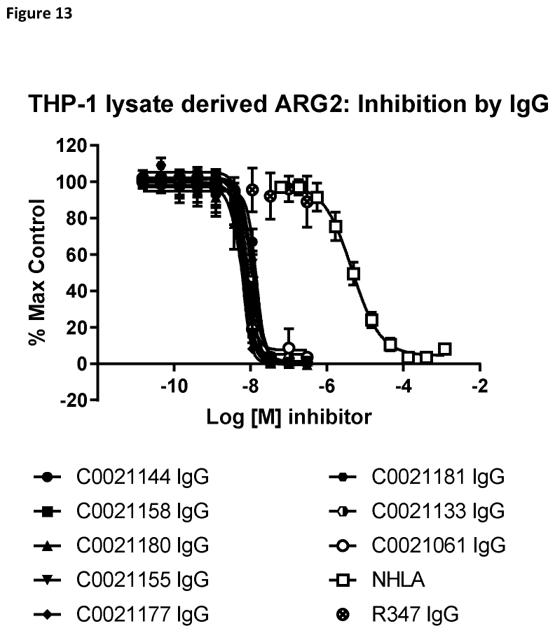

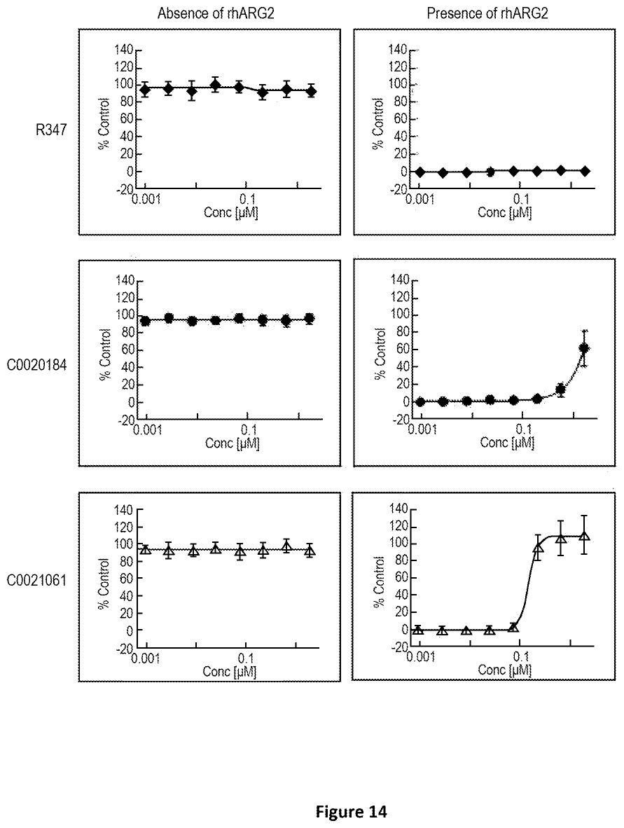

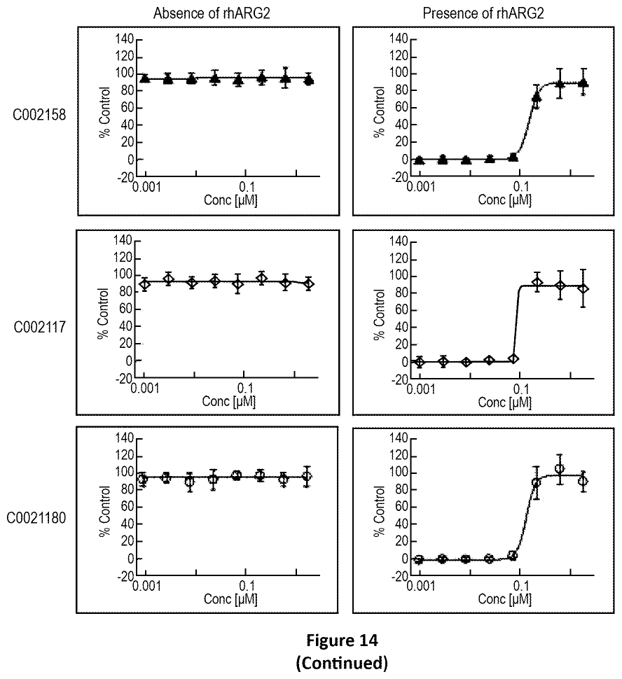

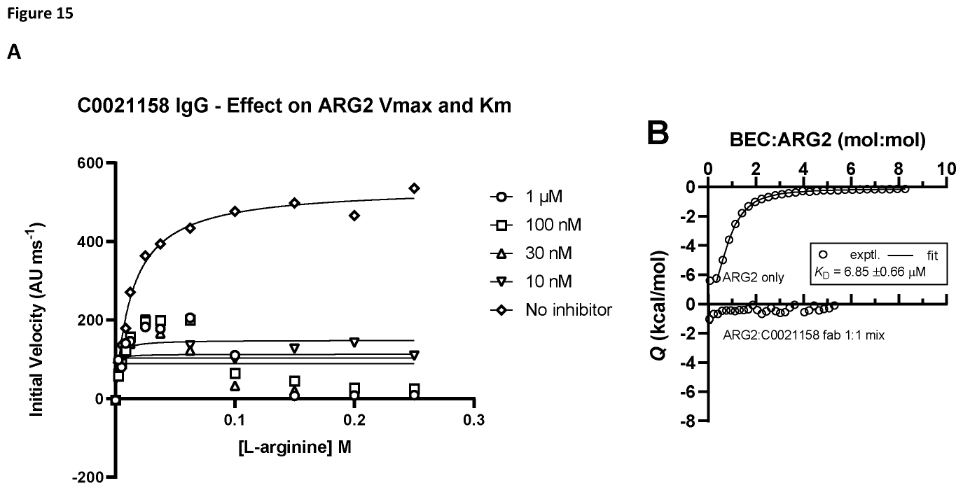

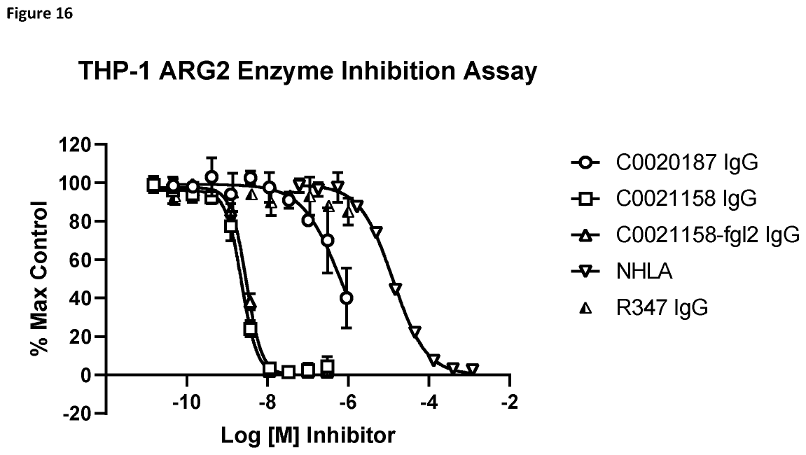

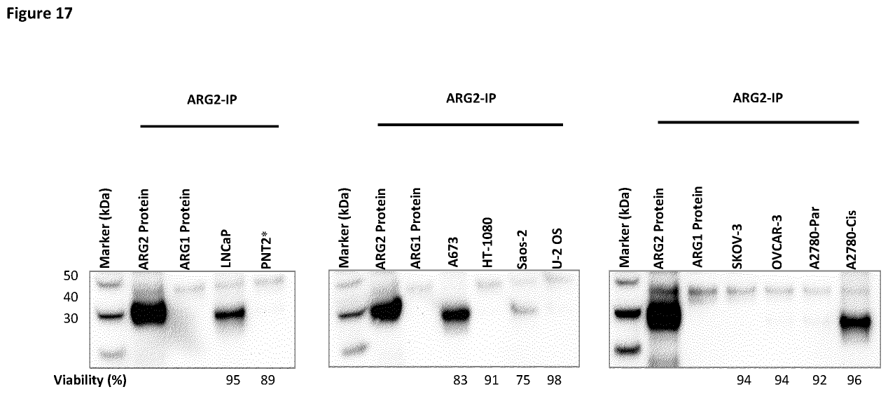

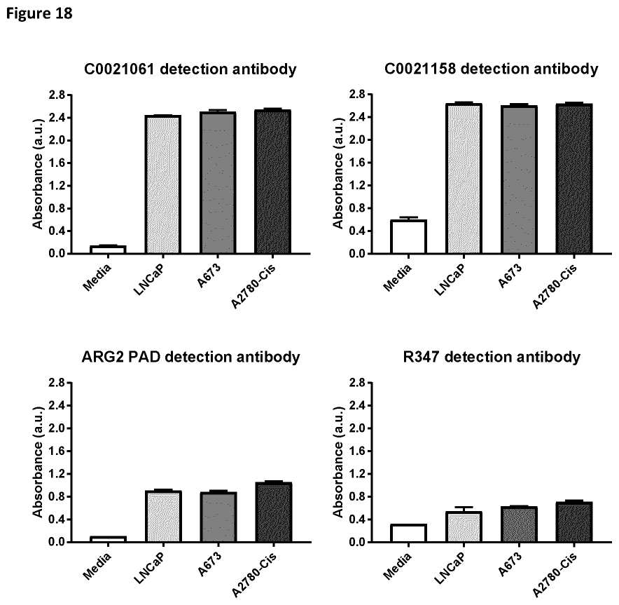

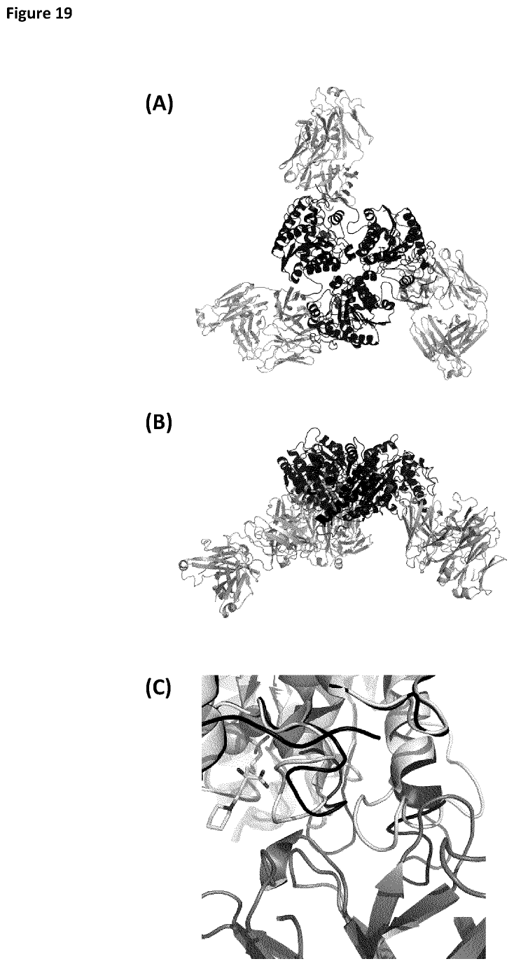

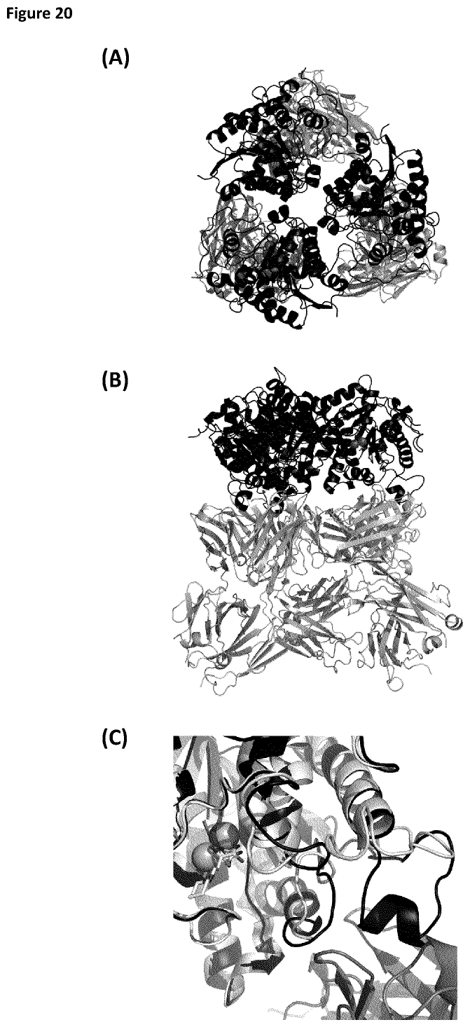

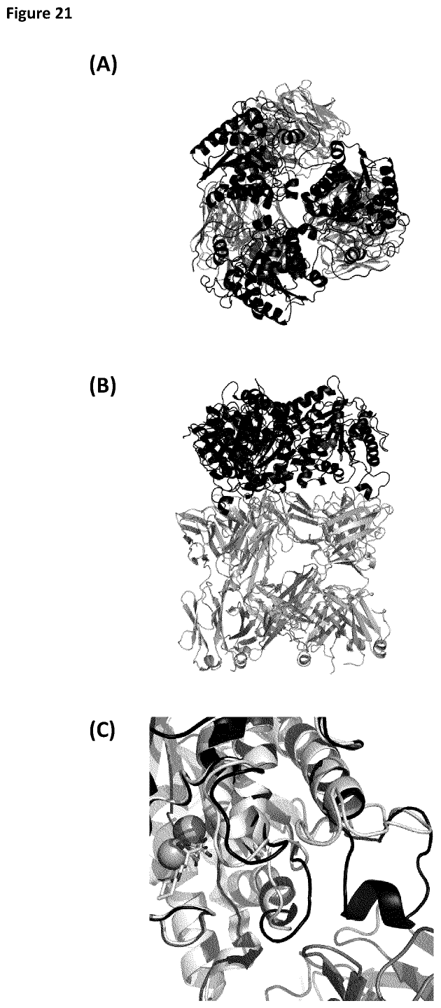

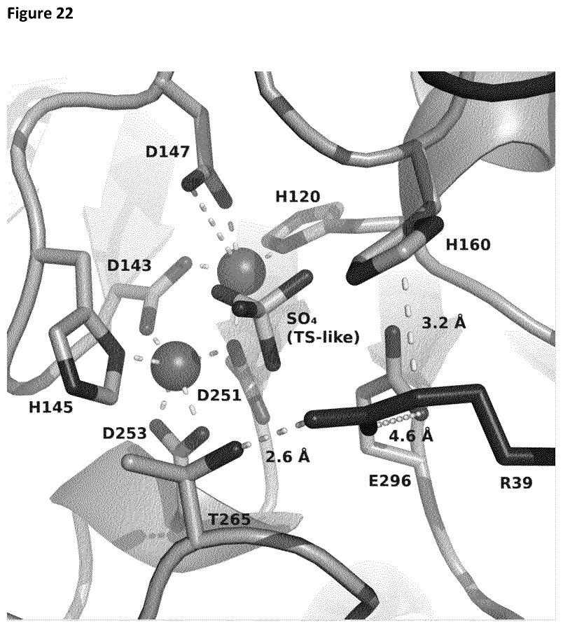

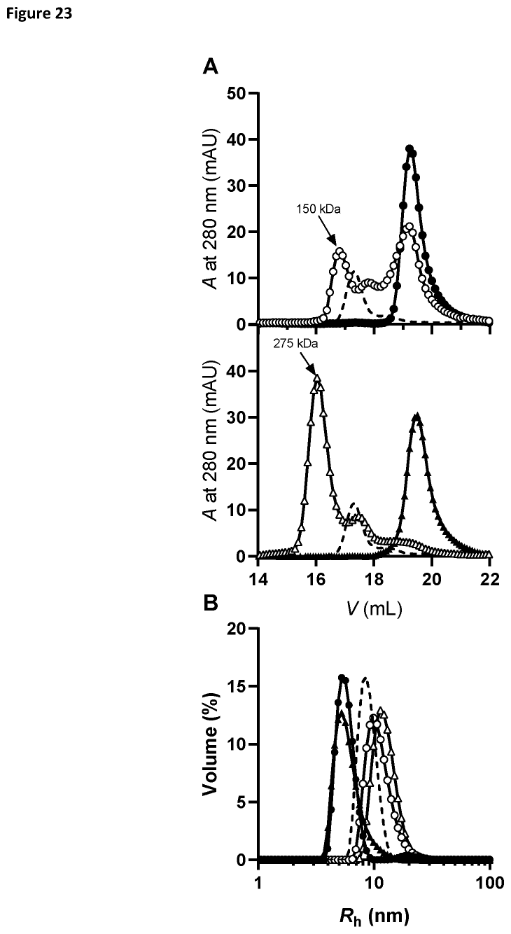

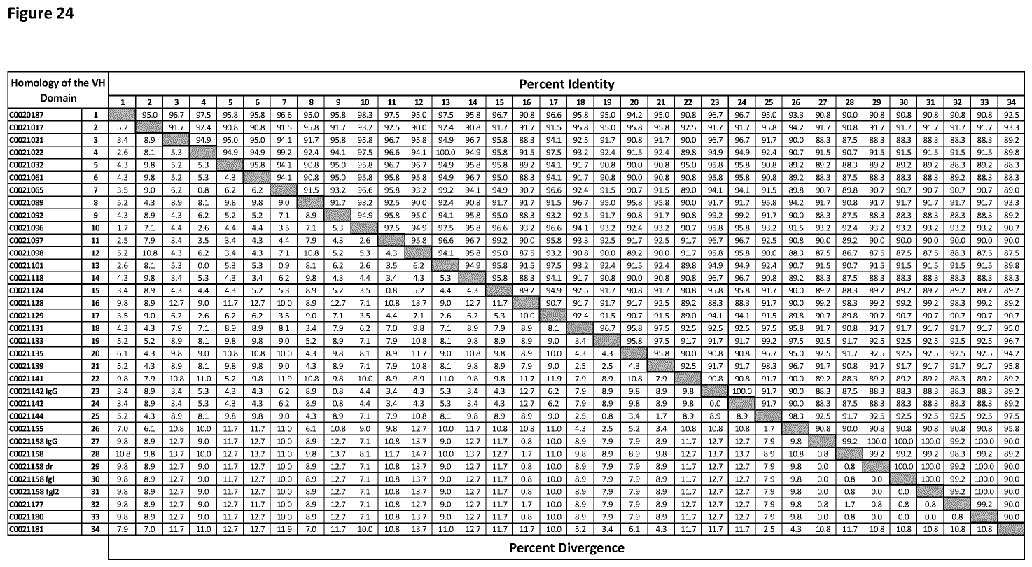

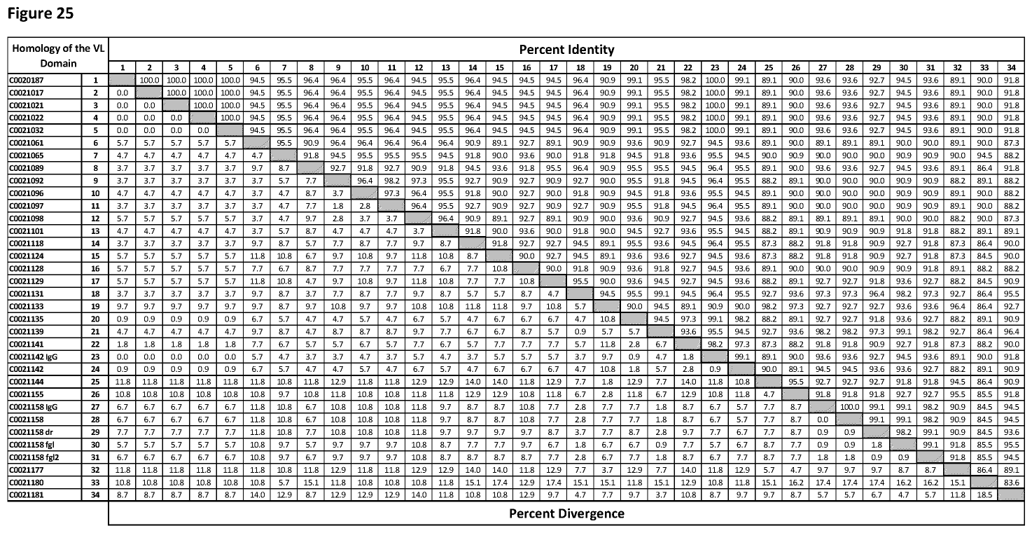

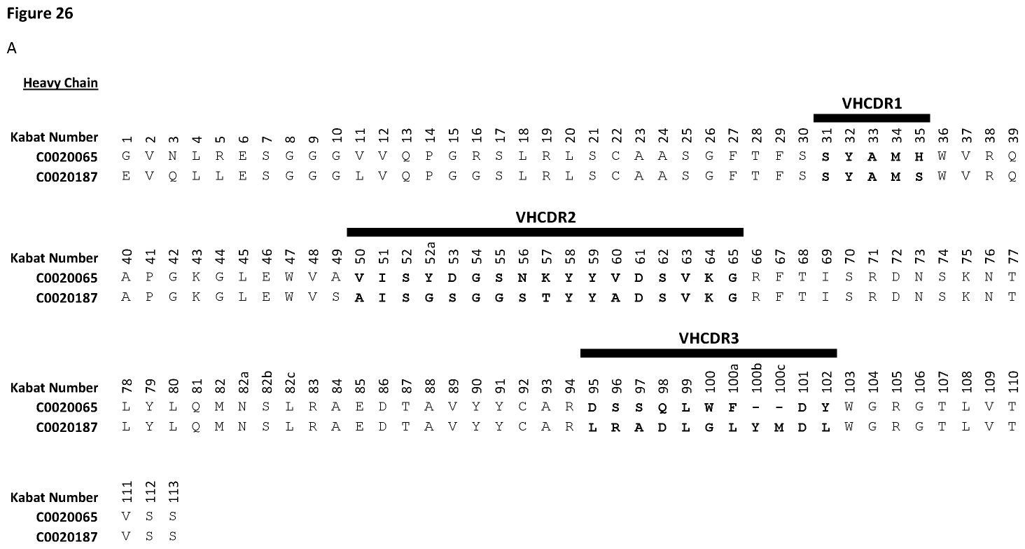

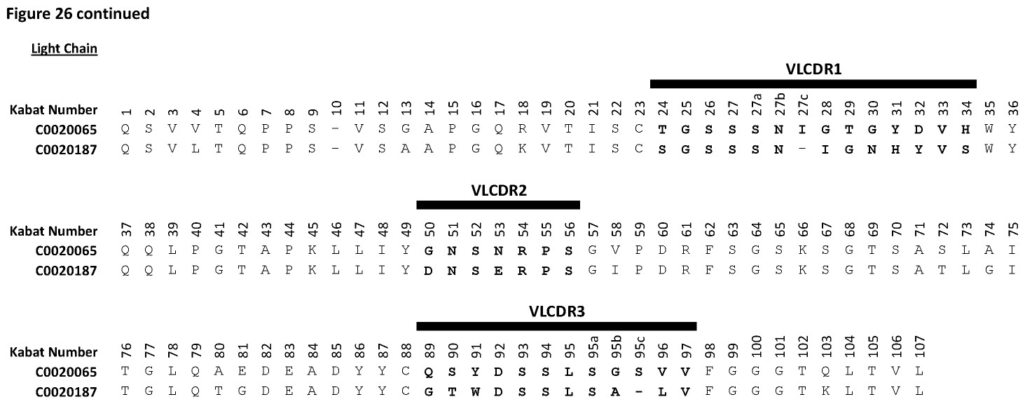

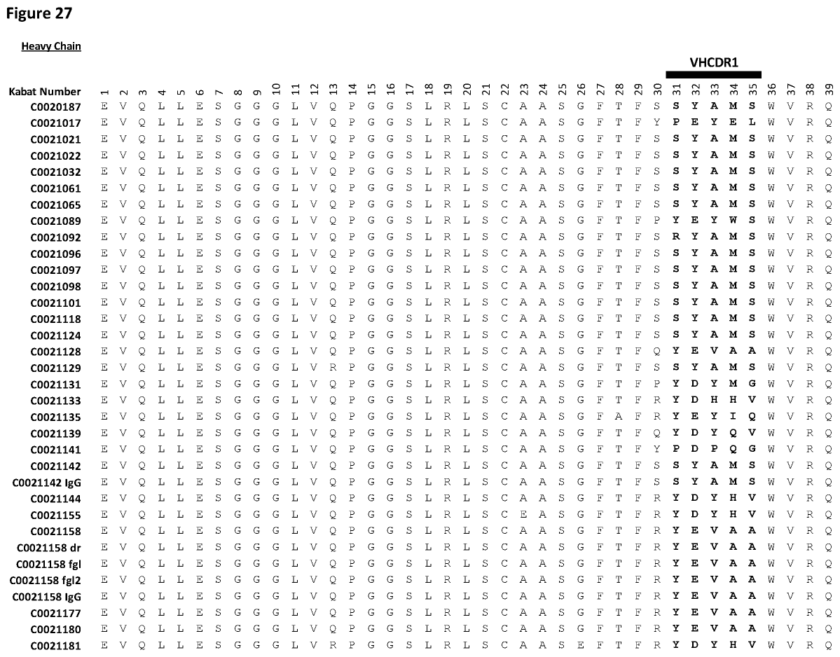

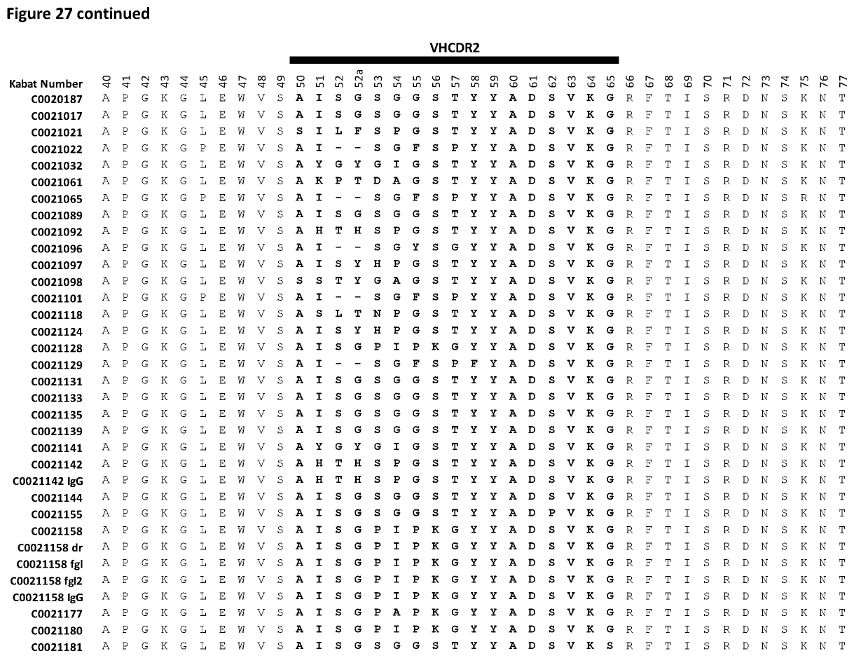

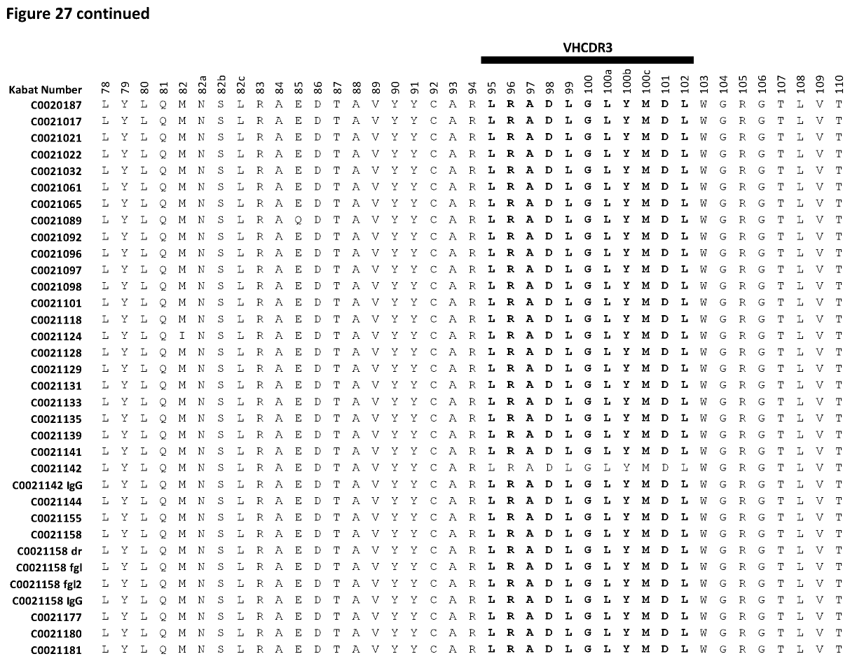

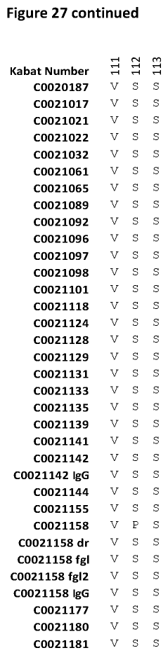

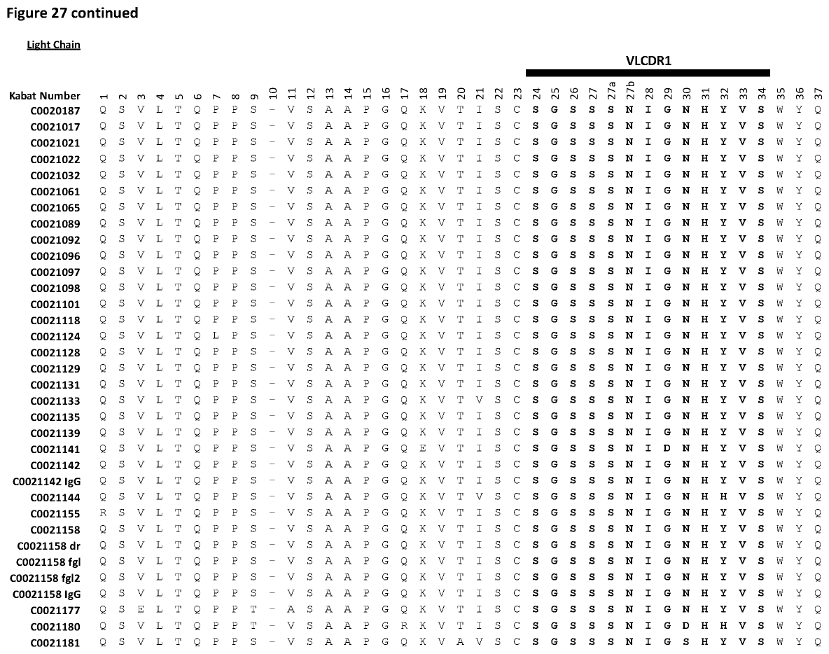

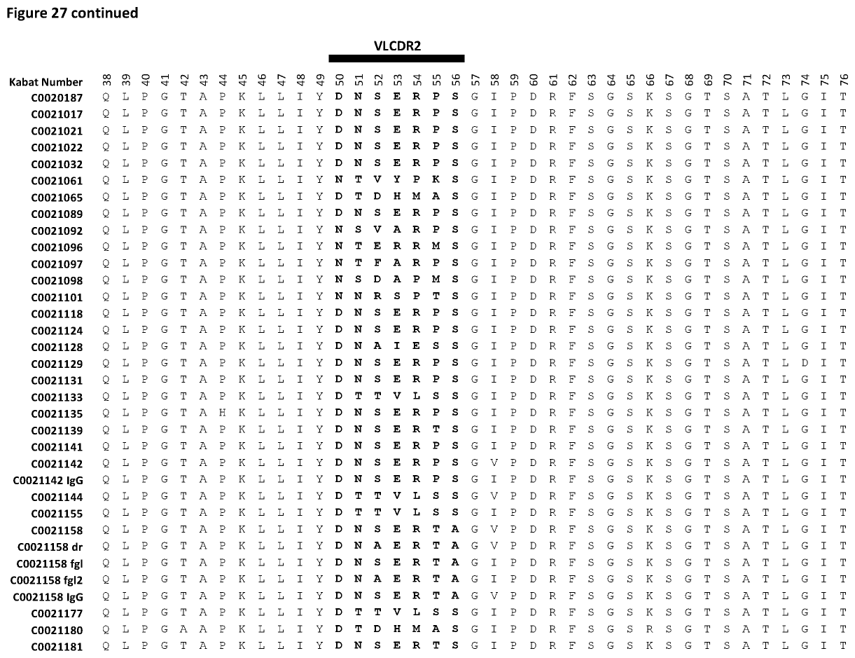

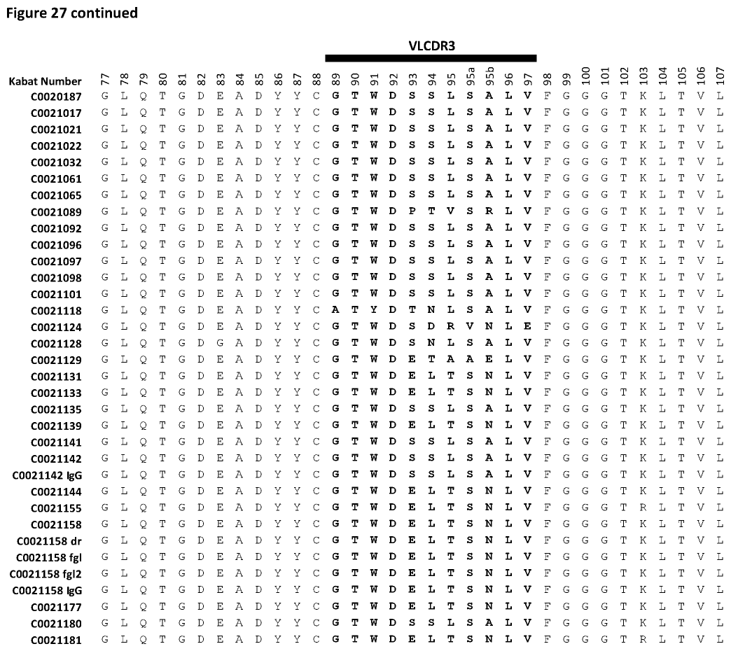

ScFv sample concentration was plotted against % Control using scientific graphing software, and any concentration-dependent responses were fitted with non-linear regression curves. IC₅₀ values were obtained from these analyses (FIG. 8, Table 11).

TABLE 11
IC₅₀ values (M) for clones in scFv format in
187 IgG epitope competition assay (FIG. 8)

| | 18° IgG epitope |
|---|---|
| | competition assay |
| Clone ID | IC50 (M) |
| C0020187 scFv | 1.58E−06 |
| C0021017 scFv | 1.22E−07 |
| C0021021 scFv | 5.87E−08 |
| C0021022 scFv | 6.59E−08 |
| C0021032 scFv | 6.70E−08 |
| C0021061 scFv | 1.01E−07 |
| C0021065 scFv | 7.47E−08 |
| C0021089 scFv | 1.33E−07 |
| C0021092 scFv | 1.30E−07 |
| C0021096 scFv | 3.52E−08 |
| C0021097 scFv | 2.75E−08 |
| C0021098 scFv | 4.83E−08 |
| C0021101 scFv | 4.08E−08 |
| C0021118 scFv | 4.17E−08 |
| C0021124 scFv | 5.05E−08 |
| C0021128 scFv | 4.45E−08 |
| C0021129 scFv | 4.50E−08 |
| C0021131 scFv | 4.57E−08 |
| C0021133 scFv | 4.33E−08 |
| C0021135 scFv | 5.20E−08 |
| C0021139 scFv | 4.01E−08 |
| C0021141 scFv | 5.11E−08 |
| C0021142 scFv | 5.78E−08 |

Reagent/Equipment sources: tris base (Sigma, UK; cat: RDD008), potassium fluoride (VWR chemicals, Belgium; cat: 26820.236), bovine serum albumin solution (Sigma, UK; cat: A7284), C0020187 IgG (produced in-house), biotinylated recombinant human trimeric ARG2 enzyme (produced in-house), Streptavidin cryptate (Cisbio, France; cat: 610SAKLB), anti-Human-IgG-XL665 (Cisbio, France; cat: 61 HFCXLB), 384-well assay plates (Greiner BioOne, Germany; cat: 784076), 384-well dilution plates (Greiner BioOne, Germany; cat: 781280), liquid handling robot (Hamilton Star™, Hamilton, USA), fluorescence plate reader (Pherastar™, BMG Labtech, USA), HTRF technology (Cisbio International, France), graphing/statistical software (Prism, GraphPad USA).

2.5 Identification of Improved Clones Using the Enzyme Inhibition Assay

The unpurified periplasmic scFv that were tested in the epitope competition assay described in Section 2.4 were simultaneously screened for their ability to inhibit enzymatic activity of trimeric human ARG2 in the assay described in section 1.5. C0020187 was also included in this assay, as an unpurified periplasmic scFv, and used as a benchmark against which improved clones could be identified. Improved clones were required to show an increase of 5% or more inhibition over the inhibition observed with C0020187.

Unpurified periplasmic scFv demonstrating significant improvement over C0020187 IgG were subjected to DNA sequencing (Osbourn et al., 1996; Vaughan et al., 1996). The scFv found to have unique protein sequences were expressed in E. coli and purified by affinity chromatography followed by buffer exchange.

The potency of each purified scFv was determined by testing a dilution series of the scFv (typically 4 pM-1200 nM) in the enzyme inhibition assay described in section 1.5. Data were again analysed by calculating % Max control values for each sample.

ScFv sample concentration was plotted against % Max control using scientific graphing software, and any concentration-dependent responses were fitted with non-linear regression curves. IC₅₀ values were obtained from these analyses (FIG. 9, Table 12).

TABLE 12
IC₅₀ values (M) for clones in scFv
format in trimeric human ARG2 EIA

| | Trimeric human ARG2 |
|---|---|
| Clone ID | EIA IC50 (M) |
| C0020187 scFv | 9.64E−07 |
| C0021017 scFv | 2.26E−07 |
| C0021021 scFv | 1.51E−07 |
| C0021022 scFv | 1.06E−07 |
| C0021032 scFv | 2.11E−07 |
| C0021061 scFv | 1.10E−07 |
| C0021065 scFv | 1.43E−07 |
| C0021089 scFv | 7.15E−08 |
| C0021092 scFv | 1.59E−07 |
| C0021096 scFv | 5.78E−08 |
| C0021097 scFv | 9.91E−08 |
| C0021098 scFv | 1.34E−07 |
| C0021101 scFv | 6.48E−08 |
| C0021118 scFv | 8.41E−08 |
| C0021124 scFv | 3.75E−07 |
| C0021128 scFv | 1.69E−07 |
| C0021129 scFv | 3.13E−07 |
| C0021131 scFv | 2.51E−07 |
| C0021133 scFv | 2.81E−07 |
| C0021135 scFv | 2.20E−07 |
| C0021139 scFv | 1.88E−07 |
| C0021141 scFv | 1.09E−07 |
| C0021142 scFv | 9.76E−08 |

2.6 Reformatting of Affinity Improved scFv to Human IgG₁

ScFv of optimised sequence were reformatted by the same procedure described for reformatting of lead clones as described in Section 1.8.

2.7 Binding of THP1 Derived ARG2 in Presence of Plasma.

The panel of improved variant antibodies was tested for binding to cell lysates from THP-1 cells overexpressing human ARG2 and parental THP-1 cell line in presence and absence of human healthy plasma (FIG. 10). The cell lysates were prepared in TBS buffer and the total protein concentration was established by BCA assay (Thermo Scientific, 23227). Human plasma was obtained from NHS BT Cambridge (batch: 15 Aug. 2016).

Six ARG2 affinity-matured IgGs (C0021022, C0021061, C0021065, C0021092, C0021133 and C0021139) were tested alongside parental clone C0020187 as well as irrelevant IgG control R347 and no IgG control. Briefly, maxisorp white plate (Nunc, 437796) was coated O/N at 4° C. with rabbit anti-human ARG2 antibody (abcam, ab137069) at 1 μg/ml in PBS. Next day, plate was washed with PBS and blocked with Casein blocker (Thermo Scientific, 37528). Cell lysates from THP-1-ARG2 positive cell line and THP-1 parental cell line were diluted in TBS buffer to 1 mg/ml and tested supplemented with 12.5% final concentration of urea depleted human healthy plasma or without plasma. 60 μl of each cell lysate were aliquoted into Greiner PP plate (Greiner, 650201). Recombinant human ARG2-HIS (PSPUR016), prepared at 1 μg/ml in TBS buffer, was used as a positive control and TBS buffer alone or 12.5% plasma in TBS were used as negative controls. In-house ARG2 specific human IgGs and irrelevant R347 IgG (all biotinylated) were added to the cell lysates, recombinant human ARG2 and TBS buffer to a final concentration of 50 μg/ml (no IgG control was also included) and incubated at RT with gentle shaking for 45 min. Blocked ELISA plate was washed with PBS and 50 μl of pre-incubated in the previous step samples was transferred to the ELISA plate and incubated stationary for 90 min at RT. Plate was washed 5 times with PBST (0.1%). 50 μl of Streptavidin-HRP (abcam, ab7403), diluted 1/10000 in casein buffer (1/20 dilution of stock) was added to each well and plate was incubated stationary at RT for 1 hour. After 5 washes with PBST, PICO ELISA substrate (Thermo Scientific, 37070) was prepared following manufacturer's instructions and 100 μl were added to each well. Plate was incubated for 1 min in the dark with gentle shaking and luminescence was read using Envision plate reader. Data were analysed using GraphPad Prism. Results are shown in FIG. 10 for the following:

1-THP1-Arg2 cell lysate (1 mg/ml)
2-THP1-parental cell lysate (1 mg/ml)
3-THP1-Arg2 cell lysate (1 mg/ml) + 12.5% human healthy plasma (urea depleted)
4-THP1-parental cell lysate (1 mg/ml) + 12.5% human healthy plasma (urea depleted)
5-TBS + 12.5% human healthy plasma (urea depleted)
6-Recombinant human Arg2 PSPUR016 (1 μg/ml in TBS)
7-TBS

All ARG2 IgGs showed specific binding to ARG2 expressing THP-1 cell lysates and recombinant human ARG2 with affinity-matured clones showing higher positive signal compared to parental C0020187.

2.8 Inhibition of THP-1 Derived ARG2 by Improved Variant IgGs

Anti-ARG2 IgGs were tested for their ability to inhibit mammalian expressed human ARG2 produced in transfected THP-1 cells. To do this cell lysates were prepared from THP-1 cells overexpressing human ARG2 and from wild type THP-1 cells. 350 million cells were suspended in 3 ml tris buffered saline (25 mM Tris+150 mM sodium chloride in dH₂O, pH 7.4) and cell lysates prepared by passing through a 26G needle 20 times. A BCA assay was used to measure the protein concentration in the lysate. To determine the IC₅₀ values of test antibodies, a dilution series of the test antibodies were created in TBS and 5 μl were added to 10 μl lysate/human plasma mixture (1.15 mg/ml lysate, 25% (v/v) urea depleted human plasma and 20 mM MnCl₂ and incubated for 2 hours at room temperature in a 384 well plate. The enzymatic reaction was started with the addition of 5 μl 100 mM L-arginine substrate and incubated for 1 hour at room temperature. Urea detection reagents, O-Phthaldialdehyde (1.5 mM O-Phthaldialdehyde, 7% H₂SO₄, 0.03% Brij L23) and N-(1-Napthyl)ethylenediamine (1.66 mM N-(1-Napthyl)ethylenediamine, 81 mM Boric acid, 21% H₂SO₄, 0.03% Brij L23) were mixed 1:1 just prior to use and 80 μl/well was added, incubated for 2 hours at room temperature and absorbance at 505 nm was read on a Pherastar plate reader. Antibodies were tested alongside N-Hydroxyl-L-Arginine (a small molecule competitive inhibitor of ARG2) as a positive control and CEA6 (irrelevant isotype control antibody) as a negative control. Data points were plotted and IC₅₀ values determined using a four parameter fit in GraphPad Prism (FIG. 11, Table 13).

TABLE 13

| | IC₅₀ (M) | |
|---|---|---|
| | THP-1 ARG2 | THP-1 ARG2 (healthy |
| Inhibitor | (no plasma) | human plasma) |
| C0021022 IgG | 5.81e−9 | 2.71e−9 |
| C0021061 IgG | 6.79e−9 | 2.90e−9 |
| C0021065 IgG | 5.99e−9 | 2.98e−9 |
| C0021092 IgG | 6.56e−9 | 2.82e−9 |
| C0021139 IgG | 5.75e−9 | 2.02e−9 |
| C0021133 IgG | 6.68e−9 | 3.70e−9 |

IC₅₀ values are averaged data from n = 2 experiments.

2.9 Restoration of T Cell Proliferation by Improved Variant ARG2 Specific IgGs in the Presence of ARG2

Anti-ARG2 antibodies were screened for their ability to restore T cell proliferation by inhibiting enzymatic activity of ARG2 and hence, the depletion of L-arginine, which is required for T cell proliferation. This assay served to provide evidence of improved functional efficacy for each affinity-matured antibody in comparison to the parent IgG, C0020187 (FIG. 12).

Lead optimisation clones were tested to an endpoint treatment time of 96 hours and the BrdU Cell Proliferation Kit (Roche, France; Cat: 11647229001) was used according to the manufacturer's instructions. R347 was used as the irrelevant control. All clones tested n=2.

TABLE 14

| | | | In Presence of rhARG2 | | | In Absence of rhARG2 | | |
|---|---|---|---|---|---|---|---|---|
| | | Top | Maximum % | Maximum % | | Maximum % | Maximum % | |
| Clone ID | Format | Conc | Proliferation | Proliferation SD | EC₅₀ | Proliferation | Proliferation SD | EC₅₀ |
| R347 | hlgG1 | 1.97 μM | −0.5 | 0.3 | >[1.97 μM] | 95 | 5 | <[0.0009 μM] |
| C0021022 | hlgG1 | 2.12 μM | 77 | 31 | 0.132 μM | 106 | 12 | <[0.001 μM] |
| C0021061 | hlgG1 | 1.97 μM | 83 | 10 | 0.113 μM | 99 | 5 | <[0.0009 μM] |
| C0021065 | hlgG1 | 2 μM | 87 | 7 | 0.136 μM | 101 | 7 | <[0.0009 μM] |
| C0021092 | hlgG1 | 2.17 μM | 77 | 11 | 0.116 μM | 92 | 7 | <[0.0009 μM] |
| C0021133 | hlgG1 | 1.6 μM | 73 | 8 | 0.101 μM | 90 | 7 | <[0.0007 μM] |
| C0021139 | hlgG1 | 2.17 μM | 67 | 10 | 0.111 μM | 84 | 9 | <[0.001 μM] |
| C0020187 | hlgG1 | 2 μM | 68 | 17 | 1.07 μM | 101 | 11 | <[0.0009 μM] |

Leukocyte cones underwent Ficoll Plaque (GE, Sweden; Cat: 17-1440-02) gradient separation, PBMC's were extracted and pan T cell population isolated by negative selection using the Human T Cell Enrichment Kit (Stemcell Technologies, France; Cat: 19051) according to the manufacturer's instructions.

40000 cells/well were seeded in complete growth media (RMPI 1640, 5% Human Serum Albumin) into a 96 well clear TC treated microplate (Greiner-Bio One, Germany; Cat: 655180). T cells were activated by the addition of ImmunoCult CD3/CD28 (Stemcell Technologies, France; Cat: 10971) at a dilution of 1:40 directly into wells and the plate incubated 37° C., 5% CO₂ for 45 minutes.

To determine EC₅₀'s of test antibodies, a dilution series was created in sterile TBS/NaCl Buffer at 10× the final assay concentration added to the assay plate containing cells. Plate was incubated for 30 minutes at 37° C., 5% CO₂.

ARG2 was added to relevant wells of the plate at a final concentration of 15 μg/ml. The equivalent volume of ARG2 vehicle and test antibody vehicle was added to relevant control wells. The total volume in each well was 200 μl. Cells were treated with test antibodies for a total of 96 hours at 37° C., 5% CO₂. Antibodies were tested alongside C0020187 (parent IgG) as a positive control and R347 (irrelevant isotype control antibody) as a negative control.

Assessment of T cell proliferation was performed using the BrdU Cell Proliferation ELISA Kit (Roche, Germany; Cat: 000000011647229001). BrdU labelling reagent was added after 78 hours of antibody treatment and the plate further incubated for 18 hours at 37° C., 5% CO₂. The remainder of the assay was performed according to the manufacturer's instructions.

Calculated data was expressed as % of control (Equation 3) and graphical plots were constructed using a four parameter fit and EC₅₀'s determined using model 205 in XLFit (IDBS, UK).

All test sample data was corrected for background (mean of no cell control)

% Control=100*((Test Sample−Mean Positive Control)/(Mean Negative Control−Mean Positive Control)) Equation 3:

Mean Positive Control=T Cells in absence of ARG2

Mean Negative Control=T Cells in presence of ARG2

2.10 Kinetic Profiling of Affinity Improved Clones as Fabs Using Bio-Layer Interferometry (BLI)

The affinities of the affinity-matured antibodies to human ARG2 were measured on the OctetRED system as described in section 1.15 using the streptavidin capture method. Typically, an analyte titration range of 1.8 to 120 nM was used. The derived affinities to human ARG2 and cynomolgus ARG2 are shown in Table 15 and 16, respectively.

TABLE 15
Determination of binding affinities of affinity-matured antibodies, as Fab, to human
ARG2 using Bio-Layer Interferometry

| | | K_{D} | K_{D} | kₐ | kₐ | k_{d} | k_{d} |
|---|---|---|---|---|---|---|---|
| Ligand | Analyte | (M) | Error | (1/Ms) | Error | (1/s) | Error |
| Human | C0021021 | 4.24E−09 | 4.40E−11 | 4.73E+05 | 4.59E+03 | 2.00E−03 | 7.36E−06 |
| ARG2 | Fab | | | | | | |
| Human | C0021022 | 2.73E−09 | 2.59E−11 | 4.12E+05 | 3.46E+03 | 1.12E−03 | 5.01E−06 |
| ARG2 | Fab | | | | | | |
| Human | C0021032 | 5.33E−09 | 5.03E−11 | 4.27E+05 | 3.81E+03 | 2.28E−03 | 6.99E−06 |
| ARG2 | Fab | | | | | | |
| Human | C0021061 | 1.30E−09 | 5.64E−12 | 2.71E+05 | 7.34E+02 | 3.51E−04 | 1.20E−06 |
| ARG2 | Fab | | | | | | |
| Human | C0021065 | 1.62E−09 | 1.34E−11 | 4.09E+05 | 2.63E+03 | 6.65E−04 | 3.46E−06 |
| ARG2 | Fab | | | | | | |
| Human | C0021092 | 2.69E−09 | 2.41E−11 | 2.80E+05 | 2.09E+03 | 7.55E−04 | 3.76E−06 |
| ARG2 | Fab | | | | | | |
| Human | C0021096 | 3.48E−09 | 3.63E−11 | 4.41E+05 | 4.22E+03 | 1.54E−03 | 6.37E−06 |
| ARG2 | Fab | | | | | | |
| Human | C0021097 | 4.95E−09 | 3.42E−11 | 2.95E+05 | 1.90E+03 | 1.46E−03 | 3.68E−06 |
| ARG2 | Fab | | | | | | |
| Human | C0021101 | 3.41E−09 | 3.47E−11 | 3.14E+05 | 2.84E+03 | 1.07E−03 | 4.93E−06 |
| ARG2 | Fab | | | | | | |
| Human | C0021118 | 8.23E−09 | 5.26E−11 | 1.39E+05 | 7.78E+02 | 1.14E−03 | 3.53E−06 |
| ARG2 | Fab | | | | | | |
| Human | C0021128 | 2.53E−09 | 8.48E−12 | 2.66E+05 | 7.38E+02 | 6.74E−04 | 1.27E−06 |
| ARG2 | Fab | | | | | | |
| Human | C0021133 | 2.05E−09 | 6.44E−12 | 2.19E+05 | 5.14E+02 | 4.49E−04 | 9.32E−07 |
| ARG2 | Fab | | | | | | |
| Human | C0021139 | 4.41E−09 | 1.78E−11 | 1.98E+05 | 7.20E+02 | 8.71E−04 | 1.50E−06 |
| ARG2 | Fab | | | | | | |
| Human | C0021141 | 3.33E−09 | 3.50E−11 | 3.26E+05 | 3.02E+03 | 1.08E−03 | 5.35E−06 |
| ARG2 | Fab | | | | | | |
| Human | C0021142 | 2.77E−09 | 2.81E−11 | 3.99E+05 | 3.54E+03 | 1.10E−03 | 5.48E−06 |
| ARG2 | Fab | | | | | | |

TABLE 16
Determination of binding affinities of a smaller panel of antibodies
to cynomolgus ARG2 using Bio-Layer Interferometry

| | | K_{D} | K_{D} | kₐ | kₐ | k_{d} | k_{d} |
|---|---|---|---|---|---|---|---|
| Ligand | Analyte | (M) | Error | (1/Ms) | Error | (1/s) | Error |
| Cyno | C0021022 | 1.66E−09 | 1.82E−11 | 7.06E+05 | 6.79E+03 | 1.17E−03 | 6.10E−06 |
| ARG2 | Fab | | | | | | |
| Cyno | C0021061 | 9.53E−10 | 5.20E−12 | 3.10E+05 | 9.14E+02 | 2.96E−04 | 1.36E−06 |
| ARG2 | Fab | | | | | | |
| Cyno | C0021065 | 1.27E−09 | 1.33E−11 | 6.17E+05 | 5.22E+03 | 7.82E−04 | 4.90E−06 |
| ARG2 | Fab | | | | | | |
| Cyno | C0021092 | 2.05E−09 | 1.93E−11 | 3.54E+05 | 2.68E+03 | 7.26E−04 | 4.09E−06 |
| ARG2 | Fab | | | | | | |
| Cyno | C0021128 | 1.77E−09 | 6.94E−12 | 4.37E+05 | 1.40E+03 | 7.71E−04 | 1.76E−06 |
| ARG2 | Fab | | | | | | |
| Cyno | C0021133 | 1.67E−09 | 5.69E−12 | 3.28E+05 | 8.32E+02 | 5.49E−04 | 1.24E−06 |
| ARG2 | Fab | | | | | | |
| Cyno | C0021139 | 2.63E−09 | 1.02E−11 | 2.75E+05 | 8.91E+02 | 7.24E−04 | 1.54E−06 |
| ARG2 | Fab | | | | | | |

2.11 Testing for Binding to Human ARG1

The binding of affinity-matured antibodies to human ARG1 was also tested on the OctetRED system using a screening assay which stably captures His-tagged ARG2 as the ligand on an anti-His surface. The Fabs were typically tested in an analyte range of 1.8 to 120 nM, with an association time of 5 minutes and a dissociation time of 10 minutes. Sensors were regenerated using 10 mM glycine pH 1.5 after each cycle. The results are shown in Table 17.

TABLE 17
Testing the binding of a smaller panel of lead antibodies,
as Fab, to human ARG1 using Bio-Layer Interferometry

| Ligand | Analyte | Results |
|---|---|---|
| Human ARG1 | C0021022 Fab | No binding observed |
| Human ARG1 | C0021061 Fab | No binding observed |
| Human ARG1 | C0021065 Fab | No binding observed |
| Human ARG1 | C0021092 Fab | No binding observed |
| Human ARG1 | C0021128 Fab | No binding observed |
| Human ARG1 | C0021133 Fab | No binding observed |
| Human ARG1 | C0021139 Fab | No binding observed |

### Example 3—Generation and Characterisation of Second-Generation Affinity-Matured ARG2 Leads (Pool Maturation by Ribosome Display)

3.1 Production of 2^{nd} Generation Affinity-Matured Leads

Second-generation error-prone libraries were built based on the scFv constructs on a pool of the top antibody candidates from the previous selection cascades. Error-prone PCR was used to introduce random mutations into the scFv region of the constructs, and the resulting libraries were used in ribosome display selections as described in Example 2. The resulting selection outputs were sub-cloned and screened as periplasmic preparations/crude lysates in a second-generation epitope competition assay. The hits were sequenced, and lead candidates were chosen based on sequence diversity and hit values. A panel of Fabs were produced and kinetically profiled using Bio-Layer Interferometry (BLI) on the Octet RED96 to rank the candidates based on binding affinities to human ARG2.

3.2 Identification of Improved Clones from Second Generation Error-Prone Library Selections (Ribosome Display) Using an Epitope Competition Assay

One thousand three hundred and twenty scFv chosen at random from selection rounds 2 and 3 of the error-prone mutagenesis approach described in section 2.2 and 2.3 were expressed in bacteria to produce unpurified periplasmic scFv. Those scFv capable of binding recombinant human trimeric ARG2 enzyme via the same epitope as C0021133 IgG were elucidated in a competition format assay, using the HTRF™ platform. Specifically, fluorescence resonance energy transfer (FRET) was measured between streptavidin cryptate (associated with biotinylated recombinant human trimeric ARG2 enzyme) and anti-human Fc XL665 (associated with C0021133 IgG) in the presence of a single concentration of each unpurified periplasmic test scFv. Successful occupation of the C0021133 IgG epitope on the enzyme by scFv resulted in a reduction in FRET, as measured on a fluorescence plate reader.

A ‘Maximum’ binding signal was determined by analysing the binding of C0021133 IgG to biotinylated recombinant human trimeric ARG2 enzyme in the absence of competitor scFv. The ‘Sample’ signals were derived from analysing the binding of C0021133 IgG to biotinylated recombinant human trimeric ARG2 enzyme in the presence of a test scFv sample. Finally, a ‘Background’ signal was determined by analysing the fluorescence generated in the absence of C0021133 IgG.

Unpurified periplasmic scFv were supplied in sample buffer consisting of 200 mM tris base, pH 7.4, 0.5 mM EDTA, and 0.5 M sucrose. 5 μl of each scFv were transferred to the ‘Sample’ wells of a black, shallow, solid bottom, non-binding 384-well assay plate using a liquid handling robot. The remaining reagents (prepared in assay buffer) were added to the assay plate by multichannel pipette in the following order: 5 μl detection cocktail, consisting of 6.6 nM streptavidin cryptate and 40 nM anti-human Fc XL665 (to all wells), 5 μl 12 nM biotinylated recombinant human trimeric ARG2 enzyme (to all wells), 5 μl 8 nM C0021133 IgG (to ‘Sample’ and ‘Maximum’ wells), and 5 μl sample buffer (to background wells). Assay plates were sealed and then incubated overnight at room temperature in the dark, prior to measuring time-resolved fluorescence at 620 and 665 nm emission wavelengths on a fluorescence plate reader.

Data were analysed by calculating % Delta F values for each sample. % Delta F was determined according to equation 1.

Equation 1:

$$\% Delta F = \frac{\begin{matrix}( sample 665 nm / 620 nm ratio ) - \\ ( negative control 665 nm / 620 nm ratio )\end{matrix}}{( negative control 665 nm / 620 nm ratio )} \times 100$$

Delta F values were subsequently used to calculate normalised binding values as described in equation 4.

$$Normalised data ( \% Control ) = \frac{\% Delta F of sample}{\% Delta F of Maximum binding control} \times 100 \qquad Equation 4$$

Unpurified periplasmic scFv demonstrating significant inhibition of C0021133 IgG binding to biotinylated recombinant human trimeric ARG2 enzyme were subjected to DNA sequencing (Osbourn et al., 1996; Vaughan et al., 1996). The scFv found to have unique protein sequences were expressed as IgG and Fab.

Reagent/Equipment sources: tris base (Sigma, UK; cat: RDD008), potassium fluoride (VWR chemicals, Belgium; cat: 26820.236), bovine serum albumin solution (Sigma, UK; cat: A7284), C0020187 IgG (produced in-house), biotinylated recombinant human trimeric ARG2 enzyme (produced in-house), Streptavidin cryptate (Cisbio, France; cat: 610SAKLB), anti-Human-IgG-XL665 (Cisbio, France; cat: 61 HFCXLB), 384-well assay plates (Greiner BioOne, Germany; cat: 784076), 384-well dilution plates (Greiner BioOne, Germany; cat: 781280), liquid handling robot (Hamilton Star™, Hamilton, USA), fluorescence plate reader (Pherastar™, BMG Labtech, USA), HTRF technology (Cisbio International, France), graphing/statistical software (Prism, GraphPad USA).

3.3 Identification of Improved Clones from Second Generation Error-Prone Library Selections (Ribosome Display) Using the Enzyme Inhibition Assay

The unpurified periplasmic scFv that were tested in the epitope competition assay described in section 2.4 were simultaneously screened for their ability to inhibit enzymatic activity of trimeric human ARG2 in the assay described in section 1.5. C0021133 was also included in this assay, as unpurified periplasmic scFv, and used as a benchmark against which improved clones could be identified. Improved clones were required to show an increase of 5% or more inhibition over the inhibition observed with C0021133.

Unpurified periplasmic scFv demonstrating significant improvement over C0021133 IgG were subjected to DNA sequencing (Osbourn et al., 1996; Vaughan et al., 1996). The scFv found to have unique protein sequences were expressed as IgG (section 1.8).

Anti-ARG2 IgGs were tested for their ability to inhibit to mammalian expressed human ARG2 produced in transfected THP-1 cells. To do this cell lysates were prepared from THP-1 cells overexpressing human ARG2 and from wild type THP-1 cells. 350 million cells were suspended in 3 ml tris buffered saline (25 mM Tris+150 mM sodium chloride in dH₂O, pH 7.4) and cell lysates prepared by passing through a 26G needle 20 times. A BCA assay was used to measure the protein concentration in the lysate. To determine IC₅₀s of test antibodies, a dilution series of the test antibodies were created in TBS and 5 μl were added to 10 μl lysate (1.19 mg/ml lysate, 20 mM MnCl₂, 50 mM Tris HCl in MilliQ, pH 7.4) and incubated for 2 h at room temperature in a 384 well plate. The enzymatic reaction was started with the addition of 5 μl 100 mM L-arginine substrate and incubated for 1 hour at room temperature. Urea detection reagents, O-Phthaldialdehyde (1.5 mM O-Phthaldialdehyde, 7% H₂SO₄, 0.03% Brij L23) and N-(1-Napthyl)ethylenediamine (1.66 mM N-(1-Napthyl)ethylenediamine, 81 mM Boric acid, 21% H₂SO₄, 0.03% Brij L23) were mixed 1:1 just prior to use and 80 μl/well were added, incubated for 1 hour at room temperature and absorbance at 505 nm was read on a Pherastar plate reader. Antibodies were tested alongside N-Hydroxyl-L-arginine (a small molecule competitive inhibitor of ARG2) as a positive control and CEA6 (irrelevant isotype control antibody) as a negative control (FIG. 13, Table 18). Data points were plotted and IC₅₀ values were determined using a four parameter fit in GraphPad Prism.

TABLE 18

| | THP-1 lysate ARG2 |
|---|---|
| Clone ID | EIA IC50 (M) |
| C0021144 | 1.09E−08 |
| C0021158 | 5.36E−09 |
| C0021180 | 4.59E−09 |
| C0021155 | 9.64E−09 |
| C0021177 | 5.17E−09 |
| C0021181 | 6.93E−09 |
| C0021133 | 7.38E−09 |
| C0021061 | 5.81E−09 |
| (First generation lead) | |
| NHLA | 4.75E−06 |
| R347 IgG | Inactive |

3.4 Restoration of T Cell Proliferation by Improved Variant ARG2 Specific IgGs in the Presence of ARG2

Anti-ARG2 antibodies were screened for their ability to restore T cell proliferation by inhibiting enzymatic activity of ARG2 and hence, the depletion of the L-arginine, which is required for T cell proliferation. This assay served to provide evidence of improved functional efficacy for each affinity-matured antibody in comparison to the parent IgG, C0020187 (FIG. 14, Table 19). R347 was used as the irrelevant control. All clones tested n=2. Reference section 2.9 for methodology.

TABLE 19

| | | | In Presence of rhARG2 | | | | In Absence of rhARG2 | | |
|---|---|---|---|---|---|---|---|---|---|
| | | Top | Maximum % | Maximum % | | | Maximum % | Maximum % | |
| Clone ID | Format | Conc | Proliferation | Proliferation SD | EC₅₀ | | Proliferation | Proliferation SD | EC₅₀ |
| R347 | hlgG1 | 2 μM | −0.5 | 2 | >[2 | μM] | 93 | 7 | <[0.0009 μM] |
| C0020187 | hlgG1 | 2 μM | 61 | 21 | >[2 | μM] | 97 | 6 | <[0.0009 μM] |
| C0021061 | hlgG1 | 2 μM | 110 | 23 | 0.149 | μM | 93 | 9 | <[0.0009 μM] |
| C0021158 | hlgG1 | 2 μM | 90 | 15 | 0.157 | μM | 94 | 7 | <[0.0009 μM] |
| C0021177 | hlgG1 | 2 μM | 84 | 22 | 0.086 | μM | 90 | 7 | <[0.0009 μM] |
| C0021180 | hlgG1 | 2 μM | 91 | 12 | 0.137 | μM | 96 | 12 | <[0.0009 μM] |

3.5 Kinetic Profiling of Second-Generation Antibodies as Fabs Using Bio-Layer Interferometry (BLI)

The affinities of the second-generation antibodies to human ARG2 were measured on the OctetRED system. A screening assay which stably captures His-tagged ARG2 as the ligand on an anti-His surface was utilised to screen the second-generation candidates as Fabs at an analyte range of 1.8 to 120 nM, with an association time of 5 minutes and a dissociation time of 10 minutes. Sensors were regenerated using 10 mM glycine pH 1.5 after each cycle. Based on this assay, six candidates with estimated affinities in the sub-nanomolar range were identified, and they were further profiled using an extended assay which is more suitable for antibodies of high affinities. In the extended assay, the association and dissociation times were extended to 10 and 40 minutes respectively, using a lower Fab analyte titration range of 0.47 to 30 nM, on a streptavidin surface which captures bio-ARG2 as the ligand. The sensorgrams were reference subtracted and fitted to the 1:1 binding model to estimate their binding affinities using the Octet analysis software. The derived kinetic parameters are shown in Table 20.

TABLE 20
Determination of binding affinities of second-generation antibodies,
as Fab, to human ARG2 using Bio-Layer Interferometry

| | | K_{D} | K_{D} | kₐ | kₐ | k_{d} | k_{d} |
|---|---|---|---|---|---|---|---|
| Ligand | Analyte | (M) | Error | (1/Ms) | Error | (1/s) | Error |
| Human | C0021144 | 3.38E−10 | <1.0E−12 | 1.95E+05 | 2.28E+02 | 6.59E−05 | <1.0E−07 |
| ARG2 | Fab | | | | | | |
| Human | C0021155 | 3.58E−10 | <1.0E−12 | 1.59E+05 | 1.74E+02 | 5.67E−05 | <1.0E−07 |
| ARG2 | Fab | | | | | | |
| Human | C0021158 | 1.73E−10 | <1.0E−12 | 2.62E+05 | 2.35E+02 | 4.54E−05 | <1.0E−07 |
| ARG2 | Fab | | | | | | |
| Human | C0021177 | 1.73E−10 | <1.0E−12 | 2.02E+05 | 2.04E+02 | 3.51E−05 | <1.0E−07 |
| ARG2 | Fab | | | | | | |
| Human | C0021180 | 4.37E−10 | 1.485E−12 | 2.78E+05 | 7.40E+02 | 1.22E−04 | 2.565E−07 |
| ARG2 | Fab | | | | | | |
| Human | C0021181 | 2.88E−10 | <1.0E−12 | 1.45E+05 | 1.24E+02 | 4.17E−05 | <1.0E−07 |
| ARG2 | Fab | | | | | | |

The affinities of some of these antibodies to cynomolgus ARG2 were determined using the same assay, and the derived kinetic parameters are shown in Table 21.

TABLE 21
Determination of binding affinities of a smaller panel of second-generation
antibodies to cyno ARG2 using Bio-Layer Interferometry

| | | K_{D} | K_{D} | kₐ | kₐ | k_{d} | k_{d} |
|---|---|---|---|---|---|---|---|
| Ligand | Analyte | (M) | Error | (1/Ms) | Error | (1/s) | Error |
| Cyno | C0021158 | 1.08E−10 | <1.0E−12 | 4.29E+05 | 4.50E+02 | 4.64E−05 | 1.039E−07 |
| ARG2 | Fab | | | | | | |
| Cyno | C0021177 | 7.54E−11 | <1.0E−12 | 4.14E+05 | 4.21 E+02 | 3.12E−05 | <1.0E−07 |
| ARG2 | Fab | | | | | | |
| Cyno | C0021180 | 3.53E−10 | 1.187E−12 | 3.81E+05 | 1.00E+03 | 1.35E−04 | 2.822E−07 |
| ARG2 | Fab | | | | | | |
| Cyno | C0021181 | 1.73E−10 | <1.0E−12 | 2.57E+05 | 2.19E+02 | 4.43E−05 | <1.0E−07 |
| ARG2 | Fab | | | | | | |

3.6 Testing IgG Binding to Human ARG1

Binding of the second-generation antibodies to human ARG was also tested on the OctetRED system using a screening assay which stably captures His-tagged ARG2 as the ligand on an anti-His surface. The Fabs were tested as analyte up to 120 nM, with an association time of 5 minutes and a dissociation time of 10 minutes. Sensors were regenerated using 10 mM glycine pH 1.5 after each cycle. The results are shown in Table 22.

TABLE 22
Testing the binding of second-generation antibodies,
as Fab, to human ARG1 using Bio-Layer Interferometry

| Ligand | Analyte | Results |
|---|---|---|
| Human ARG1 | C0021144 Fab | No binding observed |
| Human ARG1 | C0021155 Fab | No binding observed |
| Human ARG1 | C0021158 Fab | No binding observed |
| Human ARG1 | C0021177 Fab | No binding observed |
| Human ARG1 | C0021180 Fab | No binding observed |
| Human ARG1 | C0021181 Fab | No binding observed |

3.7 Understanding the Mechanism of Action of ARG2 Specific IgGs on the Enzyme ARG2

The mechanism of action of the lead identification clone C0021158 IgG was investigated using the human ARG2 (trimer) enzyme inhibition assay as well as isothermal titration calorimetry.

The enzyme inhibition assay is described in section 1.5 and is a biochemical assay that measures production of urea, the product of L-arginine turnover by ARG2. To investigate the mechanism of action of C0021158 IgG, four point titrations of C0021158 IgG (1000, 100, 30 and 10 nM) were set up in the presence of 11 concentrations of arginine (250, 200, 150, 100, 62.5, 37.5, 25, 12.5, 8.5, 5, 5 and 2.5 mM) and the reaction allowed to proceed for 80, 60, 45, 30, 15 or 5 minutes before the addition of the urea detection reagents O-Phthaldialdehyde and N-(1-Napthyl)ethylenediamine.

Raw data from each separate concentration of C0021158 IgG was plotted as a function of time for each concentration of arginine and fitted using a linear fit (GraphPad Prism) to derive initial velocities (slope values).

Initial velocity data was plotted against arginine concentration for each concentration of C0021158 IgG (FIG. 15A). Each trace was fitted to the Michaelis-Menten model (Y=Vₘₐₓ*X/(Kₘ+X)) in GraphPad Prism to derive maximum velocity (Vₘₐₓ) and enzyme/substrate affinity (Kₘ) values at each inhibitor concentration (Table 23).

TABLE 23

| | Vₘₐₓ (AU ms⁻¹) | Kₘ (mM) |
|---|---|---|
| 1 μM 00021158 IgG | 103.4 | 9.98e−11 |
| 100 nM 00021158 IgG | 112.8 | 0.59 |
| 30 nM 00021158 IgG | 89.23 | 1.71e−10 |
| 10 nM 00021158 IgG | 148.5 | 1.64 |
| No inhibitor | 537.8 | 13.78 |

The mechanism of action of enzyme inhibitors can be broadly divided into competitive, uncompetitive and non-competitive and can be identified as such based on their effects on Kₘ and Vₘₐₓ.

A competitive inhibitor binds only to free enzyme, often at the active site, causing the affinity of the enzyme for the substrate to be reduced (increased Kₘ) with no change to the maximum enzyme rate (Vₘₐₓ).

An uncompetitive inhibitor binds exclusively to the enzyme-substrate complex yielding an inactive enzyme-substrate-inhibitor complex. The enzyme's affinity for the substrate is increased (Kₘ is decreased) and the maximum enzyme rate (Vₘₐₓ) is decreased.

A non-competitive inhibitor binds equally well to free enzyme and enzyme-substrate complex. The enzyme's affinity for the substrate (Kₘ) is unchanged and the maximum enzyme rate (Vₘₐₓ) is decreased.

In the case of C0021158 IgG inhibitor Vₘₐₓ decreased in the presence of C0021158 IgG inhibitor, ruling out a competitive mode of action and pointing towards an uncompetitive or a non-competitive mode of action. To distinguish non-competitive from uncompetitive modes of action the effect on Kₘ must be assessed. Unfortunately, the initial velocity/arginine concentration plots had an unusual profile where, in the presence of inhibitor, the initial anticipated increase in initial velocity is followed by a drop as arginine concentration increases. This uncharacteristic profile led to poor fitting of the data making it difficult to derive an accurate Kₘ. The lack of accurate Kₘ data means C0021158 IgG cannot be assigned as uncompetitive or non-competitive. It is only possible to state that the IgG inhibitor is ‘not competitive’.

To assess the accessibility of the ARG2 active site, we analysed binding of the ARG2 small-molecule inhibitor S-(2-Boronoethyl)-L-cysteine hydrochloride (BEC) by isothermal titration calorimetry (ITC) using a VP ITC (MicroCal) set to 25° C. (FIG. 15B). BEC is an arginine analogue that binds to the active site of ARG2. First, volumes of 10 μl of a solution containing 0.75 mM BEC were added to 20 μM of ARG2 (monomer concentration). The resulting thermogram was integrated using NITPIC and the binding isotherm was fitted with SEDPHAT using a one-binding site model. In a second experiment, volumes of 10 μl of 0.50 mM BEC were added to a solution containing a pre-incubated 1:1 molar mixture of C0021158 fab and ARG2 (both 20 μM, incubation 1 h at 20° C.). The thermogram was integrated using NITPIC, however, the resulting heats of injection were in the range of the background suggesting drastically reduced binding of BEC to ARG2 in the presence of C0021158 fab. Together with the EIA data, this suggests that C0021158 fab prevents the substrate from binding to ARG2 as an allosteric inhibitor.

3.8 Germlining and De-Risking of Lead Antibodies

Antibodies derived from selection outputs during the lead optimisation phase can often contain spontaneous mutations in the framework regions which deviate from germline sequences. As a measure to potentially decrease immunogenicity in vivo, such mutations can be reverted to germline sequences in lead candidates. For antibody clone C0021142, an amino acid residue in the VL Framework 3 region was reverted to germline during the IgG conversion process (C0021142 IgG). Similarly, an amino acid residue in the VH framework 4 region was reverted to germline sequences during the IgG conversion process (C0021158 IgG). The IgG and Fab versions of these antibody clones would carry these sequences. Furthermore, C0021158 was further germlined in the VL framework 3 region to produce a fully-germlined (fgl) version, named C0021158fgl. In further molecular engineering, a predicted mid-risk deamidation site was also engineered out of C0021158 to produce a de-risked (dr) version, named C0021158dr. Finally, a version of C0021158 which was fully germlined and de-risked was produced, named C0021158fgl2.

3.9 Inhibition of THP-1 Derived ARG2 by Second Generation Improved Variant IgGs

Second generation improved variant anti-ARG2 IgGs were tested for their ability to inhibit mammalian expressed human ARG2 produced in transfected THP-1 cells. Methods for this assay are described in section 2.8. Results are shown in FIG. 16 and Table 24.

TABLE 24

| | THP-1 ARG2 |
|---|---|
| Clone ID | EIA IC50 (M) |
| C0020187gG | 6.49E−07 |
| C0021158 gG | 2.30E−09 |
| C0021158-fgl2 IgG | 3.00E−09 |
| NHLA | 1.29E−05 |
| R347 IgG | inactive |

3.10 Demonstration of Release of ARG2 from Cancer Cell Lines

In this study 25 cancer cell lines were chosen to represent various disease indications. Based on ARG2 expression in culture media as described below (Table 25), a panel of cell lines from three disease indications viz., prostate, sarcoma and ovarian cancer were chosen for further assessment. These cell lines were obtained either from ATCC, ECACC or Essen Biosciences.

Serum-starvation treatment: The cell lines were grown in 35 ml of providers recommended media (for details see table below*) in a T175 cm² flask in 37° C. humidified incubator with 5% CO₂ until the cells were >70% confluent. Cells were washed with 25 ml of 1×DPBS (Gibco, ref. 14190-094) and replaced with 35 ml of serum-starved medium (i.e. growth media devoid of serum). 48 h later, medium on top of the cells (now referred to as Cell Conditioned Medium or CCM) was collected in a 50 ml Falcon tube and centrifuged at 300×g at 4° C. to harvest floating/dead cells. CCM on top of the cell pellet was collected in a separate 50 ml Falcon tube and filtered through 0.22 μm Millipore Express® filters (Millipore, cat. SCGP00525) to remove cell debris. The filtered CCM was stored on ice for concentration.

Adherent cells were trypsinised with 5 ml of TrypLE-Express (Gibco, cat. 12604-013) for up to 5 minutes in 37° C. incubator. 25 ml of pre-warmed medium was added when cells appeared rounded/detached under the microscope, and gently mixed to obtain single cell suspension. Transferred cells to the Falcon tube containing harvested cells from CCM and gently mixed. The cells were collected by centrifugation at 300×g at 4° C. for 5 min. Removed supernatant, resuspended cells in 30 ml of 1×DPBS. Cell count and viability was determined using Trypan Blue staining (Invitrogen, cat. T10282) and Countess® II Automated cell counter (Life Technologies) by following manufacturers' protocol.

Concentration of CCM: The filtered CCM was concentrated by centrifugation using Amicon® Ultra-15 filter (Millipore, ref. UCF901096). 10 ml of 1×DPBS was added to pre-soak the filter and centrifuged at 4000 rpm at 20° C. for 8 min. Filtrate collected in the receptacle tube was discarded. CCM was added to the tube, centrifuged at 4000 rpm at 20° C. for 6-8 min. This step was repeated, filtrate was discarded and CCM was added each time until ˜700 μl of concentrated CCM (˜50× concentrated) remained on the filter. Concentrated CCM was collected in a pre-labelled microfuge tube, 1× Protease Halt inhibitor (ThermoFisher, cat. 1861281) was added to it and mixed well. The concentrated CCM was either at stored at −20° C. or processed directly for immunoprecipitation.

Immunoprecipitation (IP) and Western blotting (WB): The volume of concentrated CCM corresponding to 5×10⁶ cells was determined. CCM was diluted in 550 μl of wash buffer (1×TBS, 1.5 M NaCl, 0.25 M tris pH 7.4, 0.05% Tween-20). Similarly, 25 ng of recombinant human ARG1 and ARG2 protein which would serve as negative and positive control were diluted separately in wash buffer. For each IP reaction, 2 μl of ARG2 PAD antibody (Cloud Clone Corp, ref. PAD796Hu01) was diluted in 50 μl of wash buffer and added to IP tube. IP reaction tubes were left on a rotator at 11 rpm at 4° C. overnight. Next day, 25 μl of Dynabeads™ (M-280 Sheep Anti-Rabbit IgG, Invitrogen, 11203D) were washed twice with 1 ml of wash buffer. Beads were resuspended in 50 μl of wash buffer and added to each IP tube. Left tubes on a rotator, 11 rpm at room temperature for 2 h. The magnet was used to remove the liquid. The beads were washed twice with 500 μl of wash buffer.

Suspended IP beads in 15 μl 1× NuPAGE LDS sample loading buffer (Invitrogen, cat. NP0007) were incubated at room temperature for 20 min. IP beads were heated to 90° C. for 10 min and loaded onto a NuPAGE™ 4-12% Bis-Tris gel (Invitrogen, cat. NPO322BOX). Also loaded were protein marker comprising a mix of 7 μl Pre-stained protein standards (Invitrogen, LC5800) and 2 μl MagicMark™ XP (Invitrogen, LC5602). Gels were run in 1× NuPAGE™ MES SDS running buffer (Invitrogen, cat. NP0002) at 200 V, 500 mA for 35 min. Resolved proteins were transferred to PVDF membranes (Invitrogen, cat. IB24001) using iBlot® 2 (Invitrogen) at 20 V for 1 min, 23V for 4 min and 25V for 2 min. Membranes were blocked in 5% Milk-TBS-Tween20 for 1 h at room temperature on a roller at 22 rpm. ARG2 MAD antibody (Cloud Clone Corp, ref. MAD796Hu21) was diluted to 1:1000 in 5 ml of 5% Milk-TBS-Tween20 and left on roller at 4° C. overnight. Membranes were washed three times with 1×TBS-Tween20 (˜5 min each time). Anti-Mouse (Fc specific)-HRP conjugated antibody (Sigma cat. A2254) was diluted to 1:2000 in 5 ml of 5% Milk-TBS-Tween20 and incubated with mouse-HRP antibody for 1 h at room temperature. The membranes were washed three times with 1×TBS-T-Tween20. The blots were developed using ECL Prime detection reagent (GE Healthcare, RPN2232) following manufacturer's protocol.

Chemiluminescent images were captured on Gel Imager (ChemiDoc-It²). Final set of images were inverted for colour on ImageJ and annotated.

Out of the various cell lines tested, ARG2 immunoprecipitated from a variety of cell lines (Table 25) including from CCM of LNCaP, A673 and A2780-Cis cell lines (FIG. 17).

TABLE 25
Relative levels of ARG2 released into cell culture media (CCM)
by different cancer cell lines. ARG2 in CCM was detected by
Immunoprecipitation (IP-WB), ‘+’ indicates presence of ARG2,
‘+++’ indicates high levels of ARG2, ‘+/−’ ARG2 presence could
not be unambiguously established and ‘−’ no ARG2 was detected.

| | | | ARG2 |
|---|---|---|---|
| | | | detected |
| S. No. | Cell Line | Disease Indication | by IP-WB |
| 1 | A673 | Sarcoma | +++ |
| 2 | HT-1080 | Sarcoma | − |
| 3 | Saos-2 | Sarcoma | + |
| 4 | U2-OS | Sarcoma | − |
| 5 | SKOV-3 | Ovarian | − |
| 6 | OVCAR-3 | Ovarian | +/− |
| 7 | A2780-Parental | Ovarian | + |
| 8 | A2780-Cis | Ovarian | +++ |
| 9 | A2780-ADR | Ovarian | + |
| 10 | DU145 | Prostate | − |
| 11 | LNCaP | Prostate | +++ |
| 12 | PC3 | Prostate | − |
| 13 | PNT2* | Prostate* | − |
| 14 | AGS | Gastric | + |
| 15 | HuTu-80 | Gastric | + |
| 16 | KATO-III | Gastric | + |
| 17 | NCI-N87 | Gastric | − |
| 18 | SNU-1 | Gastric | − |
| 19 | SNU-5 | Gastric | +/− |
| 20 | SNU-16 | Gastric | + |
| 21 | DX3 | Skin | +/− |
| 22 | HPAF-II | Pancreatic | +/− |
| 23 | MDA-MB-231 | Breast | − |
| 24 | K562 | Haematopoietic and Lymphoid | − |
| 25 | KMS11 | Haematopoietic and Lymphoid | + |
| 26 | HL-60 | Haematopoietic and Lymphoid | +/− |

*Cell line derived from normal prostate epithelium

TABLE 26
Composition of growth media for culturing cell lines.

| Cell Lines | Composition of growth media including supplements |
|---|---|
| A673, | DMEM (Gibco, ref. 31053-044) + 1x GlutaMAX ™ (Gibco, |
| PNT2, | ref. 35050-038) + 1 mM sodium pyruvate (Gibco, ref. |
| DX3, | 11360-070) + 10% FBS (Gibco, ref. 16170-078) |
| MDA- | |
| MB-231 | |
| LNCaP, | 1x RPMI ATCC Modification (Gibco, ref. A10491-01) + |
| NCI-N87, | 10% FBS |
| SNU-1, | |
| SNU-16 | |
| HT-1080^{$}, | Hams Nutrient Mix F12 (Sigma, ref. 51615C) + 10% FBS + |
| PC3, | ^{$}0.5 μg/ml Puromycin |
| AGS | |
| Saos-2^{#}, | McCoy’s 5A with GlutaMAX ™ (Gibco, ref. 36600-21) + |
| U2-OS, | 10% FBS/15% FBS |
| SKOV-3 | |
| A2780-Par, | RPMI Medium 1640 (Gibco, ref. 32404-014) + 1x |
| KMS11, | GlutaMAX ™ + 10% FBS + |
| A2780- | 1 μM Cisplatin (Millipore, ref. 232120)/1 μM Doxorubicin |
| Cis^{£} | (Sigma, ref. D1515) |
| A2780- | |
| ADR^{¥} | |
| OVCAR-3 | 1x RPMI ATCC Modification + Recombinant human Insulin |
| | (Sigma, ref. 19278, added 0.0798 μl/ml of media) + |
| | 20% FBS |
| DU-145, | MEM (Gibco, ref. 51200) + 1x GlutaMAX ™ + 1 mM |
| HPAF-II, | sodium pyruvate + 1x MEM Non-essential amino acid |
| HuTu-80 | solution (Gibco, ref. 11140-035) + 10% FBS |
| SNU-5, | IMDM (Gibco, ref. 12440-053) + 20% FBS/10% FBS |
| KATO-III, | |
| K562, | |
| HL-60^{Δ} | |

3.11 Binding of 1^{st} and 2^{nd} Generation IgGs (C0021061 and C0021158) to ARG2 from Cancer Cell Lines

In this experiment the presence of ARG2 in CCM samples was validated using commercial ARG2 ELISA kit (Cloud-Clone Corp., cat. SED796Hu). Additionally, the protocol was modified to include 1^{st} generation IgG C0021061 and 2^{nd} generation IgG C0021158 as detection antibodies to confirm binding of ARG2 IgG's to ARG2 expressed from cancer cell lines (FIG. 18).

The commercial kit is a 96-well plate pre-coated with anti-ARG2, which uses sandwich ELISA to quantify ARG2 in samples. Standards, biotinylated detection antibody, detection, substrate, and stop reagents are all provided in the kit. The concentrated CCM samples from LNCaP, A673 and A2780-Cis cells which showed ARG2 expression (as described above, FIG. 17) and a media control were diluted 1:1 in 1×DPBS. 100 μl of diluted CCM/media control was used for each well of kit and ELISA was performed following manufacturer's protocol. Each sample was run in duplicate. In parallel, CCM/media control samples were also included to test for binding of C0021061 and C0021158 to ARG2 in CCM samples. For this, while all steps were same as the manufacturer's protocol, instead of using kit-provided biotinylated detection antibody (PAD), biotinylated-C0021061/-C0021158/-irrelevant IgG control R347 at 10 μg/ml were used. At the end of the assay, absorbance at 450 nm was read out on PheraStar (BMG). The absorbance values of standards were used to make standard curve and was used to interpolate ARG2 levels in CCM samples (where PAD detection antibody supplied with the kit was used). Similarly, absorbance reads from wells that received C0021061 and C0021158 were used to infer₁₁ and 2^{nd} generation ARG2 IgGs (C0021061 and C0021158 respectively) bind to ARG2 in CCM samples (FIG. 18).

3.12. Co-Crystallisation of Human ARG2 with ARG2 Binding Fabs C0021158 and C0021181 to Elucidate Epitope/Paratope Contact Residues

The construct used for crystal trials was ARG2 (23-354) containing a C-terminal Gly₃ linker and His₁₀-tag (ARG2+C0020187, SEQ ID NO: 355: huARG2-His10) or a simple C-terminal His₆-tag (ARG2+C0021181 and ARG2+C0021158, SEQ ID NO: 357: huARG2-His₆). Crystals suitable for structure determination were obtained using the sitting drop methodology. For ARG2+C0020187, 0.2 μl of complex solution (2.0 mg/ml, ARG2:Fab=3:1, purified by gel filtration) were mixed with 0.1 μl of crystallisation solution containing 2 M (NH₄)₂SO₄. Round, disc-shaped crystals (60×60×20 μm) appeared after 2 days. For ARG2+C0021181, 0.5 μl of complex solution (6.3 mg/ml) were mixed with 0.5 μl of a crystallisation solution containing 100 mM MMT (malic acid:MES:tris=1:2:2) pH 5.0, 20% glycerol, 10% PEG4000, 15 mM NaNO₃, 15 mM Na₂HPO₄, 15 mM (NH₄)₂SO₄. Hexagonal disc-shaped crystals (100×100×20 μm) appeared after 2 days and reached their final size after a week. For ARG2+C0021158, 0.25 μl of complex solution (7.0 mg/ml, containing 20% diluted microseeds of an ARG2+C0021181 crystal) were mixed with 0.25 μl of a crystallisation solution containing 2 M (NH₄)₂SO₄. Crystals of irregular shape (60×60×60 μm) appeared after 2 days. For the ARG2+C0020187 and ARG2+C0021158 crystals, 3 M (NH₄)₂SO₄ and 5% glycerol were added for cryo-protection, after which the crystals were cryo-cooled by plunging them into liquid N₂. Data sets were collected at DLS, beamline i04-1 (ARG2+C0020187) or BESSY II, HZB, beamline MX-14-1 (ARG2+C0021181 and ARG2+C0021158). The crystals of ARG2+C0020187 diffracted up to 3.25 Å in space group P 6₅ 2 2 with unit cell parameters a=b=138 Å, c=551 Å. The crystals of ARG2+C0021181 diffracted up to 2.9 Å in space group P 3 2 1 with unit cell parameters a=b=150 Å, c=111 Å, and exhibited twinning with fractions 0.50/0.50. The crystals of ARG2+C0021158 diffracted up to 2.4 Å in space group H 3 with unit cell parameters a=b=149 Å, c=123 Å, and exhibited twinning with fractions 0.80/0.20.

3.13 Structure Solution and Refinement

The structures were solved by molecular replacement using Phaser in the CCP4 program suite using the reported structure of ARG2 (PDB-ID 4HZE) and previously solved high-resolution structures of the individual Fabs in the molecular replacement search. For ARG2+C0020187 the asymmetric unit contained three copies each of ARG2 and the Fab, respectively, with the C_{L} and C_{H} domains being present in different orientations, one of pair which was poorly defined in the electron density. For ARG2+C0021181 and ARG2+C0021158 the asymmetric units contained one copy of ARG2 and one copy of the Fab, however for ARG2+C0021181 only the V_{L} and V_{H} domains of the Fab were well defined, with C_{L} and C_{H} domains most likely being present in two orientations (reflected by the increased B factor and overall worse electron density). Automated and manual refinement was then performed by alternating between using REFMAC5 (with twin- and TLS refinement turned on towards finalising the structures for ARG2+C0021181 and ARG2+C0021158) and COOT, respectively. Final R/R_{free} values of the ARG2+C0020187, ARG2+C0021181 and the ARG2+C0021158 structures are 0.30/0.36, 0.27/0.32 and 0.25/0.28, respectively (FIG. 19).

TABLE 27
Table indicating the percentage surface area of solvent accessible
residues within ARG2 that become buried upon complexation with
C0021181. ASA is the solvent accessible surface area of residues. BSA
is the buried (solvent inaccessible) surface area. ΔG is the predicted
solvation free energy gained upon formation of the ARG2-C0021181
interface for each residue.

| ARG2 | H-bond/ | ASA | BSA | ΔG | % | Interface |
|---|---|---|---|---|---|---|
| residue | salt bridge | (Å²) | (Å²) | (kcal/mol) | buried | to Fab |
| GLN37 | \|H\| | 79.8 | 40.8 | 0.1 | 51% | V_{H} |
| LYS38 | \|HS\| | 154.2 | 60.1 | 0.1 | 39% | V_{H} |
| LYS40 | \| \| | 47.3 | 17.7 | 0.2 | 37% | V_{H} |
| GLY41 | \| \| | 1.0 | 1.0 | 0.0 | 100% | V_{H} |
| HIS44 | \|H\| | 119.3 | 79.3 | −0.4 | 66% | V_{H} |
| ALA47 | \| \| | 44.9 | 5.7 | 0.1 | 13% | V_{H} |
| ALA48 | \| \| | 18.4 | 5.5 | 0.1 | 30% | V_{H} |
| GLU51 | \|S\| | 149.7 | 30.5 | −0.4 | 20% | V_{H} |
| ASP79 | \| \| | 89.1 | 7.8 | −0.1 | 9% | V_{H} |
| ASP80 | \| \| | 80.0 | 44.9 | 0.2 | 56% | V_{H} + V_{L} |
| LEU81 | \| \| | 132.9 | 132.9 | 1.9 | 100% | V_{H} + V_{L} |
| TYR82 | \| \| | 130.6 | 94.1 | 1.4 | 72% | V_{H} + V_{L} |
| ASN84 | \|H\| | 48.3 | 26.5 | 0.1 | 55% | V_{H} |
| LEU85 | \| \| | 97.8 | 94.5 | 1.5 | 97% | V_{H} + V_{L} |
| ILE86 | \| \| | 65.9 | 46.6 | 0.7 | 71% | V_{H} + V_{L} |
| PRO299 | \| \| | 36.7 | 34.6 | 0.3 | 95% | V_{H} |
| GLN300 | \|H\| | 122.8 | 94.4 | 0.1 | 77% | V_{H} |
| ALA302 | \| \| | 23.5 | 11.3 | −0.1 | 48% | V_{H} |
| THR303 | \| \| | 121.5 | 5.2 | 0.0 | 4% | V_{H} |
| SER304 | \| \| | 56.2 | 14.4 | 0.2 | 26% | V_{H} |
| GLU305 | \| \| | 131.9 | 34.5 | 0.4 | 26% | V_{H} |
| ALA308 | \| \| | 19.4 | 7.2 | 0.1 | 37% | V_{H} |
| total: | | 1°°1.2 | 889.° | 6.° | 50% | |

TABLE 28
Table indicating the percentage surface area of solvent accessible
residues within ARG2 that become buried upon complexation with
C0021158. ASA is the solvent accessible surface area of residues. BSA
is the buried (solvent inaccessible) surface area. ΔG is the predicted
solvation free energy gained upon formation of the ARG2-C0021158
interface for each residue.

| ARG2 | H-bond/ | ASA | BSA | ΔG | % | Interface |
|---|---|---|---|---|---|---|
| residue | salt bridge | (Å²) | (Å²) | (kcal/mol) | buried | to Fab |
| PRO32 | \| \| | 19.0 | 13.1 | 0.2 | 69% | V_{H} |
| GLN37 | \|H\| | 104.9 | 60.0 | −0.1 | 57% | V_{H} |
| LYS38 | \|H\| | 174.3 | 44.7 | 0.6 | 26% | V_{H} |
| LYS40 | \| \| | 57.4 | 35.7 | 0.4 | 62% | V_{H} |
| GLY41 | \| \| | 5.4 | 3.6 | 0.1 | 67% | V_{H} |
| GLU43 | \| \| | 34.8 | 28.3 | −0.2 | 81% | V_{H} |
| HIS44 | \|H\| | 113.8 | 103.2 | −0.1 | 91% | V_{H} |
| ALA47 | \| \| | 41.9 | 18.7 | 0.3 | 45% | V_{H} |
| ALA48 | \| \| | 20.6 | 7.8 | 0.1 | 38% | V_{H} |
| GLU51 | \|S\| | 153.6 | 38.0 | −0.5 | 25% | V_{H} |
| ASP70 | \| \| | 64.9 | 0.3 | 0.0 | 1% | V_{H} |
| SER72 | \| \| | 88.9 | 17.9 | 0.3 | 20% | V_{H} |
| PHE73 | \| \| | 28.8 | 11.1 | −0.1 | 39% | V_{H} |
| THR74 | \| \| | 73.6 | 10.4 | −0.1 | 14% | V_{H} |
| PRO75 | \| \| | 4.8 | 0.3 | 0.0 | 7% | V_{H} |
| LYS78 | \| \| | 115.21 | 16.64 | −0.42 | 14% | V_{L} |
| ASP79 | \|S\| | 94.0 | 25.6 | −0.3 | 27% | V_{H} |
| ASP80 | \| \| | 58.2 | 44.0 | 0.0 | 76% | V_{H} + V_{L} |
| LEU81 | \| \| | 136.5 | 136.5 | 2.0 | 100% | V_{H} + V_{L} |
| TYR82 | \| \| | 134.8 | 98.9 | 1.3 | 73% | V_{H} + V_{L} |
| ASN84 | \|H\| | 41.5 | 32.5 | −0.1 | 78% | V_{H} |
| LEU85 | \| \| | 82.2 | 80.9 | 1.3 | 98% | V_{H} |
| ILE86 | \| \| | 77.9 | 54.6 | 0.9 | 70% | V_{H} + V_{L} |
| PRO299 | \| \| | 29.6 | 25.9 | 0.3 | 87% | V_{H} |
| GLN300 | \|H\| | 108.6 | 75.7 | 0.0 | 70% | V_{H} |
| GLU305 | \| \| | 126.1 | 6.4 | 0.1 | 5% | V_{H} |
| ALA308 | \| \| | 21.5 | 9.5 | 0.2 | 44% | V_{H} |
| total: | | 2012.6 | 1000.3 | 6.2 | 50% | |

TABLE 29
Table indicating the percentage surface area of solvent accessible
residues within ARG2 that become buried upon complexation with
C0020187 over all three protomers in the asymmetric unit of the
crystal structure. The numbers after the residue show in how many
of the three protomers the residue is involved in the interface. ASA is
the solvent accessible surface area of residues. BSA is the buried
(solvent inaccessible) surface area. ΔG is the predicted solvation
free energy gained upon formation of the ARG2-C0020187 interface
for each residue. ASA, BSA and ΔG are averages of the protomers
in which they occur and H-bonds are indicated if at least one of the
protomers allows for one to be formed.

| ARG2 | | H-bond/ | ASA | BSA | ΔG | % | Interface |
|---|---|---|---|---|---|---|---|
| residue | | salt bridge | (Å²) | (Å²) | (kcal/mol) | buried | to Fab |
| GLN35 | 2/3 | \| \| | 32.3 | 13.7 | −0.2 | 42% | V_{H} |
| GLY36 | 1/3 | \| \| | 23.8 | 0.3 | 0.0 | 1% | V_{H} |
| GLN37 | 3/3 | \|H\| | 118.7 | 24.4 | −0.2 | 21% | V_{H} |
| LYS38 | 3/3 | \| \| | 170.6 | 36.0 | 0.3 | 21% | V_{H} |
| ARG39 | 2/3 | \|H\| | 65.8 | 21.4 | −0.3 | 33% | V_{H} |
| LYS78 | 2/3 | \|H\| | 156.9 | 16.1 | −0.1 | 10% | V_{L} |
| ASP79 | 1/3 | \| \| | 75.7 | 3.9 | 0.0 | 5% | V_{H} + V_{L} |
| ASP80 | 3/3 | \|H\| | 80.6 | 27.2 | 0.0 | 34% | V_{H} + V_{L} |
| LEU81 | 3/3 | \| \| | 109.1 | 85.8 | 1.0 | 79% | V_{H} + V_{L} |
| TYR82 | 3/3 | \| \| | 133.4 | 108.8 | 1.2 | 82% | V_{H} |
| ASN84 | 3/3 | \|H\| | 58.1 | 39.0 | −0.3 | 67% | V_{H} |
| LEU85 | 3/3 | \| \| | 115.2 | 72.5 | 0.8 | 63% | V_{H} + V_{L} |
| ILE86 | 3/3 | \| \| | 74.8 | 47.9 | 0.8 | 64% | V_{H} |
| LEU152 | 3/3 | \|H\| | 123.8 | 23.7 | −0.1 | 19% | V_{H} |
| THR153 | 3/3 | \| \| | 56.7 | 15.2 | 0.0 | 27% | V_{H} |
| THR154 | 3/3 | \|H\| | 45.0 | 14.7 | 0.1 | 33% | V_{H} |
| SER155 | 3/3 | \|H\| | 82.7 | 60.5 | 0.4 | 73% | V_{H} |
| SER156 | 3/3 | \|H\| | 51.9 | 21.8 | 0.1 | 42% | V_{H} |
| GLY157 | 3/3 | \|H\| | 35.6 | 22.2 | 0.2 | 62% | V_{H} |
| LEU178 | 1/3 | \| \| | 53.3 | 7.2 | 0.1 | 14% | V_{H} |
| PRO179 | 3/3 | \| \| | 72.3 | 16.7 | 0.3 | 23% | V_{H} |
| total: | total: | | 1° 36.3 | 6° 8.9 | 3.9 | 39% | V_{H} |

3.14 Size-Exclusion Chromatography to Analyse ARG2-Fab Binding Stoichiometry

ARG2 and Fabs were analysed at concentrations of 8 μM (referring to monomers) in SEC buffer containing 25 mM tris at pH 7.4 and 150 mM NaCl. ARG2-Fab complexes were formed by mixing both ARG2 and Fabs at concentrations of 8 μM (referring to monomers) in SEC buffer and incubation on ice for 1 h. For size-exclusion chromatography, 500 μl of sample were injected into a 100 μl loop connected to a Superose 6 Increase 10/300 GL column (GE Healthcare) which was mounted to an AKTAexpress FPLC system (GE Healthcare). The column was equilibrated in SEC buffer, and SEC was performed at a flow rate of 0.5 ml/min. Molecular-weight estimates are based on the retention volumes seen for β-amylase, bovine serum albumin and carbonic anhydrase, which were analysed on the same FPLC system under identical conditions.

3.15 Dynamic Light Scattering to Analyse ARG2-Fab Complex Sizes

ARG2 and Fabs were analysed at concentrations of 8 μM (referring to monomers) in buffer containing 25 mM tris at pH 7.4 and 150 mM NaCl (FIG. 23). ARG2-Fab complexes were formed by mixing both ARG2 and Fabs at concentrations of 8 μM (referring to monomers) and incubation on ice for 1 h. Dynamic light scattering (DLS) was performed on a Zetasizer Ultra (Malvern Panalytical) using multiangle DLS. Data was collected in a 3×3 mm cuvette (Hellma Analytics) using a sample volume of 30 μl. Volume weighted size-distributions were corrected for refractive index, viscosity, and background scatter of the buffer by the instrument software.

### CITATION LIST

1. Ash, D. E., Structure and function of arginases. J Nutr, 2004. 134(10 Suppl): p. 2760S-2764S; discussion 2765S-2767S.
2. Balkwill, F., K. A. Charles, and A. Mantovani, Smoldering and polarized inflammation in the initiation and promotion of malignant disease. Cancer Cell, 2005. 7(3): p. 211-7.
3. Bannister, D., et al., Parallel, high-throughput purification of recombinant antibodies for in vivo cell assays. Biotechnol Bioeng, 2006. 94(5): p. 931-7.
4. Bivalacqua, T. J., et al., Evaluation of nitric oxide synthase and arginase in the induction of a Peyronie's-like condition in the rat. J Androl, 2001. 22(3): p. 497-506.
5. Bron, L., et al., Prognostic value of arginase-II expression and regulatory T-cell infiltration in head and neck squamous cell carcinoma. Int J Cancer, 2013. 132(3): p. E85-93.
6. Bronte, V., et al., Boosting antitumor responses of T lymphocytes infiltrating human prostate cancers. J Exp Med, 2005. 201(8): p. 1257-68.
7. Caldwell, R. B., et al., Arginase: an old enzyme with new tricks. Trends Pharmacol Sci, 2015. 36(6): p. 395-405.
8. Caldwell, R. W., et al., Arginase: A Multifaceted Enzyme Important in Health and Disease. Physiol Rev, 2018. 98(2): p. 641-665.
9. Christianson, D. W., Arginase: structure, mechanism, and physiological role in male and female sexual arousal. Acc Chem Res, 2005. 38(3): p. 191-201.
10. Clackson, T. and H. B. Lowman, Phage display: a practical approach. Practical approach series. 2004: Oxford.
11. Costa, H., et al., Human cytomegalovirus may promote tumour progression by upregulating arginase-2. Oncotarget, 2016. 7(30): p. 47221-47231.
12. Di Costanzo, L., et al., Crystal structure of human arginase I at 1.29-A resolution and exploration of inhibition in the immune response. Proc Natl Acad Sci USA, 2005. 102(37): p. 13058-63.
13. Dunand-Sauthier, I., et al., Repression of arginase-2 expression in dendritic cells by microRNA-155 is critical for promoting T cell proliferation. J Immunol, 2014. 193(4): p. 1690-700.
14. Durante, W., F. K. Johnson, and R. A. Johnson, Arginase: a critical regulator of nitric oxide synthesis and vascular function. Clin Exp Pharmacol Physiol, 2007. 34(9): p. 906-11.
15. Gannon, P. O., et al., Androgen-regulated expression of arginase 1, arginase 2 and interleukin-8 in human prostate cancer. PLoS One, 2010. 5(8): p. e12107.
16. Groves, M. A. and J. K. Osbourn, Applications of ribosome display to antibody drug discovery. Expert Opin Biol Ther, 2005. 5(1): p. 125-35.
17. Hanes, J., L. Jermutus, and A. Pluckthun, Selecting and evolving functional proteins in vitro by ribosome display. Methods Enzymol, 2000. 328: p. 404-30.
18. Hanes, J. and A. Pluckthun, In vitro selection and evolution of functional proteins by using ribosome display. Proc Natl Acad Sci USA, 1997. 94(10): p. 4937-42.
19. Hawkins, R. E., S. J. Russell, and G. Winter, Selection of phage antibodies by binding affinity. Mimicking affinity maturation. J Mol Biol, 1992. 226(3): p. 889-96.
20. Hecker, M., et al., Inhibition of arginase by NG-hydroxy-L-arginine in alveolar macrophages: implications for the utilization of L-arginine for nitric oxide synthesis. FEBS Lett, 1995. 359(2-3): p. 251-4.
21. Ino, Y., et al., Arginase II expressed in cancer-associated fibroblasts indicates tissue hypoxia and predicts poor outcome in patients with pancreatic cancer. PLoS One, 2013. 8(2): p. e55146.
22. Jayaraman, P., et al., Tumor-expressed inducible nitric oxide synthase controls induction of functional myeloid-derived suppressor cells through modulation of vascular endothelial growth factor release. J Immunol, 2012. 188(11): p. 5365-76.
23. Jenkinson, C. P., W. W. Grody, and S. D. Cederbaum, Comparative properties of arginases. Comp Biochem Physiol B Biochem Mol Biol, 1996. 114(1): p. 107-32.
24. Kabat, E. A. and T. T. Wu, Identical V region amino acid sequences and segments of sequences in antibodies of different specificities. Relative contributions of VH and VL genes, minigenes, and complementarity-determining regions to binding of antibody-combining sites. J Immunol, 1991. 147(5): p. 1709-19.
25. Kim, S. H., et al., Impact of I-Arginine Metabolism on Immune Response and Anticancer Immunotherapy. Front Oncol, 2018. 8: p. 67.
26. Lewis, N. D., et al., Immune evasion by Helicobacter pylori is mediated by induction of macrophage arginase II. J Immunol, 2011. 186(6): p. 3632-41.
27. Maarsingh, H., et al., Arginase strongly impairs neuronal nitric oxide-mediated airway smooth muscle relaxation in allergic asthma. Respir Res, 2006. 7: p. 6.
28. Mantovani, A., et al., Macrophage polarization: tumor-associated macrophages as a paradigm for polarized M2 mononuclear phagocytes. Trends Immunol, 2002. 23(11): p. 549-55.
29. McCafferty, J., et al., Selection and rapid purification of murine antibody fragments that bind a transition-state analog by phage display. Appl Biochem Biotechnol, 1994. 47(2-3): p. 157-71; discussion 171-3.
30. Ming, X. F., et al., Arginase II Promotes Macrophage Inflammatory Responses Through Mitochondrial Reactive Oxygen Species, Contributing to Insulin Resistance and Atherogenesis. J Am Heart Assoc, 2012. 1(4): p. e000992.
31. Miraki-Moud, F., et al., Arginine deprivation using pegylated arginine deiminase has activity against primary acute myeloid leukemia cells in vivo. Blood, 2015. 125(26): p. 4060-8.
32. Monu, N. R. and A. B. Frey, Myeloid-derived suppressor cells and anti-tumor T cells: a complex relationship. Immunol Invest, 2012. 41(6-7): p. 595-613.
33. Morris, S. M., Jr., Arginine metabolism: boundaries of our knowledge. J Nutr, 2007. 137(6 Suppl 2): p. 1602S-1609S.
34. Muenst, S., et al., The immune system and cancer evasion strategies: therapeutic concepts. J Intern Med, 2016. 279(6): p. 541-62.
35. Mumenthaler, S. M., et al., Expression of arginase II in prostate cancer. Int J Oncol, 2008. 32(2): p. 357-65.
36. Munder, M., et al., Suppression of T-cell functions by human granulocyte arginase.

Blood, 2006. 108(5): p. 1627-34.
37. Munn, D. H. and V. Bronte, Immune suppressive mechanisms in the tumor microenvironment. Curr Opin Immunol, 2016. 39: p. 1-6.
38. Mussai, F., et al., Acute myeloid leukemia creates an arginase-dependent immunosuppressive microenvironment. Blood, 2013. 122(5): p. 749-58.
39. Mussai, F., et al., Arginine dependence of acute myeloid leukemia blast proliferation: a novel therapeutic target. Blood, 2015. 125(15): p. 2386-96.
40. Mussai, F., et al., Neuroblastoma Arginase Activity Creates an Immunosuppressive Microenvironment That Impairs Autologous and Engineered Immunity. Cancer Res, 2015. 75(15): p. 3043-53.
41. Odri, G. A., et al., Zoledronic acid as a new adjuvant therapeutic strategy for Ewing's sarcoma patients. Cancer Res, 2010. 70(19): p. 7610-9.
42. Osbourn, J. K., et al., Generation of a panel of related human scFv antibodies with high affinities for human CEA. Immunotechnology, 1996. 2(3): p. 181-96.
43. Pandey, D., et al., OxLDL triggers retrograde translocation of arginase2 in aortic endothelial cells via ROCK and mitochondrial processing peptidase. Circ Res, 2014. 115(4): p. 450-9.
44. Persic, L., et al., An integrated vector system for the eukaryotic expression of antibodies or their fragments after selection from phage display libraries. Gene, 1997. 187(1): p. 9-18.
45. Polat, M. F., et al., Elevated serum arginase activity levels in patients with breast cancer. Surg Today, 2003. 33(9): p. 655-61.
46. Rabinovich, G. A., D. Gabrilovich, and E. M. Sotomayor, Immunosuppressive strategies that are mediated by tumor cells. Annu Rev Immunol, 2007. 25: p. 267-96.
47. Rodriguez, P. C., et al., Arginase I production in the tumor microenvironment by mature myeloid cells inhibits T-cell receptor expression and antigen-specific T-cell responses. Cancer Res, 2004. 64(16): p. 5839-49.
48. Rodriguez, P. C., et al., Regulation of T cell receptor CD3zeta chain expression by L-arginine. J Biol Chem, 2002. 277(24): p. 21123-9.
49. Rotondo, R., et al., Arginase 2 is expressed by human lung cancer, but it neither induces immune suppression, nor affects disease progression. Int J Cancer, 2008. 123(5): p. 1108-16.
50. Sabio, G., et al., Glu-256 is a main structural determinant for oligomerisation of human arginase I. FEBS Lett, 2001. 501(2-3): p. 161-5.
51. Sankaralingam, S., H. Xu, and S. T. Davidge, Arginase contributes to endothelial cell oxidative stress in response to plasma from women with preeclampsia. Cardiovasc Res, 2010. 85(1): p. 194-203.
52. Schier, R., et al., Isolation of high-affinity monomeric human anti-c-erbB-2 single chain Fv using affinity-driven selection. J Mol Biol, 1996. 255(1): p. 28-43.
53. Setty, B. A., et al., Hypoxic Proliferation of Osteosarcoma Cells Depends on Arginase II. Cell Physiol Biochem, 2016. 39(2): p. 802-13.
54. Singh, R., et al., Proteomic identification of mitochondrial targets of arginase in human breast cancer. PLoS One, 2013. 8(11): p. e79242.
55. Sousa, M. S., et al., Arginase 2 and nitric oxide synthase: Pathways associated with the pathogenesis of thyroid tumors. Free Radic Biol Med, 2010. 49(6): p. 997-1007.
56. Steggerda, S. M., et al., Inhibition of arginase by CB-1158 blocks myeloid cell-mediated immune suppression in the tumor microenvironment. J Immunother Cancer, 2017. 5(1): p. 101.
57. Thompson, J., et al., Affinity maturation of a high-affinity human monoclonal antibody against the third hypervariable loop of human immunodeficiency virus: use of phage display to improve affinity and broaden strain reactivity. J Mol Biol, 1996. 256(1): p. 77-88.
58. Thompson, J. D., D. G. Higgins, and T. J. Gibson, CLUSTAL W: improving the sensitivity of progressive multiple sequence alignment through sequence weighting, position-specific gap penalties and weight matrix choice. Nucleic Acids Res, 1994. 22(22): p. 4673-80.
59. Timosenko, E., A. V. Hadjinicolaou, and V. Cerundolo, Modulation of cancer-specific immune responses by amino acid degrading enzymes. Immunotherapy, 2017. 9(1): p. 83-97.
60. Umansky, V., et al., Interactions among myeloid regulatory cells in cancer. Cancer Immunol Immunother, 2018.
61. Vaughan, T. J., et al., Human antibodies with sub-nanomolar affinities isolated from a large non-immunized phage display library. Nat Biotechnol, 1996. 14(3): p. 309-14.
62. Xu, W., et al., Increased arginase II and decreased NO synthesis in endothelial cells of patients with pulmonary arterial hypertension. FASEB J, 2004. 18(14): p. 1746-8.
63. Xue, J., L. D. Nelin, and B. Chen, Hypoxia induces arginase II expression and increases viable human pulmonary artery smooth muscle cell numbers via AMPKalpha1 signaling. Am J Physiol Lung Cell Mol Physiol, 2017. 312(4): p. L568-L578.
64. Yaiw, K. C., et al., Human cytomegalovirus induces upregulation of arginase II: possible implications for vasculopathies. Basic Res Cardiol, 2014. 109(2): p. 401.
65. Yepuri, G., et al., Positive crosstalk between arginase-II and S6K1 in vascular endothelial inflammation and aging. Aging Cell, 2012. 11(6): p. 1005-16.
66. Yu, Y., et al., p38 mitogen-activated protein kinase is involved in arginase-II-mediated eNOS-uncoupling in obesity. Cardiovasc Diabetol, 2014. 13: p. 113.
67. Zaytouni, T., et al., Critical role for arginase 2 in obesity-associated pancreatic cancer. Nat Commun, 2017. 8(1): p. 242.
68. Zhao, H., et al., Molecular evolution by staggered extension process (StEP) in vitro recombination. Nat Biotechnol, 1998. 16(3): p. 258-61.

Sequence Listing Free Text

| Sequence ID | Clone Name | Sequence Type |
|---|---|---|
| 1 | C0020065 | VH DNA |
| 2 | C0020065 | VH PRT |
| 3 | C0020065 | CDR1 PRT |
| 4 | C0020065 | CDR2 PRT |
| 5 | C0020065 | CDR3 PRT |
| 6 | C0020065 | VL DNA |
| 7 | C0020065 | VL PRT |
| 8 | C0020065 | CDR1 PRT |
| 9 | C0020065 | CDR2 PRT |
| 10 | C0020065 | CDR3 PRT |
| 11 | C0020187 | VH DNA |
| 12 | C0020187 | VH PRT |
| 13 | C0020187 | CDR1 PRT |
| 14 | C0020187 | CDR2 PRT |
| 15 | C0020187 | CDR3 PRT |
| 16 | C0020187 | VL DNA |
| 17 | C0020187 | VL PRT |
| 18 | C0020187 | CDR1 PRT |
| 19 | C0020187 | CDR2 PRT |
| 20 | C0020187 | CDR3 PRT |
| 21 | C0021017 | VH DNA |
| 22 | C0021017 | VH PRT |
| 23 | C0021017 | CDR1 PRT |
| 24 | C0021017 | CDR2 PRT |
| 25 | C0021017 | CDR3 PRT |
| 26 | C0021017 | VL DNA |
| 27 | C0021017 | VL PRT |
| 28 | C0021017 | CDR1 PRT |
| 29 | C0021017 | CDR2 PRT |
| 30 | C0021017 | CDR3 PRT |
| 31 | C0021021 | VH DNA |
| 32 | C0021021 | VH PRT |
| 33 | C0021021 | CDR1 PRT |
| 34 | C0021021 | CDR2 PRT |
| 35 | C0021021 | CDR3 PRT |
| 36 | C0021021 | VL DNA |
| 37 | C0021021 | VL PRT |
| 38 | C0021021 | CDR1 PRT |
| 39 | C0021021 | CDR2 PRT |
| 40 | C0021021 | CDR3 PRT |
| 41 | C0021022 | VH DNA |
| 42 | C0021022 | VH PRT |
| 43 | C0021022 | CDR1 PRT |
| 44 | C0021022 | CDR2 PRT |
| 45 | C0021022 | CDR3 PRT |
| 46 | C0021022 | VL DNA |
| 47 | C0021022 | VL PRT |
| 48 | C0021022 | CDR1 PRT |
| 49 | C0021022 | CDR2 PRT |
| 50 | C0021022 | CDR3 PRT |
| 51 | C0021032 | VH DNA |
| 52 | C0021032 | VH PRT |
| 53 | C0021032 | CDR1 PRT |
| 54 | C0021032 | CDR2 PRT |
| 55 | C0021032 | CDR3 PRT |
| 56 | C0021032 | VL DNA |
| 57 | C0021032 | VL PRT |
| 58 | C0021032 | CDR1 PRT |
| 59 | C0021032 | CDR2 PRT |
| 60 | C0021032 | CDR3 PRT |
| 61 | C0021061 | VH DNA |
| 62 | C0021061 | VH PRT |
| 63 | C0021061 | CDR1 PRT |
| 64 | C0021061 | CDR2 PRT |
| 65 | C0021061 | CDR3 PRT |
| 66 | C0021061 | VL DNA |
| 67 | C0021061 | VL PRT |
| 68 | C0021061 | CDR1 PRT |
| 69 | C0021061 | CDR2 PRT |
| 70 | C0021061 | CDR3 PRT |
| 71 | C0021065 | VH DNA |
| 72 | C0021065 | VH PRT |
| 73 | C0021065 | CDR1 PRT |
| 74 | C0021065 | CDR2 PRT |
| 75 | C0021065 | CDR3 PRT |
| 76 | C0021065 | VL DNA |
| 77 | C0021065 | VL PRT |
| 78 | C0021065 | CDR1 PRT |
| 79 | C0021065 | CDR2 PRT |
| 80 | C0021065 | CDR3 PRT |
| 81 | C0021089 | VH DNA |
| 82 | C0021089 | VH PRT |
| 83 | C0021089 | CDR1 PRT |
| 84 | C0021089 | CDR2 PRT |
| 85 | C0021089 | CDR3 PRT |
| 86 | C0021089 | VL DNA |
| 87 | C0021089 | VL PRT |
| 88 | C0021089 | CDR1 PRT |
| 89 | C0021089 | CDR2 PRT |
| 90 | C0021089 | CDR3 PRT |
| 91 | C0021092 | VH DNA |
| 92 | C0021092 | VH PRT |
| 93 | C0021092 | CDR1 PRT |
| 94 | C0021092 | CDR2 PRT |
| 95 | C0021092 | CDR3 PRT |
| 96 | C0021092 | VL DNA |
| 97 | C0021092 | VL PRT |
| 98 | C0021092 | CDR1 PRT |
| 99 | C0021092 | CDR2 PRT |
| 100 | C0021092 | CDR3 PRT |
| 101 | C0021096 | VH DNA |
| 102 | C0021096 | VH PRT |
| 103 | C0021096 | CDR1 PRT |
| 104 | C0021096 | CDR2 PRT |
| 105 | C0021096 | CDR3 PRT |
| 106 | C0021096 | VL DNA |
| 107 | C0021096 | VL PRT |
| 108 | C0021096 | CDR1 PRT |
| 109 | C0021096 | CDR2 PRT |
| 110 | C0021096 | CDR3 PRT |
| 111 | C0021097 | VH DNA |
| 112 | C0021097 | VH PRT |
| 113 | C0021097 | CDR1 PRT |
| 114 | C0021097 | CDR2 PRT |
| 115 | C0021097 | CDR3 PRT |
| 116 | C0021097 | VL DNA |
| 117 | C0021097 | VL PRT |
| 118 | C0021097 | CDR1 PRT |
| 119 | C0021097 | CDR2 PRT |
| 120 | C0021097 | CDR3 PRT |
| 121 | C0021098 | VH DNA |
| 122 | C0021098 | VH PRT |
| 123 | C0021098 | CDR1 PRT |
| 124 | C0021098 | CDR2 PRT |
| 125 | C0021098 | CDR3 PRT |
| 126 | C0021098 | VL DNA |
| 127 | C0021098 | VL PRT |
| 128 | C0021098 | CDR1 PRT |
| 129 | C0021098 | CDR2 PRT |
| 130 | C0021098 | CDR3 PRT |
| 131 | C0021101 | VH DNA |
| 132 | C0021101 | VH PRT |
| 133 | C0021101 | CDR1 PRT |
| 134 | C0021101 | CDR2 PRT |
| 135 | C0021101 | CDR3 PRT |
| 136 | C0021101 | VL DNA |
| 137 | C0021101 | VL PRT |
| 138 | C0021101 | CDR1 PRT |
| 139 | C0021101 | CDR2 PRT |
| 140 | C0021101 | CDR3 PRT |
| 141 | C0021118 | VH DNA |
| 142 | C0021118 | VH PRT |
| 143 | C0021118 | CDR1 PRT |
| 144 | C0021118 | CDR2 PRT |
| 145 | C0021118 | CDR3 PRT |
| 146 | C0021118 | VL DNA |
| 147 | C0021118 | VL PRT |
| 148 | C0021118 | CDR1 PRT |
| 149 | C0021118 | CDR2 PRT |
| 150 | C0021118 | CDR3 PRT |
| 151 | C0021124 | VH DNA |
| 152 | C0021124 | VH PRT |
| 153 | C0021124 | CDR1 PRT |
| 154 | C0021124 | CDR2 PRT |
| 155 | C0021124 | CDR3 PRT |
| 156 | C0021124 | VL DNA |
| 157 | C0021124 | VL PRT |
| 158 | C0021124 | CDR1 PRT |
| 159 | C0021124 | CDR2 PRT |
| 160 | C0021124 | CDR3 PRT |
| 161 | C0021128 | VH DNA |
| 162 | C0021128 | VH PRT |
| 163 | C0021128 | CDR1 PRT |
| 164 | C0021128 | CDR2 PRT |
| 165 | C0021128 | CDR3 PRT |
| 166 | C0021128 | VL DNA |
| 167 | C0021128 | VL PRT |
| 168 | C0021128 | CDR1 PRT |
| 169 | C0021128 | CDR2 PRT |
| 170 | C0021128 | CDR3 PRT |
| 171 | C0021129 | VH DNA |
| 172 | C0021129 | VH PRT |
| 173 | C0021129 | CDR1 PRT |
| 174 | C0021129 | CDR2 PRT |
| 175 | C0021129 | CDR3 PRT |
| 176 | C0021129 | VL DNA |
| 177 | C0021129 | VL PRT |
| 178 | C0021129 | CDR1 PRT |
| 179 | C0021129 | CDR2 PRT |
| 180 | C0021129 | CDR3 PRT |
| 181 | C0021131 | VH DNA |
| 182 | C0021131 | VH PRT |
| 183 | C0021131 | CDR1 PRT |
| 184 | C0021131 | CDR2 PRT |
| 185 | C0021131 | CDR3 PRT |
| 186 | C0021131 | VL DNA |
| 187 | C0021131 | VL PRT |
| 188 | C0021131 | CDR1 PRT |
| 189 | C0021131 | CDR2 PRT |
| 190 | C0021131 | CDR3 PRT |
| 191 | C0021133 | VH DNA |
| 192 | C0021133 | VH PRT |
| 193 | C0021133 | CDR1 PRT |
| 194 | C0021133 | CDR2 PRT |
| 195 | C0021133 | CDR3 PRT |
| 196 | C0021133 | VL DNA |
| 197 | C0021133 | VL PRT |
| 198 | C0021133 | CDR1 PRT |
| 199 | C0021133 | CDR2 PRT |
| 200 | C0021133 | CDR3 PRT |
| 201 | C0021135 | VH DNA |
| 202 | C0021135 | VH PRT |
| 203 | C0021135 | CDR1 PRT |
| 204 | C0021135 | CDR2 PRT |
| 205 | C0021135 | CDR3 PRT |
| 206 | C0021135 | VL DNA |
| 207 | C0021135 | VL PRT |
| 208 | C0021135 | CDR1 PRT |
| 209 | C0021135 | CDR2 PRT |
| 210 | C0021135 | CDR3 PRT |
| 211 | C0021139 | VH DNA |
| 212 | C0021139 | VH PRT |
| 213 | C0021139 | CDR1 PRT |
| 214 | C0021139 | CDR2 PRT |
| 215 | C0021139 | CDR3 PRT |
| 216 | C0021139 | VL DNA |
| 217 | C0021139 | VL PRT |
| 218 | C0021139 | CDR1 PRT |
| 219 | C0021139 | CDR2 PRT |
| 220 | C0021139 | CDR3 PRT |
| 221 | C0021141 | VH DNA |
| 222 | C0021141 | VH PRT |
| 223 | C0021141 | CDR1 PRT |
| 224 | C0021141 | CDR2 PRT |
| 225 | C0021141 | CDR3 PRT |
| 226 | C0021141 | VL DNA |
| 227 | C0021141 | VL PRT |
| 228 | C0021141 | CDR1 PRT |
| 229 | C0021141 | CDR2 PRT |
| 230 | C0021141 | CDR3 PRT |
| 231 | C0021142 | VH DNA |
| 232 | C0021142 | VH PRT |
| 233 | C0021142 | CDR1 PRT |
| 234 | C0021142 | CDR2 PRT |
| 235 | C0021142 | CDR3 PRT |
| 236 | C0021142 | VL DNA |
| 237 | C0021142 | VL PRT |
| 238 | C0021142 | CDR1 PRT |
| 239 | C0021142 | CDR2 PRT |
| 240 | C0021142 | CDR3 PRT |
| 241 | C0021142 IgG | VH DNA |
| 242 | C0021142 IgG | VH PRT |
| 243 | C0021142 IgG | CDR1 PRT |
| 244 | C0021142 IgG | CDR2 PRT |
| 245 | C0021142 IgG | CDR3 PRT |
| 246 | C0021142 IgG | VL DNA |
| 247 | C0021142 IgG | VL PRT |
| 248 | C0021142 IgG | CDR1 PRT |
| 249 | C0021142 IgG | CDR2 PRT |
| 250 | C0021142 IgG | CDR3 PRT |
| 251 | C0021144 | VH DNA |
| 252 | C0021144 | VH PRT |
| 253 | C0021144 | CDR1 PRT |
| 254 | C0021144 | CDR2 PRT |
| 255 | C0021144 | CDR3 PRT |
| 256 | C0021144 | VL DNA |
| 257 | C0021144 | VL PRT |
| 258 | C0021144 | CDR1 PRT |
| 259 | C0021144 | CDR2 PRT |
| 260 | C0021144 | CDR3 PRT |
| 261 | C0021155 | VH DNA |
| 262 | C0021155 | VH PRT |
| 263 | C0021155 | CDR1 PRT |
| 264 | C0021155 | CDR2 PRT |
| 265 | C0021155 | CDR3 PRT |
| 266 | C0021155 | VL DNA |
| 267 | C0021155 | VL PRT |
| 268 | C0021155 | CDR1 PRT |
| 269 | C0021155 | CDR2 PRT |
| 270 | C0021155 | CDR3 PRT |
| 271 | C0021158 | VH DNA |
| 272 | C0021158 | VH PRT |
| 273 | C0021158 | CDR1 PRT |
| 274 | C0021158 | CDR2 PRT |
| 275 | C0021158 | CDR3 PRT |
| 276 | C0021158 | VL DNA |
| 277 | C0021158 | VL PRT |
| 278 | C0021158 | CDR1 PRT |
| 279 | C0021158 | CDR2 PRT |
| 280 | C0021158 | CDR3 PRT |
| 281 | C0021158 IgG | VH DNA |
| 282 | C0021158 IgG | VH PRT |
| 283 | C0021158 IgG | CDR1 PRT |
| 284 | C0021158 IgG | CDR2 PRT |
| 285 | C0021158 IgG | CDR3 PRT |
| 286 | C0021158 IgG | VL DNA |
| 287 | C0021158 IgG | VL PRT |
| 288 | C0021158 IgG | CDR1 PRT |
| 289 | C0021158 IgG | CDR2 PRT |
| 290 | C0021158 IgG | CDR3 PRT |
| 291 | C0021158 dr | VH DNA |
| 292 | C0021158 dr | VH PRT |
| 293 | C0021158 dr | CDR1 PRT |
| 294 | C0021158 dr | CDR2 PRT |
| 295 | C0021158 dr | CDR3 PRT |
| 296 | C0021158 dr | VL DNA |
| 297 | C0021158 dr | VL PRT |
| 298 | C0021158 dr | CDR1 PRT |
| 299 | C0021158 dr | CDR2 PRT |
| 300 | C0021158 dr | CDR3 PRT |
| 301 | C0021158 fgl | VH DNA |
| 302 | C0021158 fgl | VH PRT |
| 303 | C0021158 fgl | CDR1 PRT |
| 304 | C0021158 fgl | CDR2 PRT |
| 305 | C0021158 fgl | CDR3 PRT |
| 306 | C0021158 fgl | VL DNA |
| 307 | C0021158 fgl | VL PRT |
| 308 | C0021158 fgl | CDR1 PRT |
| 309 | C0021158 fgl | CDR2 PRT |
| 310 | C0021158 fgl | CDR3 PRT |
| 311 | C0021158 fgl2 | VH DNA |
| 312 | C0021158 fgl2 | VH PRT |
| 313 | C0021158 fgl2 | CDR1 PRT |
| 314 | C0021158 fgl2 | CDR2 PRT |
| 315 | C0021158 fgl2 | CDR3 PRT |
| 316 | C0021158 fgl2 | VL DNA |
| 317 | C0021158 fgl2 | VL PRT |
| 318 | C0021158 fgl2 | CDR1 PRT |
| 319 | C0021158 fgl2 | CDR2 PRT |
| 320 | C0021158 fgl2 | CDR3 PRT |
| 321 | C0021177 | VH DNA |
| 322 | C0021177 | VH PRT |
| 323 | C0021177 | CDR1 PRT |
| 324 | C0021177 | CDR2 PRT |
| 325 | C0021177 | CDR3 PRT |
| 326 | C0021177 | VL DNA |
| 327 | C0021177 | VL PRT |
| 328 | C0021177 | CDR1 PRT |
| 329 | C0021177 | CDR2 PRT |
| 330 | C0021177 | CDR3 PRT |
| 331 | C0021180 | VH DNA |
| 332 | C0021180 | VH PRT |
| 333 | C0021180 | CDR1 PRT |
| 334 | C0021180 | CDR2 PRT |
| 335 | C0021180 | CDR3 PRT |
| 336 | C0021180 | VL DNA |
| 337 | C0021180 | VL PRT |
| 338 | C0021180 | CDR1 PRT |
| 339 | C0021180 | CDR2 PRT |
| 340 | C0021180 | CDR3 PRT |
| 341 | C0021181 | VH DNA |
| 342 | C0021181 | VH PRT |
| 343 | C0021181 | CDR1 PRT |
| 344 | C0021181 | CDR2 PRT |
| 345 | C0021181 | CDR3 PRT |
| 346 | C0021181 | VL DNA |
| 347 | C0021181 | VL PRT |
| 348 | C0021181 | CDR1 PRT |
| 349 | C0021181 | CDR2 PRT |
| 350 | C0021181 | CDR3 PRT |

ARG2 Amino Acid Sequences

SEQ ID NO: 351
N-terminal Avi tag
AGLNDIFEAQKIEWHE
SEQ ID NO: 352
Avi-huARG1-His10
MAGLNDIFEAQKIEWHEGGGMSAKSRTIGIIGAPFSKGQPRGGVEEGPTV
LRKAGLLEKLKEQECDVKDYGDLPFADIPNDSPFQIVKNPRSVGKASEQL
AGKVAEVKKNGRISLVLGGDHSLAIGSISGHARVHPDLGVIWVDAHTDIN
TPLTTTSGNLHGQPVSFLLKELKGKIPDVPGFSWVTPCISAKDIVYIGLR
DVDPGEHYILKTLGIKYFSMTEVDRLGIGKVMEETLSYLLGRKKRPIHLS
FDVDGLDPSFTPATGTPVVGGLTYREGLYITEEIYKTGLLSGLDIMEVNP
SLGKTPEEVTRTVNTAVAITLACFGLAREGNHKPIDYLNPPKGGGHHHHH
HHHHH
SEQ ID NO: 353
Avi-huARG2-His10
MAGLNDIFEAQKIEWHEGGGVHSVAVIGAPFSQGQKRKGVEHGPAAIREA
GLMKRLSSLGCHLKDFGDLSFTPVPKDDLYNNLIVNPRSVGLANQELAEV
VSRAVSDGYSCVTLGGDHSLAIGTISGHARHCPDLCVVWVDAHADINTPL
TTSSGNLHGQPVSFLLRELQDKVPQLPGFSWIKPCISSASIVYIGLRDVD
PPEHFILKNYDIQYFSMRDIDRLGIQKVMERTFDLLIGKRQRPIHLSFDI
DAFDPTLAPATGTPVVGGLTYRHGMYIAEEIHNTGLLSALDLVEVNPQLA
TSEEEAKTTANLAVDVIASSFGQTKEGGHIVYDQLPTPSSPDESENQARV
RIGGGHHHHHHHHHH
SEQ ID NO: 354
Avi-huARG2-His10 Monomer
MAGLNDIFEAQKIEWHEGGGVHSVAIVGAPFSRGQKKLGVEYGPAAIREA
GLLKRLSRLGCHLKDFGDLSFTNVPQDDPYNNLVVYPRSVGLANQELAEV
VSRAVSGGYSCVTMGGDHSLAIGTIIGHARHRPDLCVIWVDAHADINTPL
TTVSGNIHGQPLSFLIKELQDKVPQLPGFSWIKPCLSPPNIVYIGLRDVE
PPEHFILKNYDIQYFSMREIDRLGIQKVMEQTFDRLIGKRQRPIHLSFDI
DAFDPKLAPATGTPVVGGLTYRQGVYITEEIHNTGLLSALDLVEVNPHLA
TSEEEAKATARLAVDVIASSFGQTREGGHIVYDHLPTPSSPHESENEECV
RIGGGHHHHHHHHHH
SEQ ID NO: 355
huARG2-His10
MVHSVAVIGAPFSQGQKRKGVEHGPAAIREAGLMKRLSSLGCHLKDFGDL
SFTPVPKDDLYNNLIVNPRSVGLANQELAEVVSRAVSDGYSCVTLGGDHS
LAIGTISGHARHCPDLCVVWVDAHADINTPLTTSSGNLHGQPVSFLLREL
QDKVPQLPGFSWIKPCISSASIVYIGLRDVDPPEHFILKNYDIQYFSMRD
IDRLGIQKVMERTFDLLIGKRQRPIHLSFDIDAFDPTLAPATGTPVVGGL
TYREGMYIAEEIHNTGLLSALDLVEVNPQLATSEEEAKTTANLAVDVIAS
SFGQTREGGHIVYDQLPTPSSPDESENQARVRIGGGHHHHHHHHHH
SEQ ID NO: 356
Avi-cynoARG2-His10
MAGLNDIFEAQKIEWHEGGGVHSVAVIGAPFSQGQKRKGVEHGPAAIREA
GLMKRLSSLGCHLKDFGDLSFTPVPKDDLYNNLIVNPRSVGLANQELAEV
VSRAVSDGYSCVTLGGDHSLAIGTISGHARHCPDLCVVWVDAHADINTPL
TTSSGNLHGQPVSFLLRELQDKVPQLPGFSWIKPCISSPSIVYIGLRDVD
PPEHFILKNYDIQYFSMRDIDRLGIQKVMEQTFDLLIGKRQRPIHLSFDI
DAFDPTLAPATGTPVVGGLTYREGMYIAEEIHNTGLLSALDLVEVNPQLA
TSEEEAKTTANLAVDVIASSFGQTREGGHIVYGQLPTPSSPDESENQARV
RIGGGHHHHHHHHHH
SEQ ID NO: 357
huARG2-His6
MVHSVAVIGAPFSQGQKRKGVEHGPAAIREAGLMKRLSSLGCHLKDFGDL
SFTPVPKDDLYNNLIVNPRSVGLANQELAEVVSRAVSDGYSCVTLGGDHS
LAIGTISGHARHCPDLCVVWVDAHADINTPLTTSSGNLHGQPVSFLLREL
QDKVPQLPGFSWIKPCISSASIVYIGLRDVDPPEHFILKNYDIQYFSMRD
IDRLGIQKVMERTFDLLIGKRQRPIHLSFDIDAFDPTLAPATGTPVVGGL
TYREGMYIAEEIHNTGLLSALDLVEVNPQLATSEEEAKTTANLAVDVIAS
SFGQTREGGHIVYDQLPTPSSPDESENQARVRIHHHHHH

1. An isolated antigen-binding protein characterised in that it is capable of binding specifically to human Arginase II (ARG2) and inhibiting the enzyme activity of human ARG2.

2. The antigen-binding protein according to claim 1, wherein
a) the antigen-binding protein is capable of binding specifically to and inhibiting monomeric and/or trimeric human ARG2;
b) the antigen-binding protein binds trimeric human ARG2 with a dissociation constant (K_{D}) of less than 10 nM, less than 1 nM, less than 500 pM, less than 300 pM, or less than 150 pM, when assessed by Bio-Layer Interferometry (BLI);
c) the antigen-binding protein is selective for binding and inhibiting human ARG2 over human ARG1;
d) selectivity for binding human ARG2 over human ARG1 is assessed by Bio-Layer Interferometry (BLI);
e) the antigen-binding protein does not measurably bind human ARG1 when assessed by Bio-Layer Interferometry (BLI);
f) the antigen-binding protein binds human ARG2 with a 1:1 or 3:1 stoichiometry (antigen binding protein:human ARG2);
g) the antigen-binding protein is capable of binding specifically to and inhibiting the enzyme activity of cynomolgus ARG2;
h) the antigen-binding protein inhibits ARG2 by a mode of action that is not competitive; and/or,
i) the antigen-binding protein restores T-cell proliferation in vitro in the presence of ARG2.

3-10. (canceled)

11. The antigen-binding protein according to claim 1, comprising:
(a) a VH domain comprising a set of HCDRs: HCDR1, HCDR2 and HCDR3, interspersed with framework (FW) regions (HFW1-HCDR1-HFW2-HCDR2-HFW3-HCDR3-HFW4), wherein the amino acid sequence of HCDR3 (amino acids 95-102) is LRADLGLYMDL (SEQ ID NO: 315) and optionally further comprising
(b) a VL domain comprising a set of LCDRs: LCDR1, LCDR2 and LCDR3, interspersed with framework (FW) regions (LFW1-LCDR1-LFW2-LCDR2-LFW3-LCDR3-LFW4), wherein the amino acid sequence of LCDR1 (amino acids 24-34) is SGSSSNIGNHYVS (SEQ ID NO: 318), wherein the sequences are defined by Kabat nomenclature.

12. The antigen-binding protein according to claim 1, comprising:
(a) a VH domain comprising a set of HCDRs: HCDR1, HCDR2 and HCDR3, interspersed with framework regions (HFW1-HCDR1-HFW2-HCDR2-HFW3-HCDR3-HFW4), wherein the set of HCDRs is selected from those of antibody C0021158fgl2 (SEQ ID NO: 313, 314 and 315), C0021181 (SEQ ID NO: 343, 344, and 345), C0021180 (SEQ ID NO: 333, 334 and 335), C0021177 (SEQ ID NO: 323, 324 and 325), C0021158 (SEQ ID NO: 273, 274 and 275), C0021158 IgG (SEQ ID NO: 283, 284 and 285), C0021158fgl (SEQ ID NO: 303, 304 and 305), C0021158dr (SEQ ID NO: 293, 294 and 295), C0021061 (SEQ ID NO: 63, 64 and 65), C0020187 (SEQ ID NO: 13, 14 and 15), C0021155 (SEQ ID NO: 263, 264 and 265), C0021144 (SEQ ID NO: 253, 254 and 255), C0021142 (SEQ ID NO: 233, 234 and 235), C0021142 IgG (SEQ ID NO: 243, 244 and 245), C0021141 (SEQ ID NO: 223, 224 and 225), C0021139 (SEQ ID NO: 213, 214 and 215), C0021135 (SEQ ID NO: 203, 204 and 205), C0021133 (SEQ ID NO: 193, 194 and 195), C0021131 (SEQ ID NO: 183, 184 and 185), C0021129 (SEQ ID NO: 173, 174 and 175), C0021128 (SEQ ID NO: 163, 164 and 165), C0021124 (SEQ ID NO: 153, 154 and 155), C0021118 (SEQ ID NO: 143, 144 and 145), C0021101 (SEQ ID NO: 133, 134 and 135), C0021098 (SEQ ID NO: 123, 124 and 125), C0021097 (SEQ ID NO: 113, 114 and 115), C0021096 (SEQ ID NO: 103, 104 and 105), C0021092 (SEQ ID NO: 93, 94 and 95), C0021089 (SEQ ID NO: 83, 84 and 85), C0021065 (SEQ ID NO: 73, 74 and 75), C0021032 (SEQ ID NO: 53, 54 and 55), C0021022 (SEQ ID NO: 43, 44 and 45), C0021021 (SEQ ID NO: 33, 34 and 35), C0021017 (SEQ ID NO: 23, 24 and 25) and C0020065 (SEQ ID NO: 3, 4 and 5); and/or
(b) a VL domain comprising a set of LCDRs: LCDR1, LCDR2 and LCDR3, interspersed with framework regions (LFW1-LCDR1-LFW2-LCDR2-LFW3-LCDR3-LFW4), wherein the set of LCDRs is selected from those of antibody C0021158fgl2 (SEQ ID NO: 318, 319 and 320), C0021181 (SEQ ID NO: 348, 349, and 350), C0021180 (SEQ ID NO: 338, 339 and 340), C0021177 (SEQ ID NO: 328, 329 and 330), C0021158 (SEQ ID NO: 278, 279 and 280), C0021158 IgG (SEQ ID NO: 288, 289 and 290), C0021158fgl (SEQ ID NO: 308, 309 and 310), C00021158dr (SEQ ID NO: 298, 299 and 300), C0021061 (SEQ ID NO: 68, 69 and 70), C0020187 (SEQ ID NO: 18, 19 and 20), C0021155 (SEQ ID NO: 268, 269 and 270), C0021144 (SEQ ID NO: 258, 259 and 260), C0021142 (SEQ ID NO: 238, 239 and 240), C0021142 IgG (SEQ ID NO: 248, 249 and 250), C0021141 (SEQ ID NO: 228, 229 and 230), C0021139 (SEQ ID NO: 218, 219 and 220), C0021135 (SEQ ID NO: 208, 209 and 210), C0021133 (SEQ ID NO: 198, 199 and 200), C0021131 (SEQ ID NO: 188, 189 and 190), C0021129 (SEQ ID NO: 178, 179 and 180), C0021128 (SEQ ID NO: 168, 169 and 170), C0021124 (SEQ ID NO: 158, 159 and 160), C0021118 (SEQ ID NO: 148, 149 and 150), C0021101 (SEQ ID NO: 138, 139 and 140), C0021098 (SEQ ID NO: 128, 129 and 130), C0021097 (SEQ ID NO: 118, 119 and 120), C0021096 (SEQ ID NO: 108, 109 and 110), C0021092 (SEQ ID NO: 98, 99 and 100), C0021089 (SEQ ID NO: 88, 89 and 90), C0021065 (SEQ ID NO: 78, 79 and 80), C0021032 (SEQ ID NO: 58, 59 and 60), C0021022 (SEQ ID NO: 48, 49 and 50), C0021021 (SEQ ID NO: 38, 39 and 40), C0021017 SEQ ID NO: 28, 29 and 30) and C0020065 (SEQ ID NO: 8, 9 and 10); wherein the sequences are defined by Kabat nomenclature; or
(c) the antigen-binding protein comprising any one of the VH domains selected from the following:
a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021158fgl2;
a VH domain comprising HCDR1 (SEQ ID NO: 343), HCDR2 (SEQ ID NO: 344) and HCDR3 (SEQ ID NO: 345) and a VL domain comprising LCDR1 (SEQ ID NO: 348), LCDR2, (SEQ ID NO: 349) and LCDR3 (SEQ ID NO: 350) of C0021181;
a VH domain comprising HCDR1 (SEQ ID NO: 313), HCDR2 (SEQ ID NO: 314) and HCDR3 (SEQ ID NO: 315) and a VL domain comprising LCDR1 (SEQ ID NO: 318), LCDR2, (SEQ ID NO: 319) and LCDR3 (SEQ ID NO: 320) of C0021180;
a VH domain comprising HCDR1 (SEQ ID NO: 323), HCDR2 (SEQ ID NO: 324) and HCDR3 (SEQ ID NO: 325) and a VL domain comprising LCDR1 (SEQ ID NO: 328), LCDR2, (SEQ ID NO: 329) and LCDR3 (SEQ ID NO: 330) of C0021177;
a VH domain comprising HCDR1 (SEQ ID NO: 273), HCDR2 (SEQ ID NO: 274) and HCDR3 (SEQ ID NO: 275) and a VL domain comprising LCDR1 (SEQ ID NO: 278), LCDR2, (SEQ ID NO: 279) and LCDR3 (SEQ ID NO: 280) of C0021158;
a VH domain comprising HCDR1 (SEQ ID NO: 283), HCDR2 (SEQ ID NO: 284) and HCDR3 (SEQ ID NO: 285) and a VL domain comprising LCDR1 (SEQ ID NO: 288), LCDR2, (SEQ ID NO: 289) and LCDR3 (SEQ ID NO: 290) of C0021158 IgG;
a VH domain comprising HCDR1 (SEQ ID NO: 303), HCDR2 (SEQ ID NO: 304) and HCDR3 (SEQ ID NO: 305) and a VL domain comprising LCDR1 (SEQ ID NO: 308), LCDR2, (SEQ ID NO: 309) and LCDR3 (SEQ ID NO: 310) of C0021158fgl;
a VH domain comprising HCDR1 (SEQ ID NO: 293), HCDR2 (SEQ ID NO: 294) and HCDR3 (SEQ ID NO: 295) and a VL domain comprising LCDR1 (SEQ ID NO: 298), LCDR2, (SEQ ID NO: 299) and LCDR3 (SEQ ID NO: 300) of C0021158dr;
a VH domain comprising HCDR1 (SEQ ID NO: 63), HCDR2 (SEQ ID NO: 64) and HCDR3 (SEQ ID NO: 65) and a VL domain comprising LCDR1 (SEQ ID NO: 68), LCDR2, (SEQ ID NO: 69) and LCDR3 (SEQ ID NO: 70) of C0021061;
a VH domain comprising HCDR1 (SEQ ID NO: 13), HCDR2 (SEQ ID NO: 14) and HCDR3 (SEQ ID NO: 15) and a VL domain comprising LCDR1 (SEQ ID NO: 18), LCDR2, (SEQ ID NO: 19) and LCDR3 (SEO ID NO: 20) of C0020187;
a VH domain comprising HCDR1 (SEQ ID NO: 263), HCDR2 (SEQ ID NO: 264) and HCDR3 (SEQ ID NO: 265) and a VL domain comprising LCDR1 (SEQ ID NO: 268), LCDR2, (SEQ ID NO: 269) and LCDR3 (SEQ ID NO: 270) of C0021155;
a VH domain comprising HCDR1 (SEQ ID NO: 253), HCDR2 (SEQ ID NO: 254) and HCDR3 (SEQ ID NO: 255) and a VL domain comprising LCDR1 (SEQ ID NO: 258), LCDR2, (SEQ ID NO: 259) and LCDR3 (SEQ ID NO: 260) of C0021144;
a VH domain comprising HCDR1 (SEQ ID NO: 233), HCDR2 (SEQ ID NO: 234) and HCDR3 (SEQ ID NO: 235) and a VL domain comprising LCDR1 (SEQ ID NO: 238), LCDR2, (SEQ ID NO: 239) and LCDR3 (SEQ ID NO: 240) of C0021142;
a VH domain comprising HCDR1 (SEQ ID NO: 243), HCDR2 (SEQ ID NO: 244) and HCDR3 (SEQ ID NO: 245) and a VL domain comprising LCDR1 (SEQ ID NO: 248), LCDR2, (SEQ ID NO: 249) and LCDR3 (SEQ ID NO: 250) of C0021142 IgG;
a VH domain comprising HCDR1 (SEQ ID NO: 223), HCDR2 (SEQ ID NO: 224) and HCDR3 (SEQ ID NO: 225) and a VL domain comprising LCDR1 (SEQ ID NO: 228), LCDR2, (SEQ ID NO: 229) and LCDR3 (SEQ ID NO: 230) of C0021141;
a VH domain comprising HCDR1 (SEQ ID NO: 213), HCDR2 (SEQ ID NO: 214) and HCDR3 (SEQ ID NO: 215) and a VL domain comprising LCDR1 (SEQ ID NO: 218), LCDR2, (SEQ ID NO: 219) and LCDR3 (SEQ ID NO: 220) of C0021139;
a VH domain comprising HCDR1 (SEQ ID NO: 203), HCDR2 (SEQ ID NO: 204) and HCDR3 (SEQ ID NO: 205) and a VL domain comprising LCDR1 (SEQ ID NO: 208), LCDR2, (SEQ ID NO: 209) and LCDR3 (SEQ ID NO: 210) of C0021135;
a VH domain comprising HCDR1 (SEQ ID NO: 193), HCDR2 (SEQ ID NO: 194) and HCDR3 (SEQ ID NO: 195) and a VL domain comprising LCDR1 (SEQ ID NO: 198), LCDR2, (SEQ ID NO: 199) and LCDR3 (SEQ ID NO: 200) of C0021133;
a VH domain comprising HCDR1 (SEQ ID NO: 183), HCDR2 (SEQ ID NO: 184) and HCDR3 (SEQ ID NO: 185) and a VL domain comprising LCDR1 (SEQ ID NO: 188), LCDR2, (SEQ ID NO: 189) and LCDR3 (SEQ ID NO: 190) of C0021131;
a VH domain comprising HCDR1 (SEQ ID NO: 173), HCDR2 (SEQ ID NO: 174) and HCDR3 (SEQ ID NO: 175) and a VL domain comprising LCDR1 (SEQ ID NO: 178), LCDR2, (SEO ID NO: 179) and LCDR3 (SEO ID NO: 180) of C0021129;
a VH domain comprising HCDR1 (SEQ ID NO: 163), HCDR2 (SEQ ID NO: 164) and HCDR3 (SEQ ID NO: 165) and a VL domain comprising LCDR1 (SEQ ID NO: 168), LCDR2, (SEQ ID NO: 169) and LCDR3 (SEQ ID NO: 170) C0021128;
a VH domain comprising HCDR1 (SEQ ID NO: 153), HCDR2 (SEQ ID NO: 154) and HCDR3 (SEQ ID NO: 155) and a VL domain comprising LCDR1 (SEQ ID NO: 158), LCDR2, (SEQ ID NO: 159) and LCDR3 (SEQ ID NO: 160) of C0021124;
a VH domain comprising HCDR1 (SEQ ID NO: 143), HCDR2 (SEQ ID NO: 144) and HCDR3 (SEQ ID NO: 145) and a VL domain comprising LCDR1 (SEQ ID NO: 148), LCDR2, (SEQ ID NO: 149) and LCDR3 (SEQ ID NO: 150) of C0021118;
a VH domain comprising HCDR1 (SEQ ID NO: 133), HCDR2 (SEQ ID NO: 134) and HCDR3 (SEQ ID NO: 135) and a VL domain comprising LCDR1 (SEQ ID NO: 138), LCDR2, (SEQ ID NO: 139) and LCDR3 (SEQ ID NO: 140) of C0021101;
a VH domain comprising HCDR1 (SEQ ID NO: 123), HCDR2 (SEQ ID NO: 124) and HCDR3 (SEQ ID NO: 125) and a VL domain comprising LCDR1 (SEQ ID NO: 128), LCDR2, (SEQ ID NO: 129) and LCDR3 (SEQ ID NO: 130) of C0021098;
a VH domain comprising HCDR1 (SEQ ID NO: 113), HCDR2 (SEQ ID NO: 114) and HCDR3 (SEQ ID NO: 115) and a VL domain comprising LCDR1 (SEQ ID NO: 118), LCDR2, (SEQ ID NO: 119) and LCDR3 (SEQ ID NO: 120) of C0021097;
a VH domain comprising HCDR1 (SEQ ID NO: 103), HCDR2 (SEQ ID NO: 104) and HCDR3 (SEQ ID NO: 105) and a VL domain comprising LCDR1 (SEQ ID NO: 108), LCDR2, (SEQ ID NO: 109) and LCDR3 (SEQ ID NO: 110) of C0021096;
a VH domain comprising HCDR1 (SEQ ID NO: 93), HCDR2 (SEQ ID NO: 94) and HCDR3 (SEQ ID NO: 95) and a VL domain comprising LCDR1 (SEQ ID NO: 98), LCDR2, (SEQ ID NO: 99) and LCDR3 (SEQ ID NO: 100) of C0021092;
a VH domain comprising HCDR1 (SEQ ID NO: 83), HCDR2 (SEQ ID NO: 84) and HCDR3 (SEQ ID NO: 85) and a VL domain comprising LCDR1 (SEQ ID NO: 88), LCDR2, (SEQ ID NO: 89) and LCDR3 (SEQ ID NO: 90) of C0021089;
a VH domain comprising HCDR1 (SEQ ID NO: 73), HCDR2 (SEQ ID NO: 74) and HCDR3 (SEQ ID NO: 75) and a VL domain comprising LCDR1 (SEQ ID NO: 78), LCDR2, (SEQ ID NO: 79) and LCDR3 (SEO ID NO: 80) of C0021065;
a VH domain comprising HCDR1 (SEQ ID NO: 53), HCDR2 (SEQ ID NO: 54) and HCDR3 (SEQ ID NO: 55) and a VL domain comprising LCDR1 (SEQ ID NO: 58), LCDR2, (SEQ ID NO: 59) and LCDR3 (SEQ ID NO: 60) of C0021032;
a VH domain comprising HCDR1 (SEQ ID NO: 43), HCDR2 (SEQ ID NO: 44) and HCDR3 (SEQ ID NO:45) and a VL domain comprising LCDR1 (SEQ ID NO: 48), LCDR2, (SEQ ID NO: 49) and LCDR3 (SEQ ID NO: 50) of C0021022;
a VH domain comprising HCDR1 (SEQ ID NO: 33), HCDR2 (SEQ ID NO: 34) and HCDR3 (SEQ ID NO: 35) and a VL domain comprising LCDR1 (SEQ ID NO: 38), LCDR2, (SEQ ID NO: 39) and LCDR3 (SEQ ID NO: 40) of C0021021;
a VH domain comprising HCDR1 (SEQ ID NO: 23), HCDR2 (SEQ ID NO: 24) and HCDR3 (SEQ ID NO: 25) and a VL domain comprising LCDR1 (SEQ ID NO: 28), LCDR2, (SEQ ID NO: 29) and LCDR3 (SEQ ID NO: 30) of C0021017; or
a VH domain comprising HCDR1 (SEQ ID NO: 3), HCDR2 (SEQ ID NO: 4) and HCDR3 (SEQ ID NO: 5) and a VL domain comprising LCDR1 (SEQ ID NO: 8), LCDR2, (SEQ ID NO: 9) and LCDR3 (SEQ ID NO: 10) of C0020065; wherein the sequences are defined by Kabat nomenclature; or,
(d) the antigen-binding protein comprising:
(i) a VH domain selected from a VH domain of antibody C0021158 fgl2 (SEQ ID NO: 312), C0021181 (SEQ ID NO: 342), C0021180 (SEQ ID NO: 332), C0021177 (SEQ ID NO: 322), C0021158 (SEQ ID NO: 272), C0021158 IgG (SEQ ID NO: 282), C0021158fgl (SEQ ID NO: 302), C0021158dr (SEQ ID NO: 292), C0021061 (SEQ ID NO: 62), C0020187 (SEQ ID NO: 12), C0021155 (SEQ ID NO: 262), C0021144 (SEQ ID NO: 252), C0021142 (SEQ ID NO: 232), C0021142 IgG (SEQ ID NO: 242), C0021141 (SEQ ID NO: 222), C0021139 (SEQ ID NO: 212), C0021135 (SEQ ID NO: 202), C0021133 (SEQ ID NO: 192), C0021131 (SEQ ID NO: 182), C0021129 (SEQ ID NO: 172), C0021128 (SEQ ID NO: 162), C0021124 (SEQ ID NO: 152), C0021118 (SEQ ID NO: 142), C0021101 (SEQ ID NO: 132), C0021098 (SEQ ID NO: 122), C0021097 (SEQ ID NO: 112), C0021096 (SEQ ID NO: 102), C0021092 (SEQ ID NO: 92), C0021089 (SEQ ID NO: 82), C0021065 (SEQ ID NO: 72), C0021032 (SEQ ID NO: 52), C0021022 (SEQ ID NO: 42), C0021021 (SEQ ID NO: 32), C0021017 (SEQ ID NO: 22) and C0020065 (SEO ID NO: 2), or a germlined version thereof, or a VH domain with at least 80%, 81%, 82%, 83%, 84%, 85%, 86%, 87%, 88%, 89%, 90%, 91%, 92%, 93%, 94%, 95%, 96%, 97%, 98%, or 99% homology thereto; and/or
(ii) a VL domain selected from a VL domain of antibody C0021158 fgl2 (SEQ ID NO: 317), C0021181 (SEQ ID NO: 347), C0021180 (SEQ ID NO: 337), C0021177 (SEQ ID NO: 327), C0021158 (SEQ ID NO: 277), C0021158 IgG (SEQ ID NO: 287), C0021158fgl (SEQ ID NO: 307), C0021158dr (SEQ ID NO: 297), C0021061 (SEQ ID NO: 67), C0020187 (SEQ ID NO: 17), C0021155 (SEQ ID NO: 267), C0021144 (SEQ ID NO: 257), C0021142 (SEQ ID NO: 237), C0021142 IgG (SEQ ID NO: 247), C0021141 (SEQ ID NO: 227), C0021139 (SEQ ID NO: 217), C0021135 (SEQ ID NO: 207), C0021133 (SEQ ID NO: 197), C0021131 (SEQ ID NO: 187), C0021129 (SEQ ID NO: 177), C0021128 (SEQ ID NO: 167), C0021124 (SEQ ID NO: 157), C0021118 (SEQ ID NO: 147), C0021101 (SEQ ID NO: 137), C0021098 (SEQ ID NO: 127), C0021097 (SEQ ID NO: 117), C0021096 (SEQ ID NO: 107), C0021092 (SEQ ID NO: 97), C0021089 (SEQ ID NO: 87), C0021065 (SEQ ID NO: 77), C0021032 (SEQ ID NO: 57), C0021022 (SEQ ID NO: 47), C0021021 (SEQ ID NO: 37), C0021017 (SEQ ID NO: 27) and C0020065 (SEQ ID NO: 7), or a germlined version thereof, or a VL domain with at least 80%, 81%, 82%, 83%, 84%, 85%, 86%, 87%, 88%, 89%, 90%, 91%, 92%, 93%, 94%, 95%, 96%, 97%, 98%, or 99% homology thereto; wherein the sequences are defined by Kabat nomenclature; or,
(e) the antigen binding protein comprising a VH domain and a VL domain at least 80%, 81%, 82%, 83%, 84%, 85%, 86%, 87%, 88%, 89%, 90%, 91%, 92%, 93%, 94%, 95%, 96%, 97%, 98%, or 99% identical with;
a VH domain (SEQ ID NO: 312) and VL domain (SEQ ID NO: 317) of C0021158 fgl2,
a VH domain (SEQ ID NO: 342) and a VL domain (SEQ ID NO: 347) of C0021181,
a VH domain (SEQ ID NO: 332) and a VL domain (SEQ ID NO: 337) of C0021180,
a VH domain (SEQ ID NO: 322) and a VL domain (SEQ ID NO: 327) of C0021177,
a VH domain (SEQ ID NO: 272) and a VL domain (SEQ ID NO: 277) of C0021158,
a VH domain (SEQ ID NO: 282) and a VL domain (SEQ ID NO: 287) of C0021158 IgG,
a VH domain (SEQ ID NO: 302) and a VL domain (SEQ ID NO: 307) of C0021158fgl,
a VH domain (SEQ ID NO: 292) and a VL domain (SEQ ID NO: 297) of C0021158dr,
a VH domain (SEO ID NO: 62) and a VL domain (SEO ID NO: 67) of C0021061,
a VH domain (SEQ ID NO: 12) and a VL domain (SEQ ID NO: 17) of C0020187,
a VH domain (SEQ ID NO: 262) and a VL domain (SEQ ID NO: 267) of C0021155,
a VH domain (SEQ ID NO: 252) and a VL domain (SEQ ID NO: 257) of C0021144,
a VH domain (SEQ ID NO: 232) and a VL domain (SEQ ID NO: 237) of C0021142,
a VH domain (SEQ ID NO: 242) and a VL domain (SEQ ID NO: 247) of C0021142 IgG,
a VH domain (SEQ ID NO: 222) and a VL domain (SEQ ID NO: 227) of C0021141,
a VH domain (SEQ ID NO: 212) and a VL domain (SEQ ID NO: 217) of C0021139,
a VH domain (SEQ ID NO: 202) and a VL domain (SEQ ID NO: 207) of C0021135,
a VH domain (SEQ ID NO: 192) and a VL domain (SEQ ID NO: 197) of C0021133,
a VH domain (SEQ ID NO: 182) and a VL domain (SEQ ID NO: 187) of C0021131,
a VH domain (SEQ ID NO: 172) and a VL domain (SEQ ID NO: 177) of C0021129,
a VH domain (SEQ ID NO: 162) and a VL domain (SEQ ID NO: 167) C0021128,
a VH domain (SEQ ID NO: 152) and a VL domain (SEQ ID NO: 157) C0021124,
a VH domain (SEQ ID NO: 142) and a VL domain (SEQ ID NO: 147) of C0021118,
a VH domain (SEQ ID NO: 132) and a VL domain (SEQ ID NO: 137) of C0021101,
a VH domain (SEQ ID NO: 122) and a VL domain (SEQ ID NO: 127) of C0021098,
a VH domain (SEQ ID NO: 112) and a VL domain (SEQ ID NO: 117) of C0021097,
a VH domain (SEQ ID NO: 102) and a VL domain (SEQ ID NO: 107) of C0021096,
a VH domain (SEQ ID NO: 92) and a VL domain (SEQ ID NO: 97) of C0021092,
a VH domain (SEQ ID NO: 82) and a VL domain (SEQ ID NO: 87) of C0021089,
a VH domain (SEQ ID NO: 72) and a VL domain (SEQ ID NO: 77) of C0021065,
a VH domain (SEQ ID NO: 52) and a VL domain (SEQ ID NO: 57) of C0021032,
a VH domain (SEQ ID NO: 42) and a VL domain (SEQ ID NO: 47) of C0021022,
a VH domain (SEQ ID NO: 32) and a VL domain (SEQ ID NO: 37) of C0021021,
a VH domain (SEQ ID NO: 22) and a VL domain (SEQ ID NO: 27) of C0021017, or
a VH domain (SEQ ID NO: 2) and a VL domain (SEQ ID NO: 7) of C0020065: wherein the sequences are defined by Kabat nomenclature; or
(f) the antigen binding protein comprising a VH domain (SEQ ID NO: 312) and VL domain (SEQ ID NO: 317) of C0021158fgl2,
a VH domain (SEO ID NO: 342) and a VL domain (SEO ID NO: 347) of C0021181,
a VH domain (SEQ ID NO: 332) and a VL domain (SEQ ID NO: 337) of C0021180,
a VH domain (SEQ ID NO: 322) and a VL domain (SEQ ID NO: 327) of C0021177,
a VH domain (SEQ ID NO: 272) and a VL domain (SEQ ID NO: 277) of C0021158,
a VH domain (SEQ ID NO: 282) and a VL domain (SEQ ID NO: 287) of C0021158 IgG,
a VH domain (SEQ ID NO: 302) and a VL domain (SEQ ID NO: 307) of C0021158fgl,
a VH domain (SEQ ID NO: 292) and a VL domain (SEQ ID NO: 297) of C0021158dr,
a VH domain (SEQ ID NO: 62) and a VL domain (SEQ ID NO: 67) of C0021061,
a VH domain (SEQ ID NO: 12) and a VL domain (SEQ ID NO: 17) of C0020187,
a VH domain (SEQ ID NO: 262) and a VL domain (SEQ ID NO: 267) of C0021155,
a VH domain (SEQ ID NO: 252) and a VL domain (SEQ ID NO: 257) of C0021144,
a VH domain (SEQ ID NO: 232) and a VL domain (SEQ ID NO: 237) of C0021142,
a VH domain (SEQ ID NO: 242) and a VL domain (SEQ ID NO: 247) of C0021142 IgG,
a VH domain (SEQ ID NO: 222) and a VL domain (SEQ ID NO: 227) of C0021141,
a VH domain (SEQ ID NO: 212) and a VL domain (SEQ ID NO: 217) of C0021139,
a VH domain (SEQ ID NO: 202) and a VL domain (SEQ ID NO: 207) of C0021135,
a VH domain (SEQ ID NO: 192) and a VL domain (SEQ ID NO: 197) of C0021133,
a VH domain (SEQ ID NO: 182) and a VL domain (SEQ ID NO: 187) of C0021131,
a VH domain (SEQ ID NO: 172) and a VL domain (SEQ ID NO: 177) of C0021129,
a VH domain (SEQ ID NO: 162) and a VL domain (SEQ ID NO: 167) C0021128,
a VH domain (SEQ ID NO: 152) and a VL domain (SEQ ID NO: 157) C0021124,
a VH domain (SEQ ID NO: 142) and a VL domain (SEQ ID NO: 147) of C0021118,
a VH domain (SEQ ID NO: 132) and a VL domain (SEQ ID NO: 137) of C0021101,
a VH domain (SEQ ID NO: 122) and a VL domain (SEQ ID NO: 127) of C0021098,
a VH domain (SEQ ID NO: 112) and a VL domain (SEQ ID NO: 117) of C0021097,
a VH domain (SEQ ID NO: 102) and a VL domain (SEQ ID NO: 107) of C0021096,
a VH domain (SEQ ID NO: 92) and a VL domain (SEQ ID NO: 97) of C0021092,
a VH domain (SEQ ID NO: 82) and a VL domain (SEQ ID NO: 87) of C0021089,
a VH domain (SEQ ID NO: 72) and a VL domain (SEQ ID NO: 77) of C0021065,
a VH domain (SEQ ID NO: 52) and a VL domain (SEQ ID NO: 57) of C0021032,
a VH domain (SEO ID NO: 42) and a VL domain (SEO ID NO: 47) of C0021022,
a VH domain (SEQ ID NO: 32) and a VL domain (SEQ ID NO: 37) of C0021021,
a VH domain (SEQ ID NO: 22) and a VL domain (SEQ ID NO: 27) of C0021017, or
a VH domain (SEQ ID NO: 2) and a VL domain (SEQ ID NO: 7) of C0020065; or a germlined version thereof, wherein the sequences are defined by Kabat nomenclature.

13-18. (canceled)

19. An antigen-binding protein that competes for binding to human ARG2 with an antigen binding protein comprising:
(a) the VH domain of SEQ ID NO: 312 and a VL domain of SEQ ID NO: 317 (C0021158fgl2),
(b) the VH domain of SEQ ID NO: 192 and the VL domain of SEQ ID NO: 197 (C0021133),
(c) the VH domain of SEQ ID NO: 12 and VL domain of SEQ ID NO: 17 (C0020187), or
(d) the VH domain of SEQ ID NO: 2 and VL domain of SEQ ID NO: 7 (C0020065), when assessed in an epitope competition assay.

20. The antigen-binding protein according to claim 1, characterised in that:
(a) it is capable of binding specifically to an epitope of human Arginase II (ARG2) and thereby inhibiting the enzyme activity of human ARG2 by an allosteric mechanism;
(b) it is capable of binding specifically to an epitope of human Arginase II (ARG2) and thereby inducing structural remodelling of residues 33-40 within human ARG2, wherein the sequence numbering is that of the human ARG2 sequence of Uniprot ID #P78540;
(c) it is capable of binding specifically to an epitope of human Arginase II (ARG2) and thereby inhibiting the enzyme activity of human ARG2 by an allosteric mechanism, wherein the antigen-binding protein binds to an epitope on human Arg2 that induces structural or biophysical changes upon His160 of human ARG2 that confer a reduction in ARG2 enzyme activity or ability to process substrate, wherein the sequence numbering is that of the human ARG2 sequence of Uniprot ID #P78540;
(d) the antigen-binding protein binds to an epitope on human Arg2 that induces structural changes whereby Arg39 moves into closer proximity with His160 of human ARG2 thereby conferring a reduction in ARG2 enzyme activity or ability to process substrate, wherein the sequence numbering is that of the human ARG2 sequence of Uniprot ID #P78540;
(e) the antigen-binding protein binds to an epitope on human Arg2 that induces structural changes whereby the ability of His160 to act as a proton donor/acceptor and/or stabilize a catalytically competent bound orientation of the substrate is compromised thereby conferring a reduction in ARG2 enzyme activity;
wherein optionally the antigen-binding protein
(i) binds to an epitope on human ARG2 comprising a conformational epitope comprising residues from Gln35 to Arg39, residues from Lys78 to Ile86, and/or residues from Leu152 to Pro179, wherein the sequence numbering is that of the human ARG2 sequence of Uniprot ID #P78540;
(ii) binds to an epitope on human ARG2 comprising one or more residues selected from GLN35, GLY36, GLN37, LYS38, ARG39, LYS78, ASP79, ASP80, LEU81, TYR82, ASN84, LEU85, ILE86, LEU152, THR153 THR154, SER155, SER156, GLY157, LEU178 and PRO179;
(iii) binds to an epitope on human ARG2 comprising a conformational epitope comprising residues from Pro32 to Glu51, residues from Asp70 to Ile86, and/or residues from Pro299 to Ala308, wherein the sequence numbering is that of the human ARG2 sequence of Uniprot ID #P78540;
(iv) binds to an epitope on human ARG2 comprising one or more residues selected from PRO32, GLN37, LYS38, LYS40, GLY41, GLU43, HIS44, ALA47, ALA48, GLU51, ASP70, SER72, PHE73, THR74, PRO75, LYS78, ASP79, ASP80, LEU81, TYR82, ASN84, LEU85, ILE86, PRO299, GLN300, GLU305 and ALA308;
(v) binds to an epitope on human ARG2 comprising a conformational epitope comprising residues from Gln37 to Glu51, residues from Asp79 to Ile86, and/or residues from Pro299 to Ala308, wherein the sequence numbering is that of the human ARG2 sequence of Uniprot ID #P78540;
(vi) binds to an epitope on recombinant human ARG2 comprising one or more residues selected from GLN37, LYS38, LYS40, GLY41, HIS44, ALA47, ALA48, GLU51, ASP79, ASP80, LEU81, TYR82, ASN84, LEU85, ILE86, PRO299, GLN300, ALA302, THR303, SER304, GLU305 and ALA308; or,
(vii) binds to an epitope on human ARG2 comprising one or more residues selected from PRO32, GLN35, GLY36, GLN37, LYS38, ARG39, LYS40, GLY41, GLU43, HIS44, ALA47, ALA48, GLU51, ASP70, SER72, PHE73, THR74, PRO75, LYS78, ASP79, ASP80, LEU81, TYR82, ASN84, LEU85, ILE86, LEU152, THR153 THR154, SER155, SER156, GLY157, LEU178, PRO179, PRO299, GLN300, ALA302, THR303, SER304, GLU305 and ALA308;
wherein the epitope of (ii) is optionally defined as any residue within human ARG2 with a predicted change in solvent accessibility upon Fab complexation resulting from direct protection by the bound antibody as derived from X-ray structural data of ARG2 inhibitory Fab C0020187, and wherein the sequence numbering is that of the human ARG2 sequence of Uniprot ID #P78540;
wherein the epitope of (iii) or (iv) is optionally defined as any residue within human ARG2 with a predicted change in solvent accessibility upon Fab complexation resulting from direct protection by the bound antibody as derived from X-ray structural data of ARG2 inhibitory Fab C0021158, and wherein the sequence numbering is that of the human ARG2 sequence of Uniprot ID #P78540;
wherein the epitope of (vi) is optionally defined as any residue within human ARG2 with a predicted change in solvent accessibility upon Fab complexation resulting from direct protection by the bound antibody as derived from X-ray structural data of ARG2 inhibitory Fab C0021181, and wherein the sequence numbering is that of the human ARG2 sequence of Uniprot ID #P78540; and
wherein the epitope of (vii) is optionally defined as any residue within human ARG2 with a predicted change in solvent accessibility upon Fab complexation as derived from X-ray structural data of an ARG2 inhibitory Fab selected from C0020187, C0021158 and C0021181 and wherein the sequence numbering is that of the human ARG2 sequence of Uniprot ID #P78540.

21-35. (canceled)

36. The antigen-binding protein according to claim 1, wherein the antigen-binding protein is selected from the group consisting of a) an antibody or a fragment thereof, a domain antibody, a protein scaffold, or an aptamer, b) a human IgG or a modified human IgG, c) a human IgG1, IgG2, IgG4, or a modified version thereof, and d) a human IgG1 or IgG1-YTE; and/or wherein the antigen-binding protein has a modified Fc to confer effector function and/or half-life extension.

37-40. (canceled)

41. A pharmaceutical composition comprising an antigen-binding protein according to claim 1, and a pharmaceutically acceptable excipient.

42. (canceled)

43. A method of treating an individual in need thereof for a treatment to restore immunocompetancy, alleviate inflammation-triggered immune dysfunction, inflammation-associated immune suppression, promote T cell immune responses, or to prevent tumour immune escape, fibrosis, and immunopathology of infectious diseases, comprising:
administering to the individual the pharmaceutical composition of claim 41, whereby the individual in need thereof is treated.

44. The method of claim 43, wherein the treatment further comprises restoring T cell proliferation in the presence of ARG2.

45. A method of treating an individual in need thereof for a treatment of cancer, immune cell dysfunction, autoimmunity or unwanted immune deviation, comprising:
administering to the individual the pharmaceutical composition of claim 41, whereby the individual in need thereof is treated.

46. The method of claim 45, wherein the cancer is selected from acute myeloid leukemia (AML), osteosarcoma, HCMV-driven GBM, pancreatic cancer, head and neck squamous cell carcinoma, thyroid, prostate, breast or ovarian cancer.

47. A method of treating an individual in need thereof for a treatment of infection (e.g., neonate infection), endothelial dysfunction (e.g. erectile dysfunction), vascular disease, cardiovascular disease, ageing and cellular senescence, CNS disease and injury; diabetes-associated disease, cystic fibrosis or infection associated with cystic fibrosis, comprising:
administering to the individual the pharmaceutical composition of claim 41, whereby the individual in need thereof is treated.

48. (canceled)

49. An isolated nucleic acid encoding an antigen-binding protein according to claim 1.

50. A host cell in vitro transformed with nucleic acid according to claim 49.

51. A method of producing an antigen-binding protein, comprising culturing host cells according to claim 50 under conditions for production of the antigen-binding protein, optionally further comprising isolating and/or purifying the antigen-binding protein.

52. (canceled)

53. The method according to claim 51, further comprising formulating the antigen-binding protein into a composition comprising at least one additional component.

54. A method for producing an antigen-binding protein that binds specifically to and inhibits human ARG2, the method comprising providing a variant VH domain which is an amino acid sequence variant of a parent VH domain, by way of addition, deletion, substitution or insertion of one or more amino acids in the amino acid sequence of a parent VH domain comprising HCDR1, HCDR2 and HCDR3, wherein the parent VH domain HCDR1, HCDR2 and HCDR3 are a set of HCDRs selected from the set of HCDRs of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, and optionally combining the variant VH domain thus provided with one or more VL domains to provide one or more VH/VL combinations; and testing said variant VH domain which is an amino acid sequence variant of the parent VH domain or the variant VH/VL combination or combinations to identify an antigen-binding protein antigen binding domain for human ARG2; or
optionally wherein the parent VH domain is selected from C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, or a germlined version thereof; or
optionally wherein the one or more VL domains is a variant VL domain provided by way of addition, deletion, substitution or insertion of one or more amino acids in the amino acid sequence of a parent VL domain comprising LCDR1, LCDR2 and LCDR3, wherein the parent VL domain LCDR1, LCDR2 and LCDR3 are a set of LCDRs selected from the set of LCDRs of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, producing one or more variant VL domains each of which is an amino acid sequence variant of the parent VL domain; or
optionally wherein the parent VL domain is the VL domain of any of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, or a germlined version thereof.

55-57. (canceled)

58. The method according to claim 54, further comprising producing the antigen-binding protein antigen-binding domain as a component of an IgG, scFv or Fab antigen-binding protein.

59. A method for producing an antigen-binding protein that binds to and inhibits human ARG2, wherein the method comprises: providing starting nucleic acid encoding a VH domain or a starting repertoire of nucleic acids each encoding a VH domain, wherein the VH domain or VH domains either comprise a HCDR1, HCDR2 and/or HCDR3 to be replaced or lack a HCDR1, HCDR2 and/or HCDR3 encoding region; combining said starting nucleic acid or starting repertoire with donor nucleic acid or donor nucleic acids encoding the amino acid sequence of an HCDR1, HCDR2, and/or HCDR3 selected from the HCDR1, HCDR2 and/or HCDR3 of C0021158fgl2, C0021181, C0021180, C0021177, C0021158, C0021158 IgG, C0021158fgl, C0021158dr, C0021061, C0020187, C0021155, C0021144, C0021142, C0021142 IgG, C0021141, C0021139, C0021135, C0021133, C0021131, C0021129, C0021128, C0021124, C0021118, C0021101, C0021098, C0021097, C0021096, C0021092, C0021089, C0021065, C0021032, C0021022, C0021021, C0021017 and C0020065, such that said donor nucleic acid is or donor nucleic acids are inserted into the CDR1, CDR2 and/or CDR3 region in the starting nucleic acid or starting repertoire, so as to provide a product repertoire of nucleic acids encoding VH domains; expressing the nucleic acids of said product repertoire to produce product VH domains; optionally combining said product VH domains with one or more VL domains;
selecting an antigen-binding protein for ARG2, wherein the antigen-binding protein comprises a product VH domain and optionally a VL domain; and
recovering the nucleic acid encoding the antigen-binding protein or the antigen-binding protein thereof;
optionally wherein the donor nucleic acids are produced by mutation of said HCDR1 and/or HCDR2; or
optionally wherein the donor nucleic acid is produced by mutation of HCDR3; or
optionally providing the donor nucleic acid by random mutation of nucleic acid; or
optionally the method further comprises attaching a product VH domain that is comprised within the recovered antigen-binding protein to an antigen-binding protein constant region or optionally comprising providing an IgG, scFv or Fab antigen-binding protein comprising the product VH domain and a VL domain.

60-64. (canceled)


SEQUENCE LISTING (357 sequences)

SEQ ID NO: 1 - DNA, 354 bp, Homo sapiens
  C0020065
ggggtcaacttaagggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgtagactccgtgaagggccgattcaccatctccagagacaattccaagaacacgctgtatctgcaaatgaacagcctgagagctgaggacacggctgtgtattactgtgcgagagatagttcgcagctatggtttgactactggggccggggaaccctggtcaccgtctcgagt

SEQ ID NO: 2 - protein, 118 aa, Homo sapiens
  C0020065
GVNLRESGGGVVQPGRSLRLSCAASGFTFSSYAMHWVRQAPGKGLEWVAVISYDGSNKYYVDSVKGRFTISRDNSKNTLYLQMNSLRAEDTAVYYCARDSSQLWFDYWGRGTLVTVSS

SEQ ID NO: 3 - protein, 5 aa, Homo sapiens
  C0020065
SYAMH

SEQ ID NO: 4 - protein, 17 aa, Homo sapiens
  C0020065
VISYDGSNKYYVDSVKG

SEQ ID NO: 5 - protein, 9 aa, Homo sapiens
  C0020065
DSSQLWFDY

SEQ ID NO: 6 - DNA, 336 bp, Homo sapiens
  C0020065
cagtctgtcgtgacgcagccgccctcagtgtctggggccccagggcagagggtcaccatctcctgcactgggagcagctccaacatcgggacaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttctgtggtattcggcggagggacccagctcaccgtttta

SEQ ID NO: 7 - protein, 112 aa, Homo sapiens
  C0020065
QSVVTQPPSVSGAPGQRVTISCTGSSSNIGTGYDVHWYQQLPGTAPKLLIYGNSNRPSGVPDRFSGSKSGTSASLAITGLQAEDEADYYCQSYDSSLSGSVVFGGGTQLTVL

SEQ ID NO: 8 - protein, 14 aa, Homo sapiens
  C0020065
TGSSSNIGTGYDVH

SEQ ID NO: 9 - protein, 7 aa, Homo sapiens
  C0020065
GNSNRPS

SEQ ID NO: 10 - protein, 12 aa, Homo sapiens
  C0020065
QSYDSSLSGSVV

SEQ ID NO: 11 - DNA, 360 bp, Homo sapiens
  C0020187
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 12 - protein, 120 aa, Homo sapiens
  C0020187
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 13 - protein, 5 aa, Homo sapiens
  C0020187
SYAMS

SEQ ID NO: 14 - protein, 17 aa, Homo sapiens
  C0020187
AISGSGGSTYYADSVKG

SEQ ID NO: 15 - protein, 11 aa, Homo sapiens
  C0020187
LRADLGLYMDL

SEQ ID NO: 16 - DNA, 330 bp, Homo sapiens
  C0020187
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 17 - protein, 110 aa, Homo sapiens
  C0020187
QSVLTQPPSVSAAPGQKVTISCSGSSSNIGNHYVSWYQQLPGTAPKLLIYDNSERPSGIPDRFSGSKSGTSATLGITGLQTGDEADYYCGTWDSSLSALVFGGGTKLTVL

SEQ ID NO: 18 - protein, 13 aa, Homo sapiens
  C0020187
SGSSSNIGNHYVS

SEQ ID NO: 19 - protein, 7 aa, Homo sapiens
  C0020187
DNSERPS

SEQ ID NO: 20 - protein, 11 aa, Homo sapiens
  C0020187
GTWDSSLSALV

SEQ ID NO: 21 - DNA, 360 bp, Homo sapiens
  C0021017
gaggtgcagctgttggagtctgggggaggcttggtacagcctggggggtccctgagactctcctgtgcagcctctggattcaccttttatcctgaatacgagttatgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 22 - protein, 120 aa, Homo sapiens
  C0021017
EVQLLESGGGLVQPGGSLRLSCAASGFTFYPEYELWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 23 - protein, 5 aa, Homo sapiens
  C0021017
PEYEL

SEQ ID NO: 24 - protein, 17 aa, Homo sapiens
  C0021017
AISGSGGSTYYADSVKG

SEQ ID NO: 25 - protein, 11 aa, Homo sapiens
  C0021017
LRADLGLYMDL

SEQ ID NO: 26 - DNA, 330 bp, Homo sapiens; identical to SEQ ID NO: 16
  C0021017

SEQ ID NO: 27 - protein, 110 aa, Homo sapiens; identical to SEQ ID NO: 17
  C0021017

SEQ ID NO: 28 - protein, 13 aa, Homo sapiens
  C0021017
SGSSSNIGNHYVS

SEQ ID NO: 29 - protein, 7 aa, Homo sapiens
  C0021017
DNSERPS

SEQ ID NO: 30 - protein, 11 aa, Homo sapiens
  C0021017
GTWDSSLSALV

SEQ ID NO: 31 - DNA, 360 bp, Homo sapiens
  C0021021
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaagcatattattcagtccaggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 32 - protein, 120 aa, Homo sapiens
  C0021021
EVQLLESGGGLVQPGGSLRLSCAASGFTFSSYAMSWVRQAPGKGLEWVSSILFSPGSTYYADSVKGRFTISRDNSKNTLYLQMNSLRAEDTAVYYCARLRADLGLYMDLWGRGTLVTVSS

SEQ ID NO: 33 - protein, 5 aa, Homo sapiens
  C0021021
SYAMS

SEQ ID NO: 34 - protein, 17 aa, Homo sapiens
  C0021021
SILFSPGSTYYADSVKG

SEQ ID NO: 35 - protein, 11 aa, Homo sapiens
  C0021021
LRADLGLYMDL

SEQ ID NO: 36 - DNA, 330 bp, Homo sapiens; identical to SEQ ID NO: 16
  C0021021

SEQ ID NO: 37 - protein, 110 aa, Homo sapiens; identical to SEQ ID NO: 17
  C0021021

SEQ ID NO: 38 - protein, 13 aa, Homo sapiens
  C0021021
SGSSSNIGNHYVS

SEQ ID NO: 39 - protein, 7 aa, Homo sapiens
  C0021021
DNSERPS

SEQ ID NO: 40 - protein, 11 aa, Homo sapiens
  C0021021
GTWDSSLSALV

SEQ ID NO: 41 - DNA, 354 bp, Homo sapiens
  C0021022
gaggtgcagctgttggagtctgggggaggcttggtacagcctggggggtccctgagactctcctgtgcagcctctggattcacctttagcagctatgccatgagctgggtccgccaggctccagggaaggggccggagtgggtctcagctattagtggtttctccccttattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 42 - protein, 118 aa, Homo sapiens
  C0021022
EVQLLESGGGLVQPGGSLRLSCAASGFTFSSYAMSWVRQAPGKGPEWVSAISGFSPYYADSVKGRFTISRDNSKNTLYLQMNSLRAEDTAVYYCARLRADLGLYMDLWGRGTLVTVSS

SEQ ID NO: 43 - protein, 5 aa, Homo sapiens
  C0021022
SYAMS

SEQ ID NO: 44 - protein, 15 aa, Homo sapiens
  C0021022
AISGFSPYYADSVKG

SEQ ID NO: 45 - protein, 11 aa, Homo sapiens
  C0021022
LRADLGLYMDL

SEQ ID NO: 46 - DNA, 330 bp, Homo sapiens; identical to SEQ ID NO: 16
  C0021022

SEQ ID NO: 47 - protein, 110 aa, Homo sapiens; identical to SEQ ID NO: 17
  C0021022

SEQ ID NO: 48 - protein, 13 aa, Homo sapiens
  C0021022
SGSSSNIGNHYVS

SEQ ID NO: 49 - protein, 7 aa, Homo sapiens
  C0021022
DNSERPS

SEQ ID NO: 50 - protein, 11 aa, Homo sapiens
  C0021022
GTWDSSLSALV

SEQ ID NO: 51 - DNA, 360 bp, Homo sapiens
  C0021032
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcatatggatacggaataggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 52 - protein, 120 aa, Homo sapiens
  C0021032
EVQLLESGGGLVQPGGSLRLSCAASGFTFSSYAMSWVRQAPGKGLEWVSAYGYGIGSTYYADSVKGRFTISRDNSKNTLYLQMNSLRAEDTAVYYCARLRADLGLYMDLWGRGTLVTVSS

SEQ ID NO: 53 - protein, 5 aa, Homo sapiens
  C0021032
SYAMS

SEQ ID NO: 54 - protein, 17 aa, Homo sapiens
  C0021032
AYGYGIGSTYYADSVKG

SEQ ID NO: 55 - protein, 11 aa, Homo sapiens
  C0021032
LRADLGLYMDL

SEQ ID NO: 56 - DNA, 330 bp, Homo sapiens; identical to SEQ ID NO: 16
  C0021032

SEQ ID NO: 57 - protein, 110 aa, Homo sapiens; identical to SEQ ID NO: 17
  C0021032

SEQ ID NO: 58 - protein, 13 aa, Homo sapiens
  C0021032
SGSSSNIGNHYVS

SEQ ID NO: 59 - protein, 7 aa, Homo sapiens
  C0021032
DNSERPS

SEQ ID NO: 60 - protein, 11 aa, Homo sapiens
  C0021032
GTWDSSLSALV

SEQ ID NO: 61 - DNA, 360 bp, Homo sapiens
  C0021061
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctaaacctaccgatgctggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 62 - protein, 120 aa, Homo sapiens
  C0021061
EVQLLESGGGLVQPGGSLRLSCAASGFTFSSYAMSWVRQAPGKGLEWVSAKPTDAGSTYYADSVKGRFTISRDNSKNTLYLQMNSLRAEDTAVYYCARLRADLGLYMDLWGRGTLVTVSS

SEQ ID NO: 63 - protein, 5 aa, Homo sapiens
  C0021061
SYAMS

SEQ ID NO: 64 - protein, 17 aa, Homo sapiens
  C0021061
AKPTDAGSTYYADSVKG

SEQ ID NO: 65 - protein, 11 aa, Homo sapiens
  C0021061
LRADLGLYMDL

SEQ ID NO: 66 - DNA, 330 bp, Homo sapiens
  C0021061
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataacacggtgtaccccaagtcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 67 - protein, 110 aa, Homo sapiens
  C0021061
QSVLTQPPSVSAAPGQKVTISCSGSSSNIGNHYVSWYQQLPGTAPKLLIYNTVYPKSGIPDRFSGSKSGTSATLGITGLQTGDEADYYCGTWDSSLSALVFGGGTKLTVL

SEQ ID NO: 68 - protein, 13 aa, Homo sapiens
  C0021061
SGSSSNIGNHYVS

SEQ ID NO: 69 - protein, 7 aa, Homo sapiens
  C0021061
NTVYPKS

SEQ ID NO: 70 - protein, 11 aa, Homo sapiens
  C0021061
GTWDSSLSALV

SEQ ID NO: 71 - DNA, 354 bp, Homo sapiens
  C0021065
gaggtgcagctgttggagtctgggggaggcttggtacagcctggggggtccctgagactctcctgtgcagcctctggattcacctttagcagctatgccatgagctgggtccgccaggctccagggaaggggccggagtgggtctcagctattagtggtttctccccttattacgcagactccgtgaagggccggttcaccatctccagagacaattccaggaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 72 - protein, 118 aa, Homo sapiens
  C0021065
EVQLLESGGGLVQPGGSLRLSCAASGFTFSSYAMSWVRQAPGKGPEWVSAISGFSPYYADSVKGRFTISRDNSRNTLYLQMNSLRAEDTAVYYCARLRADLGLYMDLWGRGTLVTVSS

SEQ ID NO: 73 - protein, 5 aa, Homo sapiens
  C0021065
SYAMS

SEQ ID NO: 74 - protein, 15 aa, Homo sapiens
  C0021065
AISGFSPYYADSVKG

SEQ ID NO: 75 - protein, 11 aa, Homo sapiens
  C0021065
LRADLGLYMDL

SEQ ID NO: 76 - DNA, 330 bp, Homo sapiens
  C0021065
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacacggaccacatggcgtcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 77 - protein, 110 aa, Homo sapiens
  C0021065
QSVLTQPPSVSAAPGQKVTISCSGSSSNIGNHYVSWYQQLPGTAPKLLIYDTDHMASGIPDRFSGSKSGTSATLGITGLQTGDEADYYCGTWDSSLSALVFGGGTKLTVL

SEQ ID NO: 78 - protein, 13 aa, Homo sapiens
  C0021065
SGSSSNIGNHYVS

SEQ ID NO: 79 - protein, 7 aa, Homo sapiens
  C0021065
DTDHMAS

SEQ ID NO: 80 - protein, 11 aa, Homo sapiens
  C0021065
GTWDSSLSALV

SEQ ID NO: 81 - DNA, 360 bp, Homo sapiens
  C0021089
gaggtgcagctgttggagtctgggggaggcttggtacagcctggggggtccctgagactctcctgtgcagcctctggattcacctttccatacgaatattggtcttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagcccaggacacggccgtgtattactgtgcgaggcttagagcggatttagggttgtacatggacctctggggccggggcaccctggtcaccgtctcgagt

SEQ ID NO: 82 - protein, 120 aa, Homo sapiens
  C0021089
EVQLLESGGGLVQPGGSLRLSCAASGFTFPYEYWSWVRQAPGKGLEWVSAISGSGGSTYYADSVKGRFTISRDNSKNTLYLQMNSLRAQDTAVYYCARLRADLGLYMDLWGRGTLVTVSS

SEQ ID NO: 83 - protein, 5 aa, Homo sapiens
  C0021089
YEYWS

SEQ ID NO: 84 - protein, 17 aa, Homo sapiens
  C0021089
AISGSGGSTYYADSVKG

SEQ ID NO: 85 - protein, 11 aa, Homo sapiens
  C0021089
LRADLGLYMDL

SEQ ID NO: 86 - DNA, 330 bp, Homo sapiens
  C0021089
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcagggattcctgaccgattctctggctccaagtctggcacgtcagccaccctgggcatcaccggactccagactggggacgaggccgattattactgcggaacatgggacccgaccgtctcgaggttggtgttcggcggcgggaccaagctgaccgtccta

SEQ ID NO: 87 - protein, 110 aa, Homo sapiens
  C0021089
QSVLTQPPSVSAAPGQKVTISCSGSSSNIGNHYVSWYQQLPGTAPKLLIYDNSERPSGIPDRFSGSKSGTSATLGITGLQTGDEADYYCGTWDPTVSRLVFGGGTKLTVL

SEQ ID NO: 88 - protein, 13 aa, Homo sapiens
  C0021089
SGSSSNIGNHYVS

SEQ ID NO: 89 - protein, 7 aa, Homo sapiens
  C0021089
DNSERPS

SEQ ID NO: 90 - protein, 11 aa, Homo sapiens
  C0021089
GTWDPTVSRLV

SEQ ID NO: 91 - DNA, 360 bp, Homo sapiens
  C0021092
gaggtgcagctgttggagtctgggggaggcttggtacagcctggggggtccctgagactctcctgtgcagcctctggattcacctttagcagatatgccatgagctgggtccgccaggctccagggaaggggctggagtgggtctcagcacatacccactctcccggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 92 - protein, 120 aa, Homo sapiens
  C0021092
EVQLLESGGGLVQPGGSLRLSCAASGFTFSRYAMSWVRQAPGKGLEWVSAHTHSPGSTYYADSVKGRFTISRDNSKNTLYLQMNSLRAEDTAVYYCARLRADLGLYMDLWGRGTLVTVSS

SEQ ID NO: 93 - protein, 5 aa, Homo sapiens
  C0021092
RYAMS

SEQ ID NO: 94 - protein, 17 aa, Homo sapiens
  C0021092
AHTHSPGSTYYADSVKG

SEQ ID NO: 95 - protein, 11 aa, Homo sapiens
  C0021092
LRADLGLYMDL

SEQ ID NO: 96 - DNA, 330 bp, Homo sapiens
  C0021092
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataacagcgtggcgcgcccgtcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 97 - protein, 110 aa, Homo sapiens
  C0021092
QSVLTQPPSVSAAPGQKVTISCSGSSSNIGNHYVSWYQQLPGTAPKLLIYNSVARPSGIPDRFSGSKSGTSATLGITGLQTGDEADYYCGTWDSSLSALVFGGGTKLTVL

SEQ ID NO: 98 - protein, 13 aa, Homo sapiens
  C0021092
SGSSSNIGNHYVS

SEQ ID NO: 99 - protein, 7 aa, Homo sapiens
  C0021092
NSVARPS

SEQ ID NO: 100 - protein, 11 aa, Homo sapiens
  C0021092
GTWDSSLSALV

SEQ ID NO: 101 - DNA, 354 bp, Homo sapiens
  C0021096
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggttacagtggatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 102 - protein, 118 aa, Homo sapiens
  C0021096
EVQLLESGGGLVQPGGSLRLSCAASGFTFSSYAMSWVRQAPGKGLEWVSAISGYSGYYADSVKGRFTISRDNSKNTLYLQMNSLRAEDTAVYYCARLRADLGLYMDLWGRGTLVTVSS

SEQ ID NO: 103 - protein, 5 aa, Homo sapiens
  C0021096
SYAMS

SEQ ID NO: 104 - protein, 15 aa, Homo sapiens
  C0021096
AISGYSGYYADSVKG

SEQ ID NO: 105 - protein, 11 aa, Homo sapiens
  C0021096
LRADLGLYMDL

SEQ ID NO: 106 - DNA, 330 bp, Homo sapiens
  C0021096
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataacaccgagaggcggatgtcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 107 - protein, 110 aa, Homo sapiens
  C0021096
QSVLTQPPSVSAAPGQKVTISCSGSSSNIGNHYVSWYQQLPGTAPKLLIYNTERRMSGIPDRFSGSKSGTSATLGITGLQTGDEADYYCGTWDSSLSALVFGGGTKLTVL

SEQ ID NO: 108 - protein, 13 aa, Homo sapiens
  C0021096
SGSSSNIGNHYVS

SEQ ID NO: 109 - protein, 7 aa, Homo sapiens
  C0021096
NTERRMS

SEQ ID NO: 110 - protein, 11 aa, Homo sapiens
  C0021096
GTWDSSLSALV

SEQ ID NO: 111 - DNA, 360 bp, Homo sapiens
  C0021097
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagccattagttaccatcctggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 112 - protein, 120 aa, Homo sapiens
  C0021097
EVQLLESGGGLVQPGGSLRLSCAASGFTFSSYAMSWVRQAPGKGLEWVSAISYHPGSTYYADSVKGRFTISRDNSKNTLYLQMNSLRAEDTAVYYCARLRADLGLYMDLWGRGTLVTVSS

SEQ ID NO: 113 - protein, 5 aa, Homo sapiens
  C0021097
SYAMS

SEQ ID NO: 114 - protein, 17 aa, Homo sapiens
  C0021097
AISYHPGSTYYADSVKG

SEQ ID NO: 115 - protein, 11 aa, Homo sapiens
  C0021097
LRADLGLYMDL

SEQ ID NO: 116 - DNA, 330 bp, Homo sapiens
  C0021097
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataacacgttcgcgcgcccctcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 117 - protein, 110 aa, Homo sapiens
  C0021097
QSVLTQPPSVSAAPGQKVTISCSGSSSNIGNHYVSWYQQLPGTAPKLLIYNTFARPSGIPDRFSGSKSGTSATLGITGLQTGDEADYYCGTWDSSLSALVFGGGTKLTVL

SEQ ID NO: 118 - protein, 13 aa, Homo sapiens
  C0021097
SGSSSNIGNHYVS

SEQ ID NO: 119 - protein, 7 aa, Homo sapiens
  C0021097
NTFARPS

SEQ ID NO: 120 - protein, 11 aa, Homo sapiens
  C0021097
GTWDSSLSALV

SEQ ID NO: 121 - DNA, 360 bp, Homo sapiens
  C0021098
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatccagcacctatggtgccggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 122 - protein, 120 aa, Homo sapiens
  C0021098
EVQLLESGGGLVQPGGSLRLSCAASGFTFSSYAMSWVRQAPGKGLEWVSSSTYGAGSTYYADSVKGRFTISRDNSKNTLYLQMNSLRAEDTAVYYCARLRADLGLYMDLWGRGTLVTVSS

SEQ ID NO: 123 - protein, 5 aa, Homo sapiens
  C0021098
SYAMS

SEQ ID NO: 124 - protein, 17 aa, Homo sapiens
  C0021098
SSTYGAGSTYYADSVKG

SEQ ID NO: 125 - protein, 11 aa, Homo sapiens
  C0021098
LRADLGLYMDL

SEQ ID NO: 126 - DNA, 330 bp, Homo sapiens
  C0021098
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataactcggacgcccccatgtcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 127 - protein, 110 aa, Homo sapiens
  C0021098
QSVLTQPPSVSAAPGQKVTISCSGSSSNIGNHYVSWYQQLPGTAPKLLIYNSDAPMSGIPDRFSGSKSGTSATLGITGLQTGDEADYYCGTWDSSLSALVFGGGTKLTVL

SEQ ID NO: 128 - protein, 13 aa, Homo sapiens
  C0021098
SGSSSNIGNHYVS

SEQ ID NO: 129 - protein, 7 aa, Homo sapiens
  C0021098
NSDAPMS

SEQ ID NO: 130 - protein, 11 aa, Homo sapiens
  C0021098
GTWDSSLSALV

SEQ ID NO: 131 - DNA, 354 bp, Homo sapiens
  C0021101
gaggtgcaactgttggagtctgggggaggcttggtacagcctggggggtccctgagactctcctgtgcagcctctggattcacctttagcagctatgccatgagctgggtccgccaggctccagggaaggggccggagtgggtctcagctattagtggtttctccccttattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 132 - protein, 118 aa, Homo sapiens; identical to SEQ ID NO: 42
  C0021101

SEQ ID NO: 133 - protein, 5 aa, Homo sapiens
  C0021101
SYAMS

SEQ ID NO: 134 - protein, 15 aa, Homo sapiens
  C0021101
AISGFSPYYADSVKG

SEQ ID NO: 135 - protein, 11 aa, Homo sapiens
  C0021101
LRADLGLYMDL

SEQ ID NO: 136 - DNA, 330 bp, Homo sapiens
  C0021101
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttataacaaccggtccccgacctcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 137 - protein, 110 aa, Homo sapiens
  C0021101
QSVLTQPPSVSAAPGQKVTISCSGSSSNIGNHYVSWYQQLPGTAPKLLIYNNRSPTSGIPDRFSGSKSGTSATLGITGLQTGDEADYYCGTWDSSLSALVFGGGTKLTVL

SEQ ID NO: 138 - protein, 13 aa, Homo sapiens
  C0021101
SGSSSNIGNHYVS

SEQ ID NO: 139 - protein, 7 aa, Homo sapiens
  C0021101
NNRSPTS

SEQ ID NO: 140 - protein, 11 aa, Homo sapiens
  C0021101
GTWDSSLSALV

SEQ ID NO: 141 - DNA, 360 bp, Homo sapiens
  C0021118
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctagccttaccaatcctggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 142 - protein, 120 aa, Homo sapiens
  C0021118
EVQLLESGGGLVQPGGSLRLSCAASGFTFSSYAMSWVRQAPGKGLEWVSASLTNPGSTYYADSVKGRFTISRDNSKNTLYLQMNSLRAEDTAVYYCARLRADLGLYMDLWGRGTLVTVSS

SEQ ID NO: 143 - protein, 5 aa, Homo sapiens
  C0021118
SYAMS

SEQ ID NO: 144 - protein, 17 aa, Homo sapiens
  C0021118
ASLTNPGSTYYADSVKG

SEQ ID NO: 145 - protein, 11 aa, Homo sapiens
  C0021118
LRADLGLYMDL

SEQ ID NO: 146 - DNA, 330 bp, Homo sapiens
  C0021118
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcagggattcctgaccgattctctggctccaagtctggcacgtcagccaccctgggcatcaccggactccagactggggacgaggccgattattactgcgcgacgtacgacaccaacctgagtgccttggtgttcggcggcgggaccaagctgaccgtccta

SEQ ID NO: 147 - protein, 110 aa, Homo sapiens
  C0021118
QSVLTQPPSVSAAPGQKVTISCSGSSSNIGNHYVSWYQQLPGTAPKLLIYDNSERPSGIPDRFSGSKSGTSATLGITGLQTGDEADYYCATYDTNLSALVFGGGTKLTVL

SEQ ID NO: 148 - protein, 13 aa, Homo sapiens
  C0021118
SGSSSNIGNHYVS

SEQ ID NO: 149 - protein, 7 aa, Homo sapiens
  C0021118
DNSERPS

SEQ ID NO: 150 - protein, 11 aa, Homo sapiens
  C0021118
ATYDTNLSALV

SEQ ID NO: 151 - DNA, 360 bp, Homo sapiens
  C0021124
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagccattagttaccatcctggtagcacatactacgcagactccgtgaagggccggttcaccatctccagagacaattccaagaacacgctgtatctgcaaataaacagcctgagagccgaggacacggccgtgtattactgtgcgaggcttagagcggatttagggttgtacatggacctctggggccggggcaccctggtcaccgtctcgagt

SEQ ID NO: 152 - protein, 120 aa, Homo sapiens
  C0021124
EVQLLESGGGLVQPGGSLRLSCAASGFTFSSYAMSWVRQAPGKGLEWVSAISYHPGSTYYADSVKGRFTISRDNSKNTLYLQINSLRAEDTAVYYCARLRADLGLYMDLWGRGTLVTVSS

SEQ ID NO: 153 - protein, 5 aa, Homo sapiens
  C0021124
SYAMS

SEQ ID NO: 154 - protein, 17 aa, Homo sapiens
  C0021124
AISYHPGSTYYADSVKG

SEQ ID NO: 155 - protein, 11 aa, Homo sapiens
  C0021124
LRADLGLYMDL

SEQ ID NO: 156 - DNA, 330 bp, Homo sapiens
  C0021124
cagtctgtgctgacgcagctgccctcagtgtctgcggccccaggtcagaaggtcaccatctcctgctctggaagcagttccaacattggcaatcattatgtatcctggtaccagcaactacccggaacagcccccaaactcctcatttatgacaatagtgagcgaccctcagggattcctgaccgattctctggctccaagtctggcacgtcagccaccctgggcatcaccggactccagactggggacgaggccgattattactgcggaacatgggatagcgaccgggtcaacctcgagttcggcggcgggaccaagctgaccgtccta

SEQ ID NO: 157 - protein, 110 aa, Homo sapiens
  C0021124
QSVLTQLPSVSAAPGQKVTISCSGSSSNIGNHYVSWYQQLPGTAPKLLIYDNSERPSGIPDRFSGSKSGTSATLGITGLQTGDEADYYCGTWDSDRVNLEFGGGTKLTVL

SEQ ID NO: 158 - protein, 13 aa, Homo sapiens
  C0021124
SGSSSNIGNHYVS

SEQ ID NO: 159 - protein, 7 aa, Homo sapiens
  C0021124
DNSERPS

SEQ ID NO: 160 - protein, 11 aa, Homo sapiens
  C0021124
GTWDSDRVNLE

SEQ ID NO: 161 - DNA, 360 bp, Homo sapiens
  C0021128
gaggtgcagctgttggagtctgggggaggcttggtacagcctggggggtccctgagactctcctgtgcagcctctggattcacctttcaatacgaagtagccgcttgggtccgccaggctccagggaaggggctggagtgggtctcagctattagtggtcccatccctaaaggctattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 162 - protein, 120 aa, Homo sapiens
  C0021128
EVQLLESGGGLVQPGGSLRLSCAASGFTFQYEVAAWVRQAPGKGLEWVSAISGPIPKGYYADSVKGRFTISRDNSKNTLYLQMNSLRAEDTAVYYCARLRADLGLYMDLWGRGTLVTVSS

SEQ ID NO: 163 - protein, 5 aa, Homo sapiens
  C0021128
YEVAA

SEQ ID NO: 164 - protein, 17 aa, Homo sapiens
  C0021128
AISGPIPKGYYADSVKG

SEQ ID NO: 165 - protein, 11 aa, Homo sapiens
  C0021128
LRADLGLYMDL

SEQ ID NO: 166 - DNA, 330 bp, Homo sapiens
  C0021128
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaacgccatcgagagctcagggattcctgaccgattctctggctccaagtctggcacgtcagccaccctgggcatcaccggactccagactggggacggggccgattattactgcggaacatgggatagcaacctgagtgccttggtgttcggcggcgggaccaagctgaccgtccta

SEQ ID NO: 167 - protein, 110 aa, Homo sapiens
  C0021128
QSVLTQPPSVSAAPGQKVTISCSGSSSNIGNHYVSWYQQLPGTAPKLLIYDNAIESSGIPDRFSGSKSGTSATLGITGLQTGDGADYYCGTWDSNLSALVFGGGTKLTVL

SEQ ID NO: 168 - protein, 13 aa, Homo sapiens
  C0021128
SGSSSNIGNHYVS

SEQ ID NO: 169 - protein, 7 aa, Homo sapiens
  C0021128
DNAIESS

SEQ ID NO: 170 - protein, 11 aa, Homo sapiens
  C0021128
GTWDSNLSALV

SEQ ID NO: 171 - DNA, 354 bp, Homo sapiens
  C0021129
gaggtgcagctgttggagtctgggggaggcttggtacggcctggggggtccctgagactctcctgtgcagcctctggattcacctttagcagctatgccatgagctgggtccgccaggctccagggaaggggctggagtgggtctcagctattagtggtttcagtccattttacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 172 - protein, 118 aa, Homo sapiens
  C0021129
EVQLLESGGGLVRPGGSLRLSCAASGFTFSSYAMSWVRQAPGKGLEWVSAISGFSPFYADSVKGRFTISRDNSKNTLYLQMNSLRAEDTAVYYCARLRADLGLYMDLWGRGTLVTVSS

SEQ ID NO: 173 - protein, 5 aa, Homo sapiens
  C0021129
SYAMS

SEQ ID NO: 174 - protein, 15 aa, Homo sapiens
  C0021129
AISGFSPFYADSVKG

SEQ ID NO: 175 - protein, 11 aa, Homo sapiens
  C0021129
LRADLGLYMDL

SEQ ID NO: 176 - DNA, 330 bp, Homo sapiens
  C0021129
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcagggattcctgaccgattctctggctccaagtctggcacgtcagccaccctggacatcaccggactccagactggggacgaggccgattattactgcggaacatgggacgagacggcggccgagttggtgttcggcggcgggaccaagctgaccgtccta

SEQ ID NO: 177 - protein, 110 aa, Homo sapiens
  C0021129
QSVLTQPPSVSAAPGQKVTISCSGSSSNIGNHYVSWYQQLPGTAPKLLIYDNSERPSGIPDRFSGSKSGTSATLDITGLQTGDEADYYCGTWDETAAELVFGGGTKLTVL

SEQ ID NO: 178 - protein, 13 aa, Homo sapiens
  C0021129
SGSSSNIGNHYVS

SEQ ID NO: 179 - protein, 7 aa, Homo sapiens
  C0021129
DNSERPS

SEQ ID NO: 180 - protein, 11 aa, Homo sapiens
  C0021129
GTWDETAAELV

SEQ ID NO: 181 - DNA, 360 bp, Homo sapiens
  C0021131
gaggtgcagctgttggagtctgggggaggcttggtacagcctggggggtccctgagactctcctgtgcagcctctggattcacctttccatacgattatatgggttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 182 - protein, 120 aa, Homo sapiens
  C0021131
EVQLLESGGGLVQPGGSLRLSCAASGFTFPYDYMGWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 183 - protein, 5 aa, Homo sapiens
  C0021131
YDYMG

SEQ ID NO: 184 - protein, 17 aa, Homo sapiens
  C0021131
AISGSGGSTYYADSVKG

SEQ ID NO: 185 - protein, 11 aa, Homo sapiens
  C0021131
LRADLGLYMDL

SEQ ID NO: 186 - DNA, 330 bp, Homo sapiens
  C0021131
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 187 - protein, 110 aa, Homo sapiens
  C0021131
QSVLTQPPSVSAAPGQKVTISCSGSSSNIGNHYVSWYQQLPGTAPKLLIYDNSERPSGIPDRFSGSKSGTSATLGITGLQTGDEADYYCGTWDELTSNLVFGGGTKLTVL

SEQ ID NO: 188 - protein, 13 aa, Homo sapiens
  C0021131
SGSSSNIGNHYVS

SEQ ID NO: 189 - protein, 7 aa, Homo sapiens
  C0021131
DNSERPS

SEQ ID NO: 190 - protein, 11 aa, Homo sapiens
  C0021131
GTWDELTSNLV

SEQ ID NO: 191 - DNA, 360 bp, Homo sapiens
  C0021133
gaggtgcagctgttggagtctgggggaggcttggtacagcctggggggtccctgagactctcctgtgcagcctctggattcacctttcgctacgatcaccacgtttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcactgtctcgagt

SEQ ID NO: 192 - protein, 120 aa, Homo sapiens
  C0021133
EVQLLESGGGLVQPGGSLRLSCAASGFTFRYDHHVWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 193 - protein, 5 aa, Homo sapiens
  C0021133
YDHHV

SEQ ID NO: 194 - protein, 17 aa, Homo sapiens
  C0021133
AISGSGGSTYYADSVKG

SEQ ID NO: 195 - protein, 11 aa, Homo sapiens
  C0021133
LRADLGLYMDL

SEQ ID NO: 196 - DNA, 330 bp, Homo sapiens
  C0021133
cagtctgtgctgacgcagccgccctcagtgtctgcggccccaggtcagaaggtcaccgtctcctgctctggaagcagttccaacattggcaatcattatgtatcctggtaccagcaactaccaggaacagcccccaaactcctcatttatgacacgaccgtcttgagctcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 197 - protein, 110 aa, Homo sapiens
  C0021133
QSVLTQPPSVSAAPGQKVTVSCSGSSSNIGNHYVSWYQQLPGTAPKLLIYDTTVLSSGIPDRFSGSKSGTSATLGITGLQTGDEADYYCGTWDELTSNLVFGGGTKLTVL

SEQ ID NO: 198 - protein, 13 aa, Homo sapiens
  C0021133
SGSSSNIGNHYVS

SEQ ID NO: 199 - protein, 7 aa, Homo sapiens
  C0021133
DTTVLSS

SEQ ID NO: 200 - protein, 11 aa, Homo sapiens
  C0021133
GTWDELTSNLV

SEQ ID NO: 201 - DNA, 360 bp, Homo sapiens
  C0021135
gaggtgcagctgttggagtctgggggaggcttggtacagcctggggggtccctgagactctcctgtgcagcctctggattcgcctttcgatacgaatacatccaatgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 202 - protein, 120 aa, Homo sapiens
  C0021135
EVQLLESGGGLVQPGGSLRLSCAASGFAFRYEYIQWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 203 - protein, 5 aa, Homo sapiens
  C0021135
YEYIQ

SEQ ID NO: 204 - protein, 17 aa, Homo sapiens
  C0021135
AISGSGGSTYYADSVKG

SEQ ID NO: 205 - protein, 11 aa, Homo sapiens
  C0021135
LRADLGLYMDL

SEQ ID NO: 206 - DNA, 330 bp, Homo sapiens
  C0021135
cagtctgtgctgacgcagccgccctcagtgtctgcggccccaggtcagaaggtcaccatctcctgctctggaagcagttccaacattggcaatcattatgtatcctggtaccagcaactaccaggaacagcccacaaactcctcatttatgacaatagtgagcgaccctcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 207 - protein, 110 aa, Homo sapiens
  C0021135
QSVLTQPPSVSAAPGQKVTISCSGSSSNIGNHYVSWYQQLPGTAHKLLIYDNSERPSGIPDRFSGSKSGTSATLGITGLQTGDEADYYCGTWDSSLSALVFGGGTKLTVL

SEQ ID NO: 208 - protein, 13 aa, Homo sapiens
  C0021135
SGSSSNIGNHYVS

SEQ ID NO: 209 - protein, 7 aa, Homo sapiens
  C0021135
DNSERPS

SEQ ID NO: 210 - protein, 11 aa, Homo sapiens
  C0021135
GTWDSSLSALV

SEQ ID NO: 211 - DNA, 360 bp, Homo sapiens
  C0021139
gaggtgcagctgttggagtctgggggaggcttggtacagcctggggggtccctgagactctcctgtgcagcctctggattcacctttcaatacgattatcaggtttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 212 - protein, 120 aa, Homo sapiens
  C0021139
EVQLLESGGGLVQPGGSLRLSCAASGFTFQYDYQVWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 213 - protein, 5 aa, Homo sapiens
  C0021139
YDYQV

SEQ ID NO: 214 - protein, 17 aa, Homo sapiens
  C0021139
AISGSGGSTYYADSVKG

SEQ ID NO: 215 - protein, 11 aa, Homo sapiens
  C0021139
LRADLGLYMDL

SEQ ID NO: 216 - DNA, 330 bp, Homo sapiens
  C0021139
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaacctcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 217 - protein, 110 aa, Homo sapiens
  C0021139
QSVLTQPPSVSAAPGQKVTISCSGSSSNIGNHYVSWYQQLPGTAPKLLIYDNSERTSGIPDRFSGSKSGTSATLGITGLQTGDEADYYCGTWDELTSNLVFGGGTKLTVL

SEQ ID NO: 218 - protein, 13 aa, Homo sapiens
  C0021139
SGSSSNIGNHYVS

SEQ ID NO: 219 - protein, 7 aa, Homo sapiens
  C0021139
DNSERTS

SEQ ID NO: 220 - protein, 11 aa, Homo sapiens
  C0021139
GTWDELTSNLV

SEQ ID NO: 221 - DNA, 360 bp, Homo sapiens
  C0021141
gaggtgcagctgttggagtctgggggaggcttggtacagcctggggggtccctgagactctcctgtgcagcctctggattcaccttttatcccgacccacagggctgggtccgccaggctccagggaaggggctggagtgggtctcagcatatggatacggaataggtagcacatactacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 222 - protein, 120 aa, Homo sapiens
  C0021141
EVQLLESGGGLVQPGGSLRLSCAASGFTFYPDPQGWVRQAPGKGLEWVSAYGYGIGSTYYADSVKGRFTISRDNSKNTLYLQMNSLRAEDTAVYYCARLRADLGLYMDLWGRGTLVTVSS

SEQ ID NO: 223 - protein, 5 aa, Homo sapiens
  C0021141
PDPQG

SEQ ID NO: 224 - protein, 17 aa, Homo sapiens
  C0021141
AYGYGIGSTYYADSVKG

SEQ ID NO: 225 - protein, 11 aa, Homo sapiens
  C0021141
LRADLGLYMDL

SEQ ID NO: 226 - DNA, 330 bp, Homo sapiens
  C0021141
cagtctgtgctgacgcagccgccctcagtgtctgcggccccaggtcaggaggtcaccatctcctgctctggaagcagttccaacattgacaatcattatgtatcctggtaccagcaactaccaggaacagcccccaaactcctcatttatgacaatagtgagcgaccctcagggattcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 227 - protein, 110 aa, Homo sapiens
  C0021141
QSVLTQPPSVSAAPGQEVTISCSGSSSNIDNHYVSWYQQLPGTAPKLLIYDNSERPSGIPDRFSGSKSGTSATLGITGLQTGDEADYYCGTWDSSLSALVFGGGTKLTVL

SEQ ID NO: 228 - protein, 13 aa, Homo sapiens
  C0021141
SGSSSNIDNHYVS

SEQ ID NO: 229 - protein, 7 aa, Homo sapiens
  C0021141
DNSERPS

SEQ ID NO: 230 - protein, 11 aa, Homo sapiens
  C0021141
GTWDSSLSALV

SEQ ID NO: 231 - DNA, 360 bp, Homo sapiens
  C0021142
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcacatacccactctcccggtagcacatactacgcagactccgtgaagggccggttcaccatctccagagacaattccaagaacacgctgtatctgcaaatgaacagcctgagggccgaggacacggccgtgtattactgtgcgaggcttagagcggatttagggttgtacatggacctctggggccggggcaccctggtcaccgtctcgagt

SEQ ID NO: 232 - protein, 120 aa, Homo sapiens
  C0021142
EVQLLESGGGLVQPGGSLRLSCAASGFTFSSYAMSWVRQAPGKGLEWVSAHTHSPGSTYYADSVKGRFTISRDNSKNTLYLQMNSLRAEDTAVYYCARLRADLGLYMDLWGRGTLVTVSS

SEQ ID NO: 233 - protein, 5 aa, Homo sapiens
  C0021142
SYAMS

SEQ ID NO: 234 - protein, 17 aa, Homo sapiens
  C0021142
AHTHSPGSTYYADSVKG

SEQ ID NO: 235 - protein, 11 aa, Homo sapiens
  C0021142
LRADLGLYMDL

SEQ ID NO: 236 - DNA, 330 bp, Homo sapiens
  C0021142
cagtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacaatagtgagcgaccctcaggggttcctgaccgattctctggctccaagtctggcacgtcagccaccctgggcatcaccggactccagactggggacgaggccgattattactgcggaacatgggatagcagcctgagtgccttggtgttcggcggcgggaccaagctgaccgtccta

SEQ ID NO: 237 - protein, 110 aa, Homo sapiens
  C0021142
QSVLTQPPSVSAAPGQKVTISCSGSSSNIGNHYVSWYQQLPGTAPKLLIYDNSERPSGVPDRFSGSKSGTSATLGITGLQTGDEADYYCGTWDSSLSALVFGGGTKLTVL

SEQ ID NO: 238 - protein, 13 aa, Homo sapiens
  C0021142
SGSSSNIGNHYVS

SEQ ID NO: 239 - protein, 7 aa, Homo sapiens
  C0021142
DNSERPS

SEQ ID NO: 240 - protein, 11 aa, Homo sapiens
  C0021142
GTWDSSLSALV

SEQ ID NO: 241 - DNA, 360 bp, Homo sapiens; identical to SEQ ID NO: 231
  C0021142 IgG

SEQ ID NO: 242 - protein, 120 aa, Homo sapiens; identical to SEQ ID NO: 232
  C0021142 IgG

SEQ ID NO: 243 - protein, 5 aa, Homo sapiens
  C0021142 IgG
SYAMS

SEQ ID NO: 244 - protein, 17 aa, Homo sapiens
  C0021142 IgG
AHTHSPGSTYYADSVKG

SEQ ID NO: 245 - protein, 11 aa, Homo sapiens
  C0021142 IgG
LRADLGLYMDL

SEQ ID NO: 246 - DNA, 330 bp, Homo sapiens; identical to SEQ ID NO: 16
  C0021142 IgG

SEQ ID NO: 247 - protein, 110 aa, Homo sapiens; identical to SEQ ID NO: 17
  C0021142 IgG

SEQ ID NO: 248 - protein, 13 aa, Homo sapiens
  C0021142 IgG
SGSSSNIGNHYVS

SEQ ID NO: 249 - protein, 7 aa, Homo sapiens
  C0021142 IgG
DNSERPS

SEQ ID NO: 250 - protein, 11 aa, Homo sapiens
  C0021142 IgG
GTWDSSLSALV

SEQ ID NO: 251 - DNA, 360 bp, Homo sapiens
  C0021144
gaggtgcagctgttggagtctgggggaggcttggtacagcctggggggtccctgagactctcctgtgcagcttctggattcacctttcgctacgattaccacgtttgggtccgccaggctccagggaaggggctggagtgggtctcagcaattagtggtagtggtggtagcacatactacgcagactccgtgaagggccggttcaccatctccagagacaattccaagaacacgttgtatctgcagatgaacagcctgagagccgaggacacggccgtgtattactgtgcgaggcttagagcggatttagggctgtacatggacctctggggccggggcaccctggtcacagtctcgagt

SEQ ID NO: 252 - protein, 120 aa, Homo sapiens
  C0021144
EVQLLESGGGLVQPGGSLRLSCAASGFTFRYDYHVWVRQAPGKGLEWVSAISGSGGSTYYADSVKGRFTISRDNSKNTLYLQMNSLRAEDTAVYYCARLRADLGLYMDLWGRGTLVTVSS

SEQ ID NO: 253 - protein, 5 aa, Homo sapiens
  C0021144
YDYHV

SEQ ID NO: 254 - protein, 17 aa, Homo sapiens
  C0021144
AISGSGGSTYYADSVKG

SEQ ID NO: 255 - protein, 11 aa, Homo sapiens
  C0021144
LRADLGLYMDL

SEQ ID NO: 256 - DNA, 330 bp, Homo sapiens
  C0021144
cagtctgtgctgacgcagccgccctcagtgtccgcggccccaggtcagaaggtcaccgtctcctgctctggaagcagttccaacattggcaatcatcatgtatcctggtaccagcaactaccaggaacagcccccaaactcctcatttatgacacgaccgtcttgagctcaggggttcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 257 - protein, 110 aa, Homo sapiens
  C0021144
QSVLTQPPSVSAAPGQKVTVSCSGSSSNIGNHHVSWYQQLPGTAPKLLIYDTTVLSSGVPDRFSGSKSGTSATLGITGLQTGDEADYYCGTWDELTSNLVFGGGTKLTVL

SEQ ID NO: 258 - protein, 13 aa, Homo sapiens
  C0021144
SGSSSNIGNHHVS

SEQ ID NO: 259 - protein, 7 aa, Homo sapiens
  C0021144
DTTVLSS

SEQ ID NO: 260 - protein, 11 aa, Homo sapiens
  C0021144
GTWDELTSNLV

SEQ ID NO: 261 - DNA, 360 bp, Homo sapiens
  C0021155
gaggtgcagctgttggagtctgggggaggcttggtacagcctggggggtccctgagactctcctgtgaagcctctggattcacctttcgctacgattaccacgtttgggtccgccaggctccagggaaggggctggagtgggtctcagctattagtggtagtggtggtagcacatactacgcagaccccgtgaagggccggttcaccatctccagagacaattccaagaacacgctgtatctgcaaatgaacagcctgagagccgaggacacggccgtgtattactgtgcgaggcttagagcggatttggggttgtacatggacctctggggccggggcaccctggtcaccgtctcgagc

SEQ ID NO: 262 - protein, 120 aa, Homo sapiens
  C0021155
EVQLLESGGGLVQPGGSLRLSCEASGFTFRYDYHVWVRQAPGKGLEWVSAISGSGGSTYYADPVKGRFTISRDNSKNTLYLQMNSLRAEDTAVYYCARLRADLGLYMDLWGRGTLVTVSS

SEQ ID NO: 263 - protein, 5 aa, Homo sapiens
  C0021155
YDYHV

SEQ ID NO: 264 - protein, 17 aa, Homo sapiens
  C0021155
AISGSGGSTYYADPVKG

SEQ ID NO: 265 - protein, 11 aa, Homo sapiens
  C0021155
LRADLGLYMDL

SEQ ID NO: 266 - DNA, 330 bp, Homo sapiens
  C0021155
cggtctgtgctgacgcagccgccctcagtgtctgcggccccaggtcagaaggtcaccatctcctgctctggaagcagttccaacattggcaatcattatgtatcctggtaccagcaactaccaggaacagcccccaaactcctcatttatgacacgaccgtcctgagctcagggattcctgaccgattctctggctccaagtctggcacgtcagccaccctgggcatcaccggactccagactggggacgaggccgattactactgcggaacatgggacgagctgacctccaacttggtgttcggcggcgggaccaggctgaccgtccta

SEQ ID NO: 267 - protein, 110 aa, Homo sapiens
  C0021155
RSVLTQPPSVSAAPGQKVTISCSGSSSNIGNHYVSWYQQLPGTAPKLLIYDTTVLSSGIPDRFSGSKSGTSATLGITGLQTGDEADYYCGTWDELTSNLVFGGGTRLTVL

SEQ ID NO: 268 - protein, 13 aa, Homo sapiens
  C0021155
SGSSSNIGNHYVS

SEQ ID NO: 269 - protein, 7 aa, Homo sapiens
  C0021155
DTTVLSS

SEQ ID NO: 270 - protein, 11 aa, Homo sapiens
  C0021155
GTWDELTSNLV

SEQ ID NO: 271 - DNA, 360 bp, Homo sapiens
  C0021158
gaggtgcagctgttggagtctggggggggcttggtacagcctggggggtccctgagactctcctgtgcagcctctggattcacctttcgatacgaagtagccgcttgggtccgccaggctccagggaaggggctggagtgggtctcggctattagtggtcccatccctaaaggctattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtcccgagt

SEQ ID NO: 272 - protein, 120 aa, Homo sapiens
  C0021158
EVQLLESGGGLVQPGGSLRLSCAASGFTFRYEVAAWVRQAPGKGLEWVSAISGPIPKGYYADSVKGRFTISRDNSKNTLYLQMNSLRAEDTAVYYCARLRADLGLYMDLWGRGTLVTVPS

SEQ ID NO: 273 - protein, 5 aa, Homo sapiens
  C0021158
YEVAA

SEQ ID NO: 274 - protein, 17 aa, Homo sapiens
  C0021158
AISGPIPKGYYADSVKG

SEQ ID NO: 275 - protein, 11 aa, Homo sapiens
  C0021158
LRADLGLYMDL

SEQ ID NO: 276 - DNA, 330 bp, Homo sapiens
  C0021158
cagtctgtgctgacgcagccgccctcagtgtctgcggccccgggtcagaaggtcaccatctcctgctctggaagcagttccaacattggcaatcattatgtatcctggtaccagcaactaccaggaacagctcccaaactcctcatttatgacaatagtgagcgaaccgcaggggttcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 277 - protein, 110 aa, Homo sapiens
  C0021158
QSVLTQPPSVSAAPGQKVTISCSGSSSNIGNHYVSWYQQLPGTAPKLLIYDNSERTAGVPDRFSGSKSGTSATLGITGLQTGDEADYYCGTWDELTSNLVFGGGTKLTVL

SEQ ID NO: 278 - protein, 13 aa, Homo sapiens
  C0021158
SGSSSNIGNHYVS

SEQ ID NO: 279 - protein, 7 aa, Homo sapiens
  C0021158
DNSERTA

SEQ ID NO: 280 - protein, 11 aa, Homo sapiens
  C0021158
GTWDELTSNLV

SEQ ID NO: 281 - DNA, 360 bp, Homo sapiens
  C0021158 IgG
gaggtgcagctgttggagtctggggggggcttggtacagcctggggggtccctgagactctcctgtgcagcctctggattcacctttcgatacgaagtagccgcttgggtccgccaggctccagggaaggggctggagtgggtctcggctattagtggtcccatccctaaaggctattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcctca

SEQ ID NO: 282 - protein, 120 aa, Homo sapiens
  C0021158 IgG
EVQLLESGGGLVQPGGSLRLSCAASGFTFRYEVAAWVRQAPGKGLEWVSAISGPIPKGYYADSVKGRFTISRDNSKNTLYLQMNSLRAEDTAVYYCARLRADLGLYMDLWGRGTLVTVSS

SEQ ID NO: 283 - protein, 5 aa, Homo sapiens
  C0021158 IgG
YEVAA

SEQ ID NO: 284 - protein, 17 aa, Homo sapiens
  C0021158 IgG
AISGPIPKGYYADSVKG

SEQ ID NO: 285 - protein, 11 aa, Homo sapiens
  C0021158 IgG
LRADLGLYMDL

SEQ ID NO: 286 - DNA, 330 bp, Homo sapiens; identical to SEQ ID NO: 276
  C0021158 IgG

SEQ ID NO: 287 - protein, 110 aa, Homo sapiens; identical to SEQ ID NO: 277
  C0021158 IgG

SEQ ID NO: 288 - protein, 13 aa, Homo sapiens
  C0021158 IgG
SGSSSNIGNHYVS

SEQ ID NO: 289 - protein, 7 aa, Homo sapiens
  C0021158 IgG
DNSERTA

SEQ ID NO: 290 - protein, 11 aa, Homo sapiens
  C0021158 IgG
GTWDELTSNLV

SEQ ID NO: 291 - DNA, 360 bp, Homo sapiens; identical to SEQ ID NO: 281
  C0021158 dr

SEQ ID NO: 292 - protein, 120 aa, Homo sapiens; identical to SEQ ID NO: 282
  C0021158 dr

SEQ ID NO: 293 - protein, 5 aa, Homo sapiens
  C0021158 dr
YEVAA

SEQ ID NO: 294 - protein, 17 aa, Homo sapiens
  C0021158 dr
AISGPIPKGYYADSVKG

SEQ ID NO: 295 - protein, 11 aa, Homo sapiens
  C0021158 dr
LRADLGLYMDL

SEQ ID NO: 296 - DNA, 330 bp, Homo sapiens
  C0021158 dr
cagtctgtgctgacgcagccgccctcagtgtctgcggccccgggtcagaaggtcaccatctcctgctctggaagcagttccaacattggcaatcattatgtatcctggtaccagcaactaccaggaacagctcccaaactcctcatttatgacaatgctgagcgaaccgcaggggttcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 297 - protein, 110 aa, Homo sapiens
  C0021158 dr
QSVLTQPPSVSAAPGQKVTISCSGSSSNIGNHYVSWYQQLPGTAPKLLIYDNAERTAGVPDRFSGSKSGTSATLGITGLQTGDEADYYCGTWDELTSNLVFGGGTKLTVL

SEQ ID NO: 298 - protein, 13 aa, Homo sapiens
  C0021158 dr
SGSSSNIGNHYVS

SEQ ID NO: 299 - protein, 7 aa, Homo sapiens
  C0021158 dr
DNAERTA

SEQ ID NO: 300 - protein, 11 aa, Homo sapiens
  C0021158 dr
GTWDELTSNLV

SEQ ID NO: 301 - DNA, 360 bp, Homo sapiens; identical to SEQ ID NO: 281
  C0021158 fgl

SEQ ID NO: 302 - protein, 120 aa, Homo sapiens; identical to SEQ ID NO: 282
  C0021158 fgl

SEQ ID NO: 303 - protein, 5 aa, Homo sapiens
  C0021158 fgl
YEVAA

SEQ ID NO: 304 - protein, 17 aa, Homo sapiens
  C0021158 fgl
AISGPIPKGYYADSVKG

SEQ ID NO: 305 - protein, 11 aa, Homo sapiens
  C0021158 fgl
LRADLGLYMDL

SEQ ID NO: 306 - DNA, 330 bp, Homo sapiens
  C0021158 fgl
cagtctgtgctgacgcagccgccctcagtgtctgcggccccgggtcagaaggtcaccatctcctgctctggaagcagttccaacattggcaatcattatgtatcctggtaccagcaactaccaggaacagctcccaaactcctcatttatgacaatagtgagcgaaccgcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 307 - protein, 110 aa, Homo sapiens
  C0021158 fgl
QSVLTQPPSVSAAPGQKVTISCSGSSSNIGNHYVSWYQQLPGTAPKLLIYDNSERTAGIPDRFSGSKSGTSATLGITGLQTGDEADYYCGTWDELTSNLVFGGGTKLTVL

SEQ ID NO: 308 - protein, 13 aa, Homo sapiens
  C0021158 fgl
SGSSSNIGNHYVS

SEQ ID NO: 309 - protein, 7 aa, Homo sapiens
  C0021158 fgl
DNSERTA

SEQ ID NO: 310 - protein, 11 aa, Homo sapiens
  C0021158 fgl
GTWDELTSNLV

SEQ ID NO: 311 - DNA, 360 bp, Homo sapiens; identical to SEQ ID NO: 281
  C0021158 fgl2

SEQ ID NO: 312 - protein, 120 aa, Homo sapiens; identical to SEQ ID NO: 282
  C0021158 fgl2

SEQ ID NO: 313 - protein, 5 aa, Homo sapiens
  C0021158 fgl2
YEVAA

SEQ ID NO: 314 - protein, 17 aa, Homo sapiens
  C0021158 fgl2
AISGPIPKGYYADSVKG

SEQ ID NO: 315 - protein, 11 aa, Homo sapiens
  C0021158 fgl2
LRADLGLYMDL

SEQ ID NO: 316 - DNA, 330 bp, Homo sapiens
  C0021158 fgl2
cagtctgtgctgacgcagccgccctcagtgtctgcggccccgggtcagaaggtcaccatctcctgctctggaagcagttccaacattggcaatcattatgtatcctggtaccagcaactaccaggaacagctcccaaactcctcatttatgacaatgctgagcgaaccgcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 317 - protein, 110 aa, Homo sapiens
  C0021158 fgl2
QSVLTQPPSVSAAPGQKVTISCSGSSSNIGNHYVSWYQQLPGTAPKLLIYDNAERTAGIPDRFSGSKSGTSATLGITGLQTGDEADYYCGTWDELTSNLVFGGGTKLTVL

SEQ ID NO: 318 - protein, 13 aa, Homo sapiens
  C0021158 fgl2
SGSSSNIGNHYVS

SEQ ID NO: 319 - protein, 7 aa, Homo sapiens
  C0021158 fgl2
DNAERTA

SEQ ID NO: 320 - protein, 11 aa, Homo sapiens
  C0021158 fgl2
GTWDELTSNLV

SEQ ID NO: 321 - DNA, 360 bp, Homo sapiens
  C0021177
gaggtgcagctgttggagtctgggggaggcttggtacagcctggggggtccctgagactctcctgtgcagcctctggattcacctttcgatatgaggtagccgcttgggtccgccaggctccagggaaggggctggagtgggtctcagctattagtggtcccgcccctaaaggctattacgcagactccgtgaagggccggttcaccatctccagagacaattccaagaacacgctgtatctgcaaatgaacagcctgagagccgaggacacggccgtgtattactgcgcgaggcttagagcggatttagggttgtacatggacctctggggccggggcaccctggtcaccgtctcgagt

SEQ ID NO: 322 - protein, 120 aa, Homo sapiens
  C0021177
EVQLLESGGGLVQPGGSLRLSCAASGFTFRYEVAAWVRQAPGKGLEWVSAISGPAPKGYYADSVKGRFTISRDNSKNTLYLQMNSLRAEDTAVYYCARLRADLGLYMDLWGRGTLVTVSS

SEQ ID NO: 323 - protein, 5 aa, Homo sapiens
  C0021177
YEVAA

SEQ ID NO: 324 - protein, 17 aa, Homo sapiens
  C0021177
AISGPAPKGYYADSVKG

SEQ ID NO: 325 - protein, 11 aa, Homo sapiens
  C0021177
LRADLGLYMDL

SEQ ID NO: 326 - DNA, 330 bp, Homo sapiens
  C0021177
cagtctgagctgacgcagccgcccacagcgtctgcggccccaggtcagaaggtcaccatctcctgctccggaagtagttccaacattggcaatcattatgtatcctggtaccagcaactaccaggaacagcccccaaactcctcatttatgacacgaccgtcttgagctcagggattcctgaccgattctctggctccaagtctggcacgtcagccaccctgggcatcaccggactccagactggggacgaggccgattattactgcggaacatgggacgagctgacctccaacttggtgttcggcggcgggaccaagctgaccgtccta

SEQ ID NO: 327 - protein, 110 aa, Homo sapiens
  C0021177
QSELTQPPTASAAPGQKVTISCSGSSSNIGNHYVSWYQQLPGTAPKLLIYDTTVLSSGIPDRFSGSKSGTSATLGITGLQTGDEADYYCGTWDELTSNLVFGGGTKLTVL

SEQ ID NO: 328 - protein, 13 aa, Homo sapiens
  C0021177
SGSSSNIGNHYVS

SEQ ID NO: 329 - protein, 7 aa, Homo sapiens
  C0021177
DTTVLSS

SEQ ID NO: 330 - protein, 11 aa, Homo sapiens
  C0021177
GTWDELTSNLV

SEQ ID NO: 331 - DNA, 360 bp, Homo sapiens
  C0021180
gaggtgcagctgttggagtctgggggaggcttggtacagcctggggggtccctgagactctcctgtgcagcctctggattcacctttcgatacgaagtagccgcttgggtccgccaggctccagggaaggggctggagtgggtctcagctattagtggtcccatccctaaaggctattacgcagactccgtgaagggccggttcaccatctccagagacaattccaagaacacgctgtatctgcaaatgaacagcctgagagccgaggacacggccgtgtattactgtgcgaggcttagagcggatttagggttgtacatggacctctggggccggggcaccctggtcaccgtctcgagt

SEQ ID NO: 332 - protein, 120 aa, Homo sapiens; identical to SEQ ID NO: 282
  C0021180

SEQ ID NO: 333 - protein, 5 aa, Homo sapiens
  C0021180
YEVAA

SEQ ID NO: 334 - protein, 17 aa, Homo sapiens
  C0021180
AISGPIPKGYYADSVKG

SEQ ID NO: 335 - protein, 11 aa, Homo sapiens
  C0021180
LRADLGLYMDL

SEQ ID NO: 336 - DNA, 330 bp, Homo sapiens
  C0021180
cagtctgtgctgacgcagccgcccacagtgtctgcggccccaggtcggaaggtcaccatctcctgctctggaagcagttccaacattggcgatcatcatgtatcctggtaccagcaactaccaggagcagcccccaaactcctcatttatgacacggaccacatggcgtcggggattcctgaccgattctctggctccaggtctggcacgtcagccaccctgggcatcaccggactccagactggggacgaggctgattattactgcggaacatgggatagcagcctgagtgccctggtgttcggcggcgggaccaagctgaccgtccta

SEQ ID NO: 337 - protein, 110 aa, Homo sapiens
  C0021180
QSVLTQPPTVSAAPGRKVTISCSGSSSNIGDHHVSWYQQLPGAAPKLLIYDTDHMASGIPDRFSGSRSGTSATLGITGLQTGDEADYYCGTWDSSLSALVFGGGTKLTVL

SEQ ID NO: 338 - protein, 13 aa, Homo sapiens
  C0021180
SGSSSNIGDHHVS

SEQ ID NO: 339 - protein, 7 aa, Homo sapiens
  C0021180
DTDHMAS

SEQ ID NO: 340 - protein, 11 aa, Homo sapiens
  C0021180
GTWDSSLSALV

SEQ ID NO: 341 - DNA, 360 bp, Homo sapiens
  C0021181
gaggtgcagctgttggagtctgggggaggcttggtacggccaggggggtccctgagactctcctgtgcagcctctgaattcacctttcgctacgattaccacgtttgggtccgccaggctccagggaaggggctggagtgggtctcagcaattagtggtagtggtggtagcacatactacgcagactccgtgaagagccggttcaccatctccagagacaattccaagaacacgctgtatctgcagatgaacagcctgagagccgaggacacggccgtgtattactgtgcgaggcttagagcggatttagggttgtacatggacctctggggccggggcaccctggtcaccgtctcgagt

SEQ ID NO: 342 - protein, 120 aa, Homo sapiens
  C0021181
EVQLLESGGGLVRPGGSLRLSCAASEFTFRYDYHVWVRQAPGKGLEWVSAISGSGGSTYYADSVKSRFTISRDNSKNTLYLQMNSLRAEDTAVYYCARLRADLGLYMDLWGRGTLVTVSS

SEQ ID NO: 343 - protein, 5 aa, Homo sapiens
  C0021181
YDYHV

SEQ ID NO: 344 - protein, 17 aa, Homo sapiens
  C0021181
AISGSGGSTYYADSVKS

SEQ ID NO: 345 - protein, 11 aa, Homo sapiens
  C0021181
LRADLGLYMDL

SEQ ID NO: 346 - DNA, 330 bp, Homo sapiens
  C0021181
cagtctgtgctgacgcagccgccctcagtgtctgcggccccaggtcagaaggtcgccgtctcctgctctggaagcagttccaacattggcagtcattatgtatcctggtaccagcaactaccaggaacagcccccaaactcctcatttatgacaatagtgagcgaacctcagggattcctgaccgattctctggctccaagtctggcacgtcagccaccctgggcatcaccggactccaaactggggacgaggccgattattactgcggaacatgggacgagctgacctccaacttggtgttcggcggcgggaccaggctgaccgtcctg

SEQ ID NO: 347 - protein, 110 aa, Homo sapiens
  C0021181
QSVLTQPPSVSAAPGQKVAVSCSGSSSNIGSHYVSWYQQLPGTAPKLLIYDNSERTSGIPDRFSGSKSGTSATLGITGLQTGDEADYYCGTWDELTSNLVFGGGTRLTVL

SEQ ID NO: 348 - protein, 13 aa, Homo sapiens
  C0021181
SGSSSNIGSHYVS

SEQ ID NO: 349 - protein, 7 aa, Homo sapiens
  C0021181
DNSERTS

SEQ ID NO: 350 - protein, 11 aa, Homo sapiens
  C0021181
GTWDELTSNLV

SEQ ID NO: 351 - protein, 16 aa, Artificial Sequence
  N-terminal Avi tag for ARG2
AGLNDIFEAQKIEWHE

SEQ ID NO: 352 - protein, 355 aa, Artificial Sequence
  Avi-huARG1-His10
MAGLNDIFEAQKIEWHEGGGMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKGGGHHHHHHHHHH

SEQ ID NO: 353 - protein, 365 aa, Artificial Sequence
  Avi-huARG2-His10
MAGLNDIFEAQKIEWHEGGGVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYRHGMYIAEEIHNTGLLSALDLVEVNPQLATSEEEAKTTANLAVDVIASSFGQTKEGGHIVYDQLPTPSSPDESENQARVRIGGGHHHHHHHHHH

SEQ ID NO: 354 - protein, 365 aa, Artificial Sequence
  Avi-huARG2-His10 Monomer
MAGLNDIFEAQKIEWHEGGGVHSVAIVGAPFSRGQKKLGVEYGPAAIREAGLLKRLSRLGCHLKDFGDLSFTNVPQDDPYNNLVVYPRSVGLANQELAEVVSRAVSGGYSCVTMGGDHSLAIGTIIGHARHRPDLCVIWVDAHADINTPLTTVSGNIHGQPLSFLIKELQDKVPQLPGFSWIKPCLSPPNIVYIGLRDVEPPEHFILKNYDIQYFSMREIDRLGIQKVMEQTFDRLIGKRQRPIHLSFDIDAFDPKLAPATGTPVVGGLTYRQGVYITEEIHNTGLLSALDLVEVNPHLATSEEEAKATARLAVDVIASSFGQTREGGHIVYDHLPTPSSPHESENEECVRIGGGHHHHHHHHHH

SEQ ID NO: 355 - protein, 346 aa, Artificial Sequence
  huARG2-His10
MVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRIGGGHHHHHHHHHH

SEQ ID NO: 356 - protein, 365 aa, Artificial Sequence
  Avi-cynoARG2-His10
MAGLNDIFEAQKIEWHEGGGVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSPSIVYIGLRDVDPPEHFILKNYDIQYFSMRDIDRLGIQKVMEQTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYGQLPTPSSPDESENQARVRIGGGHHHHHHHHHH

SEQ ID NO: 357 - protein, 339 aa, Artificial Sequence
  huARG2-His6
MVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRIHHHHHH